(19) United States
(12) Patent Application Publication
(10) Pub. No.: US 2025/0084155 A1
(43) Pub. Date: Mar. 13, 2025

(54) ANTI-HSP70 ANTIBODIES AND THERAPEUTIC USES THEREOF

(71) Applicants: BOARD OF REGENTS, THE UNIVERSITY OF TEXAS SYSTEM, Austin, TX (US); ASYLIA THERAPEUTICS, Houston, TX (US)

(72) Inventors: Robert Z. ORLOWSKI, Houston, TX (US); Richard J. JONES, Houston, TX (US); Laura C. BOVER, Houston, TX (US); Jeno GYURIS, Houston, TX (US); Darragh MAC CANN, Magherafelt (IE); James Bernard MCCLORY, Ballynaskeagh Banbridge (IE); John MILLER, Houston, TX (US); Parth MANGROLIA, Houston, TX (US); William M. WINSTON, Jr., Newton, MA (US)

(73) Assignees: BOARD OF REGENTS, THE UNIVERSITY OF TEXAS SYSTEM, Austin, TX (US); ASYLIA THERAPEUTICS, Houston, TX (US)

(21) Appl. No.: 18/688,829

(22) PCT Filed: Sep. 29, 2022
(86) PCT No.: PCT/US2022/077266

§ 371 Date: Mar. 4, 2024

Related U.S. Application Data
(60) Provisional application No. 63/249,909, filed on Sep. 29, 2021.

(51) Int. Cl. C07K 16/18; A61K 39/00; A61P 35/00

(52) U.S. Cl. CPC C07K 16/18; A61P 35/00; A61K 2039/505; C07K 2317/34; C07K 2317/565; C07K 2317/73; C07K 2317/77; C07K 2317/92

(57) ABSTRACT

The present invention relates generally to the fields of medicine, immunology, and cancer biology. More particularly, it concerns antibodies that target HSP70 and methods of their use. Provided herein are agents such as antibodies that target HSP70. Methods of treating cancer are provided, comprising administering to a patient in need thereof an effective amount of an HSP70-targeting agent such as an HSP70-specific antibody. The HSP70-specific antibody may enhance uptake of HSP70 by antigen presenting cells.

### CROSS REFERENCE TO RELATED APPLICATIONS

This application claims the benefit of and U.S. priority to Provisional Patent Application No. 63/249,909, filed on Sep. 29, 2021, the disclosure of which is incorporated herein by reference in its entirety for all purposes.

### REFERENCE TO A SEQUENCE LISTING

This application contains a Sequence Listing XML, which has been submitted electronically and is hereby incorporated by reference in its entirety. Said Sequence Listing SML, created on Sep. 27, 2022, is named UTFCP1512WO_ST26.xml and is 371,316 bytes in size.

### BACKGROUND

### 1. Field

The present invention relates generally to the fields of medicine, immunology, and cancer biology. More particularly, it concerns antibodies that target HSP70 and methods of their use.

### 2. Description of Related Art

The development of an immune response against cancerous cells is believed to depend upon a series of reinforcing events, which have been referred to as the Cancer Immunity Cycle (Chen & Mellman, 2013), which starts with release of cancer cell antigens during cancer cell death. Dendritic cells (DCs) are believed to be key early components of this response by virtue of their ability to capture, process, and then present these tumor antigens to T cells via presentation through histocompatibility complex (MHC) class I and II molecules, which then results in the priming and activation of effector CD4+ and CD8+ T-cell responses. The crucial role of DCs is demonstrated, in part, by the many mechanisms leveraged by tumors to suppress DC activity, including hypoxia, adenosine, lactic acid, low pH, and expression of interleukin (IL)-10 and PD-L1, among others (Veglia & Gabrilovich, 2017).

Heat shock proteins (HSPs) in general, and HSP70 in particular, are believed to play a key role in this process because of their ability to link the innate and adaptive immune responses (Shevtsov & Multhoff, 2016). For example, extracellular HSP70 binds and chaperones tumor antigens and then targets antigen presenting cells, including DCs, through binding to distinct cell surface receptors, including CD91, oxidized low-density lipoprotein receptor 1 (OLR1), and scavenger receptor expressed by endothelial cells (SREC)-1, among others (McNulty et al., 2013), thereby delivering bound antigens to DCs for processing. Furthermore, extracellular HSP70 secreted from tumor cells induces inflammatory cytokines such as Interleukin (IL)-6 and Tumor necrosis factor (TNF)-α from macrophages (Vega et al., 2008), thereby enabling cross-presentation and T-cell activation, respectively. As such, HSP70 is considered to be an attractive target for cancer therapy because of its crucial intracellular role as a cytoprotective, anti-apoptotic factor that promotes cancer cell survival in the face of various stressors, including both radiation and a variety of chemotherapeutics (Boudesco et al., 2018). Furthermore, HSP70 is also considered to be an attractive target for cancer therapy because of its ability to stimulate immune responses through not just DCs, but possibly also macrophages, NK cells, and T cells (Shvetsov & Multhoff, 2016; Zininga et al., 2018).

Approaches that enhance DC uptake of HSP70-tumor antigen complexes hold the promise of enhancing anti-tumor immunity and breaking tolerance. Several pharmacologic inhibitors have been developed that target intracellular HSP70 directly, or some its co-chaperones (Boudesco et al., 2018), which may act as sensitizers to radiation or chemotherapy. Also, while membrane-bound HSP70 is usually either absent or found only at low levels on normal cells, it often shows enhanced expression on the surface of tumor cells, and in some malignancies has been associated with a more aggressive phenotype and inferior prognosis (Boudesco et al., 2018; Chatterjee & Burns, 2017). Moreover, HSP70−/− tumors have been found to be less immunogenic and more aggressive (Dodd et al., 2015). This has led to the development and testing of a variety of approaches, including ferromagnetic and gold nanoparticle-based therapies, vaccine strategies (Shvetsov & Multhoff, 2016), and monoclonal antibodies such as cmHSP70.1 (Stangl et al., 2011), that rely on HSP70 cell surface expression for their activity.

Over recent years, immune checkpoint inhibitors (ICIs), including monoclonal antibodies to cytotoxic T-lymphocyte associated protein 4 (CTLA-4) and programmed cell death 1 (PD-1) and its ligand, PD-L1, have revolutionized immunotherapy through their ability to induce durable remissions in even advanced malignancies. In general, it is believed that tumors that respond to ICIs tend to have higher immune cell infiltration and/or an interferon gene signature, or a higher tumor mutation burden (TMB), and are sometimes referred to as “hot” tumors (Maleki Vareki, 2018). In contrast, so-called “cold” tumors with low immune cell infiltrates or low TMB tend not to respond to ICIs, and include pancreatic and prostate cancers (Maleki Vareki, 2018). Despite the advances in treating various malignancies, there is still a need for new approaches that convert cold tumors into more immunogenic tumors, which may provide alternative approaches for the treatment of these tumors.

### SUMMARY

The invention is based, in part, upon the discovery of anti-HSP70 antibodies (e.g., anti-HSP70 monoclonal antibodies) or antigen binding fragments thereof. In certain circumstances, the anti-HSP70 monoclonal antibodies or antigen binding fragments thereof may, for example, target extracellular or soluble HSP70 associated with tumor-derived antigens to immune cells (e.g., dendritic cells) and thereby treat cancer and/or enhance the efficacy of a cancer therapy (e.g., a cancer immunotherapy). In certain circumstances, the anti-HSP70 monoclonal antibodies or antigen binding fragments thereof may, for example, former high order (i.e., greater than one to one) complexes with HSP70.

The invention provides antibodies (e.g., isolated antibodies) that bind human HSP70, e.g., extracellular or soluble human HSP70.

In certain aspects, the antibody comprises an immunoglobulin heavy chain comprising the amino acid sequence of SEQ ID NO: 242 (hVH-1-G1m3), and an immunoglobulin light chain variable region comprising the amino acid sequence of SEQ ID NO: 249 (hVL-1-Km3). In certain aspects, the antibody comprises an immunoglobulin heavy chain comprising the amino acid sequence of SEQ ID NO: 243 (hVH-1-G1m3-GA), and an immunoglobulin light chain variable region comprising the amino acid sequence of SEQ ID NO: 249 (hVL-1-Km3). In certain aspects, the antibody comprises an immunoglobulin heavy chain comprising the amino acid sequence of SEQ ID NO: 244 (hVH-1-G1m3-GAALIE), and an immunoglobulin light chain variable region comprising the amino acid sequence of SEQ ID NO: 249 (hVL-1-Km3). In certain aspects, the antibody comprises an immunoglobulin heavy chain comprising the amino acid sequence of SEQ ID NO: 245 (hVH-1-G1m3-YTE), and an immunoglobulin light chain variable region comprising the amino acid sequence of SEQ ID NO: 249 (hVL-1-Km3). In certain aspects, the antibody comprises an immunoglobulin heavy chain comprising the amino acid sequence of SEQ ID NO: 246 (hVH-1-G1m3-LS), and an immunoglobulin light chain variable region comprising the amino acid sequence of SEQ ID NO: 249 (hVL-1-Km3). In certain aspects, the antibody comprises an immunoglobulin heavy chain comprising the amino acid sequence of SEQ ID NO: 247 (hVH-1-G1m3-DF215), and an immunoglobulin light chain variable region comprising the amino acid sequence of SEQ ID NO: 249 (hVL-1-Km3). In certain aspects, the antibody comprises an immunoglobulin heavy chain comprising the amino acid sequence of SEQ ID NO: 248 (hVH-1-G1m3-DF228), and an immunoglobulin light chain variable region comprising the amino acid sequence of SEQ ID NO: 249 (hVL-1-Km3).

In certain aspects, the antibody binds to human HSP70 with a K_{D} of 50 nM or lower, 10 nM or lower, 5 nM or lower, 1 nM or lower, 0.75 nM or lower, 0.5 nM or lower, 0.1 nM, 0.075 nM, or 0.05 nM or lower, as measured by surface plasmon resonance or bio-layer interferometry.

In certain aspects, the antibody is capable of forming a high-order antibody:HSP70 complex. The antibody:HSP70 complex may, for example, have a molecular weight of at least about 290 kDa, 300 kDa, 580 kDa, 1,450 kDa, or 1,750 kDa or a molecular weight of about 290 kDa, 580 kDa, 1,450 kDa, or 1,750 kDa. In certain aspects, the antibody:HSP70 complex may have a molecular weight of at least or greater than about 300 kDa. In certain aspects, the ratio of antibody to HSP70 in the antibody:HSP70 complex is about 1:2. For example, the antibody:HSP70 complex may comprise: (i) about one antibody molecule and about two HSP70 molecules; (ii) about two antibody molecules and about four HSP70 molecules; (iii) about five antibody molecules and about ten HSP70 molecules; or (iv) about six antibody molecules and about twelve HSP70 molecules.

In certain aspects, the antibody:HSP70 complex binds to a human FcγR (e.g., FcγR1, FcγR2a, FcγR2b, FcγR2c, FcγR3a, and/or FcγR3b) with a K_{D} of 10 nM or lower, 5 nM or lower, 1 nM or lower, 0.75 nM or lower, 0.5 nM or lower, 0.1 nM, 0.075 nM, or 0.05 nM or lower, as measured by surface plasmon resonance or bio-layer interferometry.

In certain aspects, the antibody enhances the uptake of tumor-derived ADP-HSP70-peptide antigen complexes by immune effector cells. The uptake may, for example, be mediated by human FcγR1, FcγR2a, FcγR2b, FcγR2c, FcγR3a, and/or FcγR3b.

In some aspects, the antibody:HSP70 complex activates immune effector cells, e.g., CD8+ T cells, CD4+ T cells, NK cells, and dendritic cells, e.g., immature dendritic cells.

In another embodiment, the invention provides an antibody (e.g., an isolated antibody) that is capable of forming a high-order (i.e., greater than one to one) antibody:HSP70 complex. In certain embodiments, the antibody:HSP70 complex further comprises HSP70-associated peptides.

The antibody:HSP70 complex may, for example, have a molecular weight of at least about 290 kDa, 300 kDa, 580 kDa, 1,450 kDa, or 1,750 kDa or a molecular weight of about 290 kDa, 580 kDa, 1,450 kDa, or 1,750 kDa. For example, the antibody:HSP70 complex may have a molecular weight of at least or greater than about 300 kDa. In certain aspects, the ratio of antibody to HSP70 in the antibody:HSP70 complex is about 1:2. For example, the antibody:HSP70 complex may comprise: (i) about one antibody molecule and about two HSP70 molecules; (ii) about two antibody molecules and about four HSP70 molecules; (iii) about five antibody molecules and about ten HSP70 molecules; or (iv) about six antibody molecules and about twelve HSP70 molecules.

In certain aspects, the antibody:HSP70 complex binds to a human FcγR (e.g., FcγR1, FcγR2a, FcγR2b, FcγR2c, FcγR3a, and/or FcγR3b) with a K_{D} of 10 nM or lower, 5 nM or lower, 1 nM or lower, 0.75 nM or lower, 0.5 nM or lower, 0.1 nM, 0.075 nM, or 0.05 nM or lower, as measured by surface plasmon resonance or bio-layer interferometry.

In certain aspects, the antibody enhances the uptake of tumor-derived ADP-HSP70-peptide antigen complexes by immune effector cells. The uptake may, for example, be mediated by human FcγR1, FcγR2a, FcγR2b, FcγR2c, FcγR3a, and/or FcγR3b.

In certain aspects, the antibody binds to an epitope of HSP70 comprising K573-Q601 of SEQ ID NO: 11. In certain aspects, the antibody binds to an epitope of HSP70 comprising one, two, three, four, five, six, seven, or eight of the following residues: K573, E576, W580, H594, K595, R596, E598, and Q601 of SEQ ID NO: 11. In certain aspects, the antibody binds to an epitope of HSP70 comprising KEWHKREQ (SEQ ID NO: 250).

In certain aspects, the antibody comprises an immunoglobulin heavy chain variable region comprising a VHCDR1 comprising the amino acid sequence of SEQ ID NO: 1, a VHCDR2 comprising the amino acid sequence of SEQ ID NO: 2, and a VHCDR3 comprising the amino acid sequence of SEQ ID NO: 3, and an immunoglobulin light chain variable region comprising a VLCDR1 comprising the amino acid sequence of SEQ ID NO: 4, a VLCDR2 comprising the amino acid sequence of SEQ ID NO: 5, and a VLCDR3 comprising the amino acid sequence of SEQ ID NO: 6. In certain aspects, the antibody comprises an immunoglobulin heavy chain variable region comprising the amino acid sequence of SEQ ID NO: 12 (hVH-1), and an immunoglobulin light chain variable region comprising the amino acid sequence of SEQ ID NO: 19 (hVL-1).

In certain aspects, the antibody enhances the uptake of tumor-derived ADP-HSP70-peptide antigen complexes by immune effector cells. The uptake may, for example, be mediated by human FcγR1, FcγR2a, FcγR2b, FcγR2c, FcγR3a, and/or FcγR3b.

In another embodiment, the invention provides a high order (i.e., greater than one to one) complex comprising an antibody and HSP70.

The antibody:HSP70 complex may, for example, have a molecular weight of at least about 290 kDa, 300 kDa, 580 kDa, 1,450 kDa, or 1,750 kDa or a molecular weight of about 290 kDa, 580 kDa, 1,450 kDa, or 1,750 kDa. In certain aspects, the antibody:HSP70 complex may have a molecular weight of at least or greater than about 300 kDa. In certain aspects, the ratio of antibody to HSP70 in the antibody:HSP70 complex is about 1:2. For example, the antibody:HSP70 complex may comprise: (i) about one antibody molecule and about two HSP70 molecules; (ii) about two antibody molecules and about four HSP70 molecules; (iii) about five antibody molecules and about ten HSP70 molecules; or (iv) about six antibody molecules and about twelve HSP70 molecules.

In certain aspects, the antibody:HSP70 complex binds to a human FcγR (e.g., FcγR1, FcγR2a, FcγR2b, FcγR2c, FcγR3a, and/or FcγR3b) with a K_{D} of 10 nM or lower, 5 nM or lower, 1 nM or lower, 0.75 nM or lower, 0.5 nM or lower, 0.1 nM, 0.075 nM, or 0.05 nM or lower, as measured by surface plasmon resonance or bio-layer interferometry.

In certain aspects, the antibody binds to an epitope of HSP70 comprising K573-Q601 of SEQ ID NO: 11. In certain aspects, the antibody binds to an epitope of HSP70 comprising one, two, three, four, five, six, seven, or eight of the following residues: K573, E576, W580, H594, K595, R596, E598, and Q601 of SEQ ID NO: 11. In certain aspects, the antibody binds to an epitope of HSP70 comprising KEWHKREQ (SEQ ID NO: 250).

In certain aspects, the antibody comprises an immunoglobulin heavy chain variable region comprising a VHCDR1 comprising the amino acid sequence of SEQ ID NO: 1, a VHCDR2 comprising the amino acid sequence of SEQ ID NO: 2, and a VHCDR3 comprising the amino acid sequence of SEQ ID NO: 3, and an immunoglobulin light chain variable region comprising a VLCDR1 comprising the amino acid sequence of SEQ ID NO: 4, a VLCDR2 comprising the amino acid sequence of SEQ ID NO: 5, and a VLCDR3 comprising the amino acid sequence of SEQ ID NO: 6. In certain aspects, the antibody comprises an immunoglobulin heavy chain variable region comprising the amino acid sequence of SEQ ID NO: 12 (hVH-1), and an immunoglobulin light chain variable region comprising the amino acid sequence of SEQ ID NO: 19 (hVL-1).

In certain aspects, the antibody comprises an immunoglobulin heavy chain comprising the amino acid sequence of SEQ ID NO: 242 (hVH-1-G1m3), and an immunoglobulin light chain variable region comprising the amino acid sequence of SEQ ID NO: 249 (hVL-1-Km3). In certain aspects, the antibody comprises an immunoglobulin heavy chain comprising the amino acid sequence of SEQ ID NO: 243 (hVH-1-G1m3-GA), and an immunoglobulin light chain variable region comprising the amino acid sequence of SEQ ID NO: 249 (hVL-1-Km3). In certain aspects, the antibody comprises an immunoglobulin heavy chain comprising the amino acid sequence of SEQ ID NO: 244 (hVH-1-G1m3-GAALIE), and an immunoglobulin light chain variable region comprising the amino acid sequence of SEQ ID NO: 249 (hVL-1-Km3). In certain aspects, the antibody comprises an immunoglobulin heavy chain comprising the amino acid sequence of SEQ ID NO: 245 (hVH-1-G1m3-YTE), and an immunoglobulin light chain variable region comprising the amino acid sequence of SEQ ID NO: 249 (hVL-1-Km3). In certain aspects, the antibody comprises an immunoglobulin heavy chain comprising the amino acid sequence of SEQ ID NO: 246 (hVH-1-G1m3-LS), and an immunoglobulin light chain variable region comprising the amino acid sequence of SEQ ID NO: 249 (hVL-1-Km3). In certain aspects, the antibody comprises an immunoglobulin heavy chain comprising the amino acid sequence of SEQ ID NO: 247 (hVH-1-G1m3-DF215), and an immunoglobulin light chain variable region comprising the amino acid sequence of SEQ ID NO: 249 (hVL-1-Km3). In certain aspects, the antibody comprises an immunoglobulin heavy chain comprising the amino acid sequence of SEQ ID NO: 248 (hVH-1-G1m3-DF228), and an immunoglobulin light chain variable region comprising the amino acid sequence of SEQ ID NO: 249 (hVL-1-Km3).

In another embodiment, the invention provides an isolated nucleic acid comprising a nucleotide sequence encoding the immunoglobulin heavy chain of any of the foregoing antibodies and/or a nucleotide sequence encoding the immunoglobulin light chain of any one of the foregoing antibodies. In another embodiment, the invention provides an expression vector comprising any of the foregoing nucleic acids. In another embodiment, the invention provides a host cell comprising any of the foregoing expression vectors.

In another embodiment, the invention provides a pharmaceutical composition comprising any of the foregoing antibodies.

In another embodiment, the invention provides a method of treating cancer in a subject in need thereof. The method comprises administering to the subject any of the foregoing antibodies or pharmaceutical compositions. In some embodiments, the method further comprises radiation therapy. In certain embodiments, the radiation therapy is selected from gamma-radiation, X-ray radiation, and radioisotope therapy. In certain embodiments, the radiation therapy comprises X-ray radiation therapy.

In certain aspects, the cancer is a multiple myeloma, a breast cancer, a melanoma, a colon cancer, a pancreatic cancer or a prostate cancer. In certain aspects, the subject (or a sample from the subject) has a serum HSP70 level greater than 20 ng/mL.

In another embodiment, the invention provides a method of enhancing uptake of tumor-derived ADP-HSP70-peptide antigen complexes by immune effector cells. The method comprises contacting the cells with any of the foregoing antibodies or pharmaceutical compositions.

In another embodiment, provided herein are antibody molecules, pharmaceutical compositions, cells, or pharmaceutical compositions of any one of the present embodiments for use in treating a cancer in a subject.

In another embodiment, provided herein are uses of antibody molecules, pharmaceutical compositions, cells, or pharmaceutical compositions of any one of the present embodiments, in the manufacture of a medicament for treating a cancer in a subject.

Other objects, features and advantages of the present invention will become apparent from the following figures and detailed description. It should be understood, however, that the detailed description and the specific examples, while indicating preferred embodiments of the invention, are given by way of illustration only, since various changes and modifications within the spirit and scope of the invention will become apparent to those skilled in the art from this detailed description.

### BRIEF DESCRIPTION OF THE DRAWINGS

The following drawings form part of the present specification and are included to further demonstrate certain aspects of the present invention. The invention may be better understood by reference to one or more of these drawings in combination with the detailed description of specific embodiments presented herein.

FIG. 1. BALB/c mice injected with MOPC315.BM-luc cells received 200 μg injections twice weekly in weeks 1 through 3 of the indicated HSP70 mAbs (squares) or their IgG2B isotype controls (circles). Disease burden was monitored using whole-animal in vivo imaging and confirmed by serum light chain levels.

FIGS. 2A-C. Octet analysis showing the affinity of 77A to murine HSP70 (top panel), human HSP70 made in E. coli (middle panel), and human HSP70 made in Sf9 insect cells (bottom panel) (FIG. 2A). 77A shows preferential binding to ADP-HSP70 complexes (FIG. 2B). 77A binding to HSP70-GFP shows greatest affinity when ADP and a peptide substrate (NRL) is present (FIG. 2C). Within each group of columns in (FIG. 2C), the columns represent, from left to right, Buffer, ATP, ADP, ATP NRL, and ADP NRL.

FIGS. 3A-F. Full-length (FL) HSP70 was expressed as an N-terminal GFP fusion protein in HSP70 KO 293T cells (FIG. 3A), along with deletion mutants of the indicated length removing progressively more C-terminal amino acids. IP of cell extracts with 77A was followed by detection of proteins by Western blotting (WB) with an anti-GFP antibody (FIG. 3B). Smaller deletions were then generated for finer mapping (FIG. 3C), and the indicated IPs were performed from cell lysates (CL) or culture media supernatants (CM). Loading was confirmed with an α-light chain (LC) antibody. The putative binding domain for 77A is indicated on a molecular model of HSP70 representing the relevant region of the protein (FIG. 3D; the amino acid sequence shown corresponds to positions 595-614 of SEQ ID NO: 11). The exact amino acids that comprise the 77A epitope were determined using alanine scanning analysis, and the results are shown in (FIG. 3E), and the ribbon diagram showing the primary and secondary critical sites is shown in (FIG. 3F).

FIGS. 4A-B. Luc-labeled MM1.S human myeloma cells were injected into nude mice, and treatment was given twice weekly in weeks 2 through 5 with either an IgG2B isotype control mAb or 77A at the indicated doses. Whole animal live imaging data are shown at week 5 (FIG. 4A) with the dorsal (upper panels) and ventral (lower panels) views indicating significant tumor growth in the IgG2B-treated mice but not in 77A-treated mice, especially at the higher dose levels. The same experiment was then performed in NSG mice, and tumor growth was measured both by imaging (FIG. 4B) and by an ELISA for human light chains.

FIG. 5. Immature murine DC2.4 cells were incubated at 37° C. for 6 hours with either vehicle (left two panels) or 6×-His-tagged HSP70 (right two panels) in the presence of IgG2B or 77A as indicated. They were then stained either with control IgG (left panel) or an α-6×-His tag mAb (right three panels), and HSP70 uptake was determined by flow. Significant uptake of HSP70 is seen only in the presence of 77A (right most panel).

FIG. 6. DC2.4 cells exposed to Sf9-derived 6×-His-tagged human HSP70 in the presence of either an isotype control mAb or the 77A mAb were stained either with wheat germ agglutinin (WGA) conjugated to Alexa Fluor 594 to stain cell membranes, the Alexa Fluor 488-tagged α-6×-His-tag mAb to detect HSP70, or 4′,6-diamidino-2-phenylindole (DAPI) to stain nuclei, and individual as well as a merged images were obtained. Representative fields are shown at 200× magnification.

FIG. 7. Electron micrographs of DCs exposed to HSP70 and gold-labeled 77A. Magnification shown is 100,000×.

FIGS. 8A-C. Mouse DC2.4 cells were treated with either PBS, or 5 μg of ADP-HSP70 purified from A-375 melanoma cells, which are a good source of HSP70 since they express high levels of this protein, in combination with 10 μg of IgG2B or 77A for 48 hours. RNA was harvested and cDNA was hybridized to the qPCR array described in the text. Genes that were activated or repressed by ≥2-fold are shown for the comparison between IgG2B and PBS (FIG. 8A; top panel) as well as 77A and PBS (FIG. 8A; bottom panel). Data are shown from 3 biological replicates. Ingenuity Pathway Analysis was then performed (FIG. 8B) to identify biological processes which could be influenced by these changes. Cytokines released by the maturing DCs were then analyzed using the BioRad Bio-Plex™ Pro Mouse Cytokine Array. Notable changes induced in the HSP70 and 77A exposed cells versus the HSP70 and IgG2B exposed cells are shown in the bar graph (FIG. 8C). Within each group of columns in (FIG. 8C), the left column represents IgG control (CTRL) and the right column represents HSP70 STIMULATED.

FIGS. 9A-G. BALB/c mice were injected into the 4th right mammary gland with 7,500 luc-4T1 cells. After 12 days, when all mice had palpable and measurable tumor, 200 μg of either IgG2B (filled box) or 77A (open box) were injected IV twice per week for 3 weeks. Tumor volumes were measured using both calipers and whole animal imaging for the primary (FIG. 9A), while imaging was used to assess pulmonary metastases (FIG. 9B). Peripheral blood was collected on day 32 and assessed for CD4+ and CD8+ T-cells (FIG. 9C), and also for dually CD11c+/MHC class II+ cells as well as total MHC class II+ cells (FIG. 9D). 77A induces uptake of HSP70 into human primary CD4+ and CD8+ T cells (FIG. 9E). 77A stimulates MHC-independent cytolytic CD4 T-cell activity (FIG. 9F). 77A activity against the A375 melanoma model in nude mice (FIG. 9G).

FIG. 10. HSP70 bound to ATP in the nucleotide binding domain (NBD) has an open conformation to allow interactions with the substrate binding domain (SBD). Substrate peptide interaction with the SBD, in coordination with J-proteins and a nucleotide exchange factor (NEF), stimulates the HSP70 ATPase activity, resulting in closing of the lid, thereby stabilizing HSP70's interaction with the substrate. Adapted from (Craig & Marszalek, 2017). The approximate location of 77A binding on the HSP70 model is also shown schematically in the right panel.

FIGS. 11A-C. Homogenate from a 10 mL pellet of 4T1 cells expressing HSP70-GFP was purified over an ADP-agarose column to isolate ADP-HSP70-peptide complexes, and 10 μg was injected intraperitoneally into BALB/c mice on day −24 and boosted subcutaneously on day −10. These were then injected on day 0 with 4T1-luc cells expressing HSP70-GFP as in the legend to FIGS. 9A-D, and tumor growth was monitored by whole animal imaging (FIG. 11A). At day 37, when the animals were euthanized, spleen cells were isolated and either analyzed by fluorescence activated cell sorting (FACS) on that day or placed into culture for 7 days in the presence of irradiated 4T1 cells expressing HSP70-GFP and then analyzed by FACS. Comparisons are provided for the CD4+ (FIG. 11B) and CD8+(FIG. 11C) T-cell content at spleen isolation (day 0) and after 7 days of culture (left panels). Cytotoxic T-cell activity was also tested by exposing the indicated cell fractions to living wild-type 4T1 cells or 4T1 cells expressing HSP70-GFP followed by viability studies (right panels).

FIGS. 12A-B. Cytokine release assays were performed on CD4+ (FIG. 12A) and CD8+ (FIG. 12B) T cells isolated from murine spleens after exposure to either 4T1 cells or 4T1 cells expressing HSP70-GFP as detailed above. IFNγ secretion is shown in each top panel while IL-2 secretion is shown in the bottom panels.

FIG. 13. 77A induces uptake of HSP70 through FcγR2A and FcγR2B. The top row represents HSP70 knockout HEK 293T cells expressing human FcγR2A. The bottom row represents HSP70 knockout HEK 293T cells expressing the indicated human Fcγ receptor.

FIGS. 14A-B. The sequence of murine (SEQ ID NO: 251; position 594-614) and human HSP70 (SEQ ID NO: 11, positions 594-614) at the proposed binding site for 77A is highlighted (FIG. 14A). Amino acid differences are boxed. Potential phosphorylation sites in Murine HSP70 are at K595; potential phosphorylation sites in Human HSP70 are at K595 and K597. Potential ubiquitination sites in Murine HSP70 are at S604, S608, and Y611; potential ubiquitination sites in Human HSP70 are at S608 and Y611. Mutation of the three amino acids in human HSP70 to mimic the murine version reduces the ability of 77A to recognize human HSP70 (FIG. 14B).

FIGS. 15A-B. Tumor targets for 77A. PLD with IgG2B or 77A was evaluated in BALB/c mice orthotopically injected with 4T1 cells (A) and in a CT26-based immune-competent colon carcinoma mode(B).

FIG. 16. The antibody 77A was tested in epitope binning experiments for competition in binding to the HSP70 protein with the indicated antibodies. Numbers in FIG. 16 reflect the percent of maximal binding in the presence of the potentially competing antibody.

FIG. 17. The antibody 77A binding to ADP-HSP70 (top) and ATP-HSP70 (bottom) as measured by biolayer interferometry (BLI).

FIG. 18. The binding of antibody 77A to ADP-HSP70 and ATP-HSP70 as measured by ELISA, where No 1° and No 2° represent negative controls where no primary antibody or secondary antibody were present, respectively.

FIG. 19. Tumor volume following treatment of mice bearing CT-26 tumors with the indicated antibodies. Boxed days (14, 17, 21) indicate days when mice received treatment. *p=0.0023 relative to isotype control (IgG2B) as determined by Dunnett's multiple comparison t test.

FIGS. 20A-B. A sequence alignment of humanized variants hVH-1 through hVH-5 (FIG. 20A) and hVL-1 through hVL-5 (FIG. 20B).

FIG. 21. HSP70 uptake following incubation of cells expressing the indicated human Fcγ receptor with HSP70GFP, GFP-Nanobody Alexa-488, and the indicated antibody, as measured by total MFI (left) and % GFP positive cells (right). 253-77A=77A; HC1 LC1=h77A-1; HC2 μLC1=h77A-6; HC3 μLC1=h77A-11.

FIG. 22. HSP70 uptake following incubation of cells expressing the indicated human Fcγ receptor with HSP70GFP, GFP-Nanobody Alexa-488, and the indicated IgG2 antibody, as measured by total MFI (left) and % GFP positive cells (right). 253-77A=77A; HC1 LC1=h77A-1; HC2 μLC1=h77A-6; HC3 μLC1=h77A-11.

FIG. 23. HSP70 uptake following incubation of cells expressing the indicated mouse Fcγ receptor with HSP70GFP, GFP-Nanobody Alexa-488, and the indicated antibody, as measured by total MFI (left) and % GFP positive cells (right). 253-77A=77A; HC1 LC1=h77A-1; HC2 μLC1=h77A-6; HC3 μLC1=h77A-11.

FIG. 24. HSP70 uptake following incubation of cells expressing the indicated mouse Fcγ receptor with HSP70GFP, GFP-Nanobody Alexa-488, and the indicated IgG2 antibody, as measured by total MFI (left) and % GFP positive cells (right). 253-77A=77A; HC1 LC1=h77A-1; HC2 μLC1=h77A-6; HC3 μLC1=h77A-11.

FIG. 25. HSP70 uptake following incubation of DCs with HSP70GFP, GFP-Nanobody Alexa-488, and the indicated antibody, as measured by MFI (left) and % GFP positive cells (right). Results are shown for total cells gated against HSP70GFP. 253-77A=77A; HC1 LC1=h77A-1; HC2 μLC1=h77A-6; HC3 μLC1=h77A-11.

FIG. 26. HSP70 uptake following incubation of DCs with HSP70GFP, GFP-Nanobody Alexa-488, and the indicated antibody, as measured by % GFP positive cells (left) and MFI (right). Results are shown for plasmacytoid DC, CD303+ve, CD1C−ve cells. 253-77A=77A; HC1 μLC1=h77A-1; HC2 μLC1=h77A-6; HC3 μLC1=h77A-11.

FIG. 27. HSP70 uptake following incubation of DCs with HSP70GFP, GFP-Nanobody Alexa-488, and the indicated antibody, as measured by % GFP positive cells (left) and MFI (right). Results are shown for type 1 DC, CD141+ve, CD1c−ve cells. 253-77A=77A; HC1 LC1=h77A-1; HC2 μLC1=h77A-6; HC3 μLC1=h77A-11.

FIG. 28. HSP70 uptake following incubation of DCs with HSP70GFP, GFP-Nanobody Alexa-488, and the indicated antibody, as measured by % GFP positive cells (left) and MFI (right). Results are shown for type 2 DC, CD1C+ve, CD303-ve cells. 253-77A=77A; HC1 LC1=h77A-1; HC2 μLC1=h77A-6; HC3 μLC1=h77A-11.

FIGS. 29A-J. A sequence alignment of humanized variants hVH-1.1 through hVH-1.78 (FIGS. 29A-F) and hVL-1.1 through hVL-1.53 (FIGS. 29G-J).

FIG. 30. Size exclusion chromatography (SEC) traces for h77A-1-G1m3 labeled with CF647 dye (“G1m3-CF647”) and HSP70 fused to GFP (“HSP70-GFP”), each run alone and detected using the corresponding fluorescent channel. Peaks, corresponding retention time, and calculated approximate molecular weights (*MW) are indicated. Molecular weights for each peak were calculated from a MW standard curve.

FIG. 31. Size exclusion chromatography (SEC) traces for h77A-1-G1m3 labeled with CF647 dye (“G1m3”) and HSP70 fused to GFP (“HSP70-GFP”) following co-incubation of antibody and HSP70. The same sample was run twice, first detecting fluorescence from GFP (left) and then again from CF647 (right). Peaks, corresponding retention time, and calculated approximate molecular weights (*MW) are indicated. Molecular weights for each peak were calculated from a MW standard curve. Results show co-localization of antibody and HSP70 in detected high molecular weight peaks.

FIG. 32. Size exclusion chromatography (SEC) traces for h77A-1-G1m3 labeled with CF647 dye (“G1m3-CF647”) and HSP70 fused to GFP (“HSP70-GFP”) following co-incubation at the indicated molar ratios. Results are shown for detection of fluorescence from CF647. Peaks, corresponding retention time, and calculated approximate molecular weights (*MW) are indicated. Molecular weights for each peak were calculated from a MW standard curve.

FIG. 33. Size exclusion chromatography (SEC) traces for h77A-1-G1m3 labeled with CF647 dye (“G1m3-CF647”) and HSP70 fused to GFP (“HSP70-GFP”) following co-incubation at the indicated molar ratios. Results are shown for detection of fluorescence from GFP. Peaks, corresponding retention time, and calculated approximate molecular weights (*MW) are indicated. Molecular weights for each peak were calculated from a MW standard curve.

FIG. 34. Predicted composition of high molecular weight complexes including h77A-1-G1m3 and HSP70.

FIGS. 35A-C. Binding of murine 77A antibody in murine IgG1 (“mIgG1”; FIG. 35A), murine IgG2a (“mIgG2a”; FIG. 35B), and murine IgG2b (“mIgG2b”; FIG. 35C) formats, alone or in complex with human HSP70 (“hu HSP70”), to mouse FcγR3, as measured by ELISA. EC50s are indicated.

FIG. 36. Binding of humanized h77A-1-G1m3 antibody (“G1m(3)”), alone or in complex with HSP70, to human FcγR2A, as measured by ELISA. EC50 is indicated.

FIGS. 37A-C. Binding of murine 77A antibody to mouse FcγR3 (FIG. 37A) and humanized h77A-1-G1m3 antibody to human to FcγR2a (FIG. 37B), each alone (“antibody only”) or in complex with HSP70 (“complex”), as measured by biolayer interferometry (BLI). Kinetics of h77A-1-G1m3 binding to human FcγR2A (FIG. 37C), alone (“G1m(3)”) or in complex with HSP70 (“G1m(3) complex”), as measured by biolayer interferometry (BLI).

FIG. 38. Binding of commercially available CM710.1 and N15F2-5 antibodies to mouse FcγR3, each alone (top trace) or in complex with HSP70 (bottom trace), as measured by biolayer interferometry (BLI).

FIG. 39A. Size exclusion chromatography (SEC) traces for (i) N15F2-5 (“N15”) or murine 77A in a murine IgG1 format (“mou IgG1”), each labeled with CF647 dye, and (ii) HSP70 fused to GFP (“HSP70-GFP”), following co-incubation of antibody and HSP70. The same sample was run twice, first detecting fluorescence from GFP (left) and then again from CF647 (right). Peaks, corresponding retention time, and calculated approximate molecular weights (*MW) are indicated. Molecular weights for each peak were calculated from a MW standard curve.

FIG. 39B. Size exclusion chromatography (SEC) traces for (i) CM710.1 or murine 77A in a murine IgG1 format (“mou IgG1”), each labeled with CF647 dye, and (ii) HSP70 fused to GFP (“HSP70-GFP”), following co-incubation of antibody and HSP70. The same sample was run twice, first detecting fluorescence from GFP (left) and then again from CF647 (right). Peaks, corresponding retention time, and calculated approximate molecular weights (*MW) are indicated. Molecular weights for each peak were calculated from a MW standard curve.

FIG. 40. Uptake of antibody:HSP70-GFP complexes in 293 cells transfected with human FcγR2A, as measured by FACS. Antibodies tested were humanized h77A-1 in a human IgG2 format (“HC1-LC1”), and murine 77A in a murine IgG2b^{LALAPG} format (“LALAPG”; a reduced Fc receptor binding mutant). HEL: isotype control antibody. y-axis: percent of GFP positive cells, x-axis: antibody concentration. EC50 for humanized h77A-1 in a human IgG2 format (“HC1-LC1”) is indicated.

FIG. 41. Uptake of antibody:HSP70-GFP complexes in monocytic THP-1 cells, as measured by FACS. Antibodies tested were humanized h77A-1 in a human IgG2 format (“HC1-LC1”), and humanized h77A-1 in an IgG1 G1m3 format (“G1m(3)”). HEL: isotype control antibody. y-axis: percent of GFP positive cells, x-axis: antibody concentration.

FIG. 42. Uptake of antibody:HSP70-GFP complexes in monocytic THP-1 cells, as measured by FACS. Antibodies tested were h77A-1 in a human IgG1 G1m3-GA format (“GA”), h77A-1 in a human IgG1 G1m3-GAALIE format (“GAALIE”), h77A-1 in a human IgG2 format (“HC1-LC1”), h77A-1 in a human IgG1 G1m3 format (“G1m(3)”), and murine 77A in a murine IgG2b^{LALAPG} format (“LALAPG”). HEL: isotype control antibody. y-axis: percent of GFP positive cells, x-axis: antibody concentration.

FIG. 43. Uptake of antibody:HSP70-GFP complexes in murine RAW264.7 cells, as measured by FACS. Antibodies tested were murine 77A in murine IgG1 (“mIgG1”), IgG2a (“mIgG2a”), IgG2b (“mIgG2b”), and IgG2b^{LALAPG} (“LALAPG”) formats. HEL: isotype control antibody. y-axis: percent of live, GFP positive cells, x-axis: antibody concentration.

FIGS. 44A-B. Uptake of antibody:HSP70-GFP complexes in murine DC3.2 (FIG. 44A) and DC2.4 (FIG. 44B) cells, as measured by FACS. Antibodies tested were murine 77A in murine IgG1 (“mIgG1”), IgG2a (“mIgG2a”), IgG2B (“mIgG2b”), and IgG2b^{LALAPG} (“LALAPG”) formats. HEL: isotype control antibody. Y-axis: percent of live, GFP positive cells, x-axis: antibody concentration.

FIG. 45. Uptake of antibody:HSP70-GFP complexes in primary mouse monocytes derived from bone marrow cells, as measured by FACS. Antibodies tested were murine 77A in murine IgG1 (“mIgG1”), IgG2a (“mIgG2a”), IgG2B (“mIgG2b”), and IgG2b^{LALAPG} (“LALAPG”) formats. HEL: isotype control antibody. Y-axis: percent of live, GFP positive cells, x-axis: antibody concentration.

FIGS. 46A-C. Uptake of antibody:HSP70-GFP complexes in human macrophage cells (FIG. 46A), monocyte cells (FIG. 46B), and macrophage cells (FIG. 46C), as measured by FACS. Antibodies tested were h77A-1 in a human IgG1 G1m3 (“G1m(3)”), IgG1 G1m3-GA (“GA”), and IgG1 G1m3-GAALIE format. HEL: isotype control antibody. Y-axis: percent of live, GFP positive cells, x-axis: antibody concentration.

FIG. 47. Uptake of antibody:HSP70-GFP complexes in murine DC3.2 dendritic cells, as measured by FACS. Antibodies tested were N15F2-5, CM170.1, and murine 77A (“253-77A”) in murine IgG1, IgG2a, and IgG2b formats. HEL: isotype control antibody. X-axis: Alexa Flour 488-A to detect GFP, y-axis: FSC-A to detect viable cells. Percent live, GFP positive cells are indicated.

FIG. 48. Uptake of antibody:HSP70-GFP complexes in murine MH-S macrophage cells, as measured by FACS. Antibodies tested were N15F2-5, CM170.1, and murine 77A (“253-77A”) in murine IgG1, IgG2a, and IgG2b formats. HEL: isotype control antibody. X-axis: Alexa Flour 488-A to detect GFP, y-axis: FSC-A to detect viable cells. Percent live, GFP positive cells are indicated.

FIGS. 49A-C. Engagement by antibody with Human FcγR2A. Engineered Fc variants were evaluated in a dose-response titration for engagement with FcγR2A by monomeric antibody, or by antibody already complexed with antigen.

FIG. 50. Uptake of HSP70-GFP complexed with engineered Fc variants by primary human monocytes. The number of cells positive for GFP was gated on, and the percentage of positive cells in the live population was plotted.

FIG. 51. Uptake of HSP70-GFP complexed with engineered Fc variants by primary human macrophages. The number of cells positive for GFP was gated on, and the percentage of positive cells in the live population was plotted.

FIG. 52. Uptake of HSP70-GFP complexed with engineered Fc variants by primary human dendritic cells. The number of cells positive for GFP was gated on, and the percentage of positive cells in the live population was plotted.

FIG. 53. Proliferation of CD8+ T-cells as determined by FACS after incubation with 77A complexes.

FIG. 54. FACS analysis of CD25 and HLA_DR activation markers on CD8+ T-cells after incubation with 77A complexes. Complexes of HSP70 formed with 77A Fc variants induce expression of phenotypic markers of activation in CD8+ T-cells after co-culture.

FIG. 55. FACS analysis of immature dendritic cells for activation markers. Complexes of HSP70 formed with 77A Fc variants induce expression of CD83, a phenotypic markers of activation in immature dendritic cells after incubation with 77A complexes.

FIG. 56. Female C57BL/6 mice transgenic for human FcγRs were inoculated with the EG7-luc tumor line. Tumor growth was monitored by imaging for luciferase signal. HSP70-OVA fusion protein was co-incubated with control isotype (Iso^{PAL}), wildtype 77A IgG1 (77A^{WT}), or each of the engineered Fc mutants (77A^{GA}, 77A^{GAALIE}) and allowed to form a complex. Mice were then immunized and boosted with the proteins/complexes.

FIG. 57. Survival curves for female C57BL/6 FcγR transgenic (Tg) mice by treatment group.

FIG. 58. Female C57BL/6 mice transgenic for human FcγRs were inoculated with the E0771 tumor line. Tumor growth was monitored before and during treatment with the wildtype humanized 77A IgG1 antibody, or each of the engineered Fc variants. Tumor growth was compared to the isotype human IgG1 control while receiving treatment. The “On Tx” region indicates the time period while on treatment.

FIG. 59. Survival curves for female C57BL/6 FcγR Tg mice by treatment group. The “On Tx” region indicates the time period while on treatment.

FIG. 60. Male C57BL/6 mice transgenic for human FcγRs were inoculated with the PANO2-CRE2 tumor line. Tumor growth (in volume) was measured by caliper. Mice were then pre-conditioned with 10 Gy of direct tumor X-ray irradiation prior to treatment starting. Mice were initiated on treatment the following day with control isotype or each of the engineered Fc mutant 77A antibodies.

### DETAILED DESCRIPTION

The invention is based, in part, upon the development of anti-HSP antibodies, or antigen binding fragments thereof, that are useful in the treatment of certain indications, e.g., cancer. In certain circumstances, the anti-HSP70 antibodies (e.g., anti-HSP70 monoclonal antibodies) or antibody fragments thereof may, for example, target extracellular or soluble HSP70 associated with tumor-derived antigens to immune cells (e.g., dendritic cells) and thereby treat cancer and/or enhance the efficacy of a cancer therapy (e.g., a cancer immunotherapy). In certain circumstances, the anti-HSP70 antibodies or antibody fragments thereof may, for example, former high order (i.e., greater than one to one) complexes with HSP70.

The data provided herein show that an anti-HSP70 antibody (mAb; denoted as clone 77A) shows activity independent of surface HSP70 expression and targets extracellular HSP70 with tumor-derived antigens to DCs. This antibody can be used to better understand the role of HSP70 in immunity, and also as a therapeutic to enhance the effectiveness of cancer immunotherapy. In particular, clone 77A is a high affinity HSP70 mAb that shows anti-tumor efficacy in models of both hematologic malignancies and solid tumors in immune-competent and nude mice, but not in immune-deficient mice bearing the spontaneous Protein kinase, DNA-activated, catalytic subunit (PRKDCSCID) mutation, also known as SCID mice. The antibody enhances intracellular uptake of HSP70 by DCs in in vitro assays leading to upregulation of genes associated with DC maturation. When tested against orthotopically implanted 4T1 cells, an immunologically cold model of murine triple-negative breast cancer that does not respond to ICIs, 77A reduced primary tumor growth and inhibited the development of pulmonary and hepatic metastases. In combination with pegylated liposomal doxorubicin (PLD), an agent that causes immunogenic cell death (ICD) and enhances release of HSP70-tumor peptide complexes, 77A cured some mice in both the 4T1 model and a model of colorectal cancer. Finally, when ADP-HSP70 complexes purified from 4T1 cells were used as a vaccine with 77A, tumor growth after subsequent challenge with live 4T1 cells was inhibited compared with a mAb isotype control, and the abundance of 4T1-specific cytolytic CD4+ and CD8+ T-cell activity was enhanced. As such, enhancing the uptake of HSP70 by immune cells using clone 77A mAb augments anti-tumor immunity both alone, and in a number of rationally designed combination regimens.

The data provided herein also show that the 77A antibody and variants thereof form high molecular weight immune complexes upon binding to HSP70. These high molecular weight immune complexes bind more strongly to Fc receptors than antibody alone and show a marked enhancement of HSP70 cellular uptake relative to antibody alone. The ability to form high molecular weight immune complexes is not simply a function of HSP70 binding, because other commercially available anti-HSP70 antibodies do not appear to form such high molecular weight immune complexes. Accordingly, antibodies capable of forming high molecular weight immune complexes upon binding to HSP70 (e.g., 77A and variants thereof) are believed to be particularly useful in the treatment of certain indications, e.g., cancer.

### I. Definitions

“Nucleic acid,” “nucleic acid sequence,” “oligonucleotide,” “polynucleotide” or other grammatical equivalents as used herein means at least two nucleotides, either deoxyribonucleotides or ribonucleotides, or analogs thereof, covalently linked together. Polynucleotides are polymers of any length, including, e.g., 20, 50, 100, 200, 300, 500, 1000, 2000, 3000, 5000, 7000, 10,000, etc. A polynucleotide described herein generally contains phosphodiester bonds, although in some cases, nucleic acid analogs are included that may have at least one different linkage, e.g., phosphoramidate, phosphorothioate, phosphorodithioate, or O-methylphophoroamidite linkages, and peptide nucleic acid backbones and linkages. Mixtures of naturally occurring polynucleotides and analogs can be made; alternatively, mixtures of different polynucleotide analogs, and mixtures of naturally occurring polynucleotides and analogs may be made. The following are non-limiting examples of polynucleotides: a gene or gene fragment, exons, introns, messenger RNA (mRNA), transfer RNA, ribosomal RNA, ribozymes, cDNA, cRNA, recombinant polynucleotides, branched polynucleotides, plasmids, vectors, isolated DNA of any sequence, isolated RNA of any sequence, nucleic acid probes, and primers. A polynucleotide may comprise modified nucleotides, such as methylated nucleotides and nucleotide analogs. If present, modifications to the nucleotide structure may be imparted before or after assembly of the polymer. The sequence of nucleotides may be interrupted by non-nucleotide components. A polynucleotide may be further modified after polymerization, such as by conjugation with a labeling component. The term also includes both double- and single-stranded molecules. Unless otherwise specified or required, the term polynucleotide encompasses both the double-stranded form and each of two complementary single-stranded forms known or predicted to make up the double-stranded form. A polynucleotide is composed of a specific sequence of four nucleotide bases: adenine (A), cytosine (C), guanine (G), thymine (T), and uracil (U) for thymine when the polynucleotide is RNA. Thus, the term “polynucleotide sequence” is the alphabetical representation of a polynucleotide molecule. Unless otherwise indicated, a particular polynucleotide sequence also implicitly encompasses conservatively modified variants thereof (e.g., degenerate codon substitutions) and complementary sequences as well as the sequence explicitly indicated. Specifically, degenerate codon substitutions may be achieved by generating sequences in which the third position of one or more selected (or all) codons is substituted with mixed-base and/or deoxyinosine residues.

The terms “peptide,” “polypeptide” and “protein” used herein refer to polymers of amino acid residues. These terms also apply to amino acid polymers in which one or more amino acid residues is an artificial chemical mimetic of a corresponding naturally occurring amino acid, as well as to naturally occurring amino acid polymers, those containing modified residues, and non-naturally occurring amino acid polymers. In the present case, the term “polypeptide” encompasses an antibody or a fragment thereof.

Other terms used in the fields of recombinant nucleic acid technology, microbiology, immunology, antibody engineering, and molecular and cell biology as used herein will be generally understood by one of ordinary skill in the applicable arts.

The terms “a” and “an” as used herein mean “one or more” and include the plural unless the context is inappropriate.

The use of the term “or” in the claims is used to mean “and/or” unless explicitly indicated to refer to alternatives only or the alternatives are mutually exclusive, although the disclosure supports a definition that refers to only alternatives and “and/or.” As used herein “another” may mean at least a second or more.

As used herein, the terms “subject” and “patient” are used interchangeably and refer to an organism to be treated by the methods and compositions described herein. Such organisms preferably include, but are not limited to, mammals (e.g., murines, simians, equines, bovines, porcines, canines, felines, and the like), and more preferably include humans.

As used herein, the term “effective amount” refers to the amount of a compound (e.g., a compound of the present disclosure) sufficient to effect beneficial or desired results. An effective amount can be administered in one or more administrations, applications or dosages and is not intended to be limited to a particular formulation or administration route.

The terms “treatment” and “treating” refer to administration or application of a therapeutic agent to a subject or performance of a procedure or modality on a subject for the purpose of obtaining a therapeutic benefit of a disease or health-related condition. The terms also include such steps that impart any effect, e.g., lessening, reducing, modulating, ameliorating or eliminating, that results in the improvement of the condition, disease, disorder, and the like, or ameliorating a symptom thereof. For example, a treatment may include administration of a pharmaceutically effective amount of an antibody that targets HSP70, either alone or in combination with administration of chemotherapy, immunotherapy, or radiotherapy, performance of surgery, or any combination thereof.

The terms “therapeutic benefit” or “therapeutically effective” refer to anything that promotes or enhances the well-being of the subject with respect to the medical treatment of this condition. This includes, but is not limited to, a reduction in the frequency or severity of the signs or symptoms of a disease. For example, treatment of cancer may involve, for example, a reduction in the size of a tumor, a reduction in the invasiveness of a tumor, reduction in the growth rate of the cancer, or prevention of metastasis. Treatment of cancer may also refer to prolonging survival of a subject with cancer.

As used herein, the term “pharmaceutical composition” refers to the combination of an active agent with a pharmaceutically acceptable carrier (inert or active) making the composition especially suitable for diagnostic or therapeutic use in vivo or ex vivo

As used herein, the term “pharmaceutically acceptable carrier” refers to any of the standard pharmaceutical carriers, such as a phosphate buffered saline solution, water, emulsions (e.g., such as an oil/water or water/oil emulsions), and various types of wetting agents. The compositions also can include stabilizers and preservatives. For examples of carriers, stabilizers and adjuvants, see e.g., Adeboye Adejare, Remington: The Science and Practice of Pharmacy (23d ed. 2020).

Throughout this application, the term “about” is used to indicate that a value includes the inherent variation of error for the device, the method being employed to determine the value, the variation that exists among the study subjects, or a value that is within 10% of a stated value.

As used herein, the term “essentially free” in connection with a specified component, is used herein to mean that none of the specified component has been purposefully formulated into a composition and/or is present only as a contaminant or in trace amounts. The total amount of the specified component resulting from any unintended contamination of a composition is therefore below 0.5%, 0.1%, or 0.05%, and preferably below 0.01%. Most preferred is a composition in which no amount of the specified component can be detected with standard analytical methods.

Throughout the description, where compositions are described as having, including, or comprising specific components, or where processes and methods are described as having, including, or comprising specific steps, it is contemplated that, additionally, there are compositions of the present invention that consist essentially of, or consist of, the recited components, and that there are processes and methods according to the present invention that consist essentially of, or consist of, the recited processing steps.

In the application, where an element or component is said to be included in and/or selected from a list of recited elements or components, it should be understood that the element or component can be any one of the recited elements or components, or the element or component can be selected from a group consisting of two or more of the recited elements or components.

Further, it should be understood that elements and/or features of a composition or a method described herein can be combined in a variety of ways without departing from the spirit and scope of the present invention, whether explicit or implicit herein. For example, where reference is made to a particular compound, that compound can be used in various embodiments of compositions of the present invention and/or in methods of the present invention, unless otherwise understood from the context. In other words, within this application, embodiments have been described and depicted in a way that enables a clear and concise application to be written and drawn, but it is intended and will be appreciated that embodiments may be variously combined or separated without parting from the present teachings and invention(s). For example, it will be appreciated that all features described and depicted herein can be applicable to all aspects of the invention(s) described and depicted herein.

It should be understood that the order of steps or order for performing certain actions is immaterial so long as the present invention remain operable. Moreover, two or more steps or actions may be conducted simultaneously.

The use of any and all examples, or exemplary language herein, for example, “such as” or “including,” is intended merely to illustrate better the present invention and does not pose a limitation on the scope of the invention unless claimed. No language in the specification should be construed as indicating any non-claimed element as essential to the practice of the present invention.

The use of the term “include,” “includes,” “including,” “have,” “has,” “having,” “contain,” “contains,” or “containing,” including grammatical equivalents thereof, should be understood generally as open-ended and non-limiting, for example, not excluding additional unrecited elements or steps, unless otherwise specifically stated or understood from the context.

### II. Antibodies and Modifications of Antibodies

Provided herein are monoclonal antibodies having clone-paired CDRs from the heavy and light chains as illustrated in Tables 1, 6, 10, and 11. Such antibodies may be produced using methods described herein.

The monoclonal antibodies of the present invention have several applications, include the production of diagnostic kits for use in detecting HSP70, as well as for treating diseases associated with increased levels of HSP70. In these contexts, one may link such antibodies to diagnostic or therapeutic agents, use them as capture agents or competitors in competitive assays, or use them individually without additional agents being attached thereto. The antibodies may be mutated or modified, as discussed further below. Methods for preparing and characterizing antibodies are well known in the art (see, e.g., Antibodies: A Laboratory Manual, Cold Spring Harbor Laboratory, 1988; U.S. Pat. No. 4,196,265).

An “antibody” is an immunoglobulin molecule capable of specific binding to a target, such as a carbohydrate, polynucleotide, lipid, polypeptide, etc., through at least one antigen recognition site, located in the variable region of the immunoglobulin molecule. As used herein, the term encompasses not only intact polyclonal or monoclonal antibodies, but also fragments thereof (such as Fab, Fab′, F(ab′)₂, Fv, Fd, Fd′, single chain antibody (ScFv), diabody, linear antibody), mutants thereof, naturally occurring variants, fusion proteins comprising an antibody portion with an antigen recognition site of the required specificity, humanized antibodies, chimeric antibodies, and any other modified configuration of the immunoglobulin molecule that comprises an antigen recognition site of the required specificity.

An “isolated antibody” is an antibody that has been separated and/or recovered from a component of its natural environment. Contaminant components of its natural environment are materials that would interfere with diagnostic or therapeutic uses for the antibody, and may include enzymes, hormones, and other proteinaceous or non-proteinaceous solutes. In particular instances, the antibody is purified: (1) to greater than 95% by weight of antibody as determined by the Lowry method, and most particularly more than 99% by weight; or (2) to homogeneity by SDS-PAGE under reducing or non-reducing conditions using Coomassie blue or silver stain. An isolated antibody includes the antibody in situ within recombinant cells since at least one component of the antibody's natural environment will not be present. Ordinarily, however, an isolated antibody will be prepared by at least one purification step.

The basic four-chain antibody unit is a heterotetrameric glycoprotein composed of two identical light (L) chains and two identical heavy (H) chains. The term “heavy chain” as used herein refers to the larger immunoglobulin subunit which associates, through its amino terminal region, with the immunoglobulin light chain. The heavy chain comprises a variable region (V_{H}) and a constant region (C_{H}). The constant region further comprises the C_{H}1, hinge, C_{H}2, and C_{H}3 domains. In the case of IgE, IgM, and IgY, the heavy chain comprises a C_{H}4 domain but does not have a hinge domain. Those skilled in the art will appreciate that heavy chains are classified as gamma, mu, alpha, delta, or epsilon (γ, μ, α, δ, ε), with some subclasses among them (e.g., γ1-γ4, α1-α2). It is the nature of this chain that determines the “class” of the antibody as IgG, IgM, IgA IgD, or IgE, respectively. The immunoglobulin subclasses (isotypes), e.g., IgG1, IgG2, IgG3, IgG4, IgA1, etc. are well characterized and are known to confer functional specialization.

The term “light chain” as used herein refers to the smaller immunoglobulin subunit which associates with the amino terminal region of a heavy chain. As with a heavy chain, a light chain comprises a variable region (V_{L}) and a constant region (C_{L}). Light chains are classified as either kappa or lambda (κ, λ) based on the amino acid sequences of their constant domains (C_{L}). A pair of these can associate with a pair of any of the various heavy chains to form an immunoglobulin molecule. Also encompassed in the meaning of light chain are light chains with a lambda variable region (V-lambda) linked to a kappa constant region (C-kappa) or a kappa variable region (V-kappa) linked to a lambda constant region (C-lambda).

An IgM antibody, for example, consists of 5 basic heterotetramer units along with an additional polypeptide called J chain, and therefore contains 10 antigen binding sites, while secreted IgA antibodies can polymerize to form polyvalent assemblages comprising 2-5 of the basic 4-chain units along with J chain. In the case of IgGs, the 4-chain unit is generally about 150,000 daltons. Each L chain is linked to an H chain by one covalent disulfide bond, while the two H chains are linked to each other by one or more disulfide bonds depending on the H chain isotype. Each H and L chain also has regularly spaced intrachain disulfide bridges. Each H chain has at the N-terminus, a variable region (V_{H}) followed by three constant domains (C_{H}) for each of the alpha and gamma chains and four C_{H} domains for mu and isotypes. Each L chain has at the N-terminus, a variable region (V_{L}) followed by a constant domain (C_{L}) at its other end. The V_{L} is aligned with the V_{H} and the C_{L} is aligned with the first constant domain of the heavy chain (C_{H}1). Particular amino acid residues are believed to form an interface between the light chain and heavy chain variable regions. The pairing of a V_{H} and V_{L} together forms a single antigen-binding site. For the structure and properties of the different classes of antibodies, see, e.g., Basic and Clinical Immunology, 8th edition, Daniel P. Stites, Abba I. Terr and Tristram G. Parslow (eds.), Appleton & Lange, Norwalk, Conn., 1994, page 71, and Chapter 6.

A “variable region” of an antibody refers to the variable region of the antibody light chain or the variable region of the antibody heavy chain, either alone or in combination. The term “variable” refers to the fact that certain segments of the variable regions differ extensively in sequence among antibodies. The variable regions of both the light (V_{L}) and heavy (V_{H}) chain portions mediate antigen binding and define the specificity of a particular antibody for its particular antigen. However, the variability is not evenly distributed across the entirety of the variable regions. Instead, the variable regions consist of relatively invariant stretches called framework regions (FRs) separated by shorter regions of extreme variability called complementarity determining regions (CDRs) or hypervariable regions. The variable regions of native heavy and light chains each comprise four FRs, largely adopting a beta-sheet configuration, connected by three CDRs, which form loops connecting, and in some cases forming part of, the beta-sheet structure. The CDRs complement an antigen's shape and determine the antibody's affinity and specificity for the antigen. There are six CDRs in both V_{L} and V_{H}. The CDRs in each chain are held together in close proximity by the FRs and, with the CDRs from the other chain, contribute to the formation of the antigen-binding site of antibodies (see Kabat et al., Sequences of Proteins of Immunological Interest, 5th Ed. Public Health Service, National Institutes of Health, Bethesda, Md. (1991)).

The term “hypervariable region” when used herein refers to the amino acid residues of an antibody that are responsible for antigen binding. The hypervariable region generally comprises amino acid residues from a “complementarity determining region” or “CDR” (e.g., around about residues 24-34 (L1), 50-56 (L2) and 89-97 (L3) in the V_{L}, and around about 31-35 (H1), 50-65 (H2) and 95-102 (H3) in the V_{H} when numbered in accordance with the Kabat numbering system; Kabat et al., Sequences of Proteins of Immunological Interest, 5th Ed. Public Health Service, National Institutes of Health, Bethesda, Md. (1991)); and/or those residues from a “hypervariable loop” (e.g., residues 24-34 (L1), 50-56 (L2) and 89-97 (L3) in the V_{L}, and 26-32 (H1), 52-56 (H2) and 95-101 (H3) in the V_{H} when numbered in accordance with the Chothia numbering system; Chothia and Lesk, J. Mol. Biol. 196:901-917 (1987)); and/or those residues from a “hypervariable loop”/CDR (e.g., residues 27-38 (L1), 56-65 (L2) and 105-120 (L3) in the V_{L}, and 27-38 (H1), 56-65 (H2) and 105-120 (H3) in the V_{H} when numbered in accordance with the IMGT numbering system; Lefranc, M. P. et al. Nucl. Acids Res. 27:209-212 (1999), Ruiz, M. et al. Nucl. Acids Res. 28:219-221 (2000)). Optionally the antibody has symmetrical insertions at one or more of the following points 28, 36 (L1), 63, 74-75 (L2) and 123 (L3) in the V_{L}, and 28, 36 (H1), 63, 74-75 (H2) and 123 (H3) in the V_{H} when numbered in accordance with AHo; Honneger, A. and Plunkthun, A. J. Mol. Biol. 309:657-670 (2001)). As used herein, a CDR may refer to CDRs defined by any of these numbering approaches or by a combination of approaches or by other desirable approaches. In addition, a new definition of highly conserved core, boundary and hyper-variable regions can be used.

A “constant region” of an antibody refers to the constant region of the antibody light chain or the constant region of the antibody heavy chain, either alone or in combination. The constant regions of the light chain (C_{L}) and the heavy chain (C_{H}1, CH2 or C_{H}3, or C_{H}4 in the case of IgM and IgE) confer important biological properties such as secretion, transplacental mobility, Fc receptor binding, complement binding, and the like. By convention the numbering of the constant region domains increases as they become more distal from the antigen binding site or amino-terminus of the antibody. The constant regions are not involved directly in binding an antibody to an antigen, but exhibit various effector functions, such as participation of the antibody in antibody dependent cellular cytotoxicity (ADCC), antibody-dependent cellular phagocytosis (ADCP), antibody-dependent neutrophil phagocytosis (ADNP), and antibody-dependent complement deposition (ADCD).

The antibody may be an antibody fragment. “Antibody fragments” comprise only a portion of an intact antibody, generally including an antigen binding site of the intact antibody and thus retaining the ability to bind antigen. Examples of antibody fragments encompassed by the present definition include: (i) the Fab fragment, having V_{L}, C_{L}, V_{H} and C_{H1} domains; (ii) the Fab′ fragment, which is a Fab fragment having one or more cysteine residues at the C-terminus of the C_{H1} domain; (iii) the Fd fragment having V_{H} and C_{H}1 domains; (iv) the Fd′ fragment having V_{H} and C_{H}1 domains and one or more cysteine residues at the C-terminus of the C_{H}1 domain; (v) the Fv fragment having the V_{L} and V_{H} domains of a single antibody; (vi) the dAb fragment which consists of a V_{H} domain; (vii) isolated CDR regions; (viii) F(ab′)₂ fragments, a bivalent fragment including two Fab′ fragments linked by a disulfide bridge at the hinge region; (ix) single chain antibody molecules (e.g. single chain Fv; scFv); (x) “diabodies” with two antigen binding sites, comprising a heavy chain variable domain (V_{H}) connected to a light chain variable domain (V_{L}) in the same polypeptide chain; (xi) “linear antibodies” comprising a pair of tandem Fd segments (V_{H}-C_{H}1-V_{H}-C_{H}1) which, together with complementary light chain polypeptides, form a pair of antigen binding regions.

The antibody may be a chimeric antibody. “Chimeric antibodies” refers to those antibodies wherein one portion of each of the amino acid sequences of heavy and light chains is homologous to corresponding sequences in antibodies derived from a particular species or belonging to a particular class, while the remaining segment of the chains is homologous to corresponding sequences in another. For example, a chimeric antibody may be an antibody comprising antigen binding sequences from a non-human donor grafted to a heterologous non-human, human, or humanized sequence (e.g., framework and/or constant domain sequences). Typically, in these chimeric antibodies, the variable region of both light and heavy chains mimics the variable regions of antibodies derived from one species of mammals, while the constant portions are homologous to the sequences in antibodies derived from another. For example, methods have been developed to replace light and heavy chain constant domains of a monoclonal antibody with analogous domains of human origin, leaving the variable regions of the foreign antibody intact. Alternatively, “fully human” monoclonal antibodies can be produced in mice transgenic for human immunoglobulin genes. Methods have also been developed to convert variable domains of monoclonal antibodies to more human form by recombinantly constructing antibody variable domains having both rodent, for example, mouse, and human amino acid sequences. In “humanized” monoclonal antibodies, only the hypervariable CDR is derived from mouse monoclonal antibodies, and the framework and constant regions are derived from human amino acid sequences (see U.S. Pat. Nos. 5,091,513 and 6,881,557, incorporated herein by reference). It is thought that replacing amino acid sequences in the antibody that are characteristic of rodents with amino acid sequences found in the corresponding position of human antibodies will reduce the likelihood of adverse immune reaction during therapeutic use. A hybridoma or other cell producing an antibody may also be subject to genetic mutation or other changes, which may or may not alter the binding specificity of antibodies produced by the hybridoma.

### A. Monoclonal Antibodies

The term “monoclonal antibody” as used herein refers to an antibody obtained from a population of substantially homogeneous antibodies, i.e., the individual antibodies comprising the population are identical except for possible naturally occurring mutations that may be present in minor amounts. Monoclonal antibodies are highly specific, being directed against a single antigenic site. Furthermore, in contrast to polyclonal antibody preparations that include different antibodies directed against different determinants (epitopes), each monoclonal antibody is directed against a single determinant on the antigen. In addition to their specificity, monoclonal antibodies are advantageous in that they may be synthesized uncontaminated by other antibodies. The modifier “monoclonal” is not to be construed as requiring production of the antibody by any particular method. For example, the monoclonal antibodies useful in the present disclosure may be prepared by the hybridoma methodology first described by Kohler et al., Nature, 256:495 (1975), or may be made using recombinant DNA methods in bacterial, eukaryotic animal or plant cells (see, e.g., U.S. Pat. No. 4,816,567) after single cell sorting of an antigen specific B cell, an antigen specific plasmablast responding to an infection or immunization, or capture of linked heavy and light chains from single cells in a bulk sorted antigen specific collection. The monoclonal antibodies may also be isolated from phage antibody libraries using the techniques described in Clackson et al., Nature, 352:624-628 (1991) and Marks et al., J. Mol. Biol., 222:581-597 (1991), for example.

Methods for producing monoclonal antibodies of various types, including humanized, chimeric, and fully human, are well known in the art and highly predictable. For example, the following U.S. patents and patent applications provide enabling descriptions of such methods: U.S. Patent Application Nos. 2004/0126828 and 2002/0172677; and U.S. Pat. Nos. 3,817,837; 3,850,752; 3,939,350; 3,996,345; 4,196,265; 4,275,149; 4,277,437; 4,366,241; 4,469,797; 4,472,509; 4,606,855; 4,703,003; 4,742,159; 4,767,720; 4,816,567; 4,867,973; 4,938,948; 4,946,778; 5,021,236; 5,164,296; 5,196,066; 5,223,409; 5,403,484; 5,420,253; 5,565,332; 5,571,698; 5,627,052; 5,656,434; 5,770,376; 5,789,208; 5,821,337; 5,844,091; 5,858,657; 5,861,155; 5,871,907; 5,969,108; 6,054,297; 6,165,464; 6,365,157; 6,406,867; 6,709,659; 6,709,873; 6,753,407; 6,814,965; 6,849,259; 6,861,572; 6,875,434; and 6,891,024, each incorporated herein by reference.

### B. Single Chain Antibodies

A single chain variable fragment (scFv) is a fusion of the variable regions of the heavy and light chains of immunoglobulins, linked together with a short linker. This chimeric molecule retains the specificity of the original immunoglobulin, despite removal of the constant regions and the introduction of a linker peptide. This modification usually leaves the specificity unaltered. scFv can be created directly from subcloned heavy and light chains derived from a hybridoma or B cell. Single chain variable fragments lack the constant Fc region found in complete antibody molecules, and thus, the common binding sites (e.g., protein A/G) used to purify antibodies. These fragments can often be purified/immobilized using Protein L since Protein L interacts with the variable region of kappa light chains.

Flexible linkers generally are comprised of helix- and turn-promoting amino acid residues such as alanine, serine and glycine. However, other residues can function as well. For example, the linker may have a proline residue two residues after the V_{H} C terminus and an abundance of arginines and prolines at other positions.

A single-chain antibody may also be created by joining receptor light and heavy chains using a non-peptide linker or chemical unit. Generally, the light and heavy chains will be produced in distinct cells, purified, and subsequently linked together in an appropriate fashion (i.e., the N-terminus of the heavy chain being attached to the C-terminus of the light chain via an appropriate chemical bridge).

Cross-linking reagents are used to form molecular bridges that tie functional groups of two different molecules, e.g., a stabilizing and coagulating agent. However, it is contemplated that dimers or multimers of the same analog or heteromeric complexes comprised of different analogs can be created. To link two different compounds in a step-wise manner, hetero-bifunctional cross-linkers can be used that eliminate unwanted homopolymer formation.

An exemplary hetero-bifunctional cross-linker contains two reactive groups: one reacting with primary amine group (e.g., N-hydroxy succinimide) and the other reacting with a thiol group (e.g., pyridyl disulfide, maleimides, halogens, etc.). Through the primary amine reactive group, the cross-linker may react with the lysine residue(s) of one protein (e.g., the selected antibody or fragment) and through the thiol reactive group, the cross-linker, already tied up to the first protein, reacts with the cysteine residue (free sulfhydryl group) of the other protein (e.g., the selective agent).

It is preferred that a cross-linker having reasonable stability in blood will be employed. Numerous types of disulfide-bond containing linkers are known that can be successfully employed to conjugate targeting and therapeutic/preventative agents. Linkers that contain a disulfide bond that is sterically hindered may prove to give greater stability in vivo, preventing release of the targeting peptide prior to reaching the site of action. These linkers are thus one group of linking agents.

For example, SMPT is a bifunctional cross-linker containing a disulfide bond that is “sterically hindered” by an adjacent benzene ring and methyl groups. It is believed that steric hindrance of the disulfide bond serves a function of protecting the bond from attack by thiolate anions such as glutathione which can be present in tissues and blood, and thereby help in preventing decoupling of the conjugate prior to the delivery of the attached agent to the target site. The SMPT cross-linking reagent, as with many other known cross-linking reagents, lends the ability to cross-link functional groups such as the SH of cysteine or primary amines (e.g., the epsilon amino group of lysine). Another possible type of cross-linker includes the hetero-bifunctional photoreactive phenylazides containing a cleavable disulfide bond such as sulfosuccinimidyl-2-(p-azido salicylamido) ethyl-1,3′-dithiopropionate. The N-hydroxy-succinimidyl group reacts with primary amino groups and the phenylazide (upon photolysis) reacts non-selectively with any amino acid residue.

In addition to hindered cross-linkers, non-hindered linkers also can be employed in accordance herewith. Other useful cross-linkers, not considered to contain or generate a protected disulfide, include SATA, SPDP and 2-iminothiolane. The use of such cross-linkers is well understood in the art. Flexible linkers may also be used.

U.S. Pat. No. 4,680,338, describes bifunctional linkers useful for producing conjugates of ligands with amine-containing polymers and/or proteins, especially for forming antibody conjugates with chelators, drugs, enzymes, detectable labels and the like. U.S. Pat. Nos. 5,141,648 and 5,563,250 disclose cleavable conjugates containing a labile bond that is cleavable under a variety of mild conditions. This linker is particularly useful in that the agent of interest may be bonded directly to the linker, with cleavage resulting in release of the active agent. Particular uses include adding a free amino or free sulfhydryl group to a protein, such as an antibody, or a drug.

U.S. Pat. No. 5,856,456 provides peptide linkers for use in connecting polypeptide constituents to make fusion proteins, e.g., single chain antibodies. The linker is up to about 50 amino acids in length, contains at least one occurrence of a charged amino acid (preferably arginine or lysine) followed by a proline, and is characterized by greater stability and reduced aggregation. U.S. Pat. No. 5,880,270 discloses aminooxy-containing linkers useful in a variety of immunodiagnostic and separative techniques.

### C. Bispecific and Multispecific Antibodies

Antibodies may be bispecific or multispecific. “Bispecific antibodies” are antibodies that have binding specificities for at least two different epitopes. Exemplary bispecific antibodies may bind to two different epitopes of a single antigen. Other such antibodies may combine a first antigen binding site with a binding site for a second antigen.

Alternatively, an antigen-specific arm may be combined with an arm that binds to a triggering molecule on a leukocyte, such as a T-cell receptor molecule (e.g., CD3), or Fc receptors for IgG (FcγR), such as FcγRI (CD64), FcγRII (CD32) and Fc gamma RIII (CD16), so as to focus and localize cellular defense mechanisms to the infected cell. Bispecific antibodies may also be used to localize cytotoxic agents to infected cells. These antibodies possess an antigen-binding arm and an arm that binds the cytotoxic agent (e.g., saporin, anti-interferon-α, vinca alkaloid, ricin A chain, methotrexate or radioactive isotope hapten). Bispecific antibodies can be prepared as full-length antibodies or antibody fragments (e.g., F(ab′)₂ bispecific antibodies). Taki et al. (2015) describes a bispecific anti-HSP70/anti-CD3 antibody.

Methods for making bispecific antibodies are known in the art. Traditional production of full-length bispecific antibodies is based on the co-expression of two immunoglobulin heavy chain-light chain pairs, where the two chains have different specificities. Because of the random assortment of immunoglobulin heavy and light chains, these hybridomas (quadromas) produce a potential mixture of ten different antibody molecules, of which only one has the correct bispecific structure. Purification of the correct molecule, which is usually done by affinity chromatography steps, is rather cumbersome, and the product yields are low.

According to a different approach, antibody variable regions with the desired binding specificities (antibody-antigen combining sites) are fused to immunoglobulin constant domain sequences. Preferably, the fusion is with an Ig heavy chain constant domain, comprising at least part of the hinge, C_{H2}, and C_{H3} regions. It is preferred to have the first heavy-chain constant region (C_{H1}) containing the site necessary for light chain bonding, present in at least one of the fusions. DNAs encoding the immunoglobulin heavy chain fusions and, if desired, the immunoglobulin light chain, are inserted into separate expression vectors, and are co-transfected into a suitable host cell. This provides for greater flexibility in adjusting the mutual proportions of the three polypeptide fragments when unequal ratios of the three polypeptide chains used in the construction provide the optimum yield of the desired bispecific antibody. It is, however, possible to insert the coding sequences for two or all three polypeptide chains into a single expression vector when the expression of at least two polypeptide chains in equal ratios results in high yields or when the ratios have no significant effect on the yield of the desired chain combination.

The bispecific antibodies may be composed of a hybrid immunoglobulin heavy chain with a first binding specificity in one arm, and a hybrid immunoglobulin heavy chain-light chain pair (providing a second binding specificity) in the other arm. This asymmetric structure facilitates the separation of the desired bispecific compound from unwanted immunoglobulin chain combinations, as the presence of an immunoglobulin light chain in only one half of the bispecific molecule provides for a facile way of separation. This approach is disclosed in WO 94/04690. For further details of generating bispecific antibodies see, for example, Suresh et al., Methods in Enzymology, 121:210 (1986).

According to another approach described in U.S. Pat. No. 5,731,168, the interface between a pair of antibody molecules can be engineered to maximize the percentage of heterodimers that are recovered from recombinant cell culture. The preferred interface comprises at least a part of the C_{H3} domain. In this method, one or more small amino acid side chains from the interface of the first antibody molecule are replaced with larger side chains (e.g., tyrosine or tryptophan). Compensatory “cavities” of identical or similar size to the large side chain(s) are created on the interface of the second antibody molecule by replacing large amino acid side chains with smaller ones (e.g., alanine or threonine). This provides a mechanism for increasing the yield of the heterodimer over other unwanted end-products such as homodimers.

Bispecific antibodies include cross-linked or “heteroconjugate” antibodies. For example, one of the antibodies in the heteroconjugate can be coupled to avidin, the other to biotin. Such antibodies have, for example, been proposed to target immune system cells to unwanted cells (U.S. Pat. No. 4,676,980). Heteroconjugate antibodies may be made using any convenient cross-linking methods. Suitable cross-linking agents are well known in the art, and are disclosed in U.S. Pat. No. 4,676,980, along with a number of cross-linking techniques.

Techniques for generating bispecific antibodies from antibody fragments have also been described in the literature. For example, bispecific antibodies can be prepared using chemical linkage. Brennan et al., Science, 229: 81 (1985) describe a procedure wherein intact antibodies are proteolytically cleaved to generate F(ab′)₂ fragments. These fragments are reduced in the presence of the dithiol complexing agent, sodium arsenite, to stabilize vicinal dithiols and prevent intermolecular disulfide formation. The Fab′ fragments generated are then converted to thionitrobenzoate (TNB) derivatives. One of the Fab′-TNB derivatives is then reconverted to the Fab′-thiol by reduction with mercaptoethylamine and is mixed with an equimolar amount of the other Fab′-TNB derivative to form the bispecific antibody. The bispecific antibodies produced can be used as agents for the selective immobilization of enzymes.

Techniques exist that facilitate the direct recovery of Fab′-SH fragments from E. coli, which can be chemically coupled to form bispecific antibodies. Shalaby et al., J. Exp. Med., 175: 217-225 (1992) describe the production of a humanized bispecific antibody F(ab′)₂ molecule. Each Fab′ fragment was separately secreted from E. coli and subjected to directed chemical coupling in vitro to form the bispecific antibody. The bispecific antibody thus formed was able to bind to cells overexpressing the ErbB2 receptor and normal human T cells, as well as trigger the lytic activity of human cytotoxic lymphocytes against human breast tumor targets.

Various techniques for making and isolating bispecific antibody fragments directly from recombinant cell culture have also been described (Merchant et al., Nat. Biotechnol. 16, 677-681 (1998)). For example, bispecific antibodies have been produced using leucine zippers (Kostelny et al., J. Immunol., 148(5):1547-1553, 1992). The leucine zipper peptides from the Fos and Jun proteins were linked to the Fab′ portions of two different antibodies by gene fusion. The antibody homodimers were reduced at the hinge region to form monomers and then re-oxidized to form the antibody heterodimers. This method can also be utilized for the production of antibody homodimers. The “diabody” technology described by Hollinger et al., Proc. Natl. Acad. Sci. USA, 90:6444-6448 (1993) has provided an alternative mechanism for making bispecific antibody fragments. The fragments comprise a V_{H} connected to a V_{L} by a linker that is too short to allow pairing between the two domains on the same chain. Accordingly, the V_{H} and V_{L} domains of one fragment are forced to pair with the complementary V_{L} and V_{H} domains of another fragment, thereby forming two antigen-binding sites. Another strategy for making bispecific antibody fragments by the use of single-chain Fv (sFv) dimers has also been reported. See Gruber et al., J. Immunol., 152:5368 (1994).

A bispecific or multispecific antibody may be formed as a DOCK-AND-LOCK™ (DNL™) complex (see, e.g., U.S. Pat. Nos. 7,521,056; 7,527,787; 7,534,866; 7,550,143 and 7,666,400). Generally, the technique takes advantage of the specific and high-affinity binding interactions that occur between a dimerization and docking domain (DDD) sequence of the regulatory (R) subunits of cAMP-dependent protein kinase (PKA) and an anchor domain (AD) sequence derived from any of a variety of AKAP proteins (Baillie et al., FEBS Letters. 2005; 579: 3264; Wong and Scott, Nat. Rev. Mol. Cell Biol. 2004; 5: 959). The DDD and AD peptides may be attached to any protein, peptide or other molecule. Because the DDD sequences spontaneously dimerize and bind to the AD sequence, the technique allows the formation of complexes between any selected molecules that may be attached to DDD or AD sequences.

Antibodies with more than two valencies are contemplated. For example, trispecific antibodies can be prepared (Tutt et al., J. Immunol. 147: 60, 1991; Xu et al., Science, 358(6359):85-90, 2017). The antibodies may also involve sequences or moieties that permit dimerization or multimerization of the receptors. Such sequences include those derived from IgA, which permit formation of multimers in conjunction with the J-chain. Another multimerization domain is the Gal4 dimerization domain.

A multivalent antibody may be internalized (and/or catabolized) faster than a bivalent antibody by a cell expressing an antigen to which the antibody binds. The antibodies of the present disclosure can be multivalent antibodies with three or more antigen binding sites (e.g., tetravalent antibodies), which can be readily produced by recombinant expression of nucleic acid encoding the polypeptide chains of the antibody. The multivalent antibody can comprise a dimerization domain and three or more antigen binding sites. The preferred dimerization domain comprises (or consists of) an Fc region or a hinge region. In this scenario, the antibody will comprise an Fc region and three or more antigen binding sites amino-terminal to the Fc region. Multivalent antibodies may comprise (or consist of) three to about eight, for example four, antigen binding sites. The multivalent antibody comprises at least one polypeptide chain (and preferably two polypeptide chains), wherein the polypeptide chain(s) comprise two or more variable regions. For instance, the polypeptide chain(s) may comprise VD1-(X1).sub.n-VD2-(X2)ₙ-Fc, wherein VD1 is a first variable region, VD2 is a second variable region, Fc is one polypeptide chain of an Fc region, X1 and X2 represent an amino acid or polypeptide, and n is 0 or 1. For instance, the polypeptide chain(s) may comprise: VH-CH1-flexible linker-VH-CH1-Fc region chain; or VH-CH1-VH-CH1-Fc region chain. The multivalent antibody herein may further comprise at least two (and preferably four) light chain variable region polypeptides. The multivalent antibody herein may, for instance, comprise from about two to about eight light chain variable region polypeptides. The light chain variable region polypeptides contemplated here comprise a light chain variable region and, optionally, further comprise a C_{L} domain.

Charge modifications are particularly useful in the context of a multispecific antibody, where amino acid substitutions in Fab molecules result in reducing the mispairing of light chains with non-matching heavy chains (Bence-Jones-type side products), which can occur in the production of Fab-based bi-/multispecific antigen binding molecules with a VH/VL exchange in one (or more, in case of molecules comprising more than two antigen-binding Fab molecules) of their binding arms (see also PCT publication no. WO 2015/150447, particularly the examples therein, incorporated herein by reference in its entirety).

### D. Antibody Conjugates

Antibodies of the present disclosure may be linked to at least one agent to form an antibody conjugate. The conjugate can be, for example, an antibody conjugated to another proteinaceous, carbohydrate, lipid, or mixed moiety molecule(s). Such antibody conjugates include, but are not limited to, modifications that include linking the antibody to one or more polymers. For example, an antibody may be linked to one or more water-soluble polymers. Linkage to a water-soluble polymer reduces the likelihood that the antibody will precipitate in an aqueous environment, such as a physiological environment. One skilled in the art can select a suitable water-soluble polymer based on considerations including, but not limited to, whether the polymer/antibody conjugate will be used in the treatment of a patient and, if so, the pharmacological profile of the antibody (e.g., half-life, dosage, activity, antigenicity, and/or other factors).

In order to increase the efficacy of antibody molecules as diagnostic or therapeutic agents, it is conventional to link or covalently bind or complex at least one desired molecule or moiety. Such a molecule or moiety may be, but is not limited to, at least one effector or reporter molecule. Effector molecules comprise molecules having a desired activity, e.g., cytotoxic activity. Non-limiting examples of effector molecules which have been attached to antibodies include toxins, anti-tumor agents, therapeutic enzymes, radionuclides, antiviral agents, chelating agents, cytokines, growth factors, and oligo- or polynucleotides. By contrast, a reporter molecule is defined as any moiety which may be detected using an assay. Non-limiting examples of reporter molecules which have been conjugated to antibodies include enzymes, radiolabels, haptens, fluorescent labels, phosphorescent molecules, chemiluminescent molecules, chromophores, photoaffinity molecules, colored particles or ligands, an enzyme (e.g., that catalyzes a colorimetric or fluorometric reaction), a substrate, a solid matrix, such as biotin. An antibody may comprise one, two, or more of any of these labels.

Antibody conjugates may be used to deliver cytotoxic agents to target cells. Cytotoxic agents of this type may improve antibody-mediated cytotoxicity, and include such moieties as cytokines that directly or indirectly stimulate cell death, radioisotopes, chemotherapeutic drugs (including prodrugs), bacterial toxins (e.g., pseudomonas exotoxin, diphtheria toxin, etc.), plant toxins (e.g., ricin, gelonin, etc.), chemical conjugates (e.g., maytansinoid toxins, auristatins, α-amanitin, anthracyclines, calechaemicin, etc.), radioconjugates, enzyme conjugates (e.g., RNase conjugates, granzyme antibody-directed enzyme/prodrug therapy), and the like.

Antibody conjugates are also used as diagnostic agents. Antibody diagnostics generally fall within two classes, those for use in in vitro diagnostics, such as in a variety of immunoassays, and those for use in vivo diagnostic protocols, generally known as “antibody-directed imaging.” Many appropriate imaging agents are known in the art, as are methods for their attachment to antibodies (see, for e.g., U.S. Pat. Nos. 5,021,236, 4,938,948, and 4,472,509). The imaging moieties used can be paramagnetic ions, radioactive isotopes, fluorochromes, NMR-detectable substances, and X-ray imaging agents.

The paramagnetic ions contemplated for use as conjugates include chromium (III), manganese (II), iron (III), iron (II), cobalt (II), nickel (II), copper (II), neodymium (III), samarium (III), ytterbium (III), gadolinium (III), vanadium (II), terbium (III), dysprosium (III), holmium (III) and/or erbium (III), with gadolinium being particularly preferred. Ions useful in other contexts, such as X-ray imaging, include but are not limited to lanthanum (III), gold (III), lead (II), and bismuth (III).

The radioactive isotopes contemplated for use as conjugated include astatine²¹¹, ¹⁴carbon, ⁵¹chromium, ³⁶chlorine, ⁵⁷cobalt, ⁵⁸cobalt, copper⁶⁷, ¹⁵²Eu, gallium⁶⁷, ³hydrogen, iodine¹²³, iodine¹²⁵, iodine¹³¹, indium¹¹¹, ⁵⁹iron, ³²phosphorus, rhenium¹⁸⁶, rhenium¹⁸⁸, ⁷⁵selenium, ³⁵sulphur, technicium^{99m} and/or yttrium⁹⁰. ¹²⁵I is often being preferred. Technicium^{99m} and/or indium¹¹¹ are also often preferred due to their low energy and suitability for long range detection. Radioactively labeled monoclonal antibodies of the present disclosure may be produced according to well-known methods in the art. For instance, monoclonal antibodies can be iodinated by contact with sodium and/or potassium iodide and a chemical oxidizing agent such as sodium hypochlorite, or an enzymatic oxidizing agent, such as lactoperoxidase. Monoclonal antibodies according to the disclosure may be labeled with technetium^{99m} by ligand exchange process, for example, by reducing pertechnate with stannous solution, chelating the reduced technetium onto a Sephadex column and applying the antibody to this column. Alternatively, direct labeling techniques may be used, e.g., by incubating pertechnate, a reducing agent such as SNCl₂, a buffer solution such as sodium-potassium phthalate solution, and the antibody. Intermediary functional groups which are often used to bind radioisotopes which exist as metallic ions to antibody are diethylenetriaminepentaacetic acid (DTPA) or ethylene diaminetetracetic acid (EDTA).

The fluorescent labels contemplated for use as conjugates include Alexa 350, Alexa 430, AMCA, BODIPY 630/650, BODIPY 650/665, BODIPY-FL, BODIPY-R6G, BODIPY-TMR, BODIPY-TRX, Cascade Blue, Cy3, Cy5,6-FAM, Fluorescein Isothiocyanate, HEX, 6-JOE, Oregon Green 488, Oregon Green 500, Oregon Green 514, Pacific Blue, REG, Rhodamine Green, Rhodamine Red, Renographin, ROX, TAMRA, TET, Tetramethylrhodamine, and/or Texas Red.

Additional types of antibodies contemplated in the present disclosure are those intended primarily for use in vitro, where the antibody is linked to a secondary binding ligand and/or to an enzyme (an enzyme tag) that will generate a colored product upon contact with a chromogenic substrate. Examples of suitable enzymes include urease, alkaline phosphatase, (horseradish) hydrogen peroxidase or glucose oxidase. Preferred secondary binding ligands are biotin and avidin and streptavidin compounds.

Several methods are known in the art for the attachment or conjugation of an antibody to its conjugate moiety. Some attachment methods involve the use of a metal chelate complex employing, for example, an organic chelating agent such a diethylenetriaminepentaacetic acid anhydride (DTPA); ethylenetriaminetetraacetic acid; N-chloro-p-toluenesulfonamide; and/or tetrachloro-3α-6α-diphenylglycouril-3 attached to the antibody (U.S. Pat. Nos. 4,472,509 and 4,938,948). Monoclonal antibodies may also be reacted with an enzyme in the presence of a coupling agent such as glutaraldehyde or periodate. Conjugates with fluorescein markers are prepared in the presence of these coupling agents or by reaction with an isothiocyanate. In U.S. Pat. No. 4,938,948, imaging of breast tumors is achieved using monoclonal antibodies and the detectable imaging moieties are bound to the antibody using linkers such as methyl-p-hydroxybenzimidate or N-succinimidyl-3-(4-hydroxyphenyl)propionate.

Another known method of site-specific attachment of molecules to antibodies comprises the reaction of antibodies with hapten-based affinity labels. Essentially, hapten-based affinity labels react with amino acids in the antigen binding site, thereby destroying this site and blocking specific antigen reaction. However, this may not be advantageous since it results in loss of antigen binding by the antibody conjugate.

Molecules containing azido groups may also be used to form covalent bonds to proteins through reactive nitrene intermediates that are generated by low intensity ultraviolet light. In particular, 2- and 8-azido analogues of purine nucleotides have been used as site-directed photoprobes to identify nucleotide binding proteins in crude cell extracts. The 2- and 8-azido nucleotides have also been used to map nucleotide binding domains of purified proteins and may be used as antibody binding agents.

Derivatization of immunoglobulins by selectively introducing sulfhydryl groups in the Fc region of an immunoglobulin, using reaction conditions that do not alter the antibody combining site are also contemplated. Antibody conjugates produced according to this methodology are disclosed to exhibit improved longevity, specificity and sensitivity (U.S. Pat. No. 5,196,066). Site-specific attachment of effector or reporter molecules, wherein the reporter or effector molecule is conjugated to a carbohydrate residue in the Fc region have also been disclosed in the literature. This approach has been reported to produce diagnostically and therapeutically promising antibodies which are currently in clinical evaluation.

### E. Antibody Drug Conjugates

Antibody drug conjugates, or ADCs, are a class of highly potent biopharmaceutical drugs designed as a targeted therapy for the treatment of people with disease. ADCs are complex molecules composed of an antibody (a whole mAb or an antibody fragment, such as an scFv) linked, via a stable chemical linker with labile bonds, to a biological active cytotoxic/anti-viral payload or drug. Antibody drug conjugates are examples of bioconjugates and immunoconjugates.

By combining the unique targeting capabilities of monoclonal antibodies with the cancer-killing ability of cytotoxic drugs, antibody-drug conjugates allow sensitive discrimination between healthy and diseased tissue. This means that, in contrast to traditional systemic approaches, antibody-drug conjugates target and attack the diseased cell so that healthy cells are less severely affected.

In the development ADC-based anti-tumor therapies, an anticancer drug (e.g., a cell toxin or cytotoxin) is coupled to an antibody that specifically targets a certain cell marker (e.g., a protein that, ideally, is only to be found in or on diseased cells). Antibodies track these proteins down in the body and attach themselves to the surface of the diseased cells. The biochemical reaction between the antibody and the target protein (antigen) triggers a signal in the targeted cell, which then absorbs or internalizes the antibody together with the cytotoxin. After the ADC is internalized, the cytotoxic drug is released and kills the cell or impairs cellular replication. Due to this targeting, ideally the drug has lower side effects and gives a wider therapeutic window than other agents.

A stable link between the antibody and cytotoxic agent is a crucial aspect of an ADC. Linkers are based on chemical motifs including disulfides, hydrazones or peptides (cleavable), or thioethers (noncleavable) and control the distribution and delivery of the cytotoxic agent to the target cell. Cleavable and non-cleavable types of linkers have been proven to be safe in preclinical and clinical trials. Brentuximab vedotin includes an enzyme-sensitive cleavable linker that delivers the potent and highly toxic anti-microtubule agent Monomethyl auristatin E or MMAE, a synthetic antineoplastic agent, to human specific CD30-positive malignant cells. Because of its high toxicity MMAE, which inhibits cell division by blocking the polymerization of tubulin, cannot be used as a single-agent chemotherapeutic drug. However, the combination of MMAE linked to an anti-CD30 monoclonal antibody (cAC10, a cell membrane protein of the tumor necrosis factor or TNF receptor) proved to be stable in extracellular fluid, cleavable by cathepsin and safe for therapy. Trastuzumab emtansine, the other approved ADC, is a combination of the microtubule-formation inhibitor mertansine (DM-1), a derivative of the Maytansine, and the antibody trastuzumab (Herceptin®/Genentech/Roche) attached by a stable, non-cleavable linker.

The availability of better and more stable linkers has changed the function of the chemical bond. The type of linker, cleavable or noncleavable, lends specific properties to the cytotoxic (e.g., anti-cancer) drug. For example, a non-cleavable linker keeps the drug within the cell. As a result, the entire antibody, linker, and cytotoxic agent enter the targeted cell where the antibody is degraded to the level of amino acids. The resulting complex—amino acid, linker and cytotoxic agent—now becomes the active drug. In contrast, cleavable linkers are catalyzed by enzymes in the host cell, thereby releasing the cytotoxic agent.

Another type of cleavable linker adds an extra molecule between the cytotoxic drug and the cleavage site. This linker technology allows researchers to create ADCs with more flexibility without worrying about changing cleavage kinetics. Researchers are also developing a new method of peptide cleavage based on Edman degradation. Future direction in the development of ADCs also include the development of site-specific conjugation (TDCs) to further improve stability and therapeutic index and α-emitting immunoconjugates and antibody-conjugated nanoparticles. Production and Purification of Antibodies

The methods for generating monoclonal antibodies generally begin along the same lines as those for preparing polyclonal antibodies. The first step for both of these methods is immunization of an appropriate host. As is well known in the art, a given composition for immunization may vary in its immunogenicity. It is often necessary therefore to boost the host immune system, as may be achieved by coupling a peptide or polypeptide immunogen to a carrier. Exemplary and preferred carriers are keyhole limpet hemocyanin (KLH) and bovine serum albumin (BSA). Other albumins such as ovalbumin, mouse serum albumin or rabbit serum albumin can also be used as carriers. Means for conjugating a polypeptide to a carrier protein are well known in the art and include glutaraldehyde, m-maleimidobencoyl-N-hydroxysuccinimide ester, carbodiimyde and bis-biazotized benzidine. As also is well known in the art, the immunogenicity of a particular immunogen composition can be enhanced by the use of non-specific stimulators of the immune response, known as adjuvants. Exemplary and preferred adjuvants in animals include complete Freund's adjuvant (a non-specific stimulator of the immune response containing killed Mycobacterium tuberculosis), incomplete Freund's adjuvants and aluminum hydroxide adjuvant and in humans include alum, CpG, MFP59, and combinations of immunostimulatory molecules (“Adjuvant Systems”, such as AS01 or AS03). Additional experimental forms of inoculation to induce antigen-specific B cells are possible, including nanoparticle vaccines, or gene-encoded antigens delivered as DNA or RNA genes in a physical delivery system (such as lipid nanoparticle or on a gold biolistic bead), and delivered with needle, gene gun, or transcutaneous electroporation device. The antigen gene also can be carried as encoded by a replication competent or defective viral vector such as adenovirus, adeno-associated virus, poxvirus, herpesvirus, or alphavirus replicon, or alternatively a virus-like particle.

Methods for generating hybrids of antibody-producing cells and myeloma cells usually comprise mixing somatic cells with myeloma cells in a 2:1 proportion, though the proportion may vary from about 20:1 to about 1:1, in the presence of an agent or agents (chemical or electrical) that promote the fusion of cell membranes. In some cases, transformation of human B cells with Epstein Barr virus (EBV) as an initial step increases the size of the B cells, enhancing fusion with the relatively large-sized myeloma cells. Transformation efficiency by EBV is enhanced by using CpG and a Chk2 inhibitor drug in the transforming medium. Alternatively, human B cells can be activated by co-culture with transfected cell lines expressing CD40 μLigand (CD154) in medium containing additional soluble factors, such as IL-21 and human B cell Activating Factor (BAFF), a Type II member of the TNF superfamily. Fusion methods using Sendai virus or polyethylene glycol (PEG) are also known. The use of electrically induced fusion methods is also appropriate. Fusion procedures usually produce viable hybrids at low frequencies, about 1×10⁻⁶ to 1×10⁻⁸, but with optimized procedures one can achieve fusion efficiencies close to 1 in 200. However, relatively low efficiency of fusion does not pose a problem, as the viable, fused hybrids are differentiated from the parental, infused cells (particularly the infused myeloma cells that would normally continue to divide indefinitely) by culturing in a selective medium. The selective medium is generally one that contains an agent that blocks the de novo synthesis of nucleotides in the tissue culture medium. Exemplary and preferred agents are aminopterin, methotrexate, and azaserine. Aminopterin and methotrexate block de novo synthesis of both purines and pyrimidines, whereas azaserine blocks only purine synthesis. Where aminopterin or methotrexate is used, the medium is supplemented with hypoxanthine and thymidine as a source of nucleotides (HAT medium). Where azaserine is used, the medium is supplemented with hypoxanthine. Ouabain is added if the B cell source is an EBV-transformed human B cell line, in order to eliminate EBV-transformed lines that have not fused to the myeloma.

The preferred selection medium is HAT or HAT with ouabain. Only cells capable of operating nucleotide salvage pathways are able to survive in HAT medium. The myeloma cells are defective in key enzymes of the salvage pathway, e.g., hypoxanthine phosphoribosyl transferase (HPRT), and they cannot survive. The B cells can operate this pathway, but they have a limited life span in culture and generally die within about two weeks. Therefore, the only cells that can survive in the selective media are those hybrids formed from myeloma and B cells. When the source of B cells used for fusion is a line of EBV-transformed B cells, as here, ouabain may also be used for drug selection of hybrids as EBV-transformed B cells are susceptible to drug killing, whereas the myeloma partner used is chosen to be ouabain resistant.

Culturing provides a population of hybridomas from which specific hybridomas are selected. Typically, selection of hybridomas is performed by culturing the cells by single-clone dilution in microtiter plates, followed by testing the individual clonal supernatants (after about two to three weeks) for the desired reactivity. The assay should be sensitive, simple and rapid, such as radioimmunoassays, enzyme immunoassays, cytotoxicity assays, plaque assays dot immunobinding assays, and the like. The selected hybridomas are then serially diluted or single-cell sorted by flow cytometric sorting and cloned into individual antibody-producing cell lines, which clones can then be propagated indefinitely to provide monoclonal antibodies. The cell lines may be exploited for monoclonal antibody production in two basic ways. A sample of the hybridoma can be injected (often into the peritoneal cavity) into an animal (e.g., a mouse). Optionally, the animals are primed with a hydrocarbon, especially oils such as pristane (tetramethylpentadecane) prior to injection. When human hybridomas are used in this way, it is optimal to inject immunocompromised mice, such as SCID mice, to prevent tumor rejection. The injected animal develops tumors secreting the specific monoclonal antibody produced by the fused cell hybrid. The body fluids of the animal, such as serum or ascites fluid, can then be tapped to provide monoclonal antibodies in high concentration. The individual cell lines could also be cultured in vitro, where the monoclonal antibodies are naturally secreted into the culture medium from which they can be readily obtained in high concentrations. Alternatively, human hybridoma cells lines can be used in vitro to produce immunoglobulins in cell supernatant. The cell lines can be adapted for growth in serum-free medium to optimize the ability to recover human monoclonal immunoglobulins of high purity.

Hybridomas may be cultured, then cells lysed, and total RNA extracted. Random hexamers may be used with RT to generate cDNA copies of RNA, and then PCR performed using a multiplex mixture of PCR primers expected to amplify all human variable gene sequences. PCR product can be cloned into pGEM-T Easy vector, then sequenced by automated DNA sequencing using standard vector primers. Assay of binding and neutralization may be performed using antibodies collected from hybridoma supernatants and purified by FPLC, using Protein G columns.

Recombinant full-length IgG antibodies can be generated by subcloning heavy and light chain Fv DNAs from the cloning vector into an IgG plasmid vector, transfected into 293 (e.g., Freestyle) cells or CHO cells, and antibodies can be collected and purified from the 293 or CHO cell supernatant. Other appropriate host cells systems include bacteria, such as E. coli, insect cells (S2, Sf9, Sf29, High Five), plant cells (e.g., tobacco, with or without engineering for human-like glycans), algae, or in a variety of non-human transgenic contexts, such as mice, rats, goats or cows.

Expression of nucleic acids encoding antibodies, both for the purpose of subsequent antibody purification, and for immunization of a host, is also contemplated. Antibody coding sequences can be RNA, such as native RNA or modified RNA. Modified RNA contemplates certain chemical modifications that confer increased stability and low immunogenicity to mRNAs, thereby facilitating expression of therapeutically important proteins. For instance, N1-methyl-pseudouridine (N1mΨ) outperforms several other nucleoside modifications and their combinations in terms of translation capacity. In addition to turning off the immune/eIF2α phosphorylation-dependent inhibition of translation, incorporated N1mΨ nucleotides dramatically alter the dynamics of the translation process by increasing ribosome pausing and density on the mRNA. Increased ribosome loading of modified mRNAs renders them more permissive for initiation by favoring either ribosome recycling on the same mRNA or de novo ribosome recruitment. Such modifications could be used to enhance antibody expression in vivo following inoculation with RNA. The RNA, whether native or modified, may be delivered as naked RNA or in a delivery vehicle, such as a lipid nanoparticle.

Alternatively, DNA encoding the antibody may be employed for the same purposes. The DNA is included in an expression cassette comprising a promoter active in the host cell for which it is designed. The expression cassette is advantageously included in a replicable vector, such as a conventional plasmid or minivector. Vectors include viral vectors, such as poxviruses, adenoviruses, herpesviruses, adeno-associated viruses, and lentiviruses are contemplated. Replicons encoding antibody genes such as alphavirus replicons based on VEE virus or Sindbis virus are also contemplated. Delivery of such vectors can be performed by needle through intramuscular, subcutaneous, or intradermal routes, or by transcutaneous electroporation when in vivo expression is desired.

Alternatively, a molecular cloning approach may be used to generate monoclonal antibodies. Single B cells labeled with the antigen of interest can be sorted physically using paramagnetic bead selection or flow cytometric sorting, then RNA can be isolated from the single cells and antibody genes amplified by RT-PCR. Alternatively, antigen-specific bulk sorted populations of cells can be segregated into microvesicles and the matched heavy and light chain variable genes recovered from single cells using physical linkage of heavy and light chain amplicons, or common barcoding of heavy and light chain genes from a vesicle. Matched heavy and light chain genes form single cells also can be obtained from populations of antigen specific B cells by treating cells with cell-penetrating nanoparticles bearing RT-PCR primers and barcodes for marking transcripts with one barcode per cell. The antibody variable genes also can be isolated by RNA extraction of a hybridoma line and the antibody genes obtained by RT-PCR and cloned into an immunoglobulin expression vector. Alternatively, combinatorial immunoglobulin phagemid libraries are prepared from RNA isolated from the cell lines and phagemids expressing appropriate antibodies are selected by panning using viral antigens. The advantages of this approach over conventional hybridoma techniques are that approximately 10⁴ times as many antibodies can be produced and screened in a single round, and that new specificities are generated by H and L chain combination which further increases the chance of finding appropriate antibodies.

Other U.S. patents, each incorporated herein by reference, that teach the production of antibodies useful in the present disclosure include U.S. Pat. No. 5,565,332, which describes the production of chimeric antibodies using a combinatorial approach; U.S. Pat. No. 4,816,567 which describes recombinant immunoglobulin preparations; and U.S. Pat. No. 4,867,973 which describes antibody-therapeutic agent conjugates.

Monoclonal antibodies produced by any means may be purified, if desired, using filtration, centrifugation, and various chromatographic methods, such as FPLC or affinity chromatography. Fragments of the monoclonal antibodies of the disclosure can be obtained from the purified monoclonal antibodies by methods that include digestion with enzymes, such as pepsin or papain, and/or by cleavage of disulfide bonds by chemical reduction. Alternatively, monoclonal antibody fragments encompassed by the present disclosure can be synthesized using an automated peptide synthesizer.

The antibodies disclosed herein may be isolated or purified. The terms “isolated” and “purified,” as used herein, is intended to refer to a composition, isolatable from other components, wherein the protein is purified to any degree relative to its naturally-obtainable state. A purified protein therefore also refers to a protein, free from the environment in which it may naturally occur. Where the term “substantially purified” is used, this designation will refer to a composition in which the protein or peptide forms the major component of the composition, such as constituting about 50%, about 60%, about 70%, about 80%, about 90%, about 95% or more of the proteins in the composition.

Protein purification techniques are well known to those of skill in the art. These techniques involve, at one level, the crude fractionation of the cellular milieu to polypeptide and non-polypeptide fractions. Having separated the polypeptide from other proteins, the polypeptide of interest may be further purified using chromatographic and electrophoretic techniques to achieve partial or complete purification (or purification to homogeneity). Analytical methods particularly suited to the preparation of a pure peptide are ion-exchange chromatography, exclusion chromatography; polyacrylamide gel electrophoresis; isoelectric focusing. Other methods for protein purification include, precipitation with ammonium sulfate, PEG, antibodies and the like or by heat denaturation, followed by centrifugation; gel filtration, reverse phase, hydroxyapatite and affinity chromatography; and combinations of such and other techniques.

In purifying an antibody of the present disclosure, it may be desirable to express the polypeptide in a prokaryotic or eukaryotic expression system and extract the protein using denaturing conditions. The polypeptide may be purified from other cellular components using an affinity column, which binds to a tagged portion of the polypeptide. As is generally known in the art, it is believed that the order of conducting the various purification steps may be changed, or that certain steps may be omitted, and still result in a suitable method for the preparation of a substantially purified protein or peptide.

Commonly, complete antibodies are fractionated utilizing agents (i.e., protein A) that bind the Fc portion of the antibody. Alternatively, antigens may be used to simultaneously purify and select appropriate antibodies. Such methods often utilize the selection agent bound to a support, such as a column, filter or bead. The antibodies are bound to a support, contaminants removed (e.g., washed away), and the antibodies released by applying conditions (salt, heat, etc.).

Various methods for quantifying the degree of purification of the protein or peptide will be known to those of skill in the art in light of the present disclosure. These include, for example, determining the specific activity of an active fraction, or assessing the amount of polypeptides within a fraction by SDS/PAGE analysis. Another method for assessing the purity of a fraction is to calculate the specific activity of the fraction, to compare it to the specific activity of the initial extract, and to thus calculate the degree of purity. The actual units used to represent the amount of activity will, of course, be dependent upon the particular assay technique chosen to follow the purification and whether or not the expressed protein or peptide exhibits a detectable activity.

It is known that the migration of a polypeptide can vary, sometimes significantly, with different conditions of SDS/PAGE. It will therefore be appreciated that under differing electrophoresis conditions, the apparent molecular weights of purified or partially purified expression products may vary.

### F. Modification of Antibodies

The sequences of antibodies may be modified for a variety of reasons, such as improved expression, improved cross-reactivity, or diminished off-target binding. Modified antibodies may be made by any technique known to those of skill in the art, including expression through standard molecular biological techniques, or the chemical synthesis of polypeptides.

For example, one may wish to make modifications, such as introducing conservative changes into an antibody molecule. In making such changes, the hydropathic index of amino acids may be considered. The importance of the hydropathic amino acid index in conferring interactive biologic function on a protein is generally understood in the art (Kyte and Doolittle, 1982). It is accepted that the relative hydropathic character of the amino acid contributes to the secondary structure of the resultant protein, which in turn defines the interaction of the protein with other molecules, for example, enzymes, substrates, receptors, DNA, antibodies, antigens, and the like.

The substitution of like amino acids can be made effectively on the basis of hydrophilicity. U.S. Pat. No. 4,554,101, incorporated herein by reference, states that the greatest local average hydrophilicity of a protein, as governed by the hydrophilicity of its adjacent amino acids, correlates with a biological property of the protein. As detailed in U.S. Pat. No. 4,554,101, the following hydrophilicity values have been assigned to amino acid residues: basic amino acids: arginine (+3.0), lysine (+3.0), and histidine (−0.5); acidic amino acids: aspartate (+3.0±1), glutamate (+3.0±1), asparagine (+0.2), and glutamine (+0.2); hydrophilic, nonionic amino acids: serine (+0.3), asparagine (+0.2), glutamine (+0.2), and threonine (−0.4), sulfur containing amino acids: cysteine (−1.0) and methionine (−1.3); hydrophobic, nonaromatic amino acids: valine (−1.5), leucine (−1.8), isoleucine (−1.8), proline (−0.5±1), alanine (−0.5), and glycine (0); hydrophobic, aromatic amino acids: tryptophan (−3.4), phenylalanine (−2.5), and tyrosine (−2.3).

An amino acid can be substituted for another having a similar hydrophilicity and produce a biologically or immunologically modified protein. In such changes, the substitution of amino acids whose hydrophilicity values are within ±2 is preferred, those that are within ±1 are particularly preferred, and those within ±0.5 are even more particularly preferred.

Amino acid substitutions generally are based on the relative similarity of the amino acid side-chain substituents, for example, their hydrophobicity, hydrophilicity, charge, size, and the like. Exemplary substitutions that take into consideration the various foregoing characteristics are well known to those of skill in the art and include: arginine and lysine; glutamate and aspartate; serine and threonine; glutamine and asparagine; and valine, leucine and isoleucine.

The present disclosure also contemplates isotype modification. By modifying the Fc region to have a different isotype, different functionalities can be achieved. For example, changing to IgG₁ can increase antibody dependent cell cytotoxicity, switching to class A can improve tissue distribution, and switching to class M can improve valency.

One can design an Fc region of an antibody with altered effector function, e.g., by modifying C1q binding and/or FcγR binding and thereby changing CDC activity and/or ADCC activity. “Effector functions” are responsible for activating or diminishing a biological activity (e.g., in a subject). Examples of effector functions include, but are not limited to: C1q binding; complement dependent cytotoxicity (CDC); Fc receptor binding; antibody-dependent cell-mediated cytotoxicity (ADCC); phagocytosis; down regulation of cell surface receptors (e.g., B cell receptor; BCR), etc. Such effector functions may require the Fc region to be combined with a binding domain (e.g., an antibody variable domain) and can be assessed using various assays (e.g., Fc binding assays, ADCC assays, CDC assays, etc.).

For example, one can generate a variant Fc region of an antibody with improved C1q binding and improved FcγRIII binding (e.g., having both improved ADCC activity and improved CDC activity). Alternatively, if it is desired that effector function be reduced or ablated, a variant Fc region can be engineered with reduced CDC activity and/or reduced ADCC activity. In other embodiments, only one of these activities may be increased, and, optionally, also the other activity reduced (e.g., to generate an Fc region variant with improved ADCC activity, but reduced CDC activity and vice versa).

In certain embodiments, the Fc domain may incorporate one or more mutations or modifications, in either or both Fc polypeptide chains, that alter the binding to an Fcγ receptor (e.g., FcγRI/CD64, FcγRIIA/CD32A, FcγRIIB/CD32B, FcγRIIIIA/CD16, or FcγRIIIB). In some embodiments, a modified heavy chain constant region comprises a CH2 domain that is a wildtype CH2 domain of the IgG isotype (e.g., IgG1). A CH2 domain as used herein may also be a variant of a wildtype CH2 domain, e.g., a variant of a wildtype IgG1 CH2 domain. Exemplary variants of CH2 domains include variants that modulate a biological activity of the Fc region of an antibody, such as ADCC or CDC, or that modulate the half-life of the antibody/antibody stability. A CH2 domain may have enhanced effector function. CH2 domains may comprise one or more mutations at the following amino acids: E233, G236, G237, P238, H268, P271, L328, A330, and 1332.

In certain embodiments, the antibody can be modified to increase binding to FcγR2a and/or FcγR3a. Exemplary modifications that increase binding to FcγR2a include substitution at G236, e.g., G236A. Exemplary modifications that increase binding to FcγR3a include substitutions at G236, e.g., G236A, at A330, e.g., A330 μL, and at 1332, e.g., 1332E.

An isolated monoclonal antibody, or antigen binding fragment thereof, may contain a substantially homogeneous glycan without sialic acid, galactose, or fucose. The aforementioned substantially homogeneous glycan may be covalently attached to the heavy chain constant region.

A monoclonal antibody may have a novel Fc glycosylation pattern. Glycosylation of an Fc region is typically either N-linked or O-linked. N-linked refers to the attachment of the carbohydrate moiety to the side chain of an asparagine residue. O-linked glycosylation refers to the attachment of one of the sugars N-acetylgalactosamine, galactose, or xylose to a hydroxyamino acid, most commonly serine or threonine, although 5-hydroxyproline or 5-hydroxylysine may also be used. The recognition sequences for enzymatic attachment of the carbohydrate moiety to the asparagine side chain peptide sequences are asparagine-X-serine and asparagine-X-threonine, where X is any amino acid except proline. Thus, the presence of either of these peptide sequences in a polypeptide creates a potential glycosylation site.

The glycosylation pattern may be altered, for example, by deleting one or more glycosylation site(s) found in the polypeptide, and/or adding one or more glycosylation site(s) that are not present in the polypeptide. Addition of glycosylation sites to the Fc region of an antibody is conveniently accomplished by altering the amino acid sequence such that it contains one or more of the above-described tripeptide sequences (for N-linked glycosylation sites). An exemplary glycosylation variant has an amino acid substitution of residue Asn 297 of the heavy chain. The alteration may also be made by the addition of, or substitution by, one or more serine or threonine residues to the sequence of the original polypeptide (for O-linked glycosylation sites). Additionally, a change of Asn 297 to Ala can remove one of the glycosylation sites.

The isolated monoclonal antibody, or antigen binding fragment thereof, may be present in a substantially homogenous composition represented by the GNGN or G1/G2 glycoform, which exhibits increased binding affinity for Fc gamma RI and Fc gamma RIII compared to the same antibody without the substantially homogeneous GNGN glycoform and with G0, G1F, G2F, GNF, GNGNF or GNGNFX containing glycoforms. Fc glycosylation plays a significant role in anti-viral and anti-cancer properties of therapeutic mAbs. Elimination of core fucose dramatically improves the ADCC activity of mAbs mediated by natural killer (NK) cells but appears to have the opposite effect on the ADCC activity of polymorphonuclear cells (PMNs).

The isolated monoclonal antibody, or antigen binding fragment thereof, may be expressed in cells that express beta (1,4)-N-acetylglucosaminyltransferase III (GnT III), such that GnT III adds GlcNAc to the antibody. Methods for producing antibodies in such a fashion are provided in WO/9954342 and WO/03011878. Cell lines can be altered to enhance or reduce or eliminate certain post-translational modifications, such as glycosylation, using genome editing technology such as Clustered Regularly Interspaced Short Palindromic Repeats (CRISPR). For example, CRISPR technology can be used to eliminate genes encoding glycosylating enzymes in 293 or CHO cells used to express monoclonal antibodies.

It is possible to engineer the antibody variable gene sequences obtained from human B cells to enhance their manufacturability and safety. Potential protein sequence liabilities can be identified by searching for sequence motifs associated with sites containing:
1) Unpaired Cys residues,
2) N-linked glycosylation,
3) Asn deamidation,
4) Asp isomerization,
5) SYE truncation,
6) Met oxidation,
7) Trp oxidation,
8) N-terminal glutamate,
9) Integrin binding,
10) CD11c/CD18 binding, or
11) Fragmentation
   Such motifs can be eliminated by altering the synthetic gene comprising the cDNA encoding the antibodies.

Antibodies can be engineered to enhance solubility. For example, some hydrophilic residues such as aspartic acid, glutamic acid, and serine contribute significantly more favorably to protein solubility than other hydrophilic residues, such as asparagine, glutamine, threonine, lysine, and arginine.

B cell repertoire deep sequencing of human B cells from blood donors has been performed on a wide scale. Sequence information about a significant portion of the human antibody repertoire facilitates statistical assessment of antibody sequence features common in healthy humans. With knowledge about the antibody sequence features in a human recombined antibody variable gene reference database, the position specific degree of “Human Likeness” (HL) of an antibody sequence can be estimated. HL has been shown to be useful for the development of antibodies in clinical use, like therapeutic antibodies or antibodies as vaccines. The goal is to increase the human likeness of antibodies to reduce potential adverse effects and anti-antibody immune responses that will lead to significantly decreased efficacy of the antibody drug or can induce serious health implications. One can assess antibody characteristics of the combined antibody repertoire of three healthy human blood donors of about 400 million sequences in total and created a novel “relative Human Likeness” (rHL) score that focuses on the hypervariable region of the antibody. The rHL score allows one to easily distinguish between human (positive score) and non-human sequences (negative score). Antibodies can be engineered to eliminate residues that are not common in human repertoires.

Methods for reducing or eliminating the antigenicity of antibodies and antibody fragments are known in the art. When the antibodies are to be administered to a human, the antibodies preferably are “humanized” to reduce or eliminate antigenicity in humans. Preferably, each humanized antibody has the same or substantially the same affinity for the antigen as the non-humanized mouse antibody from which it was derived.

In one humanization approach, chimeric proteins are created in which mouse immunoglobulin constant regions are replaced with human immunoglobulin constant regions. See, e.g., Morrison et al., 1984, PROC. NAT. ACAD. SCI. 81:6851-6855, Neuberger et al., 1984, NATURE 312:604-608; U.S. Pat. No. 6,893,625 (Robinson); U.S. Pat. No. 5,500,362 (Robinson); and U.S. Pat. No. 4,816,567 (Cabilly).

In an approach known as CDR grafting, the CDRs of the light and heavy chain variable regions are grafted into frameworks from another species. For example, murine CDRs can be grafted into human FRs. In some embodiments, the CDRs of the light and heavy chain variable regions of an antibody are grafted into human FRs or consensus human FRs. To create consensus human FRs, FRs from several human heavy chain or light chain amino acid sequences are aligned to identify a consensus amino acid sequence. CDR grafting is described in U.S. Pat. No. 7,022,500 (Queen); U.S. Pat. No. 6,982,321 (Winter); U.S. Pat. No. 6,180,370 (Queen); U.S. Pat. No. 6,054,297 (Carter); U.S. Pat. No. 5,693,762 (Queen); U.S. Pat. No. 5,859,205 (Adair); U.S. Pat. No. 5,693,761 (Queen); U.S. Pat. No. 5,565,332 (Hoogenboom); U.S. Pat. No. 5,585,089 (Queen); U.S. Pat. No. 5,530,101 (Queen); Jones et al. (1986) NATURE 321: 522-525; Riechmann et al. (1988) NATURE 332: 323-327; Verhoeyen et al. (1988) SCIENCE 239: 1534-1536; and Winter (1998) FEBS LETT 430: 92-94.

In an approach called “SUPERHUMANIZATION™” human CDR sequences are chosen from human germline genes, based on the structural similarity of the human CDRs to those of the mouse antibody to be humanized. See, e.g., U.S. Pat. No. 6,881,557 (Foote); and Tan et al., 2002, J. IMMUNOL. 169:1119-1125.

Other methods to reduce immunogenicity include “reshaping,” “hyperchimerization,” and “veneering/resurfacing.” See, e.g., Vaswami et al., 1998, ANNALS OF ALLERGY, ASTHMA, & IMMUNOL. 81:105; Roguska et al., 1996, PROT. ENGINEER 9:895-904; and U.S. Pat. No. 6,072,035 (Hardman). In the veneering/resurfacing approach, the surface accessible amino acid residues in the murine antibody are replaced by amino acid residues more frequently found at the same positions in a human antibody. This type of antibody resurfacing is described, e.g., in U.S. Pat. No. 5,639,641 (Pedersen).

Another approach for converting a mouse antibody into a form suitable for medical use in humans is known as ACTIVMAB™ technology (Vaccinex, Inc., Rochester, NY), which involves a vaccinia virus-based vector to express antibodies in mammalian cells. High levels of combinatorial diversity of IgG heavy and light chains can be produced. See, e.g., U.S. Pat. No. 6,706,477 (Zauderer); U.S. Pat. No. 6,800,442 (Zauderer); and U.S. Pat. No. 6,872,518 (Zauderer). Another approach for converting a mouse antibody into a form suitable for use in humans is technology practiced commercially by KaloBios Pharmaceuticals, Inc. (Palo Alto, CA). This technology involves the use of a proprietary human “acceptor” library to produce an “epitope focused” library for antibody selection. Another approach for modifying a mouse antibody into a form suitable for medical use in humans is HUMAN ENGINEERINGC™ technology, which is practiced commercially by XOMA (US) LLC. See, e.g., International (PCT) Publication No. WO 93/11794 and U.S. Pat. No. 5,766,886 (Studnicka); U.S. Pat. No. 5,770,196 (Studnicka); U.S. Pat. No. 5,821,123 (Studnicka); and U.S. Pat. No. 5,869,619 (Studnicka).

Any suitable approach, including any of the above approaches, can be used to reduce or eliminate human immunogenicity of an antibody.

### G. Characterization of Antibodies

Antibodies according to the present disclosure may be defined, in the first instance, by their binding specificity. Those of skill in the art, by assessing the binding specificity/affinity of a given antibody using techniques well known to those of skill in the art, can determine whether such antibodies fall within the scope of the instant claims. For example, the epitope to which a given antibody binds may consist of a single contiguous sequence of 3 or more (e.g., 3, 4, 5, 6, 7, 8, 9, 10, 11, 12, 13, 14, 15, 16, 17, 18, 19, 20) amino acids located within the antigen molecule (e.g., a linear epitope in a domain). Alternatively, the epitope may consist of a plurality of non-contiguous amino acids (or amino acid sequences) located within the antigen molecule (e.g., a conformational epitope).

Various techniques known to persons of ordinary skill in the art can be used to determine whether an antibody “interacts with one or more amino acids” within a polypeptide or protein. Exemplary techniques include, for example, routine cross-blocking assays, such as that described in Antibodies, Harlow and Lane (Cold Spring Harbor Press, Cold Spring Harbor, N.Y.). Cross-blocking can be measured in various binding assays such as ELISA, biolayer interferometry, or surface plasmon resonance. Other methods include alanine scanning mutational analysis, peptide blot analysis (Reineke (2004) Methods Mol. Biol. 248: 443-63), peptide cleavage analysis, high-resolution electron microscopy techniques using single particle reconstruction, cryoEM, or tomography, crystallographic studies and NMR analysis. In addition, methods such as epitope excision, epitope extraction and chemical modification of antigens can be employed (Tomer (2000) Prot. Sci. 9: 487-496). Another method that can be used to identify the amino acids within a polypeptide with which an antibody interacts is hydrogen/deuterium exchange detected by mass spectrometry. In general terms, the hydrogen/deuterium exchange method involves deuterium-labeling the protein of interest, followed by binding the antibody to the deuterium-labeled protein. Next, the protein/antibody complex is transferred to water and exchangeable protons within amino acids that are protected by the antibody complex undergo deuterium-to-hydrogen back-exchange at a slower rate than exchangeable protons within amino acids that are not part of the interface. As a result, amino acids that form part of the protein/antibody interface may retain deuterium and therefore exhibit relatively higher mass compared to amino acids not included in the interface. After dissociation of the antibody, the target protein is subjected to protease cleavage and mass spectrometry analysis, thereby revealing the deuterium-labeled residues which correspond to the specific amino acids with which the antibody interacts. See, e.g., Ehring (1999) Analytical Biochemistry 267: 252-259; Engen and Smith (2001) Anal. Chem. 73: 256A-265A.

The term “epitope” refers to a site on an antigen to which B and/or T cells respond. B-cell epitopes can be formed both from contiguous amino acids or noncontiguous amino acids juxtaposed by tertiary folding of a protein. Epitopes formed from contiguous amino acids are typically retained on exposure to denaturing solvents, whereas epitopes formed by tertiary folding are typically lost on treatment with denaturing solvents. An epitope typically includes at least 3, and more usually, at least 5 or 8-10 amino acids in a unique spatial conformation.

Modification-Assisted Profiling (MAP), also known as Antigen Structure-based Antibody Profiling (ASAP) is a method that categorizes large numbers of monoclonal antibodies directed against the same antigen according to the similarities of the binding profile of each antibody to chemically or enzymatically modified antigen surfaces (see US 2004/0101920, herein specifically incorporated by reference in its entirety). Each category may reflect a unique epitope either distinctly different from or partially overlapping with epitope represented by another category. This technology allows rapid filtering of genetically identical antibodies, such that characterization can be focused on genetically distinct antibodies. When applied to hybridoma screening, MAP may facilitate identification of rare hybridoma clones that produce monoclonal antibodies having the desired characteristics. MAP may be used to sort the antibodies of the disclosure into groups of antibodies binding different epitopes.

The present disclosure includes antibodies that may bind to the same epitope, or a portion of the same epitope. One can easily determine whether an antibody binds to the same epitope as, or competes for binding with, a reference antibody by using routine methods known in the art. For example, to determine if a test antibody binds to the same epitope as a reference antibody, the reference antibody is allowed to bind to the target molecule under saturating conditions. Next, the ability of a test antibody to bind to the target molecule is assessed. If the test antibody is able to bind to the target molecule following saturation binding with the reference antibody, it can be concluded that the test antibody binds to a different epitope than the reference antibody. On the other hand, if the test antibody is not able to bind to the target molecule following saturation binding with the reference antibody, then the test antibody may bind to the same epitope as the epitope bound by the reference antibody.

To determine if an antibody competes for binding with, e.g., the 77A antibody, the above-described binding methodology is performed in two orientations: In a first orientation, the 77A antibody is allowed to bind to an HSP70 protein under saturating conditions followed by assessment of binding of the test antibody to the HSP70 protein. In a second orientation, the test antibody is allowed to bind to an HSP70 protein under saturating conditions followed by assessment of binding of the 77A antibody to the HSP70 protein. If, in both orientations, only the first (saturating) antibody is capable of binding to the HSP70 molecule, then it is concluded that the test antibody and the 77A antibody compete for binding to HSP70. As will be appreciated by a person of ordinary skill in the art, an antibody that competes for binding with a reference antibody may not necessarily bind to the identical epitope as the reference antibody, but may sterically block binding of the reference antibody by binding an overlapping or adjacent epitope.

Two antibodies bind to the same or overlapping epitope if each competitively inhibits (blocks) binding of the other to the antigen. That is, a 1-, 5-, 10-, 20- or 100-fold excess of one antibody inhibits binding of the other by at least 50% but preferably 75%, 90%, or even 99% as measured in a competitive binding assay (see, e.g., Junghans et al., Cancer Res. 1990 50:1495-1502). Alternatively, two antibodies have the same epitope if essentially all amino acid mutations in the antigen that reduce or eliminate binding of one antibody reduce or eliminate binding of the other. Two antibodies have overlapping epitopes if some amino acid mutations that reduce or eliminate binding of one antibody reduce or eliminate binding of the other.

Additional routine experimentation (e.g., peptide mutation and binding analyses) can then be carried out to confirm whether the observed lack of binding of the test antibody is in fact due to binding to the same epitope as the reference antibody or if steric blocking (or another phenomenon) is responsible for the lack of observed binding. Experiments of this sort can be performed using ELISA, RIA, surface plasmon resonance, flow cytometry or any other quantitative or qualitative antibody-binding assay available in the art.

In another aspect, the antibodies may be defined by their variable sequence, which include additional “framework” regions. These are provided in Tables 2, 3, 6, 9, and 10, that represent full variable regions. Furthermore, the antibodies sequences may vary from these sequences, optionally using methods discussed in greater detail below. For example, nucleic acid sequences may vary from those set out above in that (a) the variable regions may be segregated away from the constant domains of the light and heavy chains, (b) the nucleic acids may vary from those set out above while not affecting the residues encoded thereby, (c) the nucleic acids may vary from those set out above by a given percentage, e.g., 70%, 75%, 80%, 85%, 90%, 91%, 92%, 93%, 94%, 95%, 96%, 97%, 98% or 99% homology, (d) the nucleic acids may vary from those set out above by virtue of the ability to hybridize under high stringency conditions, as exemplified by low salt and/or high temperature conditions, such as provided by about 0.02 M to about 0.15 M NaCl at temperatures of about 50° C. to about 70° C., (e) the amino acids may vary from those set out above by a given percentage, e.g., 80%, 85%, 90%, 91%, 92%, 93%, 94%, 95%, 96%, 97%, 98% or 99% homology, or (f) the amino acids may vary from those set out above by permitting conservative substitutions.

When comparing polynucleotide and polypeptide sequences, two sequences are said to be “identical” if the sequence of nucleotides or amino acids in the two sequences is the same when aligned for maximum correspondence, as described below. Comparisons between two sequences are typically performed by comparing the sequences over a comparison window to identify and compare local regions of sequence similarity. A “comparison window” as used herein, refers to a segment of at least about 20 contiguous positions, usually 30 to about 75, 40 to about 50, in which a sequence may be compared to a reference sequence of the same number of contiguous positions after the two sequences are optimally aligned.

Optimal alignment of sequences for comparison may be conducted using the Megalign program in the Lasergene suite of bioinformatics software (DNASTAR, Inc., Madison, Wis.), using default parameters. Alternatively, optimal alignment of sequences for comparison may be conducted by the local identity algorithm of Smith and Waterman (1981) Add. APL. Math 2:482, by the identity alignment algorithm of Needleman and Wunsch (1970) J. Mol. Biol. 48:443, by the search for similarity methods of Pearson and Lipman (1988) Proc. Natl. Acad. Sci. USA 85: 2444, by computerized implementations of these algorithms (GAP, BESTFIT, BLAST, FASTA, and TFASTA in the Wisconsin Genetics Software Package, Genetics Computer Group (GCG), 575 Science Dr., Madison, Wis.), or by inspection.

One particular example of algorithms that are suitable for determining percent sequence identity and sequence similarity are the BLAST and BLAST 2.0 algorithms, which are described in Altschul et al. (1977) Nucl. Acids Res. 25:3389-3402 and Altschul et al. (1990) J. Mol. Biol. 215:403-410, respectively. BLAST and BLAST 2.0 can be used, for example with the parameters described herein, to determine percent sequence identity for the polynucleotides and polypeptides of the disclosure. Software for performing BLAST analyses is publicly available through the National Center for Biotechnology Information. The rearranged nature of an antibody sequence and the variable length of each gene requires multiple rounds of BLAST searches for a single antibody sequence. Also, manual assembly of different genes is difficult and error-prone. The sequence analysis tool IgBLAST (world-wide-web at ncbi.nlm.nih.gov/igblast/) identifies matches to the germline V, D and J genes, details at rearrangement junctions, the delineation of Ig V domain framework regions and complementarity determining regions. IgBLAST can analyze nucleotide or protein sequences and can process sequences in batches and allows searches against the germline gene databases and other sequence databases simultaneously to minimize the chance of missing possibly the best matching germline V gene.

In one approach, the “percentage of sequence identity” is determined by comparing two optimally aligned sequences over a window of comparison of at least 20 positions, wherein the portion of the polynucleotide or polypeptide sequence in the comparison window may comprise additions or deletions (i.e., gaps) of 20 percent or less, usually 5 to 15 percent, or 10 to 12 percent, as compared to the reference sequences (which does not comprise additions or deletions) for optimal alignment of the two sequences. The percentage is calculated by determining the number of positions at which the identical nucleic acid bases or amino acid residues occur in both sequences to yield the number of matched positions, dividing the number of matched positions by the total number of positions in the reference sequence (i.e., the window size) and multiplying the results by 100 to yield the percentage of sequence identity.

Yet another way of defining an antibody is as a “derivative” of any of the antibodies provided herein and their antigen-binding fragments. A derivative antibody or antibody fragment may be modified by chemical modifications using techniques known to those of skill in the art, including, but not limited to, specific chemical cleavage, acetylation, formulation, metabolic synthesis of tunicamycin, etc. In one embodiment, an antibody derivative will possess a similar or identical function as the parental antibody. In another embodiment, an antibody derivative will exhibit an altered activity relative to the parental antibody. For example, a derivative antibody (or fragment thereof) can bind to its epitope more tightly or be more resistant to proteolysis than the parental antibody.

The term “derivative” refers to an antibody or antigen-binding fragment thereof that immunospecifically binds to an antigen but which comprises, one, two, three, four, five or more amino acid substitutions, additions, deletions or modifications relative to a “parental” (or wild-type) molecule. Such amino acid substitutions or additions may introduce naturally occurring (i.e., DNA-encoded) or non-naturally occurring amino acid residues. The term “derivative” encompasses, for example, as variants having altered CH1, hinge, CH2, CH3 or CH4 regions, so as to form, for example antibodies, etc., having variant Fc regions that exhibit enhanced or impaired effector or binding characteristics. The term “derivative” additionally encompasses non-amino acid modifications, for example, amino acids that may be glycosylated (e.g., have altered mannose, 2-N-acetylglucosamine, galactose, fucose, glucose, sialic acid, 5-N-acetylneuraminic acid, 5-glycolneuraminic acid, etc. content), acetylated, pegylated, phosphorylated, amidated, derivatized by known protecting/blocking groups, proteolytic cleavage, linked to a cellular ligand or other protein, etc. In some embodiments, the altered carbohydrate modifications modulate one or more of the following: solubilization of the antibody, facilitation of subcellular transport and secretion of the antibody, promotion of antibody assembly, conformational integrity, and antibody-mediated effector function. In a specific embodiment, the altered carbohydrate modifications enhance antibody mediated effector function relative to the antibody lacking the carbohydrate modification. Carbohydrate modifications that lead to altered antibody mediated effector function are well known in the art.

One can determine the biophysical properties of antibodies. One can use elevated temperature to unfold antibodies to determine relative stability, using average apparent melting temperatures. Differential Scanning Calorimetry (DSC) measures the heat capacity, Cₚ, of a molecule (the heat required to warm it, per degree) as a function of temperature. One can use DSC to study the thermal stability of antibodies. DSC data for mAbs is particularly interesting because it sometimes resolves the unfolding of individual domains within the mAb structure, producing up to three peaks in the thermogram (from unfolding of the Fab, C_{H}2, and C_{H}3 domains). Typically unfolding of the Fab domain produces the strongest peak. The DSC profiles and relative stability of the Fc portion show characteristic differences for the human IgG₁, IgG₂, IgG₃, and IgG₄ subclasses (Garber and Demarest, Biochem. Biophys. Res. Commun. 355, 751-757, 2007). One also can determine average apparent melting temperature using circular dichroism (CD), performed with a CD spectrometer. Far-UV CD spectra will be measured for antibodies in the range of 200 to 260 nm at increments of 0.5 nm. The final spectra can be determined as averages of 20 accumulations. Residue ellipticity values can be calculated after background subtraction. Thermal unfolding of antibodies (0.1 mg/mL) can be monitored at 235 nm from 25-95° C. and a heating rate of 1° C./min. One can use dynamic light scattering (DLS) to assess for propensity for aggregation. DLS is used to characterize size of various particles including proteins. If the system is not disperse in size, the mean effective diameter of the particles can be determined. This measurement depends on the size of the particle core, the size of surface structures, and particle concentration. Since DLS essentially measures fluctuations in scattered light intensity due to particles, the diffusion coefficient of the particles can be determined. DLS software in commercial DLA instruments displays the particle population at different diameters. Stability studies can be done conveniently using DLS. DLS measurements of a sample can show whether the particles aggregate over time or with temperature variation by determining whether the hydrodynamic radius of the particle increases. If particles aggregate, one can see a larger population of particles with a larger radius. Stability depending on temperature can be analyzed by controlling the temperature in situ. Capillary electrophoresis (CE) techniques include proven methodologies for determining features of antibody stability. One can use an iCE approach to resolve antibody protein charge variants due to deamidation, C-terminal lysines, sialylation, oxidation, glycosylation, and any other change to the protein that can result in a change in pI of the protein. Each of the expressed antibody proteins can be evaluated by high throughput, free solution isoelectric focusing (IEF) in a capillary column (cIEF), using a Protein Simple Maurice instrument. Whole-column UV absorption detection can be performed every 30 seconds for real time monitoring of molecules focusing at the isoelectric points (pIs). This approach combines the high resolution of traditional gel IEF with the advantages of quantitation and automation found in column-based separations while eliminating the need for a mobilization step. The technique yields reproducible, quantitative analysis of identity, purity, and heterogeneity profiles for the expressed antibodies. The results identify charge heterogeneity and molecular sizing on the antibodies, with both absorbance and native fluorescence detection modes and with sensitivity of detection down to 0.7 μg/mL.

One can determine the intrinsic solubility score of antibody sequences. The intrinsic solubility scores can be calculated using CamSol Intrinsic (Sormanni et al., J Mol Biol 427, 478-490, 2015). The amino acid sequences for residues 95-102 (Kabat numbering) in HCDR3 of each antibody fragment such as a scFv can be evaluated via the online program to calculate the solubility scores. One also can determine solubility using laboratory techniques. Various techniques exist, including addition of lyophilized protein to a solution until the solution becomes saturated and the solubility limit is reached, or concentration by ultrafiltration in a microconcentrator with a suitable molecular weight cut-off. The most straightforward method is induction of amorphous precipitation, which measures protein solubility using a method involving protein precipitation using ammonium sulfate (Trevino et al., J Mol Biol, 366: 449-460, 2007). Ammonium sulfate precipitation gives quick and accurate information on relative solubility values. Ammonium sulfate precipitation produces precipitated solutions with well-defined aqueous and solid phases and requires relatively small amounts of protein. Solubility measurements performed using induction of amorphous precipitation by ammonium sulfate also can be done easily at different pH values. Protein solubility is highly pH dependent, and pH is considered the most important extrinsic factor that affects solubility.

### H. Specific Embodiments

In one embodiment, provided herein are monoclonal antibodies or antibody fragments comprising a heavy chain variable region (VH) comprising a VHCDR1 amino acid sequence of GYX₁FTX₂YG (SEQ ID NO: 214), wherein X₁ is T, S, or I, and X₂ is N or K, a VHCDR2 amino acid sequence of INTYTGEX₁(SEQ ID NO: 215), wherein X₁ is P, S, T, or A, and a VHCDR3 amino acid sequence of X₁RYDHX₂MDY (SEQ ID NO: 216), wherein X₁ is A, T, V, or G, and X₂ is A, R, F, T, P, V, S, D, N, H, L, Y, or G; and/or a light chain variable region (VL) comprising a VLCDR1 amino acid sequence of QSLX₁NSGTRKNY (SEQ ID NO: 212), wherein X₁ is L, F, or V, a VLCDR2 amino acid sequence of SEQ ID NO: 5, and a VLCDR3 amino acid sequence of KQSYX₁LYT (SEQ ID NO: 213), wherein X₁ is T, N, or S.

In one embodiment, provided herein are antibodies or antibody fragments, wherein the antibodies or antibody fragments comprise a heavy chain variable region (VH) comprising a VHCDR1 amino acid sequence selected from the group consisting of SEQ ID NOs: 1 and 164-166, a VHCDR2 amino acid sequence selected from the group consisting of SEQ ID NOs: 2 and 167-169, and a VHCDR3 amino acid sequence selected from the group consisting of SEQ ID NOs: 3 and 170-185; and/or a light chain variable region (VL) comprising a VLCDR1 amino acid sequence selected from the group consisting of SEQ ID NOs: 4 and 159-161, a VLCDR2 amino acid sequence of SEQ ID NO: 5, and a VLCDR3 amino acid sequence selected from the group consisting of SEQ ID NOs: 6, 162, and 163.

In one embodiment, provided herein are antibodies or antibody fragments, wherein the antibodies or antibody fragments comprise:
(i) a heavy chain variable region (VH) comprising a VHCDR1 amino acid sequence of SEQ ID NO: 1, a VHCDR2 amino acid sequence of SEQ ID NO: 2, and a VHCDR3 amino acid sequence of SEQ ID NO: 3; and/or a light chain variable region (VL) comprising a VLCDR1 amino acid sequence of SEQ ID NO: 4, a VLCDR2 amino acid sequence of SEQ ID NO: 5, and a VLCDR3 amino acid sequence of SEQ ID NO: 6;
(ii) a heavy chain variable region (VH) comprising a VHCDR1 amino acid sequence of SEQ ID NO: 164, a VHCDR2 amino acid sequence of SEQ ID NO: 2, and a VHCDR3 amino acid sequence of SEQ ID NO: 3; and/or a light chain variable region (VL) comprising a VLCDR1 amino acid sequence of SEQ ID NO: 4, a VLCDR2 amino acid sequence of SEQ ID NO: 5, and a VLCDR3 amino acid sequence of SEQ ID NO: 6;
(iii) a heavy chain variable region (VH) comprising a VHCDR1 amino acid sequence of SEQ ID NO: 1, a VHCDR2 amino acid sequence of SEQ ID NO: 2, and a VHCDR3 amino acid sequence of SEQ ID NO: 170; and/or a light chain variable region (VL) comprising a VLCDR1 amino acid sequence of SEQ ID NO: 4, a VLCDR2 amino acid sequence of SEQ ID NO: 5, and a VLCDR3 amino acid sequence of SEQ ID NO: 6;
(iv) a heavy chain variable region (VH) comprising a VHCDR1 amino acid sequence of SEQ ID NO: 1, a VHCDR2 amino acid sequence of SEQ ID NO: 2, and a VHCDR3 amino acid sequence of SEQ ID NO: 3; and/or a light chain variable region (VL) comprising a VLCDR1 amino acid sequence of SEQ ID NO: 4, a VLCDR2 amino acid sequence of SEQ ID NO: 5, and a VLCDR3 amino acid sequence of SEQ ID NO: 162;
(v) a heavy chain variable region (VH) comprising a VHCDR1 amino acid sequence of SEQ ID NO: 1, a VHCDR2 amino acid sequence of SEQ ID NO: 2, and a VHCDR3 amino acid sequence of SEQ ID NO: 171; and/or a light chain variable region (VL) comprising a VLCDR1 amino acid sequence of SEQ ID NO: 4, a VLCDR2 amino acid sequence of SEQ ID NO: 5, and a VLCDR3 amino acid sequence of SEQ ID NO: 6;
(vi) a heavy chain variable region (VH) comprising a VHCDR1 amino acid sequence of SEQ ID NO: 1, a VHCDR2 amino acid sequence of SEQ ID NO: 2, and a VHCDR3 amino acid sequence of SEQ ID NO: 172; and/or a light chain variable region (VL) comprising a VLCDR1 amino acid sequence of SEQ ID NO: 4, a VLCDR2 amino acid sequence of SEQ ID NO: 5, and a VLCDR3 amino acid sequence of SEQ ID NO: 6;
(vii) a heavy chain variable region (VH) comprising a VHCDR1 amino acid sequence of SEQ ID NO: 1, a VHCDR2 amino acid sequence of SEQ ID NO: 2, and a VHCDR3 amino acid sequence of SEQ ID NO: 3; and/or a light chain variable region (VL) comprising a VLCDR1 amino acid sequence of SEQ ID NO: 159, a VLCDR2 amino acid sequence of SEQ ID NO: 5, and a VLCDR3 amino acid sequence of SEQ ID NO: 6;
(viii) a heavy chain variable region (VH) comprising a VHCDR1 amino acid sequence of SEQ ID NO: 1, a VHCDR2 amino acid sequence of SEQ ID NO: 2, and a VHCDR3 amino acid sequence of SEQ ID NO: 173; and/or a light chain variable region (VL) comprising a VLCDR1 amino acid sequence of SEQ ID NO: 4, a VLCDR2 amino acid sequence of SEQ ID NO: 5, and a VLCDR3 amino acid sequence of SEQ ID NO: 6;
(ix) a heavy chain variable region (VH) comprising a VHCDR1 amino acid sequence of SEQ ID NO: 1, a VHCDR2 amino acid sequence of SEQ ID NO: 2, and a VHCDR3 amino acid sequence of SEQ ID NO: 174; and/or a light chain variable region (VL) comprising a VLCDR1 amino acid sequence of SEQ ID NO: 4, a VLCDR2 amino acid sequence of SEQ ID NO: 5, and a VLCDR3 amino acid sequence of SEQ ID NO: 6;
(x) a heavy chain variable region (VH) comprising a VHCDR1 amino acid sequence of SEQ ID NO: 164, a VHCDR2 amino acid sequence of SEQ ID NO: 2, and a VHCDR3 amino acid sequence of SEQ ID NO: 175; and/or a light chain variable region (VL) comprising a VLCDR1 amino acid sequence of SEQ ID NO: 4, a VLCDR2 amino acid sequence of SEQ ID NO: 5, and a VLCDR3 amino acid sequence of SEQ ID NO: 6;
(xi) a heavy chain variable region (VH) comprising a VHCDR1 amino acid sequence of SEQ ID NO: 1, a VHCDR2 amino acid sequence of SEQ ID NO: 2, and a VHCDR3 amino acid sequence of SEQ ID NO: 3; and/or a light chain variable region (VL) comprising a VLCDR1 amino acid sequence of SEQ ID NO: 4, a VLCDR2 amino acid sequence of SEQ ID NO: 5, and a VLCDR3 amino acid sequence of SEQ ID NO: 163;
(xii) a heavy chain variable region (VH) comprising a VHCDR1 amino acid sequence of SEQ ID NO: 1, a VHCDR2 amino acid sequence of SEQ ID NO: 2, and a VHCDR3 amino acid sequence of SEQ ID NO: 176; and/or a light chain variable region (VL) comprising a VLCDR1 amino acid sequence of SEQ ID NO: 4, a VLCDR2 amino acid sequence of SEQ ID NO: 5, and a VLCDR3 amino acid sequence of SEQ ID NO: 6;
(xiii) a heavy chain variable region (VH) comprising a VHCDR1 amino acid sequence of SEQ ID NO: 1, a VHCDR2 amino acid sequence of SEQ ID NO: 2, and a VHCDR3 amino acid sequence of SEQ ID NO: 171; and/or a light chain variable region (VL) comprising a VLCDR1 amino acid sequence of SEQ ID NO: 159, a VLCDR2 amino acid sequence of SEQ ID NO: 5, and a VLCDR3 amino acid sequence of SEQ ID NO: 6;
(xiv) a heavy chain variable region (VH) comprising a VHCDR1 amino acid sequence of SEQ ID NO: 1, a VHCDR2 amino acid sequence of SEQ ID NO: 2, and a VHCDR3 amino acid sequence of SEQ ID NO: 177; and/or a light chain variable region (VL) comprising a VLCDR1 amino acid sequence of SEQ ID NO: 4, a VLCDR2 amino acid sequence of SEQ ID NO: 5, and a VLCDR3 amino acid sequence of SEQ ID NO: 6;
(xv) a heavy chain variable region (VH) comprising a VHCDR1 amino acid sequence of SEQ ID NO: 164, a VHCDR2 amino acid sequence of SEQ ID NO: 2, and a VHCDR3 amino acid sequence of SEQ ID NO: 178; and/or a light chain variable region (VL) comprising a VLCDR1 amino acid sequence of SEQ ID NO: 4, a VLCDR2 amino acid sequence of SEQ ID NO: 5, and a VLCDR3 amino acid sequence of SEQ ID NO: 6;
(xvi) a heavy chain variable region (VH) comprising a VHCDR1 amino acid sequence of SEQ ID NO: 1, a VHCDR2 amino acid sequence of SEQ ID NO: 2, and a VHCDR3 amino acid sequence of SEQ ID NO: 179; and/or a light chain variable region (VL) comprising a VLCDR1 amino acid sequence of SEQ ID NO: 4, a VLCDR2 amino acid sequence of SEQ ID NO: 5, and a VLCDR3 amino acid sequence of SEQ ID NO: 6;
(xvii) a heavy chain variable region (VH) comprising a VHCDR1 amino acid sequence of SEQ ID NO: 1, a VHCDR2 amino acid sequence of SEQ ID NO: 2, and a VHCDR3 amino acid sequence of SEQ ID NO: 179; and/or a light chain variable region (VL) comprising a VLCDR1 amino acid sequence of SEQ ID NO: 159, a VLCDR2 amino acid sequence of SEQ ID NO: 5, and a VLCDR3 amino acid sequence of SEQ ID NO: 6;
(xviii) a heavy chain variable region (VH) comprising a VHCDR1 amino acid sequence of SEQ ID NO: 164, a VHCDR2 amino acid sequence of SEQ ID NO: 167, and a VHCDR3 amino acid sequence of SEQ ID NO: 174; and/or a light chain variable region (VL) comprising a VLCDR1 amino acid sequence of SEQ ID NO: 4, a VLCDR2 amino acid sequence of SEQ ID NO: 5, and a VLCDR3 amino acid sequence of SEQ ID NO: 6;
(xix) a heavy chain variable region (VH) comprising a VHCDR1 amino acid sequence of SEQ ID NO: 1, a VHCDR2 amino acid sequence of SEQ ID NO: 2, and a VHCDR3 amino acid sequence of SEQ ID NO: 180; and/or a light chain variable region (VL) comprising a VLCDR1 amino acid sequence of SEQ ID NO: 4, a VLCDR2 amino acid sequence of SEQ ID NO: 5, and a VLCDR3 amino acid sequence of SEQ ID NO: 6;
(xx) a heavy chain variable region (VH) comprising a VHCDR1 amino acid sequence of SEQ ID NO: 1, a VHCDR2 amino acid sequence of SEQ ID NO: 2, and a VHCDR3 amino acid sequence of SEQ ID NO: 181; and/or a light chain variable region (VL) comprising a VLCDR1 amino acid sequence of SEQ ID NO: 4, a VLCDR2 amino acid sequence of SEQ ID NO: 5, and a VLCDR3 amino acid sequence of SEQ ID NO: 162;
(xxi) a heavy chain variable region (VH) comprising a VHCDR1 amino acid sequence of SEQ ID NO: 1, a VHCDR2 amino acid sequence of SEQ ID NO: 2, and a VHCDR3 amino acid sequence of SEQ ID NO: 182; and/or a light chain variable region (VL) comprising a VLCDR1 amino acid sequence of SEQ ID NO: 4, a VLCDR2 amino acid sequence of SEQ ID NO: 5, and a VLCDR3 amino acid sequence of SEQ ID NO: 6;
(xxii) a heavy chain variable region (VH) comprising a VHCDR1 amino acid sequence of SEQ ID NO: 1, a VHCDR2 amino acid sequence of SEQ ID NO: 2, and a VHCDR3 amino acid sequence of SEQ ID NO: 183; and/or a light chain variable region (VL) comprising a VLCDR1 amino acid sequence of SEQ ID NO: 4, a VLCDR2 amino acid sequence of SEQ ID NO: 5, and a VLCDR3 amino acid sequence of SEQ ID NO: 6;
(xxiii) a heavy chain variable region (VH) comprising a VHCDR1 amino acid sequence of SEQ ID NO: 1, a VHCDR2 amino acid sequence of SEQ ID NO: 2, and a VHCDR3 amino acid sequence of SEQ ID NO: 181; and/or a light chain variable region (VL) comprising a VLCDR1 amino acid sequence of SEQ ID NO: 4, a VLCDR2 amino acid sequence of SEQ ID NO: 5, and a VLCDR3 amino acid sequence of SEQ ID NO: 6;
(xxiv) a heavy chain variable region (VH) comprising a VHCDR1 amino acid sequence of SEQ ID NO: 1, a VHCDR2 amino acid sequence of SEQ ID NO: 168, and a VHCDR3 amino acid sequence of SEQ ID NO: 184; and/or a light chain variable region (VL) comprising a VLCDR1 amino acid sequence of SEQ ID NO: 4, a VLCDR2 amino acid sequence of SEQ ID NO: 5, and a VLCDR3 amino acid sequence of SEQ ID NO: 6;
(xxv) a heavy chain variable region (VH) comprising a VHCDR1 amino acid sequence of SEQ ID NO: 165, a VHCDR2 amino acid sequence of SEQ ID NO: 2, and a VHCDR3 amino acid sequence of SEQ ID NO: 185; and/or a light chain variable region (VL) comprising a VLCDR1 amino acid sequence of SEQ ID NO: 4, a VLCDR2 amino acid sequence of SEQ ID NO: 5, and a VLCDR3 amino acid sequence of SEQ ID NO: 6;
(xxvi) a heavy chain variable region (VH) comprising a VHCDR1 amino acid sequence of SEQ ID NO: 166 a VHCDR2 amino acid sequence of SEQ ID NO: 2, and a VHCDR3 amino acid sequence of SEQ ID NO: 170; and/or a light chain variable region (VL) comprising a VLCDR1 amino acid sequence of SEQ ID NO: 4, a VLCDR2 amino acid sequence of SEQ ID NO: 5, and a VLCDR3 amino acid sequence of SEQ ID NO: 6;
(xvii) a heavy chain variable region (VH) comprising a VHCDR1 amino acid sequence of SEQ ID NO: 164, a VHCDR2 amino acid sequence of SEQ ID NO: 2, and a VHCDR3 amino acid sequence of SEQ ID NO: 170; and/or a light chain variable region (VL) comprising a VLCDR1 amino acid sequence of SEQ ID NO: 4, a VLCDR2 amino acid sequence of SEQ ID NO: 5, and a VLCDR3 amino acid sequence of SEQ ID NO: 6;
(xxviii) a heavy chain variable region (VH) comprising a VHCDR1 amino acid sequence of SEQ ID NO: 165, a VHCDR2 amino acid sequence of SEQ ID NO: 2, and a VHCDR3 amino acid sequence of SEQ ID NO: 3; and/or a light chain variable region (VL) comprising a VLCDR1 amino acid sequence of SEQ ID NO: 4, a VLCDR2 amino acid sequence of SEQ ID NO: 5, and a VLCDR3 amino acid sequence of SEQ ID NO: 6;
(xxix) a heavy chain variable region (VH) comprising a VHCDR1 amino acid sequence of SEQ ID NO: 1, a VHCDR2 amino acid sequence of SEQ ID NO: 169, and a VHCDR3 amino acid sequence of SEQ ID NO: 174; and/or a light chain variable region (VL) comprising a VLCDR1 amino acid sequence of SEQ ID NO: 160, a VLCDR2 amino acid sequence of SEQ ID NO: 5, and a VLCDR3 amino acid sequence of SEQ ID NO: 6;
(xxx) a heavy chain variable region (VH) comprising a VHCDR1 amino acid sequence of SEQ ID NO: 1, a VHCDR2 amino acid sequence of SEQ ID NO: 2, and a VHCDR3 amino acid sequence of SEQ ID NO: 184; and/or a light chain variable region (VL) comprising a VLCDR1 amino acid sequence of SEQ ID NO: 4, a VLCDR2 amino acid sequence of SEQ ID NO: 5, and a VLCDR3 amino acid sequence of SEQ ID NO: 6;
(xxxi) a heavy chain variable region (VH) comprising a VHCDR1 amino acid sequence of SEQ ID NO: 1, a VHCDR2 amino acid sequence of SEQ ID NO: 2, and a VHCDR3 amino acid sequence of SEQ ID NO: 175; and/or a light chain variable region (VL) comprising a VLCDR1 amino acid sequence of SEQ ID NO: 4, a VLCDR2 amino acid sequence of SEQ ID NO: 5, and a VLCDR3 amino acid sequence of SEQ ID NO: 6;
(xxxii) a heavy chain variable region (VH) comprising a VHCDR1 amino acid sequence of SEQ ID NO: 1, a VHCDR2 amino acid sequence of SEQ ID NO: 167, and a VHCDR3 amino acid sequence of SEQ ID NO: 3; and/or a light chain variable region (VL) comprising a VLCDR1 amino acid sequence of SEQ ID NO: 4, a VLCDR2 amino acid sequence of SEQ ID NO: 5, and a VLCDR3 amino acid sequence of SEQ ID NO: 6;
(xxxiii) a heavy chain variable region (VH) comprising a VHCDR1 amino acid sequence of SEQ ID NO: 1, a VHCDR2 amino acid sequence of SEQ ID NO: 169, and a VHCDR3 amino acid sequence of SEQ ID NO: 3; and/or a light chain variable region (VL) comprising a VLCDR1 amino acid sequence of SEQ ID NO: 4, a VLCDR2 amino acid sequence of SEQ ID NO: 5, and a VLCDR3 amino acid sequence of SEQ ID NO: 6;
(xxxiv) a heavy chain variable region (VH) comprising a VHCDR1 amino acid sequence of SEQ ID NO: 166, a VHCDR2 amino acid sequence of SEQ ID NO: 167, and a VHCDR3 amino acid sequence of SEQ ID NO: 3; and/or a light chain variable region (VL) comprising a VLCDR1 amino acid sequence of SEQ ID NO: 4, a VLCDR2 amino acid sequence of SEQ ID NO: 5, and a VLCDR3 amino acid sequence of SEQ ID NO: 6;
(xxxv) a heavy chain variable region (VH) comprising a VHCDR1 amino acid sequence of SEQ ID NO: 1, a VHCDR2 amino acid sequence of SEQ ID NO: 168, and a VHCDR3 amino acid sequence of SEQ ID NO: 3; and/or a light chain variable region (VL) comprising a VLCDR1 amino acid sequence of SEQ ID NO: 4, a VLCDR2 amino acid sequence of SEQ ID NO: 5, and a VLCDR3 amino acid sequence of SEQ ID NO: 6;
(xxxvi) a heavy chain variable region (VH) comprising a VHCDR1 amino acid sequence of SEQ ID NO: 166, a VHCDR2 amino acid sequence of SEQ ID NO: 168, and a VHCDR3 amino acid sequence of SEQ ID NO: 3; and/or a light chain variable region (VL) comprising a VLCDR1 amino acid sequence of SEQ ID NO: 4, a VLCDR2 amino acid sequence of SEQ ID NO: 5, and a VLCDR3 amino acid sequence of SEQ ID NO: 6;
(xxxvii) a heavy chain variable region (VH) comprising a VHCDR1 amino acid sequence of SEQ ID NO: 1, a VHCDR2 amino acid sequence of SEQ ID NO: 2, and a VHCDR3 amino acid sequence of SEQ ID NO: 3; and/or a light chain variable region (VL) comprising a VLCDR1 amino acid sequence of SEQ ID NO: 161, a VLCDR2 amino acid sequence of SEQ ID NO: 5, and a VLCDR3 amino acid sequence of SEQ ID NO: 6;
(xxxviii) a heavy chain variable region (VH) comprising a VHCDR1 amino acid sequence of SEQ ID NO: 1, a VHCDR2 amino acid sequence of SEQ ID NO: 168, and a VHCDR3 amino acid sequence of SEQ ID NO: 3; and/or a light chain variable region (VL) comprising a VLCDR1 amino acid sequence of SEQ ID NO: 161, a VLCDR2 amino acid sequence of SEQ ID NO: 5, and a VLCDR3 amino acid sequence of SEQ ID NO: 6;
(xxxix) a heavy chain variable region (VH) comprising a VHCDR1 amino acid sequence of SEQ ID NO: 166, a VHCDR2 amino acid sequence of SEQ ID NO: 2, and a VHCDR3 amino acid sequence of SEQ ID NO: 184; and/or a light chain variable region (VL) comprising a VLCDR1 amino acid sequence of SEQ ID NO: 4, a VLCDR2 amino acid sequence of SEQ ID NO: 5, and a VLCDR3 amino acid sequence of SEQ ID NO: 6;
(xl) a heavy chain variable region (VH) comprising a VHCDR1 amino acid sequence of SEQ ID NO: 1, a VHCDR2 amino acid sequence of SEQ ID NO: 2, and a VHCDR3 amino acid sequence of SEQ ID NO: 178; and/or a light chain variable region (VL) comprising a VLCDR1 amino acid sequence of SEQ ID NO: 4, a VLCDR2 amino acid sequence of SEQ ID NO: 5, and a VLCDR3 amino acid sequence of SEQ ID NO: 163;
(xli) a heavy chain variable region (VH) comprising a VHCDR1 amino acid sequence of SEQ ID NO: 164, a VHCDR2 amino acid sequence of SEQ ID NO: 2, and a VHCDR3 amino acid sequence of SEQ ID NO: 170; and/or a light chain variable region (VL) comprising a VLCDR1 amino acid sequence of SEQ ID NO: 4, a VLCDR2 amino acid sequence of SEQ ID NO: 5, and a VLCDR3 amino acid sequence of SEQ ID NO: 162;
(xlii) a heavy chain variable region (VH) comprising a VHCDR1 amino acid sequence of SEQ ID NO: 166, a VHCDR2 amino acid sequence of SEQ ID NO: 2, and a VHCDR3 amino acid sequence of SEQ ID NO: 183; and/or a light chain variable region (VL) comprising a VLCDR1 amino acid sequence of SEQ ID NO: 161, a VLCDR2 amino acid sequence of SEQ ID NO: 5, and a VLCDR3 amino acid sequence of SEQ ID NO: 6;
(xliii) a heavy chain variable region (VH) comprising a VHCDR1 amino acid sequence of SEQ ID NO: 165, a VHCDR2 amino acid sequence of SEQ ID NO: 2, and a VHCDR3 amino acid sequence of SEQ ID NO: 183; and/or a light chain variable region (VL) comprising a VLCDR1 amino acid sequence of SEQ ID NO: 4, a VLCDR2 amino acid sequence of SEQ ID NO: 5, and a VLCDR3 amino acid sequence of SEQ ID NO: 6;
(xliv) a heavy chain variable region (VH) comprising a VHCDR1 amino acid sequence of SEQ ID NO: 166, a VHCDR2 amino acid sequence of SEQ ID NO: 2, and a VHCDR3 amino acid sequence of SEQ ID NO: 183; and/or a light chain variable region (VL) comprising a VLCDR1 amino acid sequence of SEQ ID NO: 4, a VLCDR2 amino acid sequence of SEQ ID NO: 5, and a VLCDR3 amino acid sequence of SEQ ID NO: 6;
(xlv) a heavy chain variable region (VH) comprising a VHCDR1 amino acid sequence of SEQ ID NO: 165, a VHCDR2 amino acid sequence of SEQ ID NO: 2, and a VHCDR3 amino acid sequence of SEQ ID NO: 3; and/or a light chain variable region (VL) comprising a VLCDR1 amino acid sequence of SEQ ID NO: 161, a VLCDR2 amino acid sequence of SEQ ID NO: 5, and a VLCDR3 amino acid sequence of SEQ ID NO: 162; or
(xlvi) a heavy chain variable region (VH) comprising a VHCDR1 amino acid sequence of SEQ ID NO: 1, a VHCDR2 amino acid sequence of SEQ ID NO: 168, and a VHCDR3 amino acid sequence of SEQ ID NO: 183; and/or a light chain variable region (VL) comprising a VLCDR1 amino acid sequence of SEQ ID NO: 4, a VLCDR2 amino acid sequence of SEQ ID NO: 5, and a VLCDR3 amino acid sequence of SEQ ID NO: 6.

In one embodiment, provided herein are antibodies or antibody fragments, wherein the antibodies or antibody fragments comprise a heavy chain variable region (VH) comprising a VHCDR1 amino acid sequence selected from the group consisting of SEQ ID NOs: 192-195, a VHCDR2 amino acid sequence selected from the group consisting of SEQ ID NOs: 196-211, and a VHCDR3 amino acid sequence selected from the group consisting of SEQ ID NOs: 3 and 170-185; and/or a light chain variable region (VL) comprising a VLCDR1 amino acid sequence selected from the group consisting of SEQ ID NOs: 186-190, a VLCDR2 amino acid sequence of SEQ ID NO: 191, and a VLCDR3 amino acid sequence selected from the group consisting of SEQ ID NOs: 6, 162, and 163.

In one embodiment, provided herein are antibodies or antibody fragments, wherein the antibodies or antibody fragments comprise:
(i) a heavy chain variable region (VH) comprising a VHCDR1 amino acid sequence of SEQ ID NO: 192, a VHCDR2 amino acid sequence of SEQ ID NO: 196, and a VHCDR3 amino acid sequence of SEQ ID NO: 3; and/or a light chain variable region (VL) comprising a VLCDR1 amino acid sequence of SEQ ID NO: 186, a VLCDR2 amino acid sequence of SEQ ID NO: 191, and a VLCDR3 amino acid sequence of SEQ ID NO: 6;
(ii) a heavy chain variable region (VH) comprising a VHCDR1 amino acid sequence of SEQ ID NO: 193, a VHCDR2 amino acid sequence of SEQ ID NO: 197, and a VHCDR3 amino acid sequence of SEQ ID NO: 3; and/or a light chain variable region (VL) comprising a VLCDR1 amino acid sequence of SEQ ID NO: 186, a VLCDR2 amino acid sequence of SEQ ID NO: 191, and a VLCDR3 amino acid sequence of SEQ ID NO: 6;
(iii) a heavy chain variable region (VH) comprising a VHCDR1 amino acid sequence of SEQ ID NO: 193, a VHCDR2 amino acid sequence of SEQ ID NO: 196, and a VHCDR3 amino acid sequence of SEQ ID NO: 170; and/or a light chain variable region (VL) comprising a VLCDR1 amino acid sequence of SEQ ID NO: 186, a VLCDR2 amino acid sequence of SEQ ID NO: 191, and a VLCDR3 amino acid sequence of SEQ ID NO: 6;
(iv) a heavy chain variable region (VH) comprising a VHCDR1 amino acid sequence of SEQ ID NO: 193, a VHCDR2 amino acid sequence of SEQ ID NO: 198, and a VHCDR3 amino acid sequence of SEQ ID NO: 3; and/or a light chain variable region (VL) comprising a VLCDR1 amino acid sequence of SEQ ID NO: 186, a VLCDR2 amino acid sequence of SEQ ID NO: 191, and a VLCDR3 amino acid sequence of SEQ ID NO: 6;
(v) a heavy chain variable region (VH) comprising a VHCDR1 amino acid sequence of SEQ ID NO: 193, a VHCDR2 amino acid sequence of SEQ ID NO: 196, and a VHCDR3 amino acid sequence of SEQ ID NO: 3; and/or a light chain variable region (VL) comprising a VLCDR1 amino acid sequence of SEQ ID NO: 186, a VLCDR2 amino acid sequence of SEQ ID NO: 191, and a VLCDR3 amino acid sequence of SEQ ID NO: 162;
(vi) a heavy chain variable region (VH) comprising a VHCDR1 amino acid sequence of SEQ ID NO: 193, a VHCDR2 amino acid sequence of SEQ ID NO: 196, and a VHCDR3 amino acid sequence of SEQ ID NO: 3; and/or a light chain variable region (VL) comprising a VLCDR1 amino acid sequence of SEQ ID NO: 186, a VLCDR2 amino acid sequence of SEQ ID NO: 191, and a VLCDR3 amino acid sequence of SEQ ID NO: 6;
(vii) a heavy chain variable region (VH) comprising a VHCDR1 amino acid sequence of SEQ ID NO: 193, a VHCDR2 amino acid sequence of SEQ ID NO: 196, and a VHCDR3 amino acid sequence of SEQ ID NO: 171; and/or a light chain variable region (VL) comprising a VLCDR1 amino acid sequence of SEQ ID NO: 186, a VLCDR2 amino acid sequence of SEQ ID NO: 191, and a VLCDR3 amino acid sequence of SEQ ID NO: 6;
(viii) a heavy chain variable region (VH) comprising a VHCDR1 amino acid sequence of SEQ ID NO: 193, a VHCDR2 amino acid sequence of SEQ ID NO: 196, and a VHCDR3 amino acid sequence of SEQ ID NO: 172; and/or a light chain variable region (VL) comprising a VLCDR1 amino acid sequence of SEQ ID NO: 186, a VLCDR2 amino acid sequence of SEQ ID NO: 191, and a VLCDR3 amino acid sequence of SEQ ID NO: 6;
(ix) a heavy chain variable region (VH) comprising a VHCDR1 amino acid sequence of SEQ ID NO: 193, a VHCDR2 amino acid sequence of SEQ ID NO: 196, and a VHCDR3 amino acid sequence of SEQ ID NO: 3; and/or a light chain variable region (VL) comprising a VLCDR1 amino acid sequence of SEQ ID NO: 187, a VLCDR2 amino acid sequence of SEQ ID NO: 191, and a VLCDR3 amino acid sequence of SEQ ID NO: 6;
(x) a heavy chain variable region (VH) comprising a VHCDR1 amino acid sequence of SEQ ID NO: 193, a VHCDR2 amino acid sequence of SEQ ID NO: 196, and a VHCDR3 amino acid sequence of SEQ ID NO: 173; and/or a light chain variable region (VL) comprising a VLCDR1 amino acid sequence of SEQ ID NO: 186, a VLCDR2 amino acid sequence of SEQ ID NO: 191, and a VLCDR3 amino acid sequence of SEQ ID NO: 6;
(xi) a heavy chain variable region (VH) comprising a VHCDR1 amino acid sequence of SEQ ID NO: 193, a VHCDR2 amino acid sequence of SEQ ID NO: 199, and a VHCDR3 amino acid sequence of SEQ ID NO: 3; and/or a light chain variable region (VL) comprising a VLCDR1 amino acid sequence of SEQ ID NO: 186, a VLCDR2 amino acid sequence of SEQ ID NO: 191, and a VLCDR3 amino acid sequence of SEQ ID NO: 6;
(xii) a heavy chain variable region (VH) comprising a VHCDR1 amino acid sequence of SEQ ID NO: 193, a VHCDR2 amino acid sequence of SEQ ID NO: 196, and a VHCDR3 amino acid sequence of SEQ ID NO: 174; and/or a light chain variable region (VL) comprising a VLCDR1 amino acid sequence of SEQ ID NO: 186, a VLCDR2 amino acid sequence of SEQ ID NO: 191, and a VLCDR3 amino acid sequence of SEQ ID NO: 6;
(xiii) a heavy chain variable region (VH) comprising a VHCDR1 amino acid sequence of SEQ ID NO: 192, a VHCDR2 amino acid sequence of SEQ ID NO: 196, and a VHCDR3 amino acid sequence of SEQ ID NO: 175; and/or a light chain variable region (VL) comprising a VLCDR1 amino acid sequence of SEQ ID NO: 186, a VLCDR2 amino acid sequence of SEQ ID NO: 191, and a VLCDR3 amino acid sequence of SEQ ID NO: 6;
(xiv) a heavy chain variable region (VH) comprising a VHCDR1 amino acid sequence of SEQ ID NO: 193, a VHCDR2 amino acid sequence of SEQ ID NO: 200, and a VHCDR3 amino acid sequence of SEQ ID NO: 3; and/or a light chain variable region (VL) comprising a VLCDR1 amino acid sequence of SEQ ID NO: 186, a VLCDR2 amino acid sequence of SEQ ID NO: 191, and a VLCDR3 amino acid sequence of SEQ ID NO: 6;
(xv) a heavy chain variable region (VH) comprising a VHCDR1 amino acid sequence of SEQ ID NO: 193, a VHCDR2 amino acid sequence of SEQ ID NO: 201, and a VHCDR3 amino acid sequence of SEQ ID NO: 3; and/or a light chain variable region (VL) comprising a VLCDR1 amino acid sequence of SEQ ID NO: 186, a VLCDR2 amino acid sequence of SEQ ID NO: 191, and a VLCDR3 amino acid sequence of SEQ ID NO: 6;
(xvi) a heavy chain variable region (VH) comprising a VHCDR1 amino acid sequence of SEQ ID NO: 193, a VHCDR2 amino acid sequence of SEQ ID NO: 201, and a VHCDR3 amino acid sequence of SEQ ID NO: 3; and/or a light chain variable region (VL) comprising a VLCDR1 amino acid sequence of SEQ ID NO: 187, a VLCDR2 amino acid sequence of SEQ ID NO: 191, and a VLCDR3 amino acid sequence of SEQ ID NO: 6;
(xvii) a heavy chain variable region (VH) comprising a VHCDR1 amino acid sequence of SEQ ID NO: 193, a VHCDR2 amino acid sequence of SEQ ID NO: 196, and a VHCDR3 amino acid sequence of SEQ ID NO: 3; and/or a light chain variable region (VL) comprising a VLCDR1 amino acid sequence of SEQ ID NO: 188, a VLCDR2 amino acid sequence of SEQ ID NO: 191, and a VLCDR3 amino acid sequence of SEQ ID NO: 163;
(xviii) a heavy chain variable region (VH) comprising a VHCDR1 amino acid sequence of SEQ ID NO: 193, a VHCDR2 amino acid sequence of SEQ ID NO: 196, and a VHCDR3 amino acid sequence of SEQ ID NO: 170; and/or a light chain variable region (VL) comprising a VLCDR1 amino acid sequence of SEQ ID NO: 189, a VLCDR2 amino acid sequence of SEQ ID NO: 191, and a VLCDR3 amino acid sequence of SEQ ID NO: 6;
(xix) a heavy chain variable region (VH) comprising a VHCDR1 amino acid sequence of SEQ ID NO: 193, a VHCDR2 amino acid sequence of SEQ ID NO: 196, and a VHCDR3 amino acid sequence of SEQ ID NO: 176; and/or a light chain variable region (VL) comprising a VLCDR1 amino acid sequence of SEQ ID NO: 186, a VLCDR2 amino acid sequence of SEQ ID NO: 191, and a VLCDR3 amino acid sequence of SEQ ID NO: 6;
(xx) a heavy chain variable region (VH) comprising a VHCDR1 amino acid sequence of SEQ ID NO: 193, a VHCDR2 amino acid sequence of SEQ ID NO: 196, and a VHCDR3 amino acid sequence of SEQ ID NO: 171; and/or a light chain variable region (VL) comprising a VLCDR1 amino acid sequence of SEQ ID NO: 187, a VLCDR2 amino acid sequence of SEQ ID NO: 191, and a VLCDR3 amino acid sequence of SEQ ID NO: 6;
(xxi) a heavy chain variable region (VH) comprising a VHCDR1 amino acid sequence of SEQ ID NO: 193, a VHCDR2 amino acid sequence of SEQ ID NO: 196, and a VHCDR3 amino acid sequence of SEQ ID NO: 173; and/or a light chain variable region (VL) comprising a VLCDR1 amino acid sequence of SEQ ID NO: 186, a VLCDR2 amino acid sequence of SEQ ID NO: 191, and a VLCDR3 amino acid sequence of SEQ ID NO: 6;
(xxii) a heavy chain variable region (VH) comprising a VHCDR1 amino acid sequence of SEQ ID NO: 193, a VHCDR2 amino acid sequence of SEQ ID NO: 197, and a VHCDR3 amino acid sequence of SEQ ID NO: 177; and/or a light chain variable region (VL) comprising a VLCDR1 amino acid sequence of SEQ ID NO: 186, a VLCDR2 amino acid sequence of SEQ ID NO: 191, and a VLCDR3 amino acid sequence of SEQ ID NO: 6;
(xxiii) a heavy chain variable region (VH) comprising a VHCDR1 amino acid sequence of SEQ ID NO: 192, a VHCDR2 amino acid sequence of SEQ ID NO: 196, and a VHCDR3 amino acid sequence of SEQ ID NO: 178; and/or a light chain variable region (VL) comprising a VLCDR1 amino acid sequence of SEQ ID NO: 186, a VLCDR2 amino acid sequence of SEQ ID NO: 191, and a VLCDR3 amino acid sequence of SEQ ID NO: 6;
(xxiv) a heavy chain variable region (VH) comprising a VHCDR1 amino acid sequence of SEQ ID NO: 193, a VHCDR2 amino acid sequence of SEQ ID NO: 196, and a VHCDR3 amino acid sequence of SEQ ID NO: 179; and/or a light chain variable region (VL) comprising a VLCDR1 amino acid sequence of SEQ ID NO: 186, a VLCDR2 amino acid sequence of SEQ ID NO: 191, and a VLCDR3 amino acid sequence of SEQ ID NO: 6;
(xxv) a heavy chain variable region (VH) comprising a VHCDR1 amino acid sequence of SEQ ID NO: 193, a VHCDR2 amino acid sequence of SEQ ID NO: 196, and a VHCDR3 amino acid sequence of SEQ ID NO: 179; and/or a light chain variable region (VL) comprising a VLCDR1 amino acid sequence of SEQ ID NO: 187, a VLCDR2 amino acid sequence of SEQ ID NO: 191, and a VLCDR3 amino acid sequence of SEQ ID NO: 6;
(xxvi) a heavy chain variable region (VH) comprising a VHCDR1 amino acid sequence of SEQ ID NO: 192, a VHCDR2 amino acid sequence of SEQ ID NO: 202, and a VHCDR3 amino acid sequence of SEQ ID NO: 174; and/or a light chain variable region (VL) comprising a VLCDR1 amino acid sequence of SEQ ID NO: 186, a VLCDR2 amino acid sequence of SEQ ID NO: 191, and a VLCDR3 amino acid sequence of SEQ ID NO: 6;
(xxvii) a heavy chain variable region (VH) comprising a VHCDR1 amino acid sequence of SEQ ID NO: 193, a VHCDR2 amino acid sequence of SEQ ID NO: 196, and a VHCDR3 amino acid sequence of SEQ ID NO: 180; and/or a light chain variable region (VL) comprising a VLCDR1 amino acid sequence of SEQ ID NO: 186, a VLCDR2 amino acid sequence of SEQ ID NO: 191, and a VLCDR3 amino acid sequence of SEQ ID NO: 6;
(xxviii) a heavy chain variable region (VH) comprising a VHCDR1 amino acid sequence of SEQ ID NO: 193, a VHCDR2 amino acid sequence of SEQ ID NO: 201, and a VHCDR3 amino acid sequence of SEQ ID NO: 3; and/or a light chain variable region (VL) comprising a VLCDR1 amino acid sequence of SEQ ID NO: 186, a VLCDR2 amino acid sequence of SEQ ID NO: 191, and a VLCDR3 amino acid sequence of SEQ ID NO: 162;
(xxix) a heavy chain variable region (VH) comprising a VHCDR1 amino acid sequence of SEQ ID NO: 193, a VHCDR2 amino acid sequence of SEQ ID NO: 196, and a VHCDR3 amino acid sequence of SEQ ID NO: 181; and/or a light chain variable region (VL) comprising a VLCDR1 amino acid sequence of SEQ ID NO: 186, a VLCDR2 amino acid sequence of SEQ ID NO: 191, and a VLCDR3 amino acid sequence of SEQ ID NO: 162;
(xxx) a heavy chain variable region (VH) comprising a VHCDR1 amino acid sequence of SEQ ID NO: 193, a VHCDR2 amino acid sequence of SEQ ID NO: 196, and a VHCDR3 amino acid sequence of SEQ ID NO: 182; and/or a light chain variable region (VL) comprising a VLCDR1 amino acid sequence of SEQ ID NO: 186, a VLCDR2 amino acid sequence of SEQ ID NO: 191, and a VLCDR3 amino acid sequence of SEQ ID NO: 6;
(xxxi) a heavy chain variable region (VH) comprising a VHCDR1 amino acid sequence of SEQ ID NO: 193, a VHCDR2 amino acid sequence of SEQ ID NO: 196, and a VHCDR3 amino acid sequence of SEQ ID NO: 183; and/or a light chain variable region (VL) comprising a VLCDR1 amino acid sequence of SEQ ID NO: 186, a VLCDR2 amino acid sequence of SEQ ID NO: 191, and a VLCDR3 amino acid sequence of SEQ ID NO: 6;
(xxxii) a heavy chain variable region (VH) comprising a VHCDR1 amino acid sequence of SEQ ID NO: 193, a VHCDR2 amino acid sequence of SEQ ID NO: 196, and a VHCDR3 amino acid sequence of SEQ ID NO: 3; and/or a light chain variable region (VL) comprising a VLCDR1 amino acid sequence of SEQ ID NO: 186, a VLCDR2 amino acid sequence of SEQ ID NO: 191, and a VLCDR3 amino acid sequence of SEQ ID NO: 163;
(xxxiii) a heavy chain variable region (VH) comprising a VHCDR1 amino acid sequence of SEQ ID NO: 193, a VHCDR2 amino acid sequence of SEQ ID NO: 196, and a VHCDR3 amino acid sequence of SEQ ID NO: 181; and/or a light chain variable region (VL) comprising a VLCDR1 amino acid sequence of SEQ ID NO: 186, a VLCDR2 amino acid sequence of SEQ ID NO: 191, and a VLCDR3 amino acid sequence of SEQ ID NO: 6;
(xxxiv) a heavy chain variable region (VH) comprising a VHCDR1 amino acid sequence of SEQ ID NO: 193, a VHCDR2 amino acid sequence of SEQ ID NO: 203, and a VHCDR3 amino acid sequence of SEQ ID NO: 184; and/or a light chain variable region (VL) comprising a VLCDR1 amino acid sequence of SEQ ID NO: 186, a VLCDR2 amino acid sequence of SEQ ID NO: 191, and a VLCDR3 amino acid sequence of SEQ ID NO: 6;
(xxxv) a heavy chain variable region (VH) comprising a VHCDR1 amino acid sequence of SEQ ID NO: 194, a VHCDR2 amino acid sequence of SEQ ID NO: 196, and a VHCDR3 amino acid sequence of SEQ ID NO: 185; and/or a light chain variable region (VL) comprising a VLCDR1 amino acid sequence of SEQ ID NO: 189, a VLCDR2 amino acid sequence of SEQ ID NO: 191, and a VLCDR3 amino acid sequence of SEQ ID NO: 6;
(xxxvi) a heavy chain variable region (VH) comprising a VHCDR1 amino acid sequence of SEQ ID NO: 195, a VHCDR2 amino acid sequence of SEQ ID NO: 197, and a VHCDR3 amino acid sequence of SEQ ID NO: 170; and/or a light chain variable region (VL) comprising a VLCDR1 amino acid sequence of SEQ ID NO: 186, a VLCDR2 amino acid sequence of SEQ ID NO: 191, and a VLCDR3 amino acid sequence of SEQ ID NO: 6;
(xxxvii) a heavy chain variable region (VH) comprising a VHCDR1 amino acid sequence of SEQ ID NO: 193, a VHCDR2 amino acid sequence of SEQ ID NO: 204, and a VHCDR3 amino acid sequence of SEQ ID NO: 3; and/or a light chain variable region (VL) comprising a VLCDR1 amino acid sequence of SEQ ID NO: 186, a VLCDR2 amino acid sequence of SEQ ID NO: 191, and a VLCDR3 amino acid sequence of SEQ ID NO: 6;
(xxxviii) a heavy chain variable region (VH) comprising a VHCDR1 amino acid sequence of SEQ ID NO: 192, a VHCDR2 amino acid sequence of SEQ ID NO: 205, and a VHCDR3 amino acid sequence of SEQ ID NO: 170; and/or a light chain variable region (VL) comprising a VLCDR1 amino acid sequence of SEQ ID NO: 186, a VLCDR2 amino acid sequence of SEQ ID NO: 191, and a VLCDR3 amino acid sequence of SEQ ID NO: 6;
(xxxix) a heavy chain variable region (VH) comprising a VHCDR1 amino acid sequence of SEQ ID NO: 193, a VHCDR2 amino acid sequence of SEQ ID NO: 206, and a VHCDR3 amino acid sequence of SEQ ID NO: 171; and/or a light chain variable region (VL) comprising a VLCDR1 amino acid sequence of SEQ ID NO: 186, a VLCDR2 amino acid sequence of SEQ ID NO: 191, and a VLCDR3 amino acid sequence of SEQ ID NO: 6;
(xl) a heavy chain variable region (VH) comprising a VHCDR1 amino acid sequence of SEQ ID NO: 194, a VHCDR2 amino acid sequence of SEQ ID NO: 196, and a VHCDR3 amino acid sequence of SEQ ID NO: 3; and/or a light chain variable region (VL) comprising a VLCDR1 amino acid sequence of SEQ ID NO: 186, a VLCDR2 amino acid sequence of SEQ ID NO: 191, and a VLCDR3 amino acid sequence of SEQ ID NO: 6;
(xli) a heavy chain variable region (VH) comprising a VHCDR1 amino acid sequence of SEQ ID NO: 193, a VHCDR2 amino acid sequence of SEQ ID NO: 207, and a VHCDR3 amino acid sequence of SEQ ID NO: 174; and/or a light chain variable region (VL) comprising a VLCDR1 amino acid sequence of SEQ ID NO: 190, a VLCDR2 amino acid sequence of SEQ ID NO: 191, and a VLCDR3 amino acid sequence of SEQ ID NO: 6;
(xlii) a heavy chain variable region (VH) comprising a VHCDR1 amino acid sequence of SEQ ID NO: 193, a VHCDR2 amino acid sequence of SEQ ID NO: 196, and a VHCDR3 amino acid sequence of SEQ ID NO: 184; and/or a light chain variable region (VL) comprising a VLCDR1 amino acid sequence of SEQ ID NO: 186, a VLCDR2 amino acid sequence of SEQ ID NO: 191, and a VLCDR3 amino acid sequence of SEQ ID NO: 6;
(xliii) a heavy chain variable region (VH) comprising a VHCDR1 amino acid sequence of SEQ ID NO: 193, a VHCDR2 amino acid sequence of SEQ ID NO: 206, and a VHCDR3 amino acid sequence of SEQ ID NO: 184; and/or a light chain variable region (VL) comprising a VLCDR1 amino acid sequence of SEQ ID NO: 186, a VLCDR2 amino acid sequence of SEQ ID NO: 191, and a VLCDR3 amino acid sequence of SEQ ID NO: 6;
(xliv) a heavy chain variable region (VH) comprising a VHCDR1 amino acid sequence of SEQ ID NO: 193, a VHCDR2 amino acid sequence of SEQ ID NO: 196, and a VHCDR3 amino acid sequence of SEQ ID NO: 175; and/or a light chain variable region (VL) comprising a VLCDR1 amino acid sequence of SEQ ID NO: 186, a VLCDR2 amino acid sequence of SEQ ID NO: 191, and a VLCDR3 amino acid sequence of SEQ ID NO: 6;
(xlv) a heavy chain variable region (VH) comprising a VHCDR1 amino acid sequence of SEQ ID NO: 193, a VHCDR2 amino acid sequence of SEQ ID NO: 196, and a VHCDR3 amino acid sequence of SEQ ID NO: 3; and/or a light chain variable region (VL) comprising a VLCDR1 amino acid sequence of SEQ ID NO: 189, a VLCDR2 amino acid sequence of SEQ ID NO: 191, and a VLCDR3 amino acid sequence of SEQ ID NO: 6;
(xlvi) a heavy chain variable region (VH) comprising a VHCDR1 amino acid sequence of SEQ ID NO: 193, a VHCDR2 amino acid sequence of SEQ ID NO: 202, and a VHCDR3 amino acid sequence of SEQ ID NO: 3; and/or a light chain variable region (VL) comprising a VLCDR1 amino acid sequence of SEQ ID NO: 186, a VLCDR2 amino acid sequence of SEQ ID NO: 191, and a VLCDR3 amino acid sequence of SEQ ID NO: 6;
(xlvii) a heavy chain variable region (VH) comprising a VHCDR1 amino acid sequence of SEQ ID NO: 193, a VHCDR2 amino acid sequence of SEQ ID NO: 207, and a VHCDR3 amino acid sequence of SEQ ID NO: 3; and/or a light chain variable region (VL) comprising a VLCDR1 amino acid sequence of SEQ ID NO: 186, a VLCDR2 amino acid sequence of SEQ ID NO: 191, and a VLCDR3 amino acid sequence of SEQ ID NO: 6;
(xlviii) a heavy chain variable region (VH) comprising a VHCDR1 amino acid sequence of SEQ ID NO: 195, a VHCDR2 amino acid sequence of SEQ ID NO: 202, and a VHCDR3 amino acid sequence of SEQ ID NO: 3; and/or a light chain variable region (VL) comprising a VLCDR1 amino acid sequence of SEQ ID NO: 186, a VLCDR2 amino acid sequence of SEQ ID NO: 191, and a VLCDR3 amino acid sequence of SEQ ID NO: 6;
(xlix) a heavy chain variable region (VH) comprising a VHCDR1 amino acid sequence of SEQ ID NO: 193, a VHCDR2 amino acid sequence of SEQ ID NO: 208, and a VHCDR3 amino acid sequence of SEQ ID NO: 3; and/or a light chain variable region (VL) comprising a VLCDR1 amino acid sequence of SEQ ID NO: 186, a VLCDR2 amino acid sequence of SEQ ID NO: 191, and a VLCDR3 amino acid sequence of SEQ ID NO: 6;
(l) a heavy chain variable region (VH) comprising a VHCDR1 amino acid sequence of SEQ ID NO: 195, a VHCDR2 amino acid sequence of SEQ ID NO: 209, and a VHCDR3 amino acid sequence of SEQ ID NO: 3; and/or a light chain variable region (VL) comprising a VLCDR1 amino acid sequence of SEQ ID NO: 186, a VLCDR2 amino acid sequence of SEQ ID NO: 191, and a VLCDR3 amino acid sequence of SEQ ID NO: 6;
(li) a heavy chain variable region (VH) comprising a VHCDR1 amino acid sequence of SEQ ID NO: 193, a VHCDR2 amino acid sequence of SEQ ID NO: 209, and a VHCDR3 amino acid sequence of SEQ ID NO: 3; and/or a light chain variable region (VL) comprising a VLCDR1 amino acid sequence of SEQ ID NO: 187, a VLCDR2 amino acid sequence of SEQ ID NO: 191, and a VLCDR3 amino acid sequence of SEQ ID NO: 6;
(lii) a heavy chain variable region (VH) comprising a VHCDR1 amino acid sequence of SEQ ID NO: 195, a VHCDR2 amino acid sequence of SEQ ID NO: 206, and a VHCDR3 amino acid sequence of SEQ ID NO: 184; and/or a light chain variable region (VL) comprising a VLCDR1 amino acid sequence of SEQ ID NO: 186, a VLCDR2 amino acid sequence of SEQ ID NO: 191, and a VLCDR3 amino acid sequence of SEQ ID NO: 6;
(liii) a heavy chain variable region (VH) comprising a VHCDR1 amino acid sequence of SEQ ID NO: 193, a VHCDR2 amino acid sequence of SEQ ID NO: 210, and a VHCDR3 amino acid sequence of SEQ ID NO: 178; and/or a light chain variable region (VL) comprising a VLCDR1 amino acid sequence of SEQ ID NO: 186, a VLCDR2 amino acid sequence of SEQ ID NO: 191, and a VLCDR3 amino acid sequence of SEQ ID NO: 163;
(liv) a heavy chain variable region (VH) comprising a VHCDR1 amino acid sequence of SEQ ID NO: 192, a VHCDR2 amino acid sequence of SEQ ID NO: 197, and a VHCDR3 amino acid sequence of SEQ ID NO: 170; and/or a light chain variable region (VL) comprising a VLCDR1 amino acid sequence of SEQ ID NO: 186, a VLCDR2 amino acid sequence of SEQ ID NO: 191, and a VLCDR3 amino acid sequence of SEQ ID NO: 162;
(lv) a heavy chain variable region (VH) comprising a VHCDR1 amino acid sequence of SEQ ID NO: 193, a VHCDR2 amino acid sequence of SEQ ID NO: 210, and a VHCDR3 amino acid sequence of SEQ ID NO: 174; and/or a light chain variable region (VL) comprising a VLCDR1 amino acid sequence of SEQ ID NO: 188, a VLCDR2 amino acid sequence of SEQ ID NO: 191, and a VLCDR3 amino acid sequence of SEQ ID NO: 6;
(lvi) a heavy chain variable region (VH) comprising a VHCDR1 amino acid sequence of SEQ ID NO: 195, a VHCDR2 amino acid sequence of SEQ ID NO: 196, and a VHCDR3 amino acid sequence of SEQ ID NO: 183; and/or a light chain variable region (VL) comprising a VLCDR1 amino acid sequence of SEQ ID NO: 187, a VLCDR2 amino acid sequence of SEQ ID NO: 191, and a VLCDR3 amino acid sequence of SEQ ID NO: 6;
(lvii) a heavy chain variable region (VH) comprising a VHCDR1 amino acid sequence of SEQ ID NO: 193, a VHCDR2 amino acid sequence of SEQ ID NO: 209, and a VHCDR3 amino acid sequence of SEQ ID NO: 3; and/or a light chain variable region (VL) comprising a VLCDR1 amino acid sequence of SEQ ID NO: 186, a VLCDR2 amino acid sequence of SEQ ID NO: 191, and a VLCDR3 amino acid sequence of SEQ ID NO: 6;
(lviii) a heavy chain variable region (VH) comprising a VHCDR1 amino acid sequence of SEQ ID NO: 193, a VHCDR2 amino acid sequence of SEQ ID NO: 196, and a VHCDR3 amino acid sequence of SEQ ID NO: 184; and/or a light chain variable region (VL) comprising a VLCDR1 amino acid sequence of SEQ ID NO: 188, a VLCDR2 amino acid sequence of SEQ ID NO: 191, and a VLCDR3 amino acid sequence of SEQ ID NO: 6;
(lix) a heavy chain variable region (VH) comprising a VHCDR1 amino acid sequence of SEQ ID NO: 194, a VHCDR2 amino acid sequence of SEQ ID NO: 196, and a VHCDR3 amino acid sequence of SEQ ID NO: 183; and/or a light chain variable region (VL) comprising a VLCDR1 amino acid sequence of SEQ ID NO: 186, a VLCDR2 amino acid sequence of SEQ ID NO: 191, and a VLCDR3 amino acid sequence of SEQ ID NO: 6;
(lx) a heavy chain variable region (VH) comprising a VHCDR1 amino acid sequence of SEQ ID NO: 195, a VHCDR2 amino acid sequence of SEQ ID NO: 196, and a VHCDR3 amino acid sequence of SEQ ID NO: 183; and/or a light chain variable region (VL) comprising a VLCDR1 amino acid sequence of SEQ ID NO: 188, a VLCDR2 amino acid sequence of SEQ ID NO: 191, and a VLCDR3 amino acid sequence of SEQ ID NO: 6;
(lxi) a heavy chain variable region (VH) comprising a VHCDR1 amino acid sequence of SEQ ID NO: 192, a VHCDR2 amino acid sequence of SEQ ID NO: 211, and a VHCDR3 amino acid sequence of SEQ ID NO: 170; and/or a light chain variable region (VL) comprising a VLCDR1 amino acid sequence of SEQ ID NO: 186, a VLCDR2 amino acid sequence of SEQ ID NO: 191, and a VLCDR3 amino acid sequence of SEQ ID NO: 6;
(lxii) a heavy chain variable region (VH) comprising a VHCDR1 amino acid sequence of SEQ ID NO: 193, a VHCDR2 amino acid sequence of SEQ ID NO: 210, and a VHCDR3 amino acid sequence of SEQ ID NO: 172; and/or a light chain variable region (VL) comprising a VLCDR1 amino acid sequence of SEQ ID NO: 189, a VLCDR2 amino acid sequence of SEQ ID NO: 191, and a VLCDR3 amino acid sequence of SEQ ID NO: 6;
(lxiii) a heavy chain variable region (VH) comprising a VHCDR1 amino acid sequence of SEQ ID NO: 195, a VHCDR2 amino acid sequence of SEQ ID NO: 196, and a VHCDR3 amino acid sequence of SEQ ID NO: 183; and/or a light chain variable region (VL) comprising a VLCDR1 amino acid sequence of SEQ ID NO: 187, a VLCDR2 amino acid sequence of SEQ ID NO: 191, and a VLCDR3 amino acid sequence of SEQ ID NO: 6;
(lxiv) a heavy chain variable region (VH) comprising a VHCDR1 amino acid sequence of SEQ ID NO: 194, a VHCDR2 amino acid sequence of SEQ ID NO: 206, and a VHCDR3 amino acid sequence of SEQ ID NO: 3; and/or a light chain variable region (VL) comprising a VLCDR1 amino acid sequence of SEQ ID NO: 187, a VLCDR2 amino acid sequence of SEQ ID NO: 191, and a VLCDR3 amino acid sequence of SEQ ID NO: 162;
(lxv) a heavy chain variable region (VH) comprising a VHCDR1 amino acid sequence of SEQ ID NO: 193, a VHCDR2 amino acid sequence of SEQ ID NO: 208, and a VHCDR3 amino acid sequence of SEQ ID NO: 183; and/or a light chain variable region (VL) comprising a VLCDR1 amino acid sequence of SEQ ID NO: 186, a VLCDR2 amino acid sequence of SEQ ID NO: 191, and a VLCDR3 amino acid sequence of SEQ ID NO: 6;
(lxvi) a heavy chain variable region (VH) comprising a VHCDR1 amino acid sequence of SEQ ID NO: 193, a VHCDR2 amino acid sequence of SEQ ID NO: 199, and a VHCDR3 amino acid sequence of SEQ ID NO: 3; and/or a light chain variable region (VL) comprising a VLCDR1 amino acid sequence of SEQ ID NO: 187, a VLCDR2 amino acid sequence of SEQ ID NO: 191, and a VLCDR3 amino acid sequence of SEQ ID NO: 6; or
(lxvii) a heavy chain variable region (VH) comprising a VHCDR1 amino acid sequence of SEQ ID NO: 193, a VHCDR2 amino acid sequence of SEQ ID NO: 199, and a VHCDR3 amino acid sequence of SEQ ID NO: 184; and/or a light chain variable region (VL) comprising a VLCDR1 amino acid sequence of SEQ ID NO: 186, a VLCDR2 amino acid sequence of SEQ ID NO: 191, and a VLCDR3 amino acid sequence of SEQ ID NO: 6.

In some aspects, the antibodies or antibody fragments comprise a heavy chain variable sequence having a sequence of

(SEQ ID NO: 18)
X₁X₂QLX₃X₄SGX₅X₆X₇X₈KPGX₉SX₁₀X₁₁X₁₂SCKX₁₃SGYTFTNYGMNWVR
QAPGX₁₄GLX₁₅WX₁₆GWINTYTGEPTYADDFKGRX₁₇TX₁₈X₁₉X₂₀DX₂₁SX₂₂
X₂₃TX₂₄YX₂₅X₂₆X₂₇X₂₈X₂₉LX₃₀X₃₁X₃₂DTAVYFCARYDHAMDYWGQGTX₃₃
VTVSS,

wherein X₁ is Q or E, X₂ is I or V, X₃ is V or Q, X₄ is Q or E, X₅ is A, P, or G, X₆ is E or G, X₇ is V or L, X₈ is V or K, X₉ is A, E, G, or S, X₁₀ is V or L, X₁₁ is K or R, X₁₂ is V, L, or I, X₁₃ is A or T, X₁₄ is K or Q, X₁₅ is E or K, X₁₆ is M or V, X₁₇ is F or V, X₁₈ is F, M, or I, X₁₉ is T or S, X₂₀ is T, R, or A, X₂₁ is T, D, or E, X₂₂ is T, A, or K, X₂₃ is S or N, X₂₄ is L or A, X₂₅ is M or L, X₂₆ is E or Q, X₂₇ is L or M, X₂₈ is R, S, T, or N, X₂₉ is S or G, X₃₀ is R, K, or M, X₃₁ is S or T, X₃₂ is D or E, and X₃₃ is L, S, or T;
and/or a light chain variable sequence having a sequence of
wherein

(SEQ ID NO: 26)
X₁X₂X₃X₄TQSPX₅SLX₆X₇SX₈GX₉RX₁₀TIX₁₁CKSSQSLLNSGTRKNYLAW
YQQKX₁₂GX₁₃X₁₄PX₁₅LLIYWTSTRESGVPX₁₆RFSGSGSGTDFTLTIX₁₇
X₁₈LQX₁₉EDVAX₂₀YYCKQSYTLYTFGX₂₁GTKX₂₂EIK,

X₁ is E or D, X₂ is I or V, X₃ is V or Q, X₄ is L or M, X₅ is D or S, X₆ is A or S, X₇ is V or A, X₈ is L or V, X₉ is E or D, X₁₀ is A or V, X₁₁ is N or T, X₁₂ is A or P, X₁₃ is Q or K, X₁₄ is S, V, or P, X₁₅ is K or R, X₁₆ is D or S, X₁₇ is S, D, or N, X₁₈ is S or T, X₁₉ is A or P, X₂₀ is V or T, X₂₁ is Q or G, and X₂₂ is L or V.

In some aspects, said antibodies or antibody fragments comprise a heavy chain variable sequence having a sequence of

QIX₁LVQSGX₂EVKKPGASVKVSCKASGYX₃FTX₄YGMNWVRQAPGQGLEW
MGWINTYTGEX₅X₆YX₇DDFKGRFTFTTDTSTX₈TX₉YMX₁₀X₁₁RSLRSDD
TAVYFCX₁₂RYDHX₁₃MDYWGQGX₁₄LVTVSS

(SEQ ID NO: 104), wherein X₁ is Q or H, X₂ is A, D, T, V, S, or P, X₃ is T, S, or I, X₄ is N or K, X₅ is P, S, T, or A, X₆ is T, R, K, or I, X₇ is A, T, V, S, or G, X₈ is S, R, or T, X₉ is A, V, or G, X₁₀ is E or D, X₁₁ is L or V, X₁₂ is A, T, V, or G, X₁₃ is A, R, F, T, P, V, S, D, N, H, L, Y, or G, and X₁₄ is T or S;
and/or a light chain variable sequence having a sequence of

EIVLTQSPDSLX₁VSLGERATIX₂CKSSQSLX₃NSGTRKNYLX₄WYQX₅K
X₆GQSPX₇LX₈IYWTSTRESGVPDRFSX₉SGSGTDFTLX₁₀IDX₁₁LQX₁₂E
DVAX₁₃YYCKQSYX₁₄LYTFGGGTKVEIK

(SEQ ID NO: 158), wherein X₁ is A, T, or S, X₂ is N or K, X₃ is L, F, or V, X₄ is A, S, or T, X₅ is Q or K, X₆ is A, P, or S, X₇ is K or N, X₈ is L, V, or I, X₉ is G or A, X₁₀ is T or S, X₁₁ is S or R, X₁₂ is A or T, X₁₃ is V, I, or L, and X₁₄ is T, N, or S.

In some aspects, said antibodies or antibody fragments comprise a heavy chain variable sequence having a sequence selected from the group consisting of SEQ ID NOs: 7, 12-17, 26-103, and 225-229, or a heavy chain variable sequence having at least 70%, 80%, 90%, 95%, 96%, 97%, 98%, or 99% identity to any one of SEQ ID NOs: 7, 12-17, 26-103, and 225-229; and/or a light chain variable sequence having a sequence selected from the group consisting of SEQ ID NOs: 8, 19-24, 105-157, and 230-234, or a light chain variable sequence having at least 70%, 80%, 90%, 95%, 96%, 97%, 98%, or 99% identity to any one of SEQ ID NOs: 8, 19-24, 105-157, and 230-234.

In some aspects, said antibodies or antibody fragments comprise:
(i) a heavy chain variable sequence having a sequence according to SEQ ID NO: 7, or a heavy chain variable sequence having at least 70%, 80%, 90%, 95%, 96%, 97%, 98%, or 99% identity to SEQ ID NO: 7; and/or a light chain variable sequence having a sequence according to SEQ ID NO: 8, or a light chain variable sequence having at least 70%, 80%, 90%, 95%, 96%, 97%, 98%, or 99% identity to SEQ ID NO: 8;
(ii) a heavy chain variable sequence having a sequence according to SEQ ID NO: 12, or a heavy chain variable sequence having at least 70%, 80%, 90%, 95%, 96%, 97%, 98%, or 99% identity to SEQ ID NO: 12; and/or a light chain variable sequence having a sequence according to SEQ ID NO: 19, or a light chain variable sequence having at least 70%, 80%, 90%, 95%, 96%, 97%, 98%, or 99% identity to SEQ ID NO: 19;
(iii) a heavy chain variable sequence having a sequence according to SEQ ID NO: 12, or a heavy chain variable sequence having at least 70%, 80%, 90%, 95%, 96%, 97%, 98%, or 99% identity to SEQ ID NO: 12; and/or a light chain variable sequence having a sequence according to SEQ ID NO: 20, or a light chain variable sequence having at least 70%, 80%, 90%, 95%, 96%, 97%, 98%, or 99% identity to SEQ ID NO: 20;
(iv) a heavy chain variable sequence having a sequence according to SEQ ID NO: 12, or a heavy chain variable sequence having at least 70%, 80%, 90%, 95%, 96%, 97%, 98%, or 99% identity to SEQ ID NO: 12; and/or a light chain variable sequence having a sequence according to SEQ ID NO: 21, or a light chain variable sequence having at least 70%, 80%, 90%, 95%, 96%, 97%, 98%, or 99% identity to SEQ ID NO: 21;
(v) a heavy chain variable sequence having a sequence according to SEQ ID NO: 12, or a heavy chain variable sequence having at least 70%, 80%, 90%, 95%, 96%, 97%, 98%, or 99% identity to SEQ ID NO: 12; and/or a light chain variable sequence having a sequence according to SEQ ID NO: 22, or a light chain variable sequence having at least 70%, 80%, 90%, 95%, 96%, 97%, 98%, or 99% identity to SEQ ID NO: 22;
(vi) a heavy chain variable sequence having a sequence according to SEQ ID NO: 12, or a heavy chain variable sequence having at least 70%, 80%, 90%, 95%, 96%, 97%, 98%, or 99% identity to SEQ ID NO: 12; and/or a light chain variable sequence having a sequence according to SEQ ID NO: 23, or a light chain variable sequence having at least 70%, 80%, 90%, 95%, 96%, 97%, 98%, or 99% identity to SEQ ID NO: 23;
(vii) a heavy chain variable sequence having a sequence according to SEQ ID NO: 12, or a heavy chain variable sequence having at least 70%, 80%, 90%, 95%, 96%, 97%, 98%, or 99% identity to SEQ ID NO: 12; and/or a light chain variable sequence having a sequence according to SEQ ID NO: 24, or a light chain variable sequence having at least 70%, 80%, 90%, 95%, 96%, 97%, 98%, or 99% identity to SEQ ID NO: 24;
(viii) a heavy chain variable sequence having a sequence according to SEQ ID NO: 13, or a heavy chain variable sequence having at least 70%, 80%, 90%, 95%, 96%, 97%, 98%, or 99% identity to SEQ ID NO: 13; and/or a light chain variable sequence having a sequence according to SEQ ID NO: 19, or a light chain variable sequence having at least 70%, 80%, 90%, 95%, 96%, 97%, 98%, or 99% identity to SEQ ID NO: 19;
(ix) a heavy chain variable sequence having a sequence according to SEQ ID NO: 13, or a heavy chain variable sequence having at least 70%, 80%, 90%, 95%, 96%, 97%, 98%, or 99% identity to SEQ ID NO: 13; and/or a light chain variable sequence having a sequence according to SEQ ID NO: 20, or a light chain variable sequence having at least 70%, 80%, 90%, 95%, 96%, 97%, 98%, or 99% identity to SEQ ID NO: 20;
(x) a heavy chain variable sequence having a sequence according to SEQ ID NO: 13, or a heavy chain variable sequence having at least 70%, 80%, 90%, 95%, 96%, 97%, 98%, or 99% identity to SEQ ID NO: 13; and/or a light chain variable sequence having a sequence according to SEQ ID NO: 21, or a light chain variable sequence having at least 70%, 80%, 90%, 95%, 96%, 97%, 98%, or 99% identity to SEQ ID NO: 21;
(xi) a heavy chain variable sequence having a sequence according to SEQ ID NO: 13, or a heavy chain variable sequence having at least 70%, 80%, 90%, 95%, 96%, 97%, 98%, or 99% identity to SEQ ID NO: 13; and/or a light chain variable sequence having a sequence according to SEQ ID NO: 22, or a light chain variable sequence having at least 70%, 80%, 90%, 95%, 96%, 97%, 98%, or 99% identity to SEQ ID NO: 22;
(xii) a heavy chain variable sequence having a sequence according to SEQ ID NO: 13, or a heavy chain variable sequence having at least 70%, 80%, 90%, 95%, 96%, 97%, 98%, or 99% identity to SEQ ID NO: 13; and/or a light chain variable sequence having a sequence according to SEQ ID NO: 23, or a light chain variable sequence having at least 70%, 80%, 90%, 95%, 96%, 97%, 98%, or 99% identity to SEQ ID NO: 23;
(xiii) a heavy chain variable sequence having a sequence according to SEQ ID NO: 13, or a heavy chain variable sequence having at least 70%, 80%, 90%, 95%, 96%, 97%, 98%, or 99% identity to SEQ ID NO: 13; and/or a light chain variable sequence having a sequence according to SEQ ID NO: 24, or a light chain variable sequence having at least 70%, 80%, 90%, 95%, 96%, 97%, 98%, or 99% identity to SEQ ID NO: 24;
(xiv) a heavy chain variable sequence having a sequence according to SEQ ID NO: 14, or a heavy chain variable sequence having at least 70%, 80%, 90%, 95%, 96%, 97%, 98%, or 99% identity to SEQ ID NO: 14; and/or a light chain variable sequence having a sequence according to SEQ ID NO: 19, or a light chain variable sequence having at least 70%, 80%, 90%, 95%, 96%, 97%, 98%, or 99% identity to SEQ ID NO: 19;
(xv) a heavy chain variable sequence having a sequence according to SEQ ID NO: 14, or a heavy chain variable sequence having at least 70%, 80%, 90%, 95%, 96%, 97%, 98%, or 99% identity to SEQ ID NO: 14; and/or a light chain variable sequence having a sequence according to SEQ ID NO: 20, or a light chain variable sequence having at least 70%, 80%, 90%, 95%, 96%, 97%, 98%, or 99% identity to SEQ ID NO: 20;
(xvi) a heavy chain variable sequence having a sequence according to SEQ ID NO: 14, or a heavy chain variable sequence having at least 70%, 80%, 90%, 95%, 96%, 97%, 98%, or 99% identity to SEQ ID NO: 14; and/or a light chain variable sequence having a sequence according to SEQ ID NO: 21, or a light chain variable sequence having at least 70%, 80%, 90%, 95%, 96%, 97%, 98%, or 99% identity to SEQ ID NO: 21;
(xvii) a heavy chain variable sequence having a sequence according to SEQ ID NO: 14, or a heavy chain variable sequence having at least 70%, 80%, 90%, 95%, 96%, 97%, 98%, or 99% identity to SEQ ID NO: 14; and/or a light chain variable sequence having a sequence according to SEQ ID NO: 22, or a light chain variable sequence having at least 70%, 80%, 90%, 95%, 96%, 97%, 98%, or 99% identity to SEQ ID NO: 22;
(xviii) a heavy chain variable sequence having a sequence according to SEQ ID NO: 14, or a heavy chain variable sequence having at least 70%, 80%, 90%, 95%, 96%, 97%, 98%, or 99% identity to SEQ ID NO: 14; and/or a light chain variable sequence having a sequence according to SEQ ID NO: 23, or a light chain variable sequence having at least 70%, 80%, 90%, 95%, 96%, 97%, 98%, or 99% identity to SEQ ID NO: 23;
(xix) a heavy chain variable sequence having a sequence according to SEQ ID NO: 14, or a heavy chain variable sequence having at least 70%, 80%, 90%, 95%, 96%, 97%, 98%, or 99% identity to SEQ ID NO: 14; and/or a light chain variable sequence having a sequence according to SEQ ID NO: 24, or a light chain variable sequence having at least 70%, 80%, 90%, 95%, 96%, 97%, 98%, or 99% identity to SEQ ID NO: 24;
(xx) a heavy chain variable sequence having a sequence according to SEQ ID NO: 15, or a heavy chain variable sequence having at least 70%, 80%, 90%, 95%, 96%, 97%, 98%, or 99% identity to SEQ ID NO: 15; and/or a light chain variable sequence having a sequence according to SEQ ID NO: 19, or a light chain variable sequence having at least 70%, 80%, 90%, 95%, 96%, 97%, 98%, or 99% identity to SEQ ID NO: 19;
(xxi) a heavy chain variable sequence having a sequence according to SEQ ID NO: 15, or a heavy chain variable sequence having at least 70%, 80%, 90%, 95%, 96%, 97%, 98%, or 99% identity to SEQ ID NO: 15; and/or a light chain variable sequence having a sequence according to SEQ ID NO: 20, or a light chain variable sequence having at least 70%, 80%, 90%, 95%, 96%, 97%, 98%, or 99% identity to SEQ ID NO: 20;
(xxii) a heavy chain variable sequence having a sequence according to SEQ ID NO: 15, or a heavy chain variable sequence having at least 70%, 80%, 90%, 95%, 96%, 97%, 98%, or 99% identity to SEQ ID NO: 15; and/or a light chain variable sequence having a sequence according to SEQ ID NO: 21, or a light chain variable sequence having at least 70%, 80%, 90%, 95%, 96%, 97%, 98%, or 99% identity to SEQ ID NO: 21;
(xxiii) a heavy chain variable sequence having a sequence according to SEQ ID NO: 15, or a heavy chain variable sequence having at least 70%, 80%, 90%, 95%, 96%, 97%, 98%, or 99% identity to SEQ ID NO: 15; and/or a light chain variable sequence having a sequence according to SEQ ID NO: 22, or a light chain variable sequence having at least 70%, 80%, 90%, 95%, 96%, 97%, 98%, or 99% identity to SEQ ID NO: 22;
(xxiv) a heavy chain variable sequence having a sequence according to SEQ ID NO: 15, or a heavy chain variable sequence having at least 70%, 80%, 90%, 95%, 96%, 97%, 98%, or 99% identity to SEQ ID NO: 15; and/or a light chain variable sequence having a sequence according to SEQ ID NO: 23, or a light chain variable sequence having at least 70%, 80%, 90%, 95%, 96%, 97%, 98%, or 99% identity to SEQ ID NO: 23;
(xxv) a heavy chain variable sequence having a sequence according to SEQ ID NO: 15, or a heavy chain variable sequence having at least 70%, 80%, 90%, 95%, 96%, 97%, 98%, or 99% identity to SEQ ID NO: 15; and/or a light chain variable sequence having a sequence according to SEQ ID NO: 24, or a light chain variable sequence having at least 70%, 80%, 90%, 95%, 96%, 97%, 98%, or 99% identity to SEQ ID NO: 24;
(xxvi) a heavy chain variable sequence having a sequence according to SEQ ID NO: 16, or a heavy chain variable sequence having at least 70%, 80%, 90%, 95%, 96%, 97%, 98%, or 99% identity to SEQ ID NO: 16; and/or a light chain variable sequence having a sequence according to SEQ ID NO: 19, or a light chain variable sequence having at least 70%, 80%, 90%, 95%, 96%, 97%, 98%, or 99% identity to SEQ ID NO: 19;
(xxvii) a heavy chain variable sequence having a sequence according to SEQ ID NO: 16, or a heavy chain variable sequence having at least 70%, 80%, 90%, 95%, 96%, 97%, 98%, or 99% identity to SEQ ID NO: 16; and/or a light chain variable sequence having a sequence according to SEQ ID NO: 20, or a light chain variable sequence having at least 70%, 80%, 90%, 95%, 96%, 97%, 98%, or 99% identity to SEQ ID NO: 20;
(xxviii) a heavy chain variable sequence having a sequence according to SEQ ID NO: 16, or a heavy chain variable sequence having at least 70%, 80%, 90%, 95%, 96%, 97%, 98%, or 99% identity to SEQ ID NO: 16; and/or a light chain variable sequence having a sequence according to SEQ ID NO: 21, or a light chain variable sequence having at least 70%, 80%, 90%, 95%, 96%, 97%, 98%, or 99% identity to SEQ ID NO: 21;
(xxix) a heavy chain variable sequence having a sequence according to SEQ ID NO: 16, or a heavy chain variable sequence having at least 70%, 80%, 90%, 95%, 96%, 97%, 98%, or 99% identity to SEQ ID NO: 16; and/or a light chain variable sequence having a sequence according to SEQ ID NO: 22, or a light chain variable sequence having at least 70%, 80%, 90%, 95%, 96%, 97%, 98%, or 99% identity to SEQ ID NO: 22;
(xxx) a heavy chain variable sequence having a sequence according to SEQ ID NO: 16, or a heavy chain variable sequence having at least 70%, 80%, 90%, 95%, 96%, 97%, 98%, or 99% identity to SEQ ID NO: 16; and/or a light chain variable sequence having a sequence according to SEQ ID NO: 23, or a light chain variable sequence having at least 70%, 80%, 90%, 95%, 96%, 97%, 98%, or 99% identity to SEQ ID NO: 23;
(xxxi) a heavy chain variable sequence having a sequence according to SEQ ID NO: 16, or a heavy chain variable sequence having at least 70%, 80%, 90%, 95%, 96%, 97%, 98%, or 99% identity to SEQ ID NO: 16; and/or a light chain variable sequence having a sequence according to SEQ ID NO: 24, or a light chain variable sequence having at least 70%, 80%, 90%, 95%, 96%, 97%, 98%, or 99% identity to SEQ ID NO: 24;
(xxxii) a heavy chain variable sequence having a sequence according to SEQ ID NO: 17, or a heavy chain variable sequence having at least 70%, 80%, 90%, 95%, 96%, 97%, 98%, or 99% identity to SEQ ID NO: 17; and/or a light chain variable sequence having a sequence according to SEQ ID NO: 19, or a light chain variable sequence having at least 70%, 80%, 90%, 95%, 96%, 97%, 98%, or 99% identity to SEQ ID NO: 19;
(xxxiii) a heavy chain variable sequence having a sequence according to SEQ ID NO: 17, or a heavy chain variable sequence having at least 70%, 80%, 90%, 95%, 96%, 97%, 98%, or 99% identity to SEQ ID NO: 17; and/or a light chain variable sequence having a sequence according to SEQ ID NO: 20, or a light chain variable sequence having at least 70%, 80%, 90%, 95%, 96%, 97%, 98%, or 99% identity to SEQ ID NO: 20;
(xxxiv) a heavy chain variable sequence having a sequence according to SEQ ID NO: 17, or a heavy chain variable sequence having at least 70%, 80%, 90%, 95%, 96%, 97%, 98%, or 99% identity to SEQ ID NO: 17; and/or a light chain variable sequence having a sequence according to SEQ ID NO: 21, or a light chain variable sequence having at least 70%, 80%, 90%, 95%, 96%, 97%, 98%, or 99% identity to SEQ ID NO: 21;
(xxxv) a heavy chain variable sequence having a sequence according to SEQ ID NO: 17, or a heavy chain variable sequence having at least 70%, 80%, 90%, 95%, 96%, 97%, 98%, or 99% identity to SEQ ID NO: 17; and/or a light chain variable sequence having a sequence according to SEQ ID NO: 22, or a light chain variable sequence having at least 70%, 80%, 90%, 95%, 96%, 97%, 98%, or 99% identity to SEQ ID NO: 22;
(xxxvi) a heavy chain variable sequence having a sequence according to SEQ ID NO: 17, or a heavy chain variable sequence having at least 70%, 80%, 90%, 95%, 96%, 97%, 98%, or 99% identity to SEQ ID NO: 17; and/or a light chain variable sequence having a sequence according to SEQ ID NO: 23, or a light chain variable sequence having at least 70%, 80%, 90%, 95%, 96%, 97%, 98%, or 99% identity to SEQ ID NO: 23;
(xxxvii) a heavy chain variable sequence having a sequence according to SEQ ID NO: 17, or a heavy chain variable sequence having at least 70%, 80%, 90%, 95%, 96%, 97%, 98%, or 99% identity to SEQ ID NO: 17; and/or a light chain variable sequence having a sequence according to SEQ ID NO: 24, or a light chain variable sequence having at least 70%, 80%, 90%, 95%, 96%, 97%, 98%, or 99% identity to SEQ ID NO: 24;
(xxxviii) a heavy chain variable sequence having a sequence according to SEQ ID NO: 26, or a heavy chain variable sequence having at least 70%, 80%, 90%, 95%, 96%, 97%, 98%, or 99% identity to SEQ ID NO: 26; and/or a light chain variable sequence having a sequence according to SEQ ID NO: 105, or a light chain variable sequence having at least 70%, 80%, 90%, 95%, 96%, 97%, 98%, or 99% identity to SEQ ID NO: 105;
(xxxix) a heavy chain variable sequence having a sequence according to SEQ ID NO: 27, or a heavy chain variable sequence having at least 70%, 80%, 90%, 95%, 96%, 97%, 98%, or 99% identity to SEQ ID NO: 27; and/or a light chain variable sequence having a sequence according to SEQ ID NO: 105, or a light chain variable sequence having at least 70%, 80%, 90%, 95%, 96%, 97%, 98%, or 99% identity to SEQ ID NO: 105;
(xl) a heavy chain variable sequence having a sequence according to SEQ ID NO: 28, or a heavy chain variable sequence having at least 70%, 80%, 90%, 95%, 96%, 97%, 98%, or 99% identity to SEQ ID NO: 28; and/or a light chain variable sequence having a sequence according to SEQ ID NO: 105, or a light chain variable sequence having at least 70%, 80%, 90%, 95%, 96%, 97%, 98%, or 99% identity to SEQ ID NO: 105;
(xli) a heavy chain variable sequence having a sequence according to SEQ ID NO: 29, or a heavy chain variable sequence having at least 70%, 80%, 90%, 95%, 96%, 97%, 98%, or 99% identity to SEQ ID NO: 29; and/or a light chain variable sequence having a sequence according to SEQ ID NO: 105, or a light chain variable sequence having at least 70%, 80%, 90%, 95%, 96%, 97%, 98%, or 99% identity to SEQ ID NO: 105;
(xlii) a heavy chain variable sequence having a sequence according to SEQ ID NO: 30, or a heavy chain variable sequence having at least 70%, 80%, 90%, 95%, 96%, 97%, 98%, or 99% identity to SEQ ID NO: 30; and/or a light chain variable sequence having a sequence according to SEQ ID NO: 106, or a light chain variable sequence having at least 70%, 80%, 90%, 95%, 96%, 97%, 98%, or 99% identity to SEQ ID NO: 106;
(xliii) a heavy chain variable sequence having a sequence according to SEQ ID NO: 31, or a heavy chain variable sequence having at least 70%, 80%, 90%, 95%, 96%, 97%, 98%, or 99% identity to SEQ ID NO: 31; and/or a light chain variable sequence having a sequence according to SEQ ID NO: 105, or a light chain variable sequence having at least 70%, 80%, 90%, 95%, 96%, 97%, 98%, or 99% identity to SEQ ID NO: 105;
(xliv) a heavy chain variable sequence having a sequence according to SEQ ID NO: 32, or a heavy chain variable sequence having at least 70%, 80%, 90%, 95%, 96%, 97%, 98%, or 99% identity to SEQ ID NO: 32; and/or a light chain variable sequence having a sequence according to SEQ ID NO: 105, or a light chain variable sequence having at least 70%, 80%, 90%, 95%, 96%, 97%, 98%, or 99% identity to SEQ ID NO: 105;
(xlv) a heavy chain variable sequence having a sequence according to SEQ ID NO: 30, or a heavy chain variable sequence having at least 70%, 80%, 90%, 95%, 96%, 97%, 98%, or 99% identity to SEQ ID NO: 30; and/or a light chain variable sequence having a sequence according to SEQ ID NO: 107, or a light chain variable sequence having at least 70%, 80%, 90%, 95%, 96%, 97%, 98%, or 99% identity to SEQ ID NO: 107;
(xlvi) a heavy chain variable sequence having a sequence according to SEQ ID NO: 33, or a heavy chain variable sequence having at least 70%, 80%, 90%, 95%, 96%, 97%, 98%, or 99% identity to SEQ ID NO: 33; and/or a light chain variable sequence having a sequence according to SEQ ID NO: 105, or a light chain variable sequence having at least 70%, 80%, 90%, 95%, 96%, 97%, 98%, or 99% identity to SEQ ID NO: 105;
(xlvii) a heavy chain variable sequence having a sequence according to SEQ ID NO: 34, or a heavy chain variable sequence having at least 70%, 80%, 90%, 95%, 96%, 97%, 98%, or 99% identity to SEQ ID NO: 34; and/or a light chain variable sequence having a sequence according to SEQ ID NO: 105, or a light chain variable sequence having at least 70%, 80%, 90%, 95%, 96%, 97%, 98%, or 99% identity to SEQ ID NO: 105;
(xlviii) a heavy chain variable sequence having a sequence according to SEQ ID NO: 30, or a heavy chain variable sequence having at least 70%, 80%, 90%, 95%, 96%, 97%, 98%, or 99% identity to SEQ ID NO: 30; and/or a light chain variable sequence having a sequence according to SEQ ID NO: 108, or a light chain variable sequence having at least 70%, 80%, 90%, 95%, 96%, 97%, 98%, or 99% identity to SEQ ID NO: 108;
(xlix) a heavy chain variable sequence having a sequence according to SEQ ID NO: 30, or a heavy chain variable sequence having at least 70%, 80%, 90%, 95%, 96%, 97%, 98%, or 99% identity to SEQ ID NO: 30; and/or a light chain variable sequence having a sequence according to SEQ ID NO: 109, or a light chain variable sequence having at least 70%, 80%, 90%, 95%, 96%, 97%, 98%, or 99% identity to SEQ ID NO: 109;
(l) a heavy chain variable sequence having a sequence according to SEQ ID NO: 35, or a heavy chain variable sequence having at least 70%, 80%, 90%, 95%, 96%, 97%, 98%, or 99% identity to SEQ ID NO: 35; and/or a light chain variable sequence having a sequence according to SEQ ID NO: 105, or a light chain variable sequence having at least 70%, 80%, 90%, 95%, 96%, 97%, 98%, or 99% identity to SEQ ID NO: 105;
(li) a heavy chain variable sequence having a sequence according to SEQ ID NO: 36, or a heavy chain variable sequence having at least 70%, 80%, 90%, 95%, 96%, 97%, 98%, or 99% identity to SEQ ID NO: 36; and/or a light chain variable sequence having a sequence according to SEQ ID NO: 105, or a light chain variable sequence having at least 70%, 80%, 90%, 95%, 96%, 97%, 98%, or 99% identity to SEQ ID NO: 105;
(lii) a heavy chain variable sequence having a sequence according to SEQ ID NO: 37, or a heavy chain variable sequence having at least 70%, 80%, 90%, 95%, 96%, 97%, 98%, or 99% identity to SEQ ID NO: 37; and/or a light chain variable sequence having a sequence according to SEQ ID NO: 105, or a light chain variable sequence having at least 70%, 80%, 90%, 95%, 96%, 97%, 98%, or 99% identity to SEQ ID NO: 105;
(liii) a heavy chain variable sequence having a sequence according to SEQ ID NO: 26, or a heavy chain variable sequence having at least 70%, 80%, 90%, 95%, 96%, 97%, 98%, or 99% identity to SEQ ID NO: 26; and/or a light chain variable sequence having a sequence according to SEQ ID NO: 107, or a light chain variable sequence having at least 70%, 80%, 90%, 95%, 96%, 97%, 98%, or 99% identity to SEQ ID NO: 107;
(liv) a heavy chain variable sequence having a sequence according to SEQ ID NO: 38, or a heavy chain variable sequence having at least 70%, 80%, 90%, 95%, 96%, 97%, 98%, or 99% identity to SEQ ID NO: 38; and/or a light chain variable sequence having a sequence according to SEQ ID NO: 105, or a light chain variable sequence having at least 70%, 80%, 90%, 95%, 96%, 97%, 98%, or 99% identity to SEQ ID NO: 105;
(lv) a heavy chain variable sequence having a sequence according to SEQ ID NO: 31, or a heavy chain variable sequence having at least 70%, 80%, 90%, 95%, 96%, 97%, 98%, or 99% identity to SEQ ID NO: 31; and/or a light chain variable sequence having a sequence according to SEQ ID NO: 110, or a light chain variable sequence having at least 70%, 80%, 90%, 95%, 96%, 97%, 98%, or 99% identity to SEQ ID NO: 110;
(lvi) a heavy chain variable sequence having a sequence according to SEQ ID NO: 39, or a heavy chain variable sequence having at least 70%, 80%, 90%, 95%, 96%, 97%, 98%, or 99% identity to SEQ ID NO: 39; and/or a light chain variable sequence having a sequence according to SEQ ID NO: 105, or a light chain variable sequence having at least 70%, 80%, 90%, 95%, 96%, 97%, 98%, or 99% identity to SEQ ID NO: 105;
(lvii) a heavy chain variable sequence having a sequence according to SEQ ID NO: 40, or a heavy chain variable sequence having at least 70%, 80%, 90%, 95%, 96%, 97%, 98%, or 99% identity to SEQ ID NO: 40; and/or a light chain variable sequence having a sequence according to SEQ ID NO: 105, or a light chain variable sequence having at least 70%, 80%, 90%, 95%, 96%, 97%, 98%, or 99% identity to SEQ ID NO: 105;
(lviii) a heavy chain variable sequence having a sequence according to SEQ ID NO: 34, or a heavy chain variable sequence having at least 70%, 80%, 90%, 95%, 96%, 97%, 98%, or 99% identity to SEQ ID NO: 34; and/or a light chain variable sequence having a sequence according to SEQ ID NO: 111, or a light chain variable sequence having at least 70%, 80%, 90%, 95%, 96%, 97%, 98%, or 99% identity to SEQ ID NO: 111;
(lix) a heavy chain variable sequence having a sequence according to SEQ ID NO: 41, or a heavy chain variable sequence having at least 70%, 80%, 90%, 95%, 96%, 97%, 98%, or 99% identity to SEQ ID NO: 41; and/or a light chain variable sequence having a sequence according to SEQ ID NO: 109, or a light chain variable sequence having at least 70%, 80%, 90%, 95%, 96%, 97%, 98%, or 99% identity to SEQ ID NO: 109;
(lx) a heavy chain variable sequence having a sequence according to SEQ ID NO: 30, or a heavy chain variable sequence having at least 70%, 80%, 90%, 95%, 96%, 97%, 98%, or 99% identity to SEQ ID NO: 30; and/or a light chain variable sequence having a sequence according to SEQ ID NO: 112, or a light chain variable sequence having at least 70%, 80%, 90%, 95%, 96%, 97%, 98%, or 99% identity to SEQ ID NO: 112;
(lxi) a heavy chain variable sequence having a sequence according to SEQ ID NO: 28, or a heavy chain variable sequence having at least 70%, 80%, 90%, 95%, 96%, 97%, 98%, or 99% identity to SEQ ID NO: 28; and/or a light chain variable sequence having a sequence according to SEQ ID NO: 113, or a light chain variable sequence having at least 70%, 80%, 90%, 95%, 96%, 97%, 98%, or 99% identity to SEQ ID NO: 113;
(lxii) a heavy chain variable sequence having a sequence according to SEQ ID NO: 32, or a heavy chain variable sequence having at least 70%, 80%, 90%, 95%, 96%, 97%, 98%, or 99% identity to SEQ ID NO: 32; and/or a light chain variable sequence having a sequence according to SEQ ID NO: 114, or a light chain variable sequence having at least 70%, 80%, 90%, 95%, 96%, 97%, 98%, or 99% identity to SEQ ID NO: 114;
(lxiii) a heavy chain variable sequence having a sequence according to SEQ ID NO: 42, or a heavy chain variable sequence having at least 70%, 80%, 90%, 95%, 96%, 97%, 98%, or 99% identity to SEQ ID NO: 42; and/or a light chain variable sequence having a sequence according to SEQ ID NO: 105, or a light chain variable sequence having at least 70%, 80%, 90%, 95%, 96%, 97%, 98%, or 99% identity to SEQ ID NO: 105;
(lxiv) a heavy chain variable sequence having a sequence according to SEQ ID NO: 36, or a heavy chain variable sequence having at least 70%, 80%, 90%, 95%, 96%, 97%, 98%, or 99% identity to SEQ ID NO: 36; and/or a light chain variable sequence having a sequence according to SEQ ID NO: 115, or a light chain variable sequence having at least 70%, 80%, 90%, 95%, 96%, 97%, 98%, or 99% identity to SEQ ID NO: 115;
(lxv) a heavy chain variable sequence having a sequence according to SEQ ID NO: 43, or a heavy chain variable sequence having at least 70%, 80%, 90%, 95%, 96%, 97%, 98%, or 99% identity to SEQ ID NO: 43; and/or a light chain variable sequence having a sequence according to SEQ ID NO: 105, or a light chain variable sequence having at least 70%, 80%, 90%, 95%, 96%, 97%, 98%, or 99% identity to SEQ ID NO: 105;
(lxvi) a heavy chain variable sequence having a sequence according to SEQ ID NO: 32, or a heavy chain variable sequence having at least 70%, 80%, 90%, 95%, 96%, 97%, 98%, or 99% identity to SEQ ID NO: 32; and/or a light chain variable sequence having a sequence according to SEQ ID NO: 109, or a light chain variable sequence having at least 70%, 80%, 90%, 95%, 96%, 97%, 98%, or 99% identity to SEQ ID NO: 109;
(lxvii) a heavy chain variable sequence having a sequence according to SEQ ID NO: 44, or a heavy chain variable sequence having at least 70%, 80%, 90%, 95%, 96%, 97%, 98%, or 99% identity to SEQ ID NO: 44; and/or a light chain variable sequence having a sequence according to SEQ ID NO: 116, or a light chain variable sequence having at least 70%, 80%, 90%, 95%, 96%, 97%, 98%, or 99% identity to SEQ ID NO: 116;
(lxviii) a heavy chain variable sequence having a sequence according to SEQ ID NO: 35, or a heavy chain variable sequence having at least 70%, 80%, 90%, 95%, 96%, 97%, 98%, or 99% identity to SEQ ID NO: 35; and/or a light chain variable sequence having a sequence according to SEQ ID NO: 117, or a light chain variable sequence having at least 70%, 80%, 90%, 95%, 96%, 97%, 98%, or 99% identity to SEQ ID NO: 117;
(lxix) a heavy chain variable sequence having a sequence according to SEQ ID NO: 45, or a heavy chain variable sequence having at least 70%, 80%, 90%, 95%, 96%, 97%, 98%, or 99% identity to SEQ ID NO: 45; and/or a light chain variable sequence having a sequence according to SEQ ID NO: 105, or a light chain variable sequence having at least 70%, 80%, 90%, 95%, 96%, 97%, 98%, or 99% identity to SEQ ID NO: 105;
(lxx) a heavy chain variable sequence having a sequence according to SEQ ID NO: 46, or a heavy chain variable sequence having at least 70%, 80%, 90%, 95%, 96%, 97%, 98%, or 99% identity to SEQ ID NO: 46; and/or a light chain variable sequence having a sequence according to SEQ ID NO: 105, or a light chain variable sequence having at least 70%, 80%, 90%, 95%, 96%, 97%, 98%, or 99% identity to SEQ ID NO: 105;
(lxxi) a heavy chain variable sequence having a sequence according to SEQ ID NO: 36, or a heavy chain variable sequence having at least 70%, 80%, 90%, 95%, 96%, 97%, 98%, or 99% identity to SEQ ID NO: 36; and/or a light chain variable sequence having a sequence according to SEQ ID NO: 118, or a light chain variable sequence having at least 70%, 80%, 90%, 95%, 96%, 97%, 98%, or 99% identity to SEQ ID NO: 118;
(lxxii) a heavy chain variable sequence having a sequence according to SEQ ID NO: 47, or a heavy chain variable sequence having at least 70%, 80%, 90%, 95%, 96%, 97%, 98%, or 99% identity to SEQ ID NO: 47; and/or a light chain variable sequence having a sequence according to SEQ ID NO: 115, or a light chain variable sequence having at least 70%, 80%, 90%, 95%, 96%, 97%, 98%, or 99% identity to SEQ ID NO: 115;
(lxxiii) a heavy chain variable sequence having a sequence according to SEQ ID NO: 48, or a heavy chain variable sequence having at least 70%, 80%, 90%, 95%, 96%, 97%, 98%, or 99% identity to SEQ ID NO: 48; and/or a light chain variable sequence having a sequence according to SEQ ID NO: 109, or a light chain variable sequence having at least 70%, 80%, 90%, 95%, 96%, 97%, 98%, or 99% identity to SEQ ID NO: 109;
(lxxiv) a heavy chain variable sequence having a sequence according to SEQ ID NO: 49, or a heavy chain variable sequence having at least 70%, 80%, 90%, 95%, 96%, 97%, 98%, or 99% identity to SEQ ID NO: 49; and/or a light chain variable sequence having a sequence according to SEQ ID NO: 105, or a light chain variable sequence having at least 70%, 80%, 90%, 95%, 96%, 97%, 98%, or 99% identity to SEQ ID NO: 105;
(lxxv) a heavy chain variable sequence having a sequence according to SEQ ID NO: 50, or a heavy chain variable sequence having at least 70%, 80%, 90%, 95%, 96%, 97%, 98%, or 99% identity to SEQ ID NO: 50; and/or a light chain variable sequence having a sequence according to SEQ ID NO: 105, or a light chain variable sequence having at least 70%, 80%, 90%, 95%, 96%, 97%, 98%, or 99% identity to SEQ ID NO: 105;
(lxxvi) a heavy chain variable sequence having a sequence according to SEQ ID NO: 51, or a heavy chain variable sequence having at least 70%, 80%, 90%, 95%, 96%, 97%, 98%, or 99% identity to SEQ ID NO: 51; and/or a light chain variable sequence having a sequence according to SEQ ID NO: 106, or a light chain variable sequence having at least 70%, 80%, 90%, 95%, 96%, 97%, 98%, or 99% identity to SEQ ID NO: 106;
(lxxvii) a heavy chain variable sequence having a sequence according to SEQ ID NO: 52, or a heavy chain variable sequence having at least 70%, 80%, 90%, 95%, 96%, 97%, 98%, or 99% identity to SEQ ID NO: 52; and/or a light chain variable sequence having a sequence according to SEQ ID NO: 119, or a light chain variable sequence having at least 70%, 80%, 90%, 95%, 96%, 97%, 98%, or 99% identity to SEQ ID NO: 119;
(lxxviii) a heavy chain variable sequence having a sequence according to SEQ ID NO: 53, or a heavy chain variable sequence having at least 70%, 80%, 90%, 95%, 96%, 97%, 98%, or 99% identity to SEQ ID NO: 53; and/or a light chain variable sequence having a sequence according to SEQ ID NO: 108, or a light chain variable sequence having at least 70%, 80%, 90%, 95%, 96%, 97%, 98%, or 99% identity to SEQ ID NO: 108;
(lxxix) a heavy chain variable sequence having a sequence according to SEQ ID NO: 54, or a heavy chain variable sequence having at least 70%, 80%, 90%, 95%, 96%, 97%, 98%, or 99% identity to SEQ ID NO: 54; and/or a light chain variable sequence having a sequence according to SEQ ID NO: 105, or a light chain variable sequence having at least 70%, 80%, 90%, 95%, 96%, 97%, 98%, or 99% identity to SEQ ID NO: 105;
(lxxx) a heavy chain variable sequence having a sequence according to SEQ ID NO: 55, or a heavy chain variable sequence having at least 70%, 80%, 90%, 95%, 96%, 97%, 98%, or 99% identity to SEQ ID NO: 55; and/or a light chain variable sequence having a sequence according to SEQ ID NO: 116, or a light chain variable sequence having at least 70%, 80%, 90%, 95%, 96%, 97%, 98%, or 99% identity to SEQ ID NO: 116;
(lxxxi) a heavy chain variable sequence having a sequence according to SEQ ID NO: 56, or a heavy chain variable sequence having at least 70%, 80%, 90%, 95%, 96%, 97%, 98%, or 99% identity to SEQ ID NO: 56; and/or a light chain variable sequence having a sequence according to SEQ ID NO: 116, or a light chain variable sequence having at least 70%, 80%, 90%, 95%, 96%, 97%, 98%, or 99% identity to SEQ ID NO: 116;
(lxxxii) a heavy chain variable sequence having a sequence according to SEQ ID NO: 57, or a heavy chain variable sequence having at least 70%, 80%, 90%, 95%, 96%, 97%, 98%, or 99% identity to SEQ ID NO: 57; and/or a light chain variable sequence having a sequence according to SEQ ID NO: 120, or a light chain variable sequence having at least 70%, 80%, 90%, 95%, 96%, 97%, 98%, or 99% identity to SEQ ID NO: 120;
(lxxxiii) a heavy chain variable sequence having a sequence according to SEQ ID NO: 58, or a heavy chain variable sequence having at least 70%, 80%, 90%, 95%, 96%, 97%, 98%, or 99% identity to SEQ ID NO: 58; and/or a light chain variable sequence having a sequence according to SEQ ID NO: 121, or a light chain variable sequence having at least 70%, 80%, 90%, 95%, 96%, 97%, 98%, or 99% identity to SEQ ID NO: 121;
(lxxxiv) a heavy chain variable sequence having a sequence according to SEQ ID NO: 59, or a heavy chain variable sequence having at least 70%, 80%, 90%, 95%, 96%, 97%, 98%, or 99% identity to SEQ ID NO: 59; and/or a light chain variable sequence having a sequence according to SEQ ID NO: 122, or a light chain variable sequence having at least 70%, 80%, 90%, 95%, 96%, 97%, 98%, or 99% identity to SEQ ID NO: 122;
(lxxxv) a heavy chain variable sequence having a sequence according to SEQ ID NO: 60, or a heavy chain variable sequence having at least 70%, 80%, 90%, 95%, 96%, 97%, 98%, or 99% identity to SEQ ID NO: 60; and/or a light chain variable sequence having a sequence according to SEQ ID NO: 108, or a light chain variable sequence having at least 70%, 80%, 90%, 95%, 96%, 97%, 98%, or 99% identity to SEQ ID NO: 108;
(lxxxvi) a heavy chain variable sequence having a sequence according to SEQ ID NO: 61, or a heavy chain variable sequence having at least 70%, 80%, 90%, 95%, 96%, 97%, 98%, or 99% identity to SEQ ID NO: 61; and/or a light chain variable sequence having a sequence according to SEQ ID NO: 123, or a light chain variable sequence having at least 70%, 80%, 90%, 95%, 96%, 97%, 98%, or 99% identity to SEQ ID NO: 123;
(lxxxvii) a heavy chain variable sequence having a sequence according to SEQ ID NO: 62, or a heavy chain variable sequence having at least 70%, 80%, 90%, 95%, 96%, 97%, 98%, or 99% identity to SEQ ID NO: 62; and/or a light chain variable sequence having a sequence according to SEQ ID NO: 114, or a light chain variable sequence having at least 70%, 80%, 90%, 95%, 96%, 97%, 98%, or 99% identity to SEQ ID NO: 114;
(lxxxviii) a heavy chain variable sequence having a sequence according to SEQ ID NO: 63, or a heavy chain variable sequence having at least 70%, 80%, 90%, 95%, 96%, 97%, 98%, or 99% identity to SEQ ID NO: 63; and/or a light chain variable sequence having a sequence according to SEQ ID NO: 124, or a light chain variable sequence having at least 70%, 80%, 90%, 95%, 96%, 97%, 98%, or 99% identity to SEQ ID NO: 124;
(lxxxix) a heavy chain variable sequence having a sequence according to SEQ ID NO: 64, or a heavy chain variable sequence having at least 70%, 80%, 90%, 95%, 96%, 97%, 98%, or 99% identity to SEQ ID NO: 64; and/or a light chain variable sequence having a sequence according to SEQ ID NO: 105, or a light chain variable sequence having at least 70%, 80%, 90%, 95%, 96%, 97%, 98%, or 99% identity to SEQ ID NO: 105;
(xc) a heavy chain variable sequence having a sequence according to SEQ ID NO: 65, or a heavy chain variable sequence having at least 70%, 80%, 90%, 95%, 96%, 97%, 98%, or 99% identity to SEQ ID NO: 65; and/or a light chain variable sequence having a sequence according to SEQ ID NO: 125, or a light chain variable sequence having at least 70%, 80%, 90%, 95%, 96%, 97%, 98%, or 99% identity to SEQ ID NO: 125;
(xci) a heavy chain variable sequence having a sequence according to SEQ ID NO: 66, or a heavy chain variable sequence having at least 70%, 80%, 90%, 95%, 96%, 97%, 98%, or 99% identity to SEQ ID NO: 66; and/or a light chain variable sequence having a sequence according to SEQ ID NO: 105, or a light chain variable sequence having at least 70%, 80%, 90%, 95%, 96%, 97%, 98%, or 99% identity to SEQ ID NO: 105;
(xcii) a heavy chain variable sequence having a sequence according to SEQ ID NO: 67, or a heavy chain variable sequence having at least 70%, 80%, 90%, 95%, 96%, 97%, 98%, or 99% identity to SEQ ID NO: 67; and/or a light chain variable sequence having a sequence according to SEQ ID NO: 125, or a light chain variable sequence having at least 70%, 80%, 90%, 95%, 96%, 97%, 98%, or 99% identity to SEQ ID NO: 125;
(xciii) a heavy chain variable sequence having a sequence according to SEQ ID NO: 68, or a heavy chain variable sequence having at least 70%, 80%, 90%, 95%, 96%, 97%, 98%, or 99% identity to SEQ ID NO: 68; and/or a light chain variable sequence having a sequence according to SEQ ID NO: 126, or a light chain variable sequence having at least 70%, 80%, 90%, 95%, 96%, 97%, 98%, or 99% identity to SEQ ID NO: 126;
(xciv) a heavy chain variable sequence having a sequence according to SEQ ID NO: 69, or a heavy chain variable sequence having at least 70%, 80%, 90%, 95%, 96%, 97%, 98%, or 99% identity to SEQ ID NO: 69; and/or a light chain variable sequence having a sequence according to SEQ ID NO: 127, or a light chain variable sequence having at least 70%, 80%, 90%, 95%, 96%, 97%, 98%, or 99% identity to SEQ ID NO: 127;
(xcv) a heavy chain variable sequence having a sequence according to SEQ ID NO: 70, or a heavy chain variable sequence having at least 70%, 80%, 90%, 95%, 96%, 97%, 98%, or 99% identity to SEQ ID NO: 70; and/or a light chain variable sequence having a sequence according to SEQ ID NO: 128, or a light chain variable sequence having at least 70%, 80%, 90%, 95%, 96%, 97%, 98%, or 99% identity to SEQ ID NO: 128;
(xcvi) a heavy chain variable sequence having a sequence according to SEQ ID NO: 71, or a heavy chain variable sequence having at least 70%, 80%, 90%, 95%, 96%, 97%, 98%, or 99% identity to SEQ ID NO: 71; and/or a light chain variable sequence having a sequence according to SEQ ID NO: 117, or a light chain variable sequence having at least 70%, 80%, 90%, 95%, 96%, 97%, 98%, or 99% identity to SEQ ID NO: 117;
(xcvii) a heavy chain variable sequence having a sequence according to SEQ ID NO: 72, or a heavy chain variable sequence having at least 70%, 80%, 90%, 95%, 96%, 97%, 98%, or 99% identity to SEQ ID NO: 72; and/or a light chain variable sequence having a sequence according to SEQ ID NO: 129, or a light chain variable sequence having at least 70%, 80%, 90%, 95%, 96%, 97%, 98%, or 99% identity to SEQ ID NO: 129;
(xcviii) a heavy chain variable sequence having a sequence according to SEQ ID NO: 73, or a heavy chain variable sequence having at least 70%, 80%, 90%, 95%, 96%, 97%, 98%, or 99% identity to SEQ ID NO: 73; and/or a light chain variable sequence having a sequence according to SEQ ID NO: 130, or a light chain variable sequence having at least 70%, 80%, 90%, 95%, 96%, 97%, 98%, or 99% identity to SEQ ID NO: 130;
(xcix) a heavy chain variable sequence having a sequence according to SEQ ID NO: 74, or a heavy chain variable sequence having at least 70%, 80%, 90%, 95%, 96%, 97%, 98%, or 99% identity to SEQ ID NO: 74; and/or a light chain variable sequence having a sequence according to SEQ ID NO: 131, or a light chain variable sequence having at least 70%, 80%, 90%, 95%, 96%, 97%, 98%, or 99% identity to SEQ ID NO: 131;
(c) a heavy chain variable sequence having a sequence according to SEQ ID NO: 73, or a heavy chain variable sequence having at least 70%, 80%, 90%, 95%, 96%, 97%, 98%, or 99% identity to SEQ ID NO: 73; and/or a light chain variable sequence having a sequence according to SEQ ID NO: 132, or a light chain variable sequence having at least 70%, 80%, 90%, 95%, 96%, 97%, 98%, or 99% identity to SEQ ID NO: 132;
(ci) a heavy chain variable sequence having a sequence according to SEQ ID NO: 75, or a heavy chain variable sequence having at least 70%, 80%, 90%, 95%, 96%, 97%, 98%, or 99% identity to SEQ ID NO: 75; and/or a light chain variable sequence having a sequence according to SEQ ID NO: 133, or a light chain variable sequence having at least 70%, 80%, 90%, 95%, 96%, 97%, 98%, or 99% identity to SEQ ID NO: 133;
(cii) a heavy chain variable sequence having a sequence according to SEQ ID NO: 76, or a heavy chain variable sequence having at least 70%, 80%, 90%, 95%, 96%, 97%, 98%, or 99% identity to SEQ ID NO: 76; and/or a light chain variable sequence having a sequence according to SEQ ID NO: 134, or a light chain variable sequence having at least 70%, 80%, 90%, 95%, 96%, 97%, 98%, or 99% identity to SEQ ID NO: 134;
(ciii) a heavy chain variable sequence having a sequence according to SEQ ID NO: 77, or a heavy chain variable sequence having at least 70%, 80%, 90%, 95%, 96%, 97%, 98%, or 99% identity to SEQ ID NO: 77; and/or a light chain variable sequence having a sequence according to SEQ ID NO: 107, or a light chain variable sequence having at least 70%, 80%, 90%, 95%, 96%, 97%, 98%, or 99% identity to SEQ ID NO: 107;
(civ) a heavy chain variable sequence having a sequence according to SEQ ID NO: 78, or a heavy chain variable sequence having at least 70%, 80%, 90%, 95%, 96%, 97%, 98%, or 99% identity to SEQ ID NO: 78; and/or a light chain variable sequence having a sequence according to SEQ ID NO: 135, or a light chain variable sequence having at least 70%, 80%, 90%, 95%, 96%, 97%, 98%, or 99% identity to SEQ ID NO: 135;
(cv) a heavy chain variable sequence having a sequence according to SEQ ID NO: 79, or a heavy chain variable sequence having at least 70%, 80%, 90%, 95%, 96%, 97%, 98%, or 99% identity to SEQ ID NO: 79; and/or a light chain variable sequence having a sequence according to SEQ ID NO: 136, or a light chain variable sequence having at least 70%, 80%, 90%, 95%, 96%, 97%, 98%, or 99% identity to SEQ ID NO: 136;
(cvi) a heavy chain variable sequence having a sequence according to SEQ ID NO: 80, or a heavy chain variable sequence having at least 70%, 80%, 90%, 95%, 96%, 97%, 98%, or 99% identity to SEQ ID NO: 80; and/or a light chain variable sequence having a sequence according to SEQ ID NO: 137, or a light chain variable sequence having at least 70%, 80%, 90%, 95%, 96%, 97%, 98%, or 99% identity to SEQ ID NO: 137;
(cvii) a heavy chain variable sequence having a sequence according to SEQ ID NO: 41, or a heavy chain variable sequence having at least 70%, 80%, 90%, 95%, 96%, 97%, 98%, or 99% identity to SEQ ID NO: 41; and/or a light chain variable sequence having a sequence according to SEQ ID NO: 138, or a light chain variable sequence having at least 70%, 80%, 90%, 95%, 96%, 97%, 98%, or 99% identity to SEQ ID NO: 138;
(cviii) a heavy chain variable sequence having a sequence according to SEQ ID NO: 81, or a heavy chain variable sequence having at least 70%, 80%, 90%, 95%, 96%, 97%, 98%, or 99% identity to SEQ ID NO: 31; and/or a light chain variable sequence having a sequence according to SEQ ID NO: 139, or a light chain variable sequence having at least 70%, 80%, 90%, 95%, 96%, 97%, 98%, or 99% identity to SEQ ID NO: 139;
(cix) a heavy chain variable sequence having a sequence according to SEQ ID NO: 82, or a heavy chain variable sequence having at least 70%, 80%, 90%, 95%, 96%, 97%, 98%, or 99% identity to SEQ ID NO: 82; and/or a light chain variable sequence having a sequence according to SEQ ID NO: 105, or a light chain variable sequence having at least 70%, 80%, 90%, 95%, 96%, 97%, 98%, or 99% identity to SEQ ID NO: 105;
(cx) a heavy chain variable sequence having a sequence according to SEQ ID NO: 83, or a heavy chain variable sequence having at least 70%, 80%, 90%, 95%, 96%, 97%, 98%, or 99% identity to SEQ ID NO: 83; and/or a light chain variable sequence having a sequence according to SEQ ID NO: 126, or a light chain variable sequence having at least 70%, 80%, 90%, 95%, 96%, 97%, 98%, or 99% identity to SEQ ID NO: 126;
(cxi) a heavy chain variable sequence having a sequence according to SEQ ID NO: 84, or a heavy chain variable sequence having at least 70%, 80%, 90%, 95%, 96%, 97%, 98%, or 99% identity to SEQ ID NO: 84; and/or a light chain variable sequence having a sequence according to SEQ ID NO: 140, or a light chain variable sequence having at least 70%, 80%, 90%, 95%, 96%, 97%, 98%, or 99% identity to SEQ ID NO: 140;
(cxii) a heavy chain variable sequence having a sequence according to SEQ ID NO: 85, or a heavy chain variable sequence having at least 70%, 80%, 90%, 95%, 96%, 97%, 98%, or 99% identity to SEQ ID NO: 85; and/or a light chain variable sequence having a sequence according to SEQ ID NO: 141, or a light chain variable sequence having at least 70%, 80%, 90%, 95%, 96%, 97%, 98%, or 99% identity to SEQ ID NO: 141;
(cxiii) a heavy chain variable sequence having a sequence according to SEQ ID NO: 86, or a heavy chain variable sequence having at least 70%, 80%, 90%, 95%, 96%, 97%, 98%, or 99% identity to SEQ ID NO: 86; and/or a light chain variable sequence having a sequence according to SEQ ID NO: 141, or a light chain variable sequence having at least 70%, 80%, 90%, 95%, 96%, 97%, 98%, or 99% identity to SEQ ID NO: 141;
(cxiv) a heavy chain variable sequence having a sequence according to SEQ ID NO: 87, or a heavy chain variable sequence having at least 70%, 80%, 90%, 95%, 96%, 97%, 98%, or 99% identity to SEQ ID NO: 87; and/or a light chain variable sequence having a sequence according to SEQ ID NO: 117, or a light chain variable sequence having at least 70%, 80%, 90%, 95%, 96%, 97%, 98%, or 99% identity to SEQ ID NO: 117;
(cxv) a heavy chain variable sequence having a sequence according to SEQ ID NO: 88, or a heavy chain variable sequence having at least 70%, 80%, 90%, 95%, 96%, 97%, 98%, or 99% identity to SEQ ID NO: 88; and/or a light chain variable sequence having a sequence according to SEQ ID NO: 142, or a light chain variable sequence having at least 70%, 80%, 90%, 95%, 96%, 97%, 98%, or 99% identity to SEQ ID NO: 142;
(cxvi) a heavy chain variable sequence having a sequence according to SEQ ID NO: 89, or a heavy chain variable sequence having at least 70%, 80%, 90%, 95%, 96%, 97%, 98%, or 99% identity to SEQ ID NO: 89; and/or a light chain variable sequence having a sequence according to SEQ ID NO: 143, or a light chain variable sequence having at least 70%, 80%, 90%, 95%, 96%, 97%, 98%, or 99% identity to SEQ ID NO: 143;
(cxvii) a heavy chain variable sequence having a sequence according to SEQ ID NO: 90, or a heavy chain variable sequence having at least 70%, 80%, 90%, 95%, 96%, 97%, 98%, or 99% identity to SEQ ID NO: 90; and/or a light chain variable sequence having a sequence according to SEQ ID NO: 144, or a light chain variable sequence having at least 70%, 80%, 90%, 95%, 96%, 97%, 98%, or 99% identity to SEQ ID NO: 144;
(cxviii) a heavy chain variable sequence having a sequence according to SEQ ID NO: 91, or a heavy chain variable sequence having at least 70%, 80%, 90%, 95%, 96%, 97%, 98%, or 99% identity to SEQ ID NO: 91; and/or a light chain variable sequence having a sequence according to SEQ ID NO: 109, or a light chain variable sequence having at least 70%, 80%, 90%, 95%, 96%, 97%, 98%, or 99% identity to SEQ ID NO: 109;
(cxix) a heavy chain variable sequence having a sequence according to SEQ ID NO: 92, or a heavy chain variable sequence having at least 70%, 80%, 90%, 95%, 96%, 97%, 98%, or 99% identity to SEQ ID NO: 92; and/or a light chain variable sequence having a sequence according to SEQ ID NO: 145, or a light chain variable sequence having at least 70%, 80%, 90%, 95%, 96%, 97%, 98%, or 99% identity to SEQ ID NO: 145;
(cxx) a heavy chain variable sequence having a sequence according to SEQ ID NO: 93, or a heavy chain variable sequence having at least 70%, 80%, 90%, 95%, 96%, 97%, 98%, or 99% identity to SEQ ID NO: 93; and/or a light chain variable sequence having a sequence according to SEQ ID NO: 146, or a light chain variable sequence having at least 70%, 80%, 90%, 95%, 96%, 97%, 98%, or 99% identity to SEQ ID NO: 146;
(cxxi) a heavy chain variable sequence having a sequence according to SEQ ID NO: 94, or a heavy chain variable sequence having at least 70%, 80%, 90%, 95%, 96%, 97%, 98%, or 99% identity to SEQ ID NO: 94; and/or a light chain variable sequence having a sequence according to SEQ ID NO: 147, or a light chain variable sequence having at least 70%, 80%, 90%, 95%, 96%, 97%, 98%, or 99% identity to SEQ ID NO: 147;
(cxxii) a heavy chain variable sequence having a sequence according to SEQ ID NO: 95, or a heavy chain variable sequence having at least 70%, 80%, 90%, 95%, 96%, 97%, 98%, or 99% identity to SEQ ID NO: 95; and/or a light chain variable sequence having a sequence according to SEQ ID NO: 148, or a light chain variable sequence having at least 70%, 80%, 90%, 95%, 96%, 97%, 98%, or 99% identity to SEQ ID NO: 148;
(cxxiii) a heavy chain variable sequence having a sequence according to SEQ ID NO: 96, or a heavy chain variable sequence having at least 70%, 80%, 90%, 95%, 96%, 97%, 98%, or 99% identity to SEQ ID NO: 96; and/or a light chain variable sequence having a sequence according to SEQ ID NO: 149, or a light chain variable sequence having at least 70%, 80%, 90%, 95%, 96%, 97%, 98%, or 99% identity to SEQ ID NO: 149;
(cxxiv) a heavy chain variable sequence having a sequence according to SEQ ID NO: 97, or a heavy chain variable sequence having at least 70%, 80%, 90%, 95%, 96%, 97%, 98%, or 99% identity to SEQ ID NO: 97; and/or a light chain variable sequence having a sequence according to SEQ ID NO: 150, or a light chain variable sequence having at least 70%, 80%, 90%, 95%, 96%, 97%, 98%, or 99% identity to SEQ ID NO: 150;
(cxxv) a heavy chain variable sequence having a sequence according to SEQ ID NO: 98, or a heavy chain variable sequence having at least 70%, 80%, 90%, 95%, 96%, 97%, 98%, or 99% identity to SEQ ID NO: 98; and/or a light chain variable sequence having a sequence according to SEQ ID NO: 151, or a light chain variable sequence having at least 70%, 80%, 90%, 95%, 96%, 97%, 98%, or 99% identity to SEQ ID NO: 151;
(cxxvi) a heavy chain variable sequence having a sequence according to SEQ ID NO: 99, or a heavy chain variable sequence having at least 70%, 80%, 90%, 95%, 96%, 97%, 98%, or 99% identity to SEQ ID NO: 99; and/or a light chain variable sequence having a sequence according to SEQ ID NO: 152, or a light chain variable sequence having at least 70%, 80%, 90%, 95%, 96%, 97%, 98%, or 99% identity to SEQ ID NO: 152;
(cxxvii) a heavy chain variable sequence having a sequence according to SEQ ID NO: 100, or a heavy chain variable sequence having at least 70%, 80%, 90%, 95%, 96%, 97%, 98%, or 99% identity to SEQ ID NO: 100; and/or a light chain variable sequence having a sequence according to SEQ ID NO: 136, or a light chain variable sequence having at least 70%, 80%, 90%, 95%, 96%, 97%, 98%, or 99% identity to SEQ ID NO: 136;
(cxxviii) a heavy chain variable sequence having a sequence according to SEQ ID NO: 91, or a heavy chain variable sequence having at least 70%, 80%, 90%, 95%, 96%, 97%, 98%, or 99% identity to SEQ ID NO: 91; and/or a light chain variable sequence having a sequence according to SEQ ID NO: 153, or a light chain variable sequence having at least 70%, 80%, 90%, 95%, 96%, 97%, 98%, or 99% identity to SEQ ID NO: 153;
(cxxix) a heavy chain variable sequence having a sequence according to SEQ ID NO: 101, or a heavy chain variable sequence having at least 70%, 80%, 90%, 95%, 96%, 97%, 98%, or 99% identity to SEQ ID NO: 101; and/or a light chain variable sequence having a sequence according to SEQ ID NO: 154, or a light chain variable sequence having at least 70%, 80%, 90%, 95%, 96%, 97%, 98%, or 99% identity to SEQ ID NO: 154;
(cxxx) a heavy chain variable sequence having a sequence according to SEQ ID NO: 102, or a heavy chain variable sequence having at least 70%, 80%, 90%, 95%, 96%, 97%, 98%, or 99% identity to SEQ ID NO: 102; and/or a light chain variable sequence having a sequence according to SEQ ID NO: 155, or a light chain variable sequence having at least 70%, 80%, 90%, 95%, 96%, 97%, 98%, or 99% identity to SEQ ID NO: 155;
(cxxxi) a heavy chain variable sequence having a sequence according to SEQ ID NO: 36, or a heavy chain variable sequence having at least 70%, 80%, 90%, 95%, 96%, 97%, 98%, or 99% identity to SEQ ID NO: 36; and/or a light chain variable sequence having a sequence according to SEQ ID NO: 156, or a light chain variable sequence having at least 70%, 80%, 90%, 95%, 96%, 97%, 98%, or 99% identity to SEQ ID NO: 156; or
(cxxxii) a heavy chain variable sequence having a sequence according to SEQ ID NO: 103, or a heavy chain variable sequence having at least 70%, 80%, 90%, 95%, 96%, 97%, 98%, or 99% identity to SEQ ID NO: 103; and/or a light chain variable sequence having a sequence according to SEQ ID NO: 157, or a light chain variable sequence having at least 70%, 80%, 90%, 95%, 96%, 97%, 98%, or 99% identity to SEQ ID NO: 157.

In some aspects, the antibodies or antibody fragments comprise:
(a) an immunoglobulin heavy chain variable region comprising a VHCDR1 comprising the amino acid sequence of SEQ ID NO: 1, a VHCDR2 comprising the amino acid sequence of SEQ ID NO: 2, and a VHCDR3 comprising the amino acid sequence of SEQ ID NO: 3, and having at least 70%, 80%, 90%, 95%, 96%, 97%, 98%, or 99% identity to SEQ ID NO: 12;
and
(b) and an immunoglobulin light chain variable region comprising a VLCDR1 comprising the amino acid sequence of SEQ ID NO: 4, a VLCDR2 comprising the amino acid sequence of SEQ ID NO: 5, and a VLCDR3 comprising the amino acid sequence of SEQ ID NO: 6, and having at least 70%, 80%, 90%, 95%, 96%, 97%, 98%, or 99% identity to SEQ ID NO: 19.

In one embodiment, provided herein are antibodies or antibody fragments, which compete for binding to the same epitope on HSP70 as the antibodies or antibody fragments according to any one of the present embodiments. In one embodiment, provided herein are antibodies or antibody fragments that bind, or are capable of binding, to an epitope on HSP70 recognized by an antibody or antibody fragment of any one of the present embodiments.

In one embodiment, provided herein are antibodies or antibody fragments, wherein the antibodies or antibody fragments bind to an epitope of HSP70 defined by a peptide corresponding to K573-Q601 of SEQ ID NO: 11. In some aspects, when bound to HSP70, the antibodies or antibody fragments bind to one or two of the following residues: H594, K595, and Q601 of SEQ ID NO:11. In some aspects, when bound to HSP70, the antibodies or antibody fragments bind to all of the following residues: H594, K595, and Q601 of SEQ ID NO:11. In some aspects, when bound to HSP70, the antibodies or antibody fragments additionally bind to at least one of the following residues: K573, E576, W580, R596, and E598 of SEQ ID NO:11. In some aspects, when bound to HSP70, the antibodies or antibody fragments additionally bind to at least two, three, four, or five of the following residues: K573, E576, W580, R596, and E598 of SEQ ID NO:11. In some aspects, when bound to HSP70, the antibodies or antibody fragments bind to all of the following residues: K573, E576, W580, H594, K595, R596, E598, and Q601 of SEQ ID NO:11. In some aspects, when bound to HSP70, the antibodies or antibody fragments bind to an epitope of HSP70 comprising KEWHKREQ (SEQ ID NO: 250).

In some aspects of any of the present embodiments, the antibodies bind, or are capable of binding, to HSP70. In some aspects of any of the present embodiments, the antibodies bind, or are capable of binding, to HSP70 in its ADP-bound form. In some aspects of any of the present embodiments, the antibodies bind, or are capable of binding, to HSP70 in its peptide-bound form. In some aspects of any of the present embodiments, the antibodies bind, or are capable of binding, to HSP70 in its ADP-bound and peptide-bound form. In some aspects of any of the present embodiments, the antibodies display altered, e.g., enhanced, antibody-dependent cellular cytotoxicity. In some aspects of any of the present embodiments, the antibodies display altered, e.g., enhanced, complement-dependent cellular cytotoxicity. In some aspects of any of the present embodiments, the antibodies enhance HSP70 uptake by immune effector cells, such as, for example, monocytes/macrophages and dendritic cells. In some aspects, the uptake is mediated by human FcγR1, FcγR2a, FcγR2b, FcγR2c, FcγR3a, and/or FcγR3b. In some aspects, the antibodies bind, or are capable of binding, to HSP70. In some aspects, the antibodies bind to human HSP70 (e.g., HSP70 in its ADP-bound and/or peptide-bound form) with a K_{D} less than about 50, 40, 30, 20, 10, 9, 8, 7, 6, 5, 4, 3, 2, 1, 0.9, 0.85, 0.8, 0.75, 0.7, 0.6, 0.5, 0.1, 0.05 nM, or 20, 10, 9, 8, 7, 6, 5, 4, 3, 2, 1, 0.9, 0.85, 0.8, 0.75, 0.7, 0.6, 0.5, 0.1, or 0.05 pM, as determined by surface plasmon resonance or Octet bio-layer interferometry (BLI) analysis. In some aspects, the antibodies bind to human HSP70 (e.g., HSP70 in its ADP-bound and/or peptide-bound form) with a K_{D} of from about 50 nM to about 0.05 nM, from about 50 nM to about 0.075 nM, from about 50 nM to about 0.1 nM, from about 50 nM to about 0.5 nM, from about 50 nM to about 1 nM, from about 40 nM to about 0.05 nM, from about 40 nM to about 0.075 nM, from about 40 nM to about 0.1 nM, from about 40 nM to about 0.5 nM, from about 40 nM to about 1 nM, from about 30 nM to about 0.05 nM, from about 30 nM to about 0.075 nM, from about 30 nM to about 0.1 nM, from about 30 nM to about 0.5 nM, from about 30 nM to about 1 nM, from about 20 nM to about 0.05 nM, from about 20 nM to about 0.075 nM, from about 20 nM to about 0.1 nM, from about 20 nM to about 0.5 nM, from about 20 nM to about 1 nM, from about 10 nM to about 0.05 nM, from about 10 nM to about 0.075 nM, from about 10 nM to about 0.1 nM, from about 10 nM to about 0.5 nM, from about 10 nM to about 1 nM, from about 5 nM to about 0.05 nM, from about 5 nM to about 0.075 nM, from about 5 nM to about 0.1 nM, from about 5 nM to about 0.5 nM, from about 5 nM to about 1 nM, from about 3 nM to about 0.05 nM, from about 3 nM to about 0.075 nM, from about 3 nM to about 0.1 nM, from about 3 nM to about 0.5 nM, from about 3 nM to about 1 nM, from about 3 nM to about 2 nM, from about 2 nM to about 0.05 nM, from about 2 nM to about 0.075 nM, from about 2 nM to about 0.1 nM, from about 2 nM to about 0.5 nM, from about 2 nM to about 1 nM, from about 1 nM to about 0.05 nM, from about 1 nM to about 0.075 nM, from about 1 nM to about 0.1 nM, from about 1 nM to about 0.5 nM, from about 0.5 nM to about 0.05 nM, from about 0.5 nM to about 0.075 nM, from about 0.5 nM to about 0.1 nM, from about 0.1 nM to about 0.05 nM, from about 0.1 nM to about 0.075 nM, or from about 0.075 nM to about 0.05 nM, as determined by surface plasmon resonance or Octet bio-layer interferometry (BLI) analysis. In some aspects, the antibodies bind to human HSP70 (e.g., HSP70 in its ADP-bound and/or peptide-bound form) with a K_{D} of from about 20 pM to about 0.05 pM, from about 20 pM to about 0.075 pM, from about 20 pM to about 0.1 pM, from about 20 pM to about 0.5 pM, from about 20 pM to about 1 pM, from about 10 pM to about 0.05 pM, from about 10 pM to about 0.075 pM, from about 10 pM to about 0.1 pM, from about 10 pM to about 0.5 pM, from about 10 pM to about 1 pM, from about 5 pM to about 0.05 pM, from about 5 pM to about 0.075 pM, from about 5 pM to about 0.1 pM, from about 5 pM to about 0.5 pM, from about 5 pM to about 1 pM, from about 3 pM to about 0.05 pM, from about 3 pM to about 0.075 pM, from about 3 pM to about 0.1 pM, from about 3 pM to about 0.5 pM, from about 3 pM to about 1 pM, from about 3 pM to about 2 pM, from about 2 pM to about 0.05 pM, from about 2 pM to about 0.075 pM, from about 2 pM to about 0.1 pM, from about 2 pM to about 0.5 pM, from about 2 pM to about 1 pM, from about 1 pM to about 0.05 pM, from about 1 pM to about 0.075 pM, from about 1 pM to about 0.1 pM, from about 1 pM to about 0.5 pM, from about 0.5 pM to about 0.05 pM, from about 0.5 pM to about 0.075 pM, from about 0.5 pM to about 0.1 pM, from about 0.1 pM to about 0.05 pM, from about 0.1 pM to about 0.075 pM, or from about 0.075 pM to about 0.05 pM, as determined by surface plasmon resonance or Octet bio-layer interferometry (BLI) analysis.

In some aspects, the antibodies form, or are capable of forming, a high order complex upon binding to HSP70. As used herein, the term “high order complex” refers to any complex other than a 1:1 antibody:HSP70 complex (e.g., a 1:2, 2:1, 2:2, 2:4, 5:10, or 6:12 complex). The antibody:HSP70 complex may, for example, have a molecular weight of at least (or greater than) about 250 kDa, 290 kDa, 300 kDa, 400 kDa, 500 kDa, 580 kDa, 600 kDa, 700 kDa, 800 kDa, 900 kDa, 1,000 kDa, 1,100 kDa, 1,200 kDa, 1,300 kDa, 1,400 kDa, 1,450 kDa, 1,500 kDa, 1,600 kDa, 1,700 kDa, 1,750 kDa, 1,800 kDa, 1,900 kDa, or 2,000 kDa. The antibody:HSP70 complex may, for example, have a molecular weight of about 250 kDa, 290 kDa, 300 kDa, 400 kDa, 500 kDa, 580 kDa, 600 kDa, 700 kDa, 800 kDa, 900 kDa, 1,000 kDa, 1,100 kDa, 1,200 kDa, 1,300 kDa, 1,400 kDa, 1,450 kDa, 1,500 kDa, 1,600 kDa, 1,700 kDa, 1,750 kDa, 1,800 kDa, 1,900 kDa, or 2,000 kDa. In certain aspects, the ratio of antibody to HSP70 in the antibody:HSP70 complex is about 1:2. For example, the antibody:HSP70 complex may comprise: about one antibody molecule and about two HSP70 molecules; about two antibody molecules and about four HSP70 molecules; about three antibody molecules and about six HSP70 molecules; about four antibody molecules and about eight HSP70 molecules; about five antibody molecules and about ten HSP70 molecules; or about six antibody molecules and about twelve HSP70 molecules. High order complex formation may be measured by any method known in the art, including, for example, size exclusion chromatography, for example, as described in Example 18 herein.

In some aspects, an antibody:HSP70 complex as provided herein (e.g., a complex comprising 77A or an Fc variant thereof), activates immune effector cells, e.g., human immune effector cells. In some embodiments, immune effector cells, e.g., human immune effector cells, comprise CD8+ T cells, CD4+ T cells, NK cells, and dendritic cells, e.g., immature dendritic cells.

In some embodiments, an antibody:HSP70 complex as provided herein (e.g., a complex comprising 77A or an Fc variant thereof) activates CD8+ T cells. In certain embodiments, such activation of CD8+ T cells increases cytokine synthesis and secretion of activating Th-1 biased cytokines, e.g., a cytokine from Table 18, in complex-treated CD8+ T cells relative to control expression (e.g., untreated T cells). In certain embodiments, such activation of CD8+ T cells increases cytokine synthesis and secretion of activating Th-1 biased cytokines, e.g., IL-12, IFB-γ, IFN-α, IL-8, G-CSF, GM-CSF, IFN-α2, and TNF-α, in complex-treated CD8+ T cells relative to control expression (e.g., untreated T cells). In certain embodiments, cytokine secretion is increased at least 10%, 20%, 30%, 40%, 50%, 60%, 70%, 80%, 90%, 100%, 200%, 300%, 400%, or 500% relative to control (e.g., untreated cells). Methods of quantifying cytokine secretion are known in the art and include, for example and without limitation, cytokine array.

In some embodiments, an antibody:HSP70 complex as provided herein (e.g., a complex comprising 77A or an Fc variant thereof) activates CD4+ T cells. In certain embodiments, such activation of CD4+ T cells increases cytokine synthesis and secretion of activating cytokines, e.g., a cytokine from Table 20, in complex-treated CD4+ T cells relative to control expression (e.g., untreated T cells). In certain embodiments, such activation of CD4+ T cells increases cytokine synthesis and secretion of activating cytokines, e.g., IL-12, IL-10, IL-17A, IL-2, IL-8, IFN-γ, and IL-1b, in the CD4+ T cells relative to control expression (e.g., untreated T cells). In certain embodiments, cytokine secretion is increased at least 10%, 20%, 30%, 40%, 50%, 60%, 70%, 80%, 90%, 100%, 200%, 300%, 400%, or 500% relative to control (e.g., untreated cells). Methods of quantifying cytokine secretion are known in the art and include, for example and without limitation, cytokine array.

In some embodiments, an antibody:HSP70 complex as provided herein (e.g., a complex comprising 77A or an Fc variant thereof) activates cytokine release in NK cells. In certain embodiments, such activation of NK cells increases cytokine synthesis and secretion of activating cytokines, e.g., a cytokine from Table 19, in complex-treated NK cells relative to control expression (e.g., untreated NK cells). In certain embodiments, such activation of NK cells increases cytokine synthesis and secretion of activating cytokines, e.g., IL-2, IL-2Ra, and M-CSF, in the NK cells relative to control expression (e.g., untreated NK cells). In certain embodiments, cytokine secretion is increased at least 10%, 20%, 30%, 40%, 50%, 60%, 70%, 80%, 90%, 100%, 200%, 300%, 400%, or 500% relative to control (e.g., untreated cells). Methods of quantifying cytokine secretion are known in the art and include, for example and without limitation, cytokine array.

In some embodiments, an antibody:HSP70 complex as provided herein (e.g., a complex comprising 77A or an Fc variant thereof) activates dendritic cells, e.g., immature and/or mature dendritic cells. In certain embodiments, such activation of dendritic cells, e.g., immature dendritic cells, increases expression of CD83 activation marker, e.g., on CD11c dendritic cells relative to control expression (e.g., untreated dendritic cells, e.g., immature dendritic cells). In certain embodiments, expression of CD83 is increased at least 10%, 20%, 30%, 40%, 50%, 60%, 70%, 80%, 90%, 100%, 200%, 300%, 400%, or 500% relative to control (e.g., untreated cells).

In some aspects, the antibodies (alone or in complex with HSP70) bind, or are capable of binding, to a human FcγR (e.g., FcγR1, FcγR2a, FcγR2b, FcγR2c, FcγR3a, and/or FcγR3b). In some aspects, the antibodies (alone or in complex with HSP70, e.g., HSP70 in its ADP-bound and/or peptide-bound form) bind to a human FcγR (e.g., FcγR1, FcγR2a, FcγR2b, FcγR2c, FcγR3a, and/or FcγR3b) with a K_{D} less than about 50, 40, 30, 20, 10, 9, 8, 7, 6, 5, 4, 3, 2, 1, 0.9, 0.85, 0.8, 0.75, 0.7, 0.6, 0.5, 0.1, 0.05 nM, or 20, 10, 9, 8, 7, 6, 5, 4, 3, 2, 1, 0.9, 0.85, 0.8, 0.75, 0.7, 0.6, 0.5, 0.1, or 0.05 pM, as determined by surface plasmon resonance or Octet bio-layer interferometry (BLI) analysis. In some aspects, the antibodies (alone or in complex with HSP70, e.g., HSP70 in its ADP-bound and/or peptide-bound form) bind to a human FcγR (e.g., FcγR1, FcγR2a, FcγR2b, FcγR2c, FcγR3a, and/or FcγR3b) with a K_{D} of from about 50 nM to about 0.05 nM, from about 50 nM to about 0.075 nM, from about 50 nM to about 0.1 nM, from about 50 nM to about 0.5 nM, from about 50 nM to about 1 nM, from about 40 nM to about 0.05 nM, from about 40 nM to about 0.075 nM, from about 40 nM to about 0.1 nM, from about 40 nM to about 0.5 nM, from about 40 nM to about 1 nM, from about 30 nM to about 0.05 nM, from about 30 nM to about 0.075 nM, from about 30 nM to about 0.1 nM, from about 30 nM to about 0.5 nM, from about 30 nM to about 1 nM, 20 nM to about 0.05 nM, from about 20 nM to about 0.075 nM, from about 20 nM to about 0.1 nM, from about 20 nM to about 0.5 nM, from about 20 nM to about 1 nM, from about 10 nM to about 0.05 nM, from about 10 nM to about 0.075 nM, from about 10 nM to about 0.1 nM, from about 10 nM to about 0.5 nM, from about 10 nM to about 1 nM, from about 5 nM to about 0.05 nM, from about 5 nM to about 0.075 nM, from about 5 nM to about 0.1 nM, from about 5 nM to about 0.5 nM, from about 5 nM to about 1 nM, from about 3 nM to about 0.05 nM, from about 3 nM to about 0.075 nM, from about 3 nM to about 0.1 nM, from about 3 nM to about 0.5 nM, from about 3 nM to about 1 nM, from about 3 nM to about 2 nM, from about 2 nM to about 0.05 nM, from about 2 nM to about 0.075 nM, from about 2 nM to about 0.1 nM, from about 2 nM to about 0.5 nM, from about 2 nM to about 1 nM, from about 1 nM to about 0.05 nM, from about 1 nM to about 0.075 nM, from about 1 nM to about 0.1 nM, from about 1 nM to about 0.5 nM, from about 0.5 nM to about 0.05 nM, from about 0.5 nM to about 0.075 nM, from about 0.5 nM to about 0.1 nM, from about 0.1 nM to about 0.05 nM, from about 0.1 nM to about 0.075 nM, or from about 0.075 nM to about 0.05 nM, as determined by surface plasmon resonance or Octet bio-layer interferometry (BLI) analysis.

In some aspects, it is contemplated that a heavy chain variable region sequence, for example, the VH sequence of any one of SEQ ID NOs: 7, 12-17, and 26-103, or any variants thereof, may be covalently linked to a variety of heavy chain constant region sequences known in the art. Similarly, it is contemplated that a light chain variable region sequence, for example, the VL of any one of SEQ ID NOs: 8, 19-24, and 105-157, or any variants thereof, may be covalently linked to a variety of light chain constant region sequences known in the art.

For example, an antibody or antibody fragment may have a heavy chain constant region chosen from, e.g., the heavy chain constant regions of IgG1, IgG2, IgG3, IgG4, IgM, IgA1, IgA2, IgD, and IgE; particularly, chosen from, e.g., the (e.g., human) heavy chain constant regions of IgG1, IgG2, IgG3, and IgG4. In another embodiment, the antibody or antibody fragment has a light chain constant region chosen from, e.g., the (e.g., human) light chain constant regions of kappa or lambda. The constant region can be altered, e.g., mutated, to modify the properties of the antibody or antibody fragment (e.g., to increase or decrease one or more of: Fc receptor binding, antibody glycosylation, the number of cysteine residues, effector cell function, and/or complement function). In one embodiment the antibody or antibody fragment has effector function and can fix complement.

In some aspects, the constant region of the heavy chain of the antibody or antibody fragment is a human IgG1 isotype, having an amino acid sequence:

(SEQ ID NO: 235)
ASTKGPSVFPLAPSSKSTSGGTAALGCLVKDYFPEPVTVSWNSGALTSGVHTFPAVLQSS

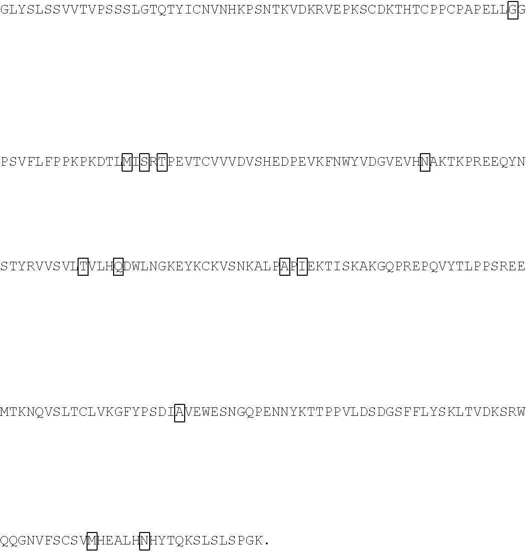

In some aspects, the human IgG1 constant region is modified at amino acid Gly236 (position 119 in SEQ ID NO: 235, and boxed in SEQ ID NO: 235 in the preceding paragraph), for example Gly236Ala (G236A), for example, the IgG1 constant region comprises the amino acid sequence of SEQ ID NO: 236 (GA). In some aspects, the human IgG1 constant region is modified at (i) amino acid Gly236 (at position 119 in SEQ ID NO: 235, and boxed in SEQ ID NO: 235 in the preceding paragraph), for example Gly236Ala (G236A), (ii) amino acid Ala330 (position 213 in SEQ ID NO: 235, and boxed in SEQ ID NO: 235 in the preceding paragraph), for example Ala330Leu (A330 μL), and/or (iii) amino acid Ile332 (position 215 in SEQ ID NO: 235, and boxed in SEQ ID NO: 235 in the preceding paragraph), for example Ile332Glu (I332E), for example, the IgG1 constant region comprises the amino acid sequence of SEQ ID NO: 237 (GAALIE). In some aspects, the human IgG1 constant region is modified at (i) amino acid Met252 (at position 135 in SEQ ID NO: 235, and boxed in SEQ ID NO: 235 in the preceding paragraph), for example Met252Tyr (M252Y), (ii) amino acid Ser254 (position 137 in SEQ ID NO: 235, and boxed in SEQ ID NO: 235 in the preceding paragraph), for example Ser254Thr (S254T), and/or (iii) amino acid Thr256 (position 139 in SEQ ID NO: 235, and boxed in SEQ ID NO: 235 in the preceding paragraph), for example Thr256Glu (T256E), for example, the IgG1 constant region comprises the amino acid sequence of SEQ ID NO: 238 (YTE). In some aspects, the human IgG1 constant region is modified at (i) amino acid Met428 (at position 311 in SEQ ID NO: 235, and boxed in SEQ ID NO: 235 in the preceding paragraph), for example Met428Leu (M428 μL), and/or (ii) amino acid Asn434 (position 317 in SEQ ID NO: 235, and boxed in SEQ ID NO: 235 in the preceding paragraph), for example Asn434Ser (N434S), for example, the IgG1 constant region comprises the amino acid sequence of SEQ ID NO: 239 (LS). In some aspects, the human IgG1 constant region is modified at (i) amino acid Thr307 (at position 190 in SEQ ID NO: 235, and boxed in SEQ ID NO: 235 in the preceding paragraph), for example Thr307Gln (T307Q), (ii) amino acid Gln311 (position 194 in SEQ ID NO: 235, and boxed in SEQ ID NO: 235 in the preceding paragraph), for example Gln311Val (Q311V), and/or (iii) amino acid Ala378 (position 261 in SEQ ID NO: 235, and boxed in SEQ ID NO: 235 in the preceding paragraph), for example Ala378Val (A378V), for example, the IgG1 constant region comprises the amino acid sequence of SEQ ID NO: 240 (DF215). In some aspects, the human IgG1 constant region is modified at (i) amino acid Thr256 (position 139 in SEQ ID NO: 235, and boxed in SEQ ID NO: 235 in the preceding paragraph), for example Thr256Asp (T256D), (ii) amino acid Asn286 (position 169 in SEQ ID NO: 235, and boxed in SEQ ID NO: 235 in the preceding paragraph), for example Asn286Asp (N286D), (iii) amino acid Thr307 (at position 190 in SEQ ID NO: 235, and boxed in SEQ ID NO: 235 in the preceding paragraph), for example Thr307Arg (T307R), (iv) amino acid Gln311 (position 194 in SEQ ID NO: 235, and boxed in SEQ ID NO: 235 in the preceding paragraph), for example Gln311 Val (Q311V), and/or (v) amino acid Ala378 (position 261 in SEQ ID NO: 235, and boxed in SEQ ID NO: 235 in the preceding paragraph), for example Ala378Val (A378V), for example, the IgG1 constant region comprises the amino acid sequence of SEQ ID NO: 241 (DF228). Unless indicated otherwise, all residue numbers are according to EU numbering (Kabat, E. A., et al. (1991) SEQUENCES OF PROTEINS OF IMMUNOLOGICAL INTEREST, FIFTH EDITION, U.S. Department of Health and Human Services, NIH Publication No. 91-3242).

In some aspects, the constant region of the heavy chain of the antibody or antibody fragment is a human IgG1 isotype, having an amino acid sequence:

(SEQ ID NO: 217)
ASTKGPSVFPLAPSSKSTSGGTAALGCLVKDYFPEPVTVSWNSGALTSGVHTFPAVLQSS

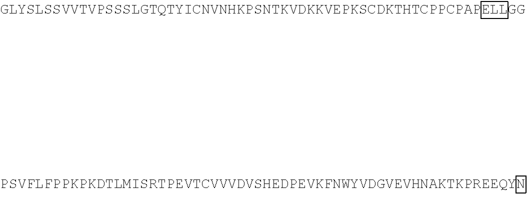

STYRVVSVLTVLHQDWLNGKEYKCKVSNKALPAPIEKTISKAKGQPREPQVYTLPPSRDE
LTKNQVSLTCLVKGFYPSDIAVEWESNGQPENNYKTTPPVLDSDGSFFLYSKLTVDKSRW
QQGNVFSCSVMHEALHNHYTQKSLSLSPGK.

In some aspects, the human IgG1 constant region is modified at amino acid Asn297 (boxed in SEQ ID NO: 217 in the preceding paragraph) to prevent to glycosylation of the antibody, for example Asn297Ala (N297A). In some aspects, the constant region of the antibody is modified at amino acid Leu235 (boxed in SEQ ID NO: 217 in the preceding paragraph) to alter Fc receptor interactions, for example Leu235Glu (L235E) or Leu235Ala (L235A). In some aspects, the constant region of the antibody is modified at amino acid Leu234 (boxed in SEQ ID NO: 217 in the preceding paragraph) to alter Fc receptor interactions, e.g., Leu234Ala (L234A). In some aspects, the constant region of the antibody is modified at amino acid Glu233 (boxed in SEQ ID NO: 217 in the preceding paragraph), e.g., Glu233Pro (E233P). In some aspects, the constant region of the antibody is altered at both amino acid 234 and 235, for example Leu234Ala and Leu235Ala (L234A/L235A). In some aspects, the constant region of the antibody is altered at amino acids 233, 234, and 234, for example, Glu233Pro, Leu234Ala, and Leu235Ala (E233P L234A/L235A) (Armour KL. et al. (1999) EUR. J. IMMUNOL. 29(8):2613-24). All residue numbers are according to EU numbering (Kabat, E. A., et al. (1991) SEQUENCES OF PROTEINS OF IMMUNOLOGICAL INTEREST, FIFTH EDITION, U.S. Department of Health and Human Services, NIH Publication No. 91-3242).

In some aspects, the constant region of the heavy chain of the antibody is a human IgG1 isotype, having an amino acid sequence:

(SEQ ID NO: 218)
ASTKGPSVFPLAPSSKSTSGGTAALGCLVKDYFPEPVTVSWNSGALTSGVHTFPAVLQSS

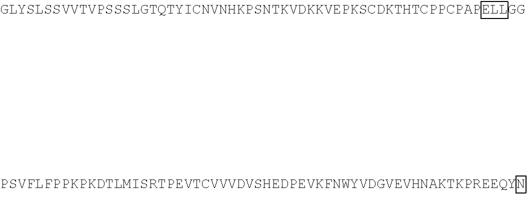

STYRVVSVLTVLHQDWLNGKEYKCKVSNKALPAPIEKTISKAKGQPREPQVYTLPPSREE
MTKNQVSLTCLVKGFYPSDIAVEWESNGQPENNYKTTPPVLDSDGSFFLYSKLTVDKSRW
QQGNVFSCSVMHEALHNHYTQKSLSLSPGK.

In some aspects, the human IgG1 constant region is modified at amino acid Asn297 (boxed in SEQ ID NO: 218 in the preceding paragraph) to prevent to glycosylation of the antibody, for example Asn297Ala (N297A). In some aspects, the constant region of the antibody is modified at amino acid Leu235 (boxed in SEQ ID NO: 218 in the preceding paragraph) to alter Fc receptor interactions, for example Leu235Glu (L235E) or Leu235Ala (L235A). In some aspects, the constant region of the antibody is modified at amino acid Leu234 (boxed in SEQ ID NO: 218 in the preceding paragraph) to alter Fc receptor interactions, e.g., Leu234Ala (L234A). In some aspects, the constant region of the antibody is modified at amino acid Glu233 (boxed in SEQ ID NO: 218 in the preceding paragraph), e.g., Glu233Pro (E233P). In some aspects, the constant region of the antibody is altered at both amino acid 234 and 235, for example Leu234Ala and Leu235Ala (L234A/L235A). In some aspects, the constant region of the antibody is altered at amino acids 233, 234, and 234, for example, Glu233Pro, Leu234Ala, and Leu235Ala (E233P L234A/L235A) (Armour KL. et al. (1999) EUR. J. IMMUNOL. 29(8):2613-24). All residue numbers are according to EU numbering (Kabat, E. A., et al., supra).

In some aspects, the human IgG1 constant region is modified to comprise either a “knob” mutation, e.g., T366Y, or a “hole” mutation, e.g., Y407T, for heterodimerization with a second constant region (residue numbers according to EU numbering (Kabat, E. A., et al., supra)).

In some aspects, the constant region of the heavy chain of the antibody is a human IgG1 isotype, e.g., an allotype of the human IgG1 isotype, e.g., the IgG1 G1m3 allotype. Exemplary human IgG1 allotypes are described in Magdelaine-Beuzelin et al. (2009) PHARMACOGENET. GENOMICS 19(5):383-7.

In some aspects, the constant region of the heavy chain of the antibody is a human IgG2 isotype, having an amino acid sequence:

(SEQ ID NO: 219)
ASTKGPSVFPLAPCSRSTSESTAALGCLVKDYFPEPVTVSWNSGALTSGVHTFPAVLQSS
GLYSLSSVVTVPSSNFGTQTYTCNVDHKPSNTKVDKTVERKCCVECPPCPAPPVAGPSVF

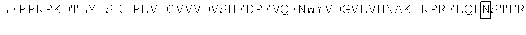

VVSVLTVVHQDWLNGKEYKCKVSNKGLPAPIEKTISKTKGQPREPQVYTLPPSREEMTKN
QVSLTCLVKGFYPSDISVEWESNGQPENNYKTTPPMLDSDGSFFLYSKLTVDKSRWQQGN
VFSCSVMHEALHNHYTQKSLSLSPGK.

In some aspects, the human IgG2 constant region is modified at amino acid Asn297 (boxed in SEQ ID NO: 219 in the preceding paragraph) to prevent to glycosylation of the antibody, e.g., Asn297Ala (N297A), where the residue numbers are according to EU numbering (Kabat, E. A., et al., supra).

In some aspects, the constant region of the heavy chain of the antibody is an human IgG3 isotype, having an amino acid sequence:

(SEQ ID NO: 220)
ASTKGPSVFPLAPCSRSTSGGTAALGCLVKDYFPEPVTVSWNSGALTSGVHTFPAVLQSS
GLYSLSSVVTVPSSSLGTQTYTCNVNHKPSNTKVDKRVELKTPLGDTTHTCPRCPEPKSC
DTPPPCPRCPEPKSCDTPPPCPRCPEPKSCDTPPPCPRCPAPELLGGPSVFLFPPKPKDT

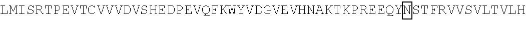

QDWLNGKEYKCKVSNKALPAPIEKTISKTKGQPREPQVYTLPPSREEMTKNQVSLTCLVK
GFYPSDIAVEWESSGQPENNYNTTPPMLDSDGSFFLYSKLTVDKSRWQQGNIFSCSVMHE

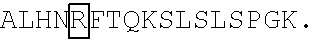

In some aspects, the human IgG3 constant region is modified at amino acid Asn297 (boxed in SEQ ID NO: 220 in the preceding paragraph) to prevent to glycosylation of the antibody, e.g., Asn297Ala (N297A). In some aspects, the human IgG3 constant region is modified at amino acid Arg435 (boxed in SEQ ID NO: 220 in the preceding paragraph) to extend the half-life, e.g., Arg435H (R435H). All residue numbers are according to EU numbering (Kabat, E. A., et al., supra).

In some aspects, the constant region of the heavy chain of the antibody is an human IgG4 isotype, having an amino acid sequence:

(SEQ ID NO: 221)
ASTKGPSVFPLAPCSRSTSESTAALGCLVKDYFPEPVTVSWNSGALTSGVHTFPAVLQSS

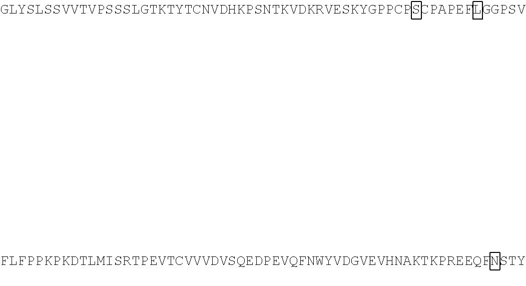

RVVSVLTVLHQDWLNGKEYKCKVSNKGLPSSIEKTISKAKGQPREPQVYTLPPSQEEMTK
NQVSLTCLVKGFYPSDIAVEWESNGQPENNYKTTPPVLDSDGSFFLYSRLTVDKSRWQEG
NVFSCSVMHEALHNHYTQKSLSLSLGK.

In some aspects, the human IgG4 constant region is modified within the hinge region to prevent or reduce strand exchange, e.g., in some aspects human IgG4 constant region is modified at Ser228 (boxed in SEQ ID NO: 221 in the preceding paragraph), e.g., Ser228Pro (S228P). In other embodiments, the human IgG4 constant region is modified at amino acid Leu235 (boxed in SEQ ID NO: 221 in the preceding paragraph) to alter Fc receptor interactions, e.g., Leu235Glu (L235E). In some aspects, the human IgG4 constant region is modified at both Ser228 and Leu335, e.g., Ser228Pro and Leu235Glu (S228P/L235E). In some aspects, the human IgG4 constant region is modified at amino acid Asn297 (boxed in SEQ ID NO: 221 in the preceding paragraph) to prevent to glycosylation of the antibody, e.g., Asn297Ala (N297A). All residue numbers are according to EU numbering (Kabat, E. A., et al., supra).

In some aspects, the human IgG constant region is modified to enhance FcRn binding. Examples of Fc mutations that enhance binding to FcRn are Met252Tyr, Ser254Thr, Thr256Glu (M252Y, S254T, T256E, respectively) (Dall'Acqua et al. (2006) J. BIOL. CHEM. 281(33): 23514-23524), or Met428Leu and Asn434Ser (M428 μL, N434S) (Zalevsky et al. (2010) NATURE BIOTECH. 28(2): 157-159). All residue numbers are according to EU numbering (Kabat, E. A., et al., supra).

In some aspects, the human IgG constant region is modified to alter antibody-dependent cellular cytotoxicity (ADCC) and/or complement-dependent cytotoxicity (CDC), e.g., the amino acid modifications described in Natsume et al. (2008) CANCER RES. 68(10): 3863-72; Idusogie et al. (2001) J. IMMUNOL. 166(4): 2571-5; Moore et al. (2010) MABS 2(2): 181-189; Lazar et al. (2006) PROC. NATL. ACAD. SCI. USA 103(11): 4005-4010, Shields et al. (2001) J. BIOL. CHEM. 276(9): 6591-6604; Stavenhagen et al. (2007) CANCER RES. 67(18): 8882-8890; Stavenhagen et al. (2008) ADVAN. ENZYME REGUL. 48: 152-164; Alegre et al. (1992) J. IMMUNOL. 148: 3461-3468.

In some aspects, the human IgG constant region is modified to induce heterodimerization. For example, a heavy chain having an amino acid modification within the CH3 domain at Thr366, e.g., a substitution with a more bulky amino acid, e.g., Tyr (T366W), is able to preferentially pair with a second heavy chain having a CH3 domain having amino acid modifications to less bulky amino acids at positions Thr366, Leu368, and Tyr407, e.g., Ser, Ala and Val, respectively (T366S/L368A/Y407V). Heterodimerization via CH3 modifications can be further stabilized by the introduction of a disulfide bond, for example by changing Ser354 to Cys (S354C) and Y349 to Cys (Y349C) on opposite CH3 domains (see, Carter (2001) J. IMMUNOL. METHODS 248: 7-15).

In some aspects, the constant region of the light chain of the antibody is a human kappa constant region, e.g., a human kappa constant region having the amino acid sequence:

(SEQ ID NO: 222)
TVAAPSVFIFPPSDEQLKSGTASVVCLLNNFYPREAKVQWKVDNALQSG
NSQESVTEQDSKDSTYSLSSTLTLSKADYEKHKVYACEVTHQGLSSPVT
KSFNRGEC,

In some aspects, the constant region of the light chain of the antibody is a human kappa constant region, e.g., a human kappa constant region having the amino acid sequence:

(SEQ ID NO: 223)
RTVAAPSVFIFPPSDEQLKSGTASVVCLLNNFYPREAKVQWKVDNALQS
GNSQESVTEQDSKDSTYSLSSTLTLSKADYEKHKVYACEVTHQGLSSPV
TKSFNRGEC.

In some aspects, the constant region of the light chain of the antibody is a human lambda constant region, e.g., a human lambda constant region having the amino acid sequence:

(SEQ ID NO: 224)
GQPKANPTVTLFPPSSEELQANKATLVCLISDFYPGAVTVAWKADGSPV
KAGVETTKPSKQSNNKYAASSYLSLTPEQWKSHRSYSCQVTHEGSTVEK
TVAPTEC.

In some aspects, the antibodies or antibody fragments comprise:

(a) an immunoglobulin heavy chain variable region comprising a VHCDR1 comprising the amino acid sequence of SEQ ID NO: 1, a VHCDR2 comprising the amino acid sequence of SEQ ID NO: 2, and a VHCDR3 comprising the amino acid sequence of SEQ ID NO: 3, and an immunoglobulin light chain variable region comprising a VLCDR1 comprising the amino acid sequence of SEQ ID NO: 4, a VLCDR2 comprising the amino acid sequence of SEQ ID NO: 5, and a VLCDR3 comprising the amino acid sequence of SEQ ID NO: 6; and
(b) (i) a heavy chain having at least 70%, 80%, 90%, 95%, 96%, 97%, 98%, or 99% identity to SEQ ID NO: 242; and/or a light chain having at least 70%, 80%, 90%, 95%, 96%, 97%, 98%, or 99% identity to SEQ ID NO: 249;
(ii) a heavy chain having at least 70%, 80%, 90%, 95%, 96%, 97%, 98%, or 99% identity to SEQ ID NO: 243; and/or a light chain having at least 70%, 80%, 90%, 95%, 96%, 97%, 98%, or 99% identity to SEQ ID NO: 249;
(iii) a heavy chain having at least 70%, 80%, 90%, 95%, 96%, 97%, 98%, or 99% identity to SEQ ID NO: 244; and/or a light chain having at least 70%, 80%, 90%, 95%, 96%, 97%, 98%, or 99% identity to SEQ ID NO: 249;
(iv) a heavy chain having at least 70%, 80%, 90%, 95%, 96%, 97%, 98%, or 99% identity to SEQ ID NO: 245; and/or a light chain having at least 70%, 80%, 90%, 95%, 96%, 97%, 98%, or 99% identity to SEQ ID NO: 249;
(v) a heavy chain having at least 70%, 80%, 90%, 95%, 96%, 97%, 98%, or 99% identity to SEQ ID NO: 246; and/or a light chain having at least 70%, 80%, 90%, 95%, 96%, 97%, 98%, or 99% identity to SEQ ID NO: 249;
(vi) a heavy chain having at least 70%, 80%, 90%, 95%, 96%, 97%, 98%, or 99% identity to SEQ ID NO: 247; and/or a light chain having at least 70%, 80%, 90%, 95%, 96%, 97%, 98%, or 99% identity to SEQ ID NO: 249; or
(vii) a heavy chain having at least 70%, 80%, 90%, 95%, 96%, 97%, 98%, or 99% identity to SEQ ID NO: 248; and/or a light chain having at least 70%, 80%, 90%, 95%, 96%, 97%, 98%, or 99% identity to SEQ ID NO: 249.

In some aspects, the antibodies or antibody fragments comprise:
(i) a heavy chain having a sequence according to SEQ ID NO: 242, or a heavy chain having at least 70%, 80%, 90%, 95%, 96%, 97%, 98%, or 99% identity to SEQ ID NO: 242; and/or a light chain having a sequence according to SEQ ID NO: 249, or a light chain having at least 70%, 80%, 90%, 95%, 96%, 97%, 98%, or 99% identity to SEQ ID NO: 249;
(ii) a heavy chain having a sequence according to SEQ ID NO: 243, or a heavy chain having at least 70%, 80%, 90%, 95%, 96%, 97%, 98%, or 99% identity to SEQ ID NO: 243; and/or a light chain having a sequence according to SEQ ID NO: 249, or a light chain having at least 70%, 80%, 90%, 95%, 96%, 97%, 98%, or 99% identity to SEQ ID NO: 249;
(iii) a heavy chain having a sequence according to SEQ ID NO: 244, or a heavy chain having at least 70%, 80%, 90%, 95%, 96%, 97%, 98%, or 99% identity to SEQ ID NO: 244; and/or a light chain having a sequence according to SEQ ID NO: 249, or a light chain having at least 70%, 80%, 90%, 95%, 96%, 97%, 98%, or 99% identity to SEQ ID NO: 249;
(iv) a heavy chain having a sequence according to SEQ ID NO: 245, or a heavy chain having at least 70%, 80%, 90%, 95%, 96%, 97%, 98%, or 99% identity to SEQ ID NO: 245; and/or a light chain having a sequence according to SEQ ID NO: 249, or a light chain having at least 70%, 80%, 90%, 95%, 96%, 97%, 98%, or 99% identity to SEQ ID NO: 249;
(v) a heavy chain having a sequence according to SEQ ID NO: 246, or a heavy chain having at least 70%, 80%, 90%, 95%, 96%, 97%, 98%, or 99% identity to SEQ ID NO: 246; and/or a light chain having a sequence according to SEQ ID NO: 249, or a light chain having at least 70%, 80%, 90%, 95%, 96%, 97%, 98%, or 99% identity to SEQ ID NO: 249;
(vi) a heavy chain having a sequence according to SEQ ID NO: 247, or a heavy chain having at least 70%, 80%, 90%, 95%, 96%, 97%, 98%, or 99% identity to SEQ ID NO: 247; and/or a light chain having a sequence according to SEQ ID NO: 249, or a light chain having at least 70%, 80%, 90%, 95%, 96%, 97%, 98%, or 99% identity to SEQ ID NO: 249; or
(vii) a heavy chain having a sequence according to SEQ ID NO: 248, or a heavy chain having at least 70%, 80%, 90%, 95%, 96%, 97%, 98%, or 99% identity to SEQ ID NO: 248; and/or a light chain having a sequence according to SEQ ID NO: 249, or a light chain having at least 70%, 80%, 90%, 95%, 96%, 97%, 98%, or 99% identity to SEQ ID NO: 249.

### III. Pharmaceutical Formulations

The present disclosure provides pharmaceutical compositions comprising antibodies that selectively target HSP70. Such compositions comprise a prophylactically or therapeutically effective amount of an antibody or a fragment thereof and a pharmaceutically acceptable carrier.

The phrases “pharmaceutical or pharmacologically acceptable” refers to molecular entities and compositions that do not produce an adverse, allergic, or other untoward reaction when administered to an animal, such as a human, as appropriate. The preparation of a pharmaceutical composition comprising an antibody or additional active ingredient will be known to those of skill in the art in light of the present disclosure. Moreover, for animal (e.g., human) administration, it will be understood that preparations should meet sterility, pyrogenicity, general safety, and purity standards as required by FDA Office of Biological Standards.

The active ingredients can be formulated for parenteral administration, e.g., formulated for injection via the intravenous, intramuscular, sub-cutaneous, or even intraperitoneal routes. Typically, such compositions can be prepared as either liquid solutions or suspensions; solid forms suitable for use to prepare solutions or suspensions upon the addition of a liquid prior to injection can also be prepared; and, the preparations can also be emulsified.

The therapeutic compositions of the present embodiments are advantageously administered in the form of injectable compositions either as liquid solutions or suspensions; solid forms suitable for solution in, or suspension in, liquid prior to injection may also be prepared. These preparations also may be emulsified.

The pharmaceutical forms suitable for injectable use include sterile aqueous solutions or dispersions; formulations including sesame oil, peanut oil, or aqueous propylene glycol; and sterile powders for the extemporaneous preparation of sterile injectable solutions or dispersions. In all cases the form must be sterile and must be fluid to the extent that it may be easily injected. It also should be stable under the conditions of manufacture and storage and must be preserved against the contaminating action of microorganisms, such as bacteria and fungi.

The proteinaceous compositions may be formulated into a neutral or salt form. Pharmaceutically acceptable salts, include the acid addition salts (formed with the free amino groups of the protein) and which are formed with inorganic acids such as, for example, hydrochloric or phosphoric acids, or such organic acids as acetic, oxalic, tartaric, mandelic, and the like. Salts formed with the free carboxyl groups can also be derived from inorganic bases such as, for example, sodium, potassium, ammonium, calcium, or ferric hydroxides, and such organic bases as isopropylamine, trimethylamine, histidine, procaine and the like.

A pharmaceutical composition can include a solvent or dispersion medium containing, for example, water, ethanol, polyol (for example, glycerol, propylene glycol, and liquid polyethylene glycol, and the like), suitable mixtures thereof, and vegetable oils. The proper fluidity can be maintained, for example, by the use of a coating, such as lecithin, by the maintenance of the required particle size in the case of dispersion, and by the use of surfactants. The prevention of the action of microorganisms can be brought about by various antibacterial and antifungal agents, for example, parabens, chlorobutanol, phenol, sorbic acid, thimerosal, and the like. In many cases, it will be preferable to include isotonic agents, for example, sugars or sodium chloride. Prolonged absorption of the injectable compositions can be brought about by the use in the compositions of agents delaying absorption, for example, aluminum monostearate and gelatin.

The composition, if desired, can also contain minor amounts of wetting or emulsifying agents, or pH buffering agents. These compositions can take the form of solutions, suspensions, emulsion, tablets, pills, capsules, powders, sustained-release formulations and the like. Oral formulations can include standard carriers such as pharmaceutical grades of mannitol, lactose, starch, magnesium stearate, sodium saccharine, cellulose, magnesium carbonate, etc. Examples of suitable pharmaceutical agents are described in Remington's Pharmaceutical Sciences. Such compositions will contain a prophylactically or therapeutically effective amount of the antibody or fragment thereof, preferably in purified form, together with a suitable amount of carrier so as to provide the form for proper administration to the patient.

Passive transfer of antibodies generally will involve the use of intravenous or intramuscular injections. The resulting immunity generally lasts for only a short period of time, and there is also a potential risk for hypersensitivity reactions, and serum sickness, especially from gamma globulin of non-human origin. The antibodies may be formulated in a carrier suitable for injection, i.e., sterile and syringeable.

Generally, the ingredients of compositions of the disclosure are supplied either separately or mixed together in unit dosage form, for example, as a dry lyophilized powder or water-free concentrate in a hermetically sealed container such as an ampoule or sachette indicating the quantity of active agent. Where the composition is to be administered by infusion, it can be dispensed with an infusion bottle containing sterile pharmaceutical grade water or saline. Where the composition is administered by injection, an ampoule of sterile water for injection or saline can be provided so that the ingredients may be mixed prior to administration.

In certain embodiments, pharmaceutical compositions may comprise, for example, at least about 0.1% of an active ingredient. In other embodiments, an active ingredient may comprise between about 2% to about 75% of the weight of the unit, or between about 25% to about 60%, for example, and any range derivable therein.

The term “unit dose” or “dosage” refers to physically discrete units suitable for use in a subject, each unit containing a predetermined quantity of the therapeutic composition calculated to produce the desired responses discussed above in association with its administration, i.e., the appropriate route and treatment regimen. The quantity to be administered, both according to number of treatments and unit dose, depends on the effect desired. The actual dosage amount of a composition of the present embodiments administered to a patient or subject can be determined by physical and physiological factors, such as body weight, the age, health, and sex of the subject, the type of disease being treated, the extent of disease penetration, previous or concurrent therapeutic interventions, idiopathy of the patient, the route of administration, and the potency, stability, and toxicity of the particular therapeutic substance. For example, a dose may also comprise from about 1 μg/kg/body weight to about 1000 mg/kg/body weight (this such range includes intervening doses) or more per administration, and any range derivable therein. In non-limiting examples of a derivable range from the numbers listed herein, a range of about 5 μg/kg/body weight to about 100 mg/kg/body weight, about 5 μg/kg/body weight to about 500 mg/kg/body weight, etc., can be administered. The practitioner responsible for administration will, in any event, determine the concentration of active ingredient(s) in a composition and appropriate dose(s) for the individual subject.

### IV. Methods of Treatment

Certain aspects of the present embodiments can be used to prevent or treat a disease or disorder associated with elevated levels of HSP70, such as cancer, such as lung cancer, prostate cancer, stomach cancer, thyroid cancer, breast cancer multiple myeloma, melanoma, colon cancer, or pancreatic cancer. Functioning of HSP70 may be reduced by any suitable drugs e.g., an anti-HSP70 antibody.

The term “cancer,” as used herein, may be used to describe a solid tumor, metastatic cancer, or non-metastatic cancer. In certain embodiments, the cancer may originate in the bladder, blood, bone, bone marrow, brain, breast, colon, esophagus, duodenum, small intestine, large intestine, colon, rectum, anus, gum, head, kidney, liver, lung, nasopharynx, neck, ovary, pancreas, prostate, skin, stomach, testis, tongue, or uterus.

The cancer may specifically be of the following histological type, though it is not limited to these: neoplasm, malignant; carcinoma; carcinoma, undifferentiated; giant and spindle cell carcinoma; small cell carcinoma; papillary carcinoma; squamous cell carcinoma; lymphoepithelial carcinoma; basal cell carcinoma; pilomatrix carcinoma; transitional cell carcinoma; papillary transitional cell carcinoma; adenocarcinoma; gastrinoma, malignant; cholangiocarcinoma; hepatocellular carcinoma; combined hepatocellular carcinoma and cholangiocarcinoma; trabecular adenocarcinoma; adenoid cystic carcinoma; adenocarcinoma in adenomatous polyp; adenocarcinoma, familial polyposis coli; solid carcinoma; carcinoid tumor, malignant; branchiolo-alveolar adenocarcinoma; papillary adenocarcinoma; chromophobe carcinoma; acidophil carcinoma; oxyphilic adenocarcinoma; basophil carcinoma; clear cell adenocarcinoma; granular cell carcinoma; follicular adenocarcinoma; papillary and follicular adenocarcinoma; nonencapsulating sclerosing carcinoma; adrenal cortical carcinoma; endometroid carcinoma; skin appendage carcinoma; apocrine adenocarcinoma; sebaceous adenocarcinoma; ceruminous adenocarcinoma; mucoepidermoid carcinoma; cystadenocarcinoma; papillary cystadenocarcinoma; papillary serous cystadenocarcinoma; mucinous cystadenocarcinoma; mucinous adenocarcinoma; signet ring cell carcinoma; infiltrating duct carcinoma; medullary carcinoma; lobular carcinoma; inflammatory carcinoma; paget's disease, mammary; acinar cell carcinoma; adenosquamous carcinoma; adenocarcinoma w/squamous metaplasia; thymoma, malignant; ovarian stromal tumor, malignant; thecoma, malignant; granulosa cell tumor, malignant; androblastoma, malignant; sertoli cell carcinoma; leydig cell tumor, malignant; lipid cell tumor, malignant; paraganglioma, malignant; extra-mammary paraganglioma, malignant; pheochromocytoma; glomangiosarcoma; malignant melanoma; amelanotic melanoma; superficial spreading melanoma; malignant melanoma in giant pigmented nevus; epithelioid cell melanoma; blue nevus, malignant; sarcoma; fibrosarcoma; fibrous histiocytoma, malignant; myxosarcoma; liposarcoma; leiomyosarcoma; rhabdomyosarcoma; embryonal rhabdomyosarcoma; alveolar rhabdomyosarcoma; stromal sarcoma; mixed tumor, malignant; mullerian mixed tumor; nephroblastoma; hepatoblastoma; carcinosarcoma; mesenchymoma, malignant; brenner tumor, malignant; phyllodes tumor, malignant; synovial sarcoma; mesothelioma, malignant; dysgerminoma; embryonal carcinoma; teratoma, malignant; struma ovarii, malignant; choriocarcinoma; mesonephroma, malignant; hemangiosarcoma; hemangioendothelioma, malignant; kaposi's sarcoma; hemangiopericytoma, malignant; lymphangiosarcoma; osteosarcoma; juxtacortical osteosarcoma; chondrosarcoma; chondroblastoma, malignant; mesenchymal chondrosarcoma; giant cell tumor of bone; ewing's sarcoma; odontogenic tumor, malignant; ameloblastic odontosarcoma; ameloblastoma, malignant; ameloblastic fibrosarcoma; pinealoma, malignant; chordoma; glioma, malignant; ependymoma; astrocytoma; protoplasmic astrocytoma; fibrillary astrocytoma; astroblastoma; glioblastoma; oligodendroglioma; oligodendroblastoma; primitive neuroectodermal; cerebellar sarcoma; ganglioneuroblastoma; neuroblastoma; retinoblastoma; olfactory neurogenic tumor; meningioma, malignant; neurofibrosarcoma; neurilemmoma, malignant; granular cell tumor, malignant; malignant lymphoma; hodgkin's disease; hodgkin's; paragranuloma; malignant lymphoma, small lymphocytic; malignant lymphoma, large cell, diffuse; malignant lymphoma, follicular; mycosis fungoides; other specified non-hodgkin's lymphomas; malignant histiocytosis; multiple myeloma; mast cell sarcoma; immunoproliferative small intestinal disease; leukemia; lymphoid leukemia; plasma cell leukemia; erythroleukemia; lymphosarcoma cell leukemia; myeloid leukemia; basophilic leukemia; eosinophilic leukemia; monocytic leukemia; mast cell leukemia; megakaryoblastic leukemia; myeloid sarcoma; and hairy cell leukemia.

In certain embodiments, the serum of a subject to be treated with a disclosed composition or method (or a serum sample from the subject) has an HSP70 level greater than 5 ng/ml, 10 ng/ml, 15 ng/ml, 20 ng/ml, 25 ng/ml, or 30 ng/ml.

Nonetheless, it is also recognized that the present invention may also be used to treat a non-cancerous disease (e.g., a fungal infection, a bacterial infection, a viral infection, a neurodegenerative disease, and/or a genetic disorder).

In certain embodiments, the compositions and methods of the present embodiments involve an antibody or an antibody fragment against HSP70, in combination with a second or additional therapy, such as chemotherapy or immunotherapy. Such therapy can be applied in the treatment of any disease that is associated with elevated HSP70. For example, the disease may be a cancer.

The methods and compositions, including combination therapies, enhance the therapeutic or protective effect, and/or increase the therapeutic effect of another anti-cancer or anti-hyperproliferative therapy. Therapeutic and prophylactic methods and compositions can be provided in a combined amount effective to achieve the desired effect, such as the killing of a cancer cell and/or the inhibition of cellular hyperproliferation. This process may involve contacting the cells with both an antibody or antibody fragment and a second therapy. A tissue, tumor, or cell can be contacted with one or more compositions or pharmacological formulation(s) comprising one or more of the agents (i.e., antibody or antibody fragment or an anti-cancer agent), or by contacting the tissue, tumor, and/or cell with two or more distinct compositions or formulations, wherein one composition provides 1) an antibody or antibody fragment, 2) an anti-cancer agent, or 3) both an antibody or antibody fragment and an anti-cancer agent. Also, it is contemplated that such a combination therapy can be used in conjunction with chemotherapy, radiotherapy, surgical therapy, or immunotherapy.

The terms “contacted” and “exposed,” when applied to a cell, are used herein to describe the process by which a therapeutic construct and a chemotherapeutic or radiotherapeutic agent are delivered to a target cell or are placed in direct juxtaposition with the target cell. To achieve cell killing, for example, both agents are delivered to a cell in a combined amount effective to kill the cell or prevent it from dividing.

An antibody may be administered before, during, after, or in various combinations relative to an anti-cancer treatment. The administrations may be in intervals ranging from concurrently to minutes to days to weeks. In embodiments where the antibody or antibody fragment is provided to a patient separately from an anti-cancer agent, one would generally ensure that a significant period of time did not expire between the time of each delivery, such that the two compounds would still be able to exert an advantageously combined effect on the patient. In such instances, it is contemplated that one may provide a patient with the antibody therapy and the anti-cancer therapy within about 12 to 24 or 72 h of each other and, more particularly, within about 6-12 h of each other. In some situations it may be desirable to extend the time period for treatment significantly where several days (2, 3, 4, 5, 6, or 7) to several weeks (1, 2, 3, 4, 5, 6, 7, or 8) lapse between respective administrations.

In certain embodiments, a course of treatment will last 1-90 days or more (this such range includes intervening days). It is contemplated that one agent may be given on any day of day 1 to day 90 (this such range includes intervening days) or any combination thereof, and another agent is given on any day of day 1 to day 90 (this such range includes intervening days) or any combination thereof. Within a single day (24-hour period), the patient may be given one or multiple administrations of the agent(s). Moreover, after a course of treatment, it is contemplated that there is a period of time at which no anti-cancer treatment is administered. This time period may last 1-7 days, and/or 1-5 weeks, and/or 1-12 months or more (this such range includes intervening days), depending on the condition of the patient, such as their prognosis, strength, health, etc. It is expected that the treatment cycles would be repeated as necessary.

Various combinations may be employed. For the example below an antibody therapy is “A” and an anti-cancer therapy is “B”:

A/B/A B/A/B B/B/A A/A/B A/B/B B/A/A A/B/B/B B/A/B/B
B/B/B/A B/B/A/B A/A/B/B A/B/A/B A/B/B/A B/B/A/A
B/A/B/A B/A/A/B A/A/A/B B/A/A/A A/B/A/A A/A/B/A

Administration of any compound or therapy of the present embodiments to a patient will follow general protocols for the administration of such compounds, taking into account the toxicity, if any, of the agents. Therefore, in some embodiments there is a step of monitoring toxicity that is attributable to combination therapy.

### A. Chemotherapy

A wide variety of chemotherapeutic agents may be used in accordance with the present embodiments. The term “chemotherapy” refers to the use of drugs to treat cancer. A “chemotherapeutic agent” is used to connote a compound or composition that is administered in the treatment of cancer. These agents or drugs are categorized by their mode of activity within a cell, for example, whether and at what stage they affect the cell cycle. Alternatively, an agent may be characterized based on its ability to directly cross-link DNA, to intercalate into DNA, or to induce chromosomal and mitotic aberrations by affecting nucleic acid synthesis.

Examples of chemotherapeutic agents include alkylating agents, such as thiotepa and cyclosphosphamide; alkyl sulfonates, such as busulfan, improsulfan, and piposulfan; aziridines, such as benzodopa, carboquone, meturedopa, and uredopa; ethylenimines and methylamelamines, including altretamine, triethylenemelamine, trietylenephosphoramide, triethiylenethiophosphoramide, and trimethylolomelamine; acetogenins (especially bullatacin and bullatacinone); a camptothecin (including the synthetic analogue topotecan); bryostatin; callystatin; CC-1065 (including its adozelesin, carzelesin and bizelesin synthetic analogues); cryptophycins (particularly cryptophycin 1 and cryptophycin 8); dolastatin; duocarmycin (including the synthetic analogues, KW-2189 and CB1-TM1); eleutherobin; pancratistatin; a sarcodictyin; spongistatin; nitrogen mustards, such as chlorambucil, chlornaphazine, cholophosphamide, estramustine, ifosfamide, mechlorethamine, mechlorethamine oxide hydrochloride, melphalan, novembichin, phenesterine, prednimustine, trofosfamide, and uracil mustard; nitrosureas, such as carmustine, chlorozotocin, fotemustine, lomustine, nimustine, and ranimnustine; antibiotics, such as the enediyne antibiotics (e.g., calicheamicin, especially calicheamicin gammalI and calicheamicin omegaIl); dynemicin, including dynemicin A; bisphosphonates, such as clodronate; an esperamicin; as well as neocarzinostatin chromophore and related chromoprotein enediyne antibiotic chromophores, aclacinomysins, actinomycin, authrarnycin, azaserine, bleomycins, cactinomycin, carabicin, carminomycin, carzinophilin, chromomycinis, dactinomycin, daunorubicin, detorubicin, 6-diazo-5-oxo-L-norleucine, doxorubicin (including morpholino-doxorubicin, cyanomorpholino-doxorubicin, 2-pyrrolino-doxorubicin and deoxydoxorubicin), epirubicin, esorubicin, idarubicin, marcellomycin, mitomycins, such as mitomycin C, mycophenolic acid, nogalarnycin, olivomycins, peplomycin, potfiromycin, puromycin, quelamycin, rodorubicin, streptonigrin, streptozocin, tubercidin, ubenimex, zinostatin, and zorubicin; anti-metabolites, such as methotrexate and 5-fluorouracil (5-FU); folic acid analogues, such as denopterin, pteropterin, and trimetrexate; purine analogs, such as fludarabine, 6-mercaptopurine, thiamiprine, and thioguanine; pyrimidine analogs, such as ancitabine, azacitidine, 6-azauridine, carmofur, cytarabine, dideoxyuridine, doxifluridine, enocitabine, and floxuridine; androgens, such as calusterone, dromostanolone propionate, epitiostanol, mepitiostane, and testolactone; anti-adrenals, such as mitotane and trilostane; folic acid replenisher, such as frolinic acid; aceglatone; aldophosphamide glycoside; aminolevulinic acid; eniluracil; amsacrine; bestrabucil; bisantrene; edatraxate; defofamine; demecolcine; diaziquone; elformithine; elliptinium acetate; an epothilone; etoglucid; gallium nitrate; hydroxyurea; lentinan; lonidainine; maytansinoids, such as maytansine and ansamitocins; mitoguazone; mitoxantrone; mopidanmol; nitraerine; pentostatin; phenamet; pirarubicin; losoxantrone; podophyllinic acid; 2-ethylhydrazide; procarbazine; PSKpolysaccharide complex; razoxane; rhizoxin; sizofiran; spirogermanium; tenuazonic acid; triaziquone; 2,2′,2″-trichlorotriethylamine; trichothecenes (especially T-2 toxin, verracurin A, roridin A and anguidine); urethan; vindesine; dacarbazine; mannomustine; mitobronitol; mitolactol; pipobroman; gacytosine; arabinoside (“Ara-C”); cyclophosphamide; taxoids, e.g., paclitaxel and docetaxel gemcitabine; 6-thioguanine; mercaptopurine; platinum coordination complexes, such as cisplatin, oxaliplatin, and carboplatin; vinblastine; platinum; etoposide (VP-16); ifosfamide; mitoxantrone; vincristine; vinorelbine; novantrone; teniposide; edatrexate; daunomycin; aminopterin; xeloda; ibandronate; irinotecan (e.g., CPT-11); topoisomerase inhibitor RFS 2000; difluorometlhylornithine (DFMO); retinoids, such as retinoic acid; capecitabine; carboplatin, procarbazine, plicomycin, gemcitabien, navelbine, farnesyl-protein transferase inhibitors, transplatinum, and pharmaceutically acceptable salts, acids, or derivatives of any of the above.

### B. Radiotherapy

Other factors that cause DNA damage and have been used extensively include what are commonly known as γ-rays, X-rays, and/or the directed delivery of radioisotopes to tumor cells. Other forms of DNA damaging factors are also contemplated, such as microwaves, proton beam irradiation (U.S. Pat. Nos. 5,760,395 and 4,870,287), and UV-irradiation. It is most likely that all of these factors affect a broad range of damage on DNA, on the precursors of DNA, on the replication and repair of DNA, and on the assembly and maintenance of chromosomes. Dosage ranges for X-rays range from daily doses of 50 to 200 roentgens for prolonged periods of time (3 to 4 wk), to single doses of 2,000 to 6,000 roentgens. Dosage ranges for radioisotopes vary widely, and depend on the half-life of the isotope, the strength and type of radiation emitted, and the uptake by the neoplastic cells. In certain embodiments, an antibody of the present disclosure is used in combination with a radiotherapy, e.g., γ-rays, X-rays, and/or the directed delivery of radioisotopes to tumor cells.

### C. Immunotherapy

The skilled artisan will understand that immunotherapies may be used in combination or in conjunction with methods of the embodiments. In the context of cancer treatment, immunotherapeutics, generally, rely on the use of immune effector cells and molecules to target and destroy cancer cells. Rituximab (RITUXAN®) is such an example. The immune effector may be, for example, an antibody specific for some marker on the surface of a tumor cell. The antibody alone may serve as an effector of therapy or it may recruit other cells to actually affect cell killing. The antibody also may be conjugated to a drug or toxin (chemotherapeutic, radionuclide, ricin A chain, cholera toxin, pertussis toxin, etc.) and serve merely as a targeting agent. Alternatively, the effector may be a lymphocyte carrying a surface molecule that interacts, either directly or indirectly, with a tumor cell target. Various effector cells include cytotoxic T cells and NK cells.

In one aspect of immunotherapy, the tumor cell must bear some marker that is amenable to targeting, i.e., is not present on the majority of other cells. Many tumor markers exist and any of these may be suitable for targeting in the context of the present embodiments. Common tumor markers include B-cell maturation antigen, CD20, carcinoembryonic antigen, tyrosinase (p97), gp68, GPRC5D, TAG-72, HMFG, Sialyl Lewis Antigen, MucA, MucB, PLAP, laminin receptor, erb B, and p155. An alternative aspect of immunotherapy is to combine anticancer effects with immune stimulatory effects. Immune stimulating molecules also exist including: cytokines, such as IL-2, IL-4, IL-12, GM-CSF, gamma-IFN, chemokines, such as MIP-1, MCP-1, IL-8, and growth factors, such as FLT3 ligand.

Examples of immunotherapies currently under investigation or in use are immune adjuvants, e.g., Mycobacterium bovis, Plasmodium falciparum, dinitrochlorobenzene, and aromatic compounds (U.S. Pat. Nos. 5,801,005 and 5,739,169; Hui and Hashimoto, 1998; Christodoulides et al., 1998); cytokine therapy, e.g., interferons α, β, and γ, IL-1, GM-CSF, and TNF (Bukowski et al., 1998; Davidson et al., 1998; Hellstrand et al., 1998); gene therapy, e.g., TNF, IL-1, IL-2, and p53 (Qin et al., 1998; Austin-Ward and Villaseca, 1998; U.S. Pat. Nos. 5,830,880 and 5,846,945); and monoclonal antibodies, e.g., anti-CD20, anti-ganglioside GM2, and anti-p185 (Hollander, 2012; Hanibuchi et al., 1998; U.S. Pat. No. 5,824,311). It is contemplated that one or more anti-cancer therapies may be employed with the antibody therapies described herein.

In some aspects, a combination described herein includes an agent that decreases tumor immunosuppression, such as a chemokine (C-X-C motif) receptor 2 (CXCR2) inhibitor. In some embodiments, the CXCR2 inhibitor is danirixin (CAS Registry Number: 954126-98-8). Danirixin is also known as GSK1325756 or 1-(4-chloro-2-hydroxy-3-piperidin-3-ylsulfonylphenyl)-3-(3-fluoro-2-methylphenyl)urea. Danirixin is disclosed, e.g., in Miller et al. Eur J Drug Metab Pharmacokinet (2014) 39:173-181; and Miller et al. BMC Pharmacology and Toxicology (2015), 16:18. In some embodiments, the CXCR2 inhibitor is reparixin (CAS Registry Number: 266359-83-5). Reparixin is also known as repertaxin or (2R)-2-[4-(2-methylpropyl)phenyl]-N-methylsulfonylpropanamide. Reparixin is a non-competitive allosteric inhibitor of CXCR1/2. Reparixin is disclosed, e.g., in Zarbock et al. British Journal of Pharmacology (2008), 1-8. In some embodiments, the CXCR2 inhibitor is navarixin. Navarixin is also known as MK-7123, SCH527123, PS291822, or 2-hydroxy-N,N-dimethyl-3-[[2-[[(1R)-1-(5-methylfuran-2-yl)propyl]amino]-3,4-dioxocyclobuten-1-yl]amino]benzamide Navarixin is disclosed, e.g., in Ning et al. Mol Cancer Ther. 2012; 11(6):1353-64. In some embodiments, the CXCR2 inhibitor is AZD5069, also known as N-[2-[[(2,3-difluoropheny)methyl]thio]-6-{[(1 R,2S)-2,3-dihydroxy-1-methylpropyl]oxy}-4-pyrimidinyl]-1-azetidinesulfonamide. In some embodiments, the CXCR2 inhibitor is an anti-CXCR2 antibody, such as those disclosed in WO2020/028479.

In some aspects, a combination described herein includes an agent that activates dendritic cells, such as, for example, a TLR agonist. A “TLR agonist” as defined herein is any molecule which activates a toll-like receptor as described in Bauer et al., 2001, Proc. Natl. Acad. Sci. USA 98: 9237-9242. A TLR agonist may be a small molecule, a recombinant protein, an antibody or antibody fragment, a nucleic acid, or a protein. In certain embodiments, the TLR agonist is recombinant, a natural ligand, an immunostimulatory nucleotide sequence, a small molecule, a purified bacterial extract or an inactivated bacteria preparation.

Several agonists of TLR derived from microbes have been described, such as lipopolysaccharides, peptidoglycans, flagellin and lipoteichoic acid (Aderem et al., 2000, Nature 406:782-787; Akira et al., 2001, Nat. Immunol. 2: 675-680) Some of these ligands can activate different dendritic cell subsets, that express distinct patterns of TLRs (Kadowaki et al., 2001, J. Exp. Med. 194: 863-869). Therefore, a TLR agonist could be any preparation of a microbial agent that possesses TLR agonist properties. Certain types of untranslated DNA have been shown to stimulate immune responses by activating TLRs. In particular, immunostimulatory oligonucleotides containing CpG motifs have been widely disclosed and reported to activate lymphocytes (see, U.S. Pat. No. 6,194,388). A “CpG motif” as used herein is defined as an unmethylated cytosine-guanine (CpG) dinucleotide. Immunostimulatory oligonucleotides which contain CpG motifs can also be used as TLR agonists according to the methods of the present invention. The immunostimulatory nucleotide sequence may be stabilized by structure modification such as phosphorothioate modification or may be encapsulated in cationic liposomes to improve in vivo pharmacokinetics and tumor targeting.

In some embodiments, the immunotherapy may be an immune checkpoint inhibitor. Immune checkpoints either turn up a signal (e.g., co-stimulatory molecules) or turn down a signal. Immune checkpoints either turn up a signal (e.g., co-stimulatory molecules) or turn down a signal. Immune checkpoint proteins that may be targeted by immune checkpoint blockade include adenosine A2A receptor (A2AR), B7-H3 (also known as CD276), B and T lymphocyte attenuator (BTLA), CCL5, CD27, CD38, CD8A, CMKLR1, cytotoxic T-lymphocyte-associated protein 4 (CTLA-4, also known as CD152), CXCL9, CXCR5, glucocorticoid-induced tumour necrosis factor receptor-related protein (GITR), HLA-DRB1, ICOS (also known as CD278), HLA-DQA1, HLA-E, indoleamine 2,3-dioxygenase 1 (IDO1), killer-cell immunoglobulin (KIR), lymphocyte activation gene-3 (LAG-3, also known as CD223), Mer tyrosine kinase (MerTK), NKG7, OX40 (also known as CD134), programmed death 1 (PD-1), programmed death-ligand 1 (PD-L1, also known as CD274), PDCD1LG2, PSMB10, STAT1, T cell immunoreceptor with Ig and ITIM domains (TIGIT), T-cell immunoglobulin domain and mucin domain 3 (TIM-3), and V-domain Ig suppressor of T cell activation (VISTA, also known as C10orf54). In particular, the immune checkpoint inhibitors target the PD-1 axis and/or CTLA-4.

The immune checkpoint inhibitors may be drugs, such as small molecules, recombinant forms of ligand or receptors, or antibodies, such as human antibodies (e.g., International Patent Publication WO2015/016718; Pardoll, Nat Rev Cancer, 12(4): 252-264, 2012; both incorporated herein by reference). Known inhibitors of the immune checkpoint proteins or analogs thereof may be used, in particular chimerized, humanized, or human forms of antibodies may be used. As the skilled person will know, alternative and/or equivalent names may be in use for certain antibodies mentioned in the present disclosure. Such alternative and/or equivalent names are interchangeable in the context of the present disclosure. For example, it is known that lambrolizumab is also known under the alternative and equivalent names MK-3475 and pembrolizumab.

In some embodiments, a PD-1 binding antagonist is a molecule that inhibits the binding of PD-1 to its ligand binding partners. In a specific aspect, the PD-1 ligand binding partners are PD-L1 and/or PD-L2. In another embodiment, a PD-L1 binding antagonist is a molecule that inhibits the binding of PD-L1 to its binding partners. In a specific aspect, PD-L1 binding partners are PD-1 and/or B7-1. In another embodiment, a PD-L2 binding antagonist is a molecule that inhibits the binding of PD-L2 to its binding partners. In a specific aspect, a PD-L2 binding partner is PD-1. The antagonist may be an antibody, an antigen binding fragment thereof, an immunoadhesin, a fusion protein, or an oligopeptide. Exemplary antibodies are described in U.S. Pat. Nos. 8,735,553, 8,354,509, and 8,008,449, all of which are incorporated herein by reference. Other PD-1 axis antagonists for use in the methods provided herein are known in the art, such as described in U.S. Patent Application Publication Nos. 2014/0294898, 2014/022021, and 2011/0008369, all of which are incorporated herein by reference.

In some embodiments, a PD-1 binding antagonist is an anti-PD-1 antibody (e.g., a human antibody, a humanized antibody, or a chimeric antibody). In some embodiments, the anti-PD-1 antibody is selected from the group consisting of nivolumab, pembrolizumab, and CT-011. In some embodiments, the PD-1 binding antagonist is an immunoadhesin (e.g., an immunoadhesin comprising an extracellular or PD-1 binding portion of PD-L1 or PD-L2 fused to a constant region (e.g., an Fc region of an immunoglobulin sequence)). In some embodiments, the PD-1 binding antagonist is AMP-224. Nivolumab, also known as MDX-1106-04, MDX-1106, ONO-4538, BMS-936558, and OPDIVO®, is an anti-PD-1 antibody described in WO2006/121168. Pembrolizumab, also known as MK-3475, Merck 3475, lambrolizumab, KEYTRUDA®, and SCH-900475, is an anti-PD-1 antibody described in WO2009/114335. CT-011, also known as hBAT or hBAT-1, is an anti-PD-1 antibody described in WO2009/101611. AMP-224, also known as B7-DCIg, is a PD-L2-Fc fusion soluble receptor described in WO2010/027827 and WO2011/066342.

Another immune checkpoint protein that can be targeted in the methods provided herein is the cytotoxic T-lymphocyte-associated protein 4 (CTLA-4), also known as CD152. The complete cDNA sequence of human CTLA-4 has the Genbank accession number L15006. CTLA-4 is found on the surface of T cells and acts as an “off” switch when bound to CD80 or CD86 on the surface of antigen-presenting cells. CTLA-4 is similar to the T-cell co-stimulatory protein, CD28, and both molecules bind to CD80 and CD86, also called B7-1 and B7-2 respectively, on antigen-presenting cells. CTLA-4 transmits an inhibitory signal to T cells, whereas CD28 transmits a stimulatory signal. Intracellular CTLA-4 is also found in regulatory T cells and may be important to their function. T cell activation through the T cell receptor and CD28 leads to increased expression of CTLA-4, an inhibitory receptor for B7 molecules.

In some embodiments, the immune checkpoint inhibitor is an anti-CTLA-4 antibody (e.g., a human antibody, a humanized antibody, or a chimeric antibody), an antigen binding fragment thereof, an immunoadhesin, a fusion protein, or oligopeptide. Anti-human-CTLA-4 antibodies (or VH and/or VL domains derived therefrom) suitable for use in the present methods can be generated using methods well known in the art. Alternatively, art recognized anti-CTLA-4 antibodies can be used. For example, the anti-CTLA-4 antibodies disclosed in U.S. Pat. No. 8,119,129; PCT Publn. Nos. WO 01/14424, WO 98/42752, WO 00/37504 (CP675,206, also known as tremelimumab; formerly ticilimumab); U.S. Pat. No. 6,207,156; Hurwitz et al. (1998) Proc Natl Acad Sci USA, 95(17): 10067-10071; Camacho et al. (2004) J Clin Oncology, 22(145): Abstract No. 2505 (antibody CP-675206); and Mokyr et al. (1998) Cancer Res, 58:5301-5304 can be used in the methods disclosed herein. The teachings of each of the aforementioned publications are hereby incorporated by reference. Antibodies that compete with any of these art-recognized antibodies for binding to CTLA-4 also can be used. For example, a humanized CTLA-4 antibody is described in International Patent Application No. WO2001/014424, WO2000/037504, and U.S. Pat. No. 8,017,114; all incorporated herein by reference.

An exemplary anti-CTLA-4 antibody is ipilimumab (also known as 10D1, MDX-010, MDX-101, and Yervoy®) or antigen binding fragments and variants thereof (see, e.g., WO 01/14424). In other embodiments, the antibody comprises the heavy and light chain CDRs or VRs of ipilimumab. Accordingly, in one embodiment, the antibody comprises the CDR1, CDR2, and CDR3 domains of the VH region of ipilimumab, and the CDR1, CDR2, and CDR3 domains of the VL region of ipilimumab. In another embodiment, the antibody competes for binding with and/or binds to the same epitope on CTLA-4 as the above-mentioned antibodies. In another embodiment, the antibody has an at least about 90% variable region amino acid sequence identity with the above-mentioned antibodies (e.g., at least about 90%, 95%, or 99% variable region identity with ipilimumab). Other molecules for modulating CTLA-4 include CTLA-4 ligands and receptors such as described in U.S. Pat. Nos. 5,844,905, 5,885,796 and International Patent Application Nos. WO1995001994 and WO1998042752; all incorporated herein by reference, and immunoadhesins such as described in U.S. Pat. No. 8,329,867, incorporated herein by reference.

Another immune checkpoint protein that can be targeted in the methods provided herein is lymphocyte-activation gene 3 (LAG-3), also known as CD223. The complete protein sequence of human LAG-3 has the Genbank accession number NP-002277. LAG-3 is found on the surface of activated T cells, natural killer cells, B cells, and plasmacytoid dendritic cells. LAG-3 acts as an “off” switch when bound to MHC class II on the surface of antigen-presenting cells. Inhibition of LAG-3 both activates effector T cells and inhibitor regulatory T cells. In some embodiments, the immune checkpoint inhibitor is an anti-LAG-3 antibody (e.g., a human antibody, a humanized antibody, or a chimeric antibody), an antigen binding fragment thereof, an immunoadhesin, a fusion protein, or oligopeptide. Anti-human-LAG-3 antibodies (or VH and/or VL domains derived therefrom) suitable for use in the present methods can be generated using methods well known in the art. Alternatively, art recognized anti-LAG-3 antibodies can be used. An exemplary anti-LAG-3 antibody is relatlimab (also known as BMS-986016) or antigen binding fragments and variants thereof (see, e.g., WO 2015/116539). Other exemplary anti-LAG-3 antibodies include TSR-033 (see, e.g., WO 2018/201096), MK-4280, and REGN3767. MGD013 is an anti-LAG-3/PD-1 bispecific antibody described in WO 2017/019846. FS118 is an anti-LAG-3/PD-L1 bispecific antibody described in WO 2017/220569.

Another immune checkpoint protein that can be targeted in the methods provided herein is V-domain Ig suppressor of T cell activation (VISTA), also known as C10orf54. The complete protein sequence of human VISTA has the Genbank accession number NP_071436. VISTA is found on white blood cells and inhibits T cell effector function. In some embodiments, the immune checkpoint inhibitor is an anti-VISTA3 antibody (e.g., a human antibody, a humanized antibody, or a chimeric antibody), an antigen binding fragment thereof, an immunoadhesin, a fusion protein, or oligopeptide. Anti-human-VISTA antibodies (or V_{H} and/or VL domains derived therefrom) suitable for use in the present methods can be generated using methods well known in the art. Alternatively, art recognized anti-VISTA antibodies can be used. An exemplary anti-VISTA antibody is JNJ-61610588 (also known as onvatilimab) (see, e.g., WO 2015/097536, WO 2016/207717, WO 2017/137830, WO 2017/175058). VISTA can also be inhibited with the small molecule CA-170, which selectively targets both PD-L1 and VISTA (see, e.g., WO 2015/033299, WO 2015/033301).

Another immune checkpoint protein that can be targeted in the methods provided herein is indoleamine 2,3-dioxygenase (IDO). The complete protein sequence of human IDO has Genbank accession number NP_002155. In some embodiments, the immune checkpoint inhibitor is a small molecule IDO inhibitor. Exemplary small molecules include BMS-986205, epacadostat (INCB24360), and navoximod (GDC-0919).

Another immune checkpoint protein that can be targeted in the methods provided herein is CD38. The complete protein sequence of human CD38 has Genbank accession number NP_001766. In some embodiments, the immune checkpoint inhibitor is an anti-CD38 antibody (e.g., a human antibody, a humanized antibody, or a chimeric antibody), an antigen binding fragment thereof, an immunoadhesin, a fusion protein, or oligopeptide. Anti-human-CD38 antibodies (or VH and/or VL domains derived therefrom) suitable for use in the present methods can be generated using methods well known in the art. Alternatively, art recognized anti-CD38 antibodies can be used. An exemplary anti-CD38 antibody is daratumumab (see, e.g., U.S. Pat. No. 7,829,673).

Another immune checkpoint protein that can be targeted in the methods provided herein is ICOS, also known as CD278. The complete protein sequence of human ICOS has Genbank accession number NP_036224. In some embodiments, the immune checkpoint inhibitor is an anti-ICOS antibody (e.g., a human antibody, a humanized antibody, or a chimeric antibody), an antigen binding fragment thereof, an immunoadhesin, a fusion protein, or oligopeptide. Anti-human-ICOS antibodies (or VH and/or VL domains derived therefrom) suitable for use in the present methods can be generated using methods well known in the art. Alternatively, art recognized anti-ICOS antibodies can be used. Exemplary anti-ICOS antibodies include JTX-2011 (see, e.g., WO 2016/154177, WO 2018/187191) and GSK3359609 (see, e.g., WO 2016/059602).

Another immune checkpoint protein that can be targeted in the methods provided herein is T cell immunoreceptor with Ig and ITIM domains (TIGIT). The complete protein sequence of human TIGIT has Genbank accession number NP_776160. In some embodiments, the immune checkpoint inhibitor is an anti-TIGIT antibody (e.g., a human antibody, a humanized antibody, or a chimeric antibody), an antigen binding fragment thereof, an immunoadhesin, a fusion protein, or oligopeptide. Anti-human-TIGIT antibodies (or VH and/or VL domains derived therefrom) suitable for use in the present methods can be generated using methods well known in the art. Alternatively, art recognized anti-TIGIT antibodies can be used. An exemplary anti-TIGIT antibody is MK-7684 (see, e.g., WO 2017/030823, WO 2016/028656).

Another immune checkpoint protein that can be targeted in the methods provided herein is OX40, also known as CD134. The complete protein sequence of human OX40 has Genbank accession number NP_003318. In some embodiments, the immune checkpoint inhibitor is an anti-OX40 antibody (e.g., a human antibody, a humanized antibody, or a chimeric antibody), an antigen binding fragment thereof, an immunoadhesin, a fusion protein, or oligopeptide. Anti-human-OX40 antibodies (or VH and/or VL domains derived therefrom) suitable for use in the present methods can be generated using methods well known in the art. Alternatively, art recognized anti-OX40 antibodies can be used. An exemplary anti-OX40 antibody is PF-04518600 (see, e.g., WO 2017/130076). ATOR-1015 is a bispecific antibody targeting CTLA4 and OX40 (see, e.g., WO 2017/182672, WO 2018/091740, WO 2018/202649, WO 2018/002339).

Another immune checkpoint protein that can be targeted in the methods provided herein is glucocorticoid-induced tumour necrosis factor receptor-related protein (GITR), also known as TNFRSF18 and AITR. The complete protein sequence of human GITR has Genbank accession number NP_004186. In some embodiments, the immune checkpoint inhibitor is an anti-GITR antibody (e.g., a human antibody, a humanized antibody, or a chimeric antibody), an antigen binding fragment thereof, an immunoadhesin, a fusion protein, or oligopeptide. Anti-human-GITR antibodies (or VH and/or VL domains derived therefrom) suitable for use in the present methods can be generated using methods well known in the art. Alternatively, art recognized anti-GITR antibodies can be used. An exemplary anti-GITR antibody is TRX518 (see, e.g., WO 2006/105021).

In some embodiment, the immune therapy could be adoptive immunotherapy, which involves the transfer of autologous antigen-specific T cells generated ex vivo. The T cells used for adoptive immunotherapy can be generated either by expansion of antigen-specific T cells or redirection of T cells through genetic engineering (Park, Rosenberg et al. 2011). Isolation and transfer of tumor specific T cells has been shown to be successful in treating melanoma. Novel specificities in T cells have been successfully generated through the genetic transfer of transgenic T cell receptors or chimeric antigen receptors (CARs) (Jena, Dotti et al. 2010). CARs are synthetic receptors consisting of a targeting moiety that is associated with one or more signaling domains in a single fusion molecule. In general, the binding moiety of a CAR consists of an antigen-binding domain of a single-chain antibody (scFv), comprising the light and variable fragments of a monoclonal antibody joined by a flexible linker. Binding moieties based on receptor or ligand domains have also been used successfully. The signaling domains for first generation CARs are derived from the cytoplasmic region of the CD3zeta or the Fc receptor gamma chains. CARs have successfully allowed T cells to be redirected against antigens expressed at the surface of tumor cells from various malignancies including lymphomas and solid tumors (Jena, Dotti et al. 2010).

In one embodiment, the present application provides for a combination therapy for the treatment of cancer wherein the combination therapy comprises adoptive T cell therapy and a checkpoint inhibitor. In one aspect, the adoptive T cell therapy comprises autologous and/or allogenic T-cells. In another aspect, the autologous and/or allogenic T-cells are targeted against tumor antigens.

### D. Surgery

Approximately 60% of persons with cancer will undergo surgery of some type, which includes preventative, diagnostic or staging, curative, and palliative surgery. Curative surgery includes resection in which all or part of cancerous tissue is physically removed, excised, and/or destroyed and may be used in conjunction with other therapies, such as the treatment of the present embodiments, chemotherapy, radiotherapy, hormonal therapy, gene therapy, immunotherapy, and/or alternative therapies. Tumor resection refers to physical removal of at least part of a tumor. In addition to tumor resection, treatment by surgery includes laser surgery, cryosurgery, electrosurgery, and microscopically-controlled surgery (Mohs' surgery).

Upon excision of part or all of cancerous cells, tissue, or tumor, a cavity may be formed in the body. Treatment may be accomplished by perfusion, direct injection, or local application of the area with an additional anti-cancer therapy. Such treatment may be repeated, for example, every 1, 2, 3, 4, 5, 6, or 7 days, or every 1, 2, 3, 4, and 5 weeks or every 1, 2, 3, 4, 5, 6, 7, 8, 9, 10, 11, or 12 months. These treatments may be of varying dosages as well.

### E. Other Agents

It is contemplated that other agents may be used in combination with certain aspects of the present embodiments to improve the therapeutic efficacy of treatment. These additional agents include agents that affect the upregulation of cell surface receptors and GAP junctions, cytostatic and differentiation agents, inhibitors of cell adhesion, agents that increase the sensitivity of the hyperproliferative cells to apoptotic inducers, or other biological agents. Increases in intercellular signaling by elevating the number of GAP junctions would increase the anti-hyperproliferative effects on the neighboring hyperproliferative cell population. In other embodiments, cytostatic or differentiation agents can be used in combination with certain aspects of the present embodiments to improve the anti-hyperproliferative efficacy of the treatments. Inhibitors of cell adhesion are contemplated to improve the efficacy of the present embodiments. Examples of cell adhesion inhibitors are focal adhesion kinase (FAKs) inhibitors and Lovastatin. It is further contemplated that other agents that increase the sensitivity of a hyperproliferative cell to apoptosis, such as the antibody c225, could be used in combination with certain aspects of the present embodiments to improve the treatment efficacy.

### V. Methods of Detection

In some aspects, the present disclosure concerns immunodetection methods for detecting expression of HSP70. A wide variety of assay formats are contemplated for detecting protein products, including immunohistochemistry, enzyme linked immunosorbent assay (ELISA), radioimmunoassay (RIA), immunoradiometric assay, fluoroimmunoassay, chemiluminescent assay, bioluminescent assay, dot blotting, FACS analyses, and Western blot to mention a few. The steps of various useful immunodetection methods have been described in the scientific literature. In general, the immunobinding methods include obtaining a sample, and contacting the sample with an antibody specific for the protein to be detected, as the case may be, under conditions effective to allow the formation of immunocomplexes. In general, the detection of immunocomplex formation is well known in the art and may be achieved through the application of numerous approaches. These methods are generally based upon the detection of a label or marker, such as any of those radioactive, fluorescent, biological and enzymatic tags. Of course, one may find additional advantages through the use of a secondary binding ligand such as a second antibody and/or a biotin/avidin ligand binding arrangement, as is known in the art.

The antibody employed in the detection may itself be linked to a detectable label, wherein one would then simply detect this label, thereby allowing the amount of the primary immune complexes in the composition to be determined. Alternatively, the first antibody that becomes bound within the primary immune complexes may be detected by means of a second binding ligand that has binding affinity for the antibody. In these cases, the second binding ligand may be linked to a detectable label. The second binding ligand is itself often an antibody, which may thus be termed a “secondary” antibody. The primary immune complexes are contacted with the labeled, secondary binding ligand, or antibody, under effective conditions and for a period of time sufficient to allow the formation of secondary immune complexes. The secondary immune complexes are then generally washed to remove any non-specifically bound labeled secondary antibodies or ligands, and the remaining label in the secondary immune complexes is then detected.

As used herein, the term “sample” refers to any sample suitable for the detection methods provided by the present invention. The sample may be any sample that includes material suitable for detection or isolation. Sources of samples include blood, pleural fluid, peritoneal fluid, urine, saliva, malignant ascites, broncho-alveolar lavage fluid, synovial fluid, and bronchial washes. In one aspect, the sample is a blood sample, including, for example, whole blood or any fraction or component thereof. A blood sample suitable for use with the present invention may be extracted from any source known that includes blood cells or components thereof, such as venous, arterial, peripheral, tissue, cord, and the like. For example, a sample may be obtained and processed using well-known and routine clinical methods (e.g., procedures for drawing and processing whole blood). In one aspect, an exemplary sample may be peripheral blood drawn from a subject with cancer. In some aspects, the biological sample comprises a plurality of cells. In certain aspects, the biological sample comprises fresh or frozen tissue. In specific aspects, the biological sample comprises formalin fixed, paraffin embedded tissue. In some aspects, the biological sample is a tissue biopsy, fine needle aspirate, blood, serum, plasma, cerebral spinal fluid, urine, stool, saliva, circulating tumor cells, exosomes, or aspirates and bodily secretions, such as sweat. In some aspects, the biological sample contains cell-free DNA.

### VI. Kits

In various aspects of the embodiments, a kit is envisioned containing therapeutic agents and/or other therapeutic and delivery agents. In some embodiments, a kit is provided for preparing and/or administering a therapy of the embodiments. The kit may comprise one or more sealed vials containing any of the pharmaceutical compositions of the present embodiments. The kit may include, for example, at least one HSP70 antibody, as well as reagents to prepare, formulate, and/or administer the components of the embodiments or perform one or more steps of the inventive methods. In some embodiments, the kit may also comprise a suitable container, which is a container that will not react with components of the kit, such as an eppendorf tube, an assay plate, a syringe, a bottle, or a tube. The container may be made from sterilizable materials such as plastic or glass.

The kit may further include an instruction sheet that outlines the procedural steps of the methods set forth herein, and will follow substantially the same procedures as described herein or are known to those of ordinary skill in the art. The instruction information may be in a computer readable media containing machine-readable instructions that, when executed using a computer, cause the display of a real or virtual procedure of delivering a pharmaceutically effective amount of a therapeutic agent.

### VII. Examples

The following examples are included to demonstrate preferred embodiments of the invention. It should be appreciated by those of skill in the art that the techniques disclosed in the examples which follow represent techniques discovered by the inventor to function well in the practice of the invention, and thus can be considered to constitute preferred modes for its practice. However, those of skill in the art should, in light of the present disclosure, appreciate that many changes can be made in the specific embodiments which are disclosed and still obtain a like or similar result without departing from the spirit and scope of the invention.

### Example 1. —Generation of Therapeutic Antibodies Targeting HSP70

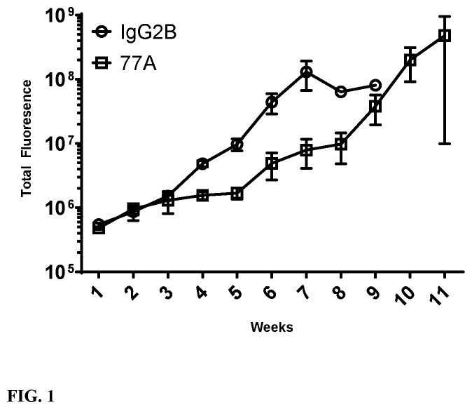

To develop an anti-HSP70 mAb, murine fibroblast L-cells (Willert et al., 2003) expressing human HSP70 fused to Green fluorescent protein (GFP) were generated and injected into the footpads of BALB/c mice in collaboration with the MD Anderson Monoclonal Antibody Core Facility. After an initial series of four injections every three days, and two boost injections on days 13 and 15, murine spleen cells were fused with Sp2/0-Ag14 murine myeloma cells (Shulman et al., 1978) to generate hybridomas. Single-cell cloning narrowed the initial output of 96 clones in mixed culture to 46 clones, followed by screening with a dry-cell ELISA to identify mAbs recognizing L-cells expressing HSP70-GFP but not vector-bearing wild-type, or GFP-expressing L-cells. The mAbs made by the remaining 28 hybridoma clones were next characterized by their ability to recognize HSP70 on intact MM1.S myeloma cells by flow, and in MM1.S cell extracts by Western blotting, which indicated that 17 clones had interesting properties. These were further screened for their binding to HSP70 versus to the closely homologous HSP70 family members (Daugaard et al., 2007) using RPMI 8226 human myeloma cells and 8226 cells in which HSP70 had been knocked out using CRISPR/Cas9-mediated genome editing, leading to the conclusion that six mAbs had the highest specificity for human HSP70. As a final screen, the antibody's anti-tumor activity was evaluated in immune-competent BALB/c mice injected with luciferase (luc)—expressing MOPC315.BM murine myeloma cells in a model that recapitulates much of the natural history of human myeloma, including the development of osteolytic bony disease (Hofgaard et al., 2012). This was possible because of the very close, 95% homology between murine and human HSP70 at the amino acid level (Hunt & Calderwood, 1990), and the finding that the mAbs bound both proteins. Clone 77A showed anti-tumor activity in pilot studies (FIG. 1), and clone 77A (hereafter referred to as 77A) was selected for further study because ⅖ treated mice showed complete myeloma resolution without recurrence at 100 days.

The complementarity-determining regions (CDR) and variable regions of the 77A antibody are provided in Tables 1-3.

TABLE 1
CDRs of heavy and light chain variable sequences of the 77A antibody.

| | CDR1 | CDR2 | CDR3 |
|---|---|---|---|
| Chain | (SEQ ID NO:) | (SEQ ID NO:) | (SEQ ID NO:) |
| Heavy | GYTFTNYG | INTYTGEP | ARYDHAMDY |
| | (SEQ ID NO: 1) | (SEQ ID NO: 2) | (SEQ ID NO: 3) |
| Light | QSLLNSGTRKNY | WTS | KQSYTLYT |
| | (SEQ ID NO: 4) | (SEQ ID NO: 5) | (SEQ ID NO: 6) |

TABLE 2
Amino acid sequences encoding the 77A antibody variable regions.

| | | SEQ ID |
|---|---|---|
| Chain | Variable Sequence | NO: |
| Heavy | QIQLVQSGPELKKPGETVKISCKASGYTFTNYGMNWVKQAPG | 7 |
| | KGLKWMGWINTYTGEPTYADDFKGRFAFSLETSATTAYLQIN | |
| | NLKNEDTATYFCARYDHAMDYWGQGTSVTVSS | |
| Light | DIVMSQSPSSLAVSAGEKVTMSCKSSQSLLNSGTRKNYLAWY | 8 |
| | QQKPGQSPKLLIYWTSTRESGVPDRFTGSGSGTDFTLTINSV | |
| | QAEDLAVYYCKQSYTLYTFGGGTKLEIK | |

TABLE 3
Nucleotide sequences encoding the 77A antibody variable regions.

| | | SEQ ID |
|---|---|---|
| Chain | Variable Sequence | NO: |
| Heavy | CAGATCCAGTTGGTGCAGTCTGGACCTGAGCTGAAGAAGCCTGGA | 9 |
| | GAGACAGTCAAGATCTCCTGCAAGGCTTCTGGGTATACCTTCACA | |
| | AACTATGGAATGAACTGGGTGAAGCAGGCTCCAGGAAAGGGTTTA | |
| | AAGTGGATGGGCTGGATAAACACCTACACTGGAGAGCCAACATAT | |
| | GCTGATGACTTCAAGGGACGGTTTGCCTTCTCTTTGGAAACCTCT | |
| | GCCACCACTGCCTATTTGCAGATCAACAACCTCAAAAATGAGGAC | |
| | ACGGCTACATATTTCTGTGCAAGGTACGACCATGCTATGGACTAC | |
| | TGGGGTCAAGGAACCTCAGTCACCGTCTCCTCAG | |
| Light | GACATTGTGATGTCACAGTCTCCATCCTCCCTGGCTGTGTCAGCT | 10 |
| | GGAGAGAAGGTCACTATGAGCTGCAAATCCAGTCAGAGTCTGCTC | |
| | AACAGTGGAACCCGAAAGAACTACTTGGCTTGGTACCAGCAGAAA | |
| | CCAGGGCAGTCTCCTAAACTGCTGATCTACTGGACATCCACTAGG | |
| | GAATCTGGGGTCCCTGATCGCTTCACAGGCAGTGGATCTGGGACA | |
| | GATTTCACTCTCACCATCAACAGTGTGCAGGCTGAAGACCTGGCA | |
| | GTTTATTACTGCAAGCAATCTTATACTCTGTACACGTTCGGAGGG | |
| | GGGACCAAGCTGGAAATAAAAC | |

### Example 2. —mAb 77A Shows Strong Affinity for Human HSP70

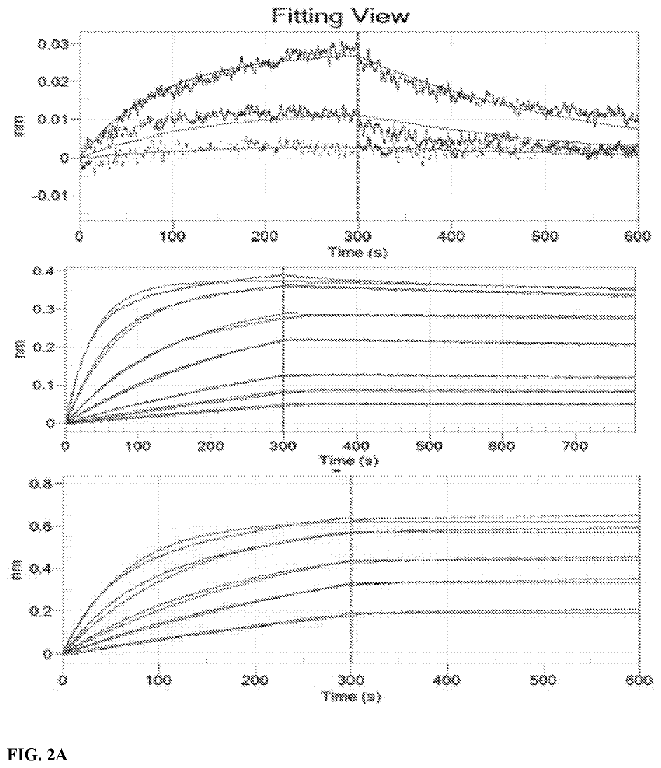

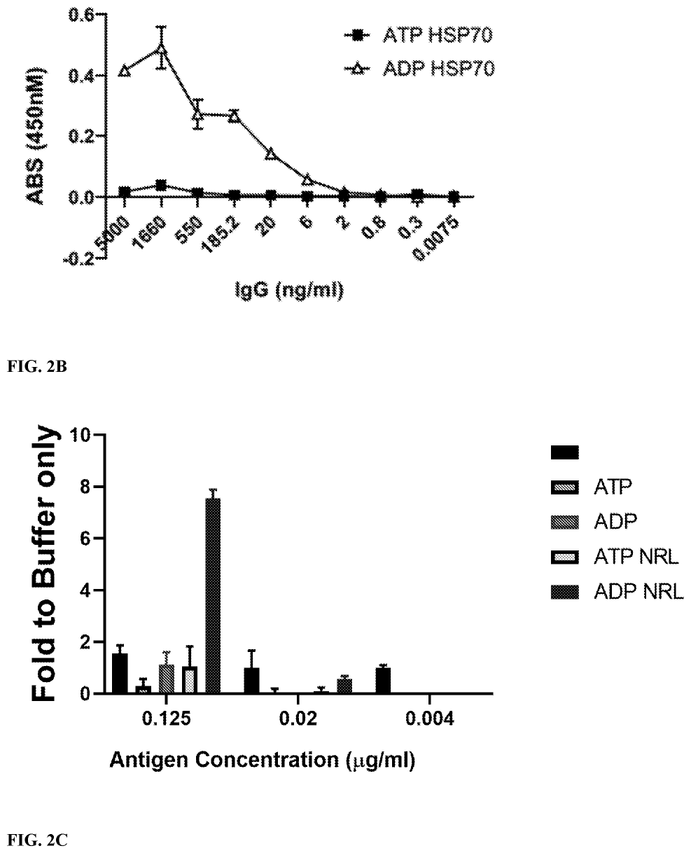

Octet analysis was pursued to study the affinity and kinetics of 77A binding (FIG. 2A). The dissociation constants (K_{D}) for murine HSP70 and human HSP70 made in E. coli were 9.88×10⁻⁷ and 8.50×10⁻¹⁰, respectively, while that for HSP70 made in eukaryotic insect Sf9 cells was below the limit of detection (1.0×10⁻¹²). To provide some context, the 0.85 nM affinity of 77A compares favorably to that of the anti-CD20 mAb rituximab (5.2 nM) (Malviay et al., 2012) and the anti-CD38 mAb daratumumab (4.36 nM) (van de Donk et al., 2016), which are in clinical use for B-cell lymphoma and myeloma, respectively. Therefore, 77A has strong affinity especially for human HSP70, and the difference between human HSP70 made in prokaryotic versus eukaryotic cells suggests that it may target an epitope that is either differently folded or post-translationally modified.

The studies to date have focused on ADP-HSP70 by purification of HSP70 over ADP-agarose as this is the form that has higher affinity for peptides and is more immunogenic (Greene et al., 2018; Peng et al., 1997). In fact, 77A shows preferential binding to ADP-HSP70 complexes, which would contain tumor-derived peptide antigens (FIG. 2B). Furthermore, an ELISA study of 77A binding to HSP70-GFP shows greatest affinity when ADP and a peptide substrate (NRL) is present (FIG. 2C). This preferential binding to HSP70 in its ADP-/peptide-bound form likely enhances delivery of tumor-associated antigens from the tumor microenvironment.

### Example 3. —Mapping of the 77A Epitope Using HSP70 Deletion Mutants

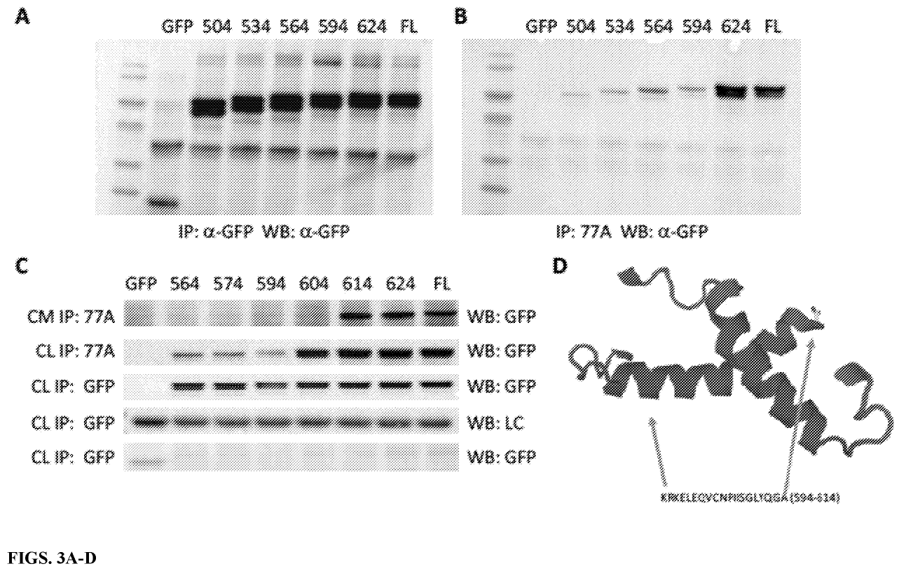

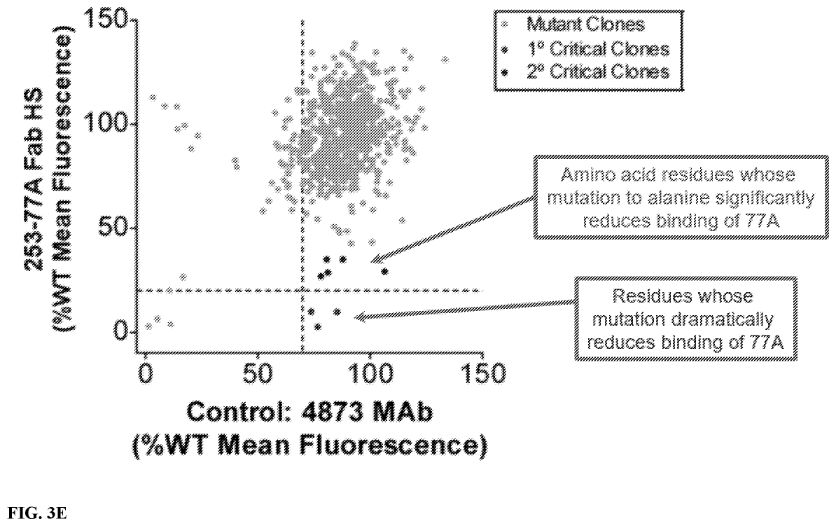

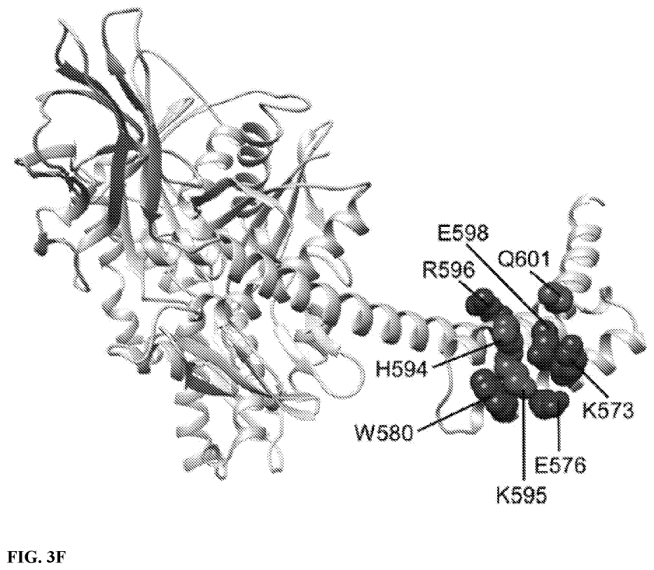

To better understand the binding of 77A to HSP70, HSP70 KO human embryonic kidney (HEK) 293T cells were generated using CRISPR/Cas9 editing, and then full-length HSP70 was expressed with an N-terminal GFP fusion, or various HSP70 deletion mutants (FIG. 3A). Immunoprecipitation (IP) of cell extracts with 77A, followed by Western blotting with an α-GFP antibody, indicated that 77A bound the 641 amino acid (aa) full-length HSP70, and the 624 aa protein with 17 amino acids (aas) deleted from the C-terminus, but binding decreased with an additional 30 aa deletion (FIG. 3B). These findings were confirmed through flow studies of 77A binding to 293T cells expressing these mutants. Smaller deletions showed a reduction in the ability of 77A to recognize secreted HSP70 from cell culture supernatants when aas 604-614 were deleted (FIG. 3C). Interestingly, when HSP70 from cell lysates was used as the target, deletion of 594-604 was associated with decreased recognition. These findings suggest that 77A recognizes an epitope in amino acids 594-614 (FIG. 3D), which is not a target for previously described mAbs, and that there may be differential post-translational modification of secreted HSP70 that impacts recognition by 77A or that 77A binds HSP70 in the context of co-chaperones. Alanine scanning analysis was performed to identify key amino acids in HSP70 with which 77A interacts (FIG. 3E). This analysis determined that 77A recognizes a conformational epitope of HSP70 that includes K573, E576, W580, R596, and E598 as secondary critical sites and H594, K595, and Q601 as primary critical sites (FIG. 3F), with the sites corresponding to the amino acids in SEQ ID NO: 11. These data support a model where 77A binds to a novel HSP70 domain when HSP70 is in its ADP- and antigenic peptide-bound form to lock in substrate binding and increase uptake.

### Example 4. —Differential Activity of 77A in Immune-Deficient Myeloma Models

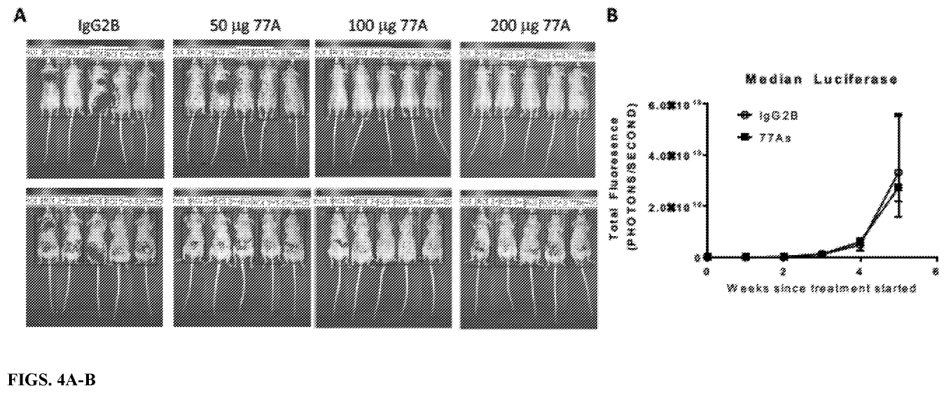

Since 77A bound strongly to human HSP70 from eukaryotic cells (FIG. 2A), its activity was studied in vivo against human myeloma cells. In a model of luc-labeled MM1.S cells in nude mice, 77A showed dose-dependent anti-tumor activity (FIG. 4A), with some tumor growth reduction when given as a 50 μg injection twice weekly, and stronger activity at 100 and 200 μg doses. This was accompanied by a reduction in human light chain levels as measured by an ELISA. Notably, when this study was repeated with MM1.S cells in SCID mice and in non-obese diabetic severe combined immunodeficient IL-2 receptor 7-null (NOD/SCID IL-2R^{γnull} (NSG)) mice, 77A did not show activity by in vivo imaging (FIG. 4B) or light chain quantitation. Together, these contrasting results suggested that 77A is dependent on an immune effector cell retained by nude but absent in NSG mice. Moreover, in vitro assays utilizing MM1.S cells and 77A did not show a direct cytotoxic activity, and 77A did not induce antibody- or complement-dependent cellular cytotoxicity (ADCC, CDC).

### Example 5. —77A Enhances HSP70 Uptake by Dendritic Cells

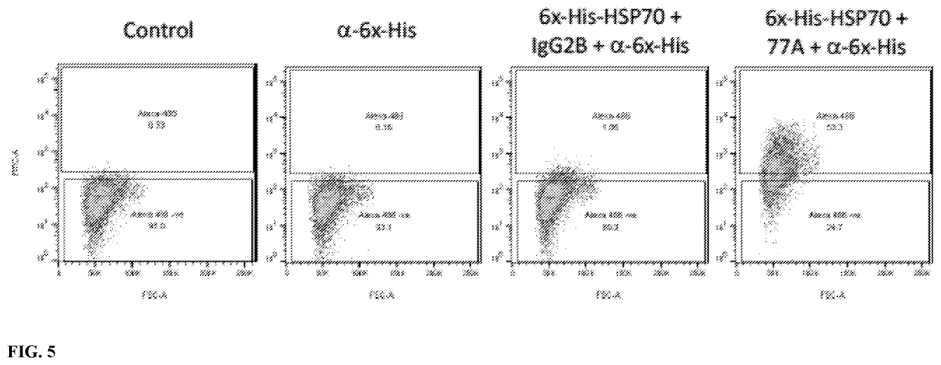

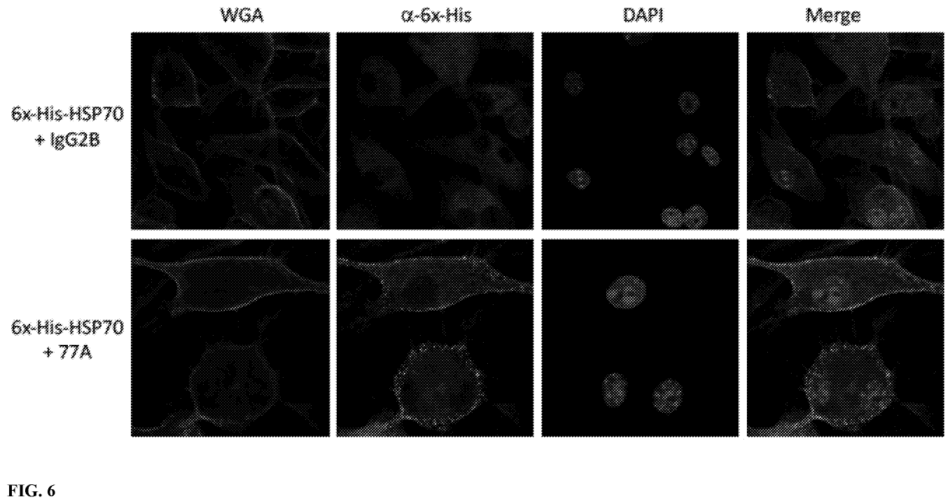

DCs, macrophages, and natural killer (NK) cells are defective in NSG mice (Shultz et al., 1995) whereas these cells are present and functional in nude mice. While there are other differences between NSG and nude mice, DCs were focused on first since HSP70 secreted from tumor cells is known to facilitate delivery of antigen to DCs (Shevtsov & Multhoff, 2016; Binder, 2008). Therefore, purified hexa-(6×)-histidine-tagged human HSP70 made in Sf9 cells was used and an Alexa Fluor 488-tagged α-6×-His-tag mAb was used to study uptake of 6×-His-HSP70 by DC2.4 immature DCs, an immortalized murine line created by transducing bone marrow isolates of C57BL/6 mice with retrovirus vectors expressing murine Granulocyte-macrophage CSF (GM-CSF) and the MYC and RAF oncogenes (Shen et al., 1997). 77A substantially increased HSP70 uptake at 4° C. and at 37° C. compared to a control isotype mAb (referred to as IgG2B) directed against hen egg lysozyme (FIG. 5), while other α-HSP70 mAbs did not have this activity. Enhanced HSP70 uptake in the presence of 77A could also be demonstrated by immunofluorescence staining of DC2.4 cells (FIG. 6). Finally, HSP70 uptake into DCs was examined using 77A conjugated to 15 nM gold particles by electron microscopy (EM). 77A enhanced HSP70 uptake versus IgG2B by EM as it had in prior assays (FIG. 5 and FIG. 6), and HSP70 was found in intracellular cytoplasmic membrane-bound bodies resembling phagolysosomes (FIG. 7; red arrows).

Given the substantial difference in affinity of 77A for murine and human HSP70 (FIG. 5), site-directed mutagenesis was used to express human HSP70 with single changes (K->E, Q->R, N->S) to each amino acid that is different between the two variants (FIG. 14A), each of the possible two amino acid changes, and one with all three. These were expressed in HSP70 KO 293T cells, and the ability of 77A to bind were examined in IP studies (as in FIG. 3). The ability of DC2.4 cells to take up these mutants in the presence of IgG2B and 77A will be studied by FACS (as in FIG. 5). Mutation of the human HSP70 sequence in the 594-614 region to match the murine sequence reduces the affinity of 77A for HSP70 and its ability to induce uptake by DC2.4 cells (FIG. 14B).

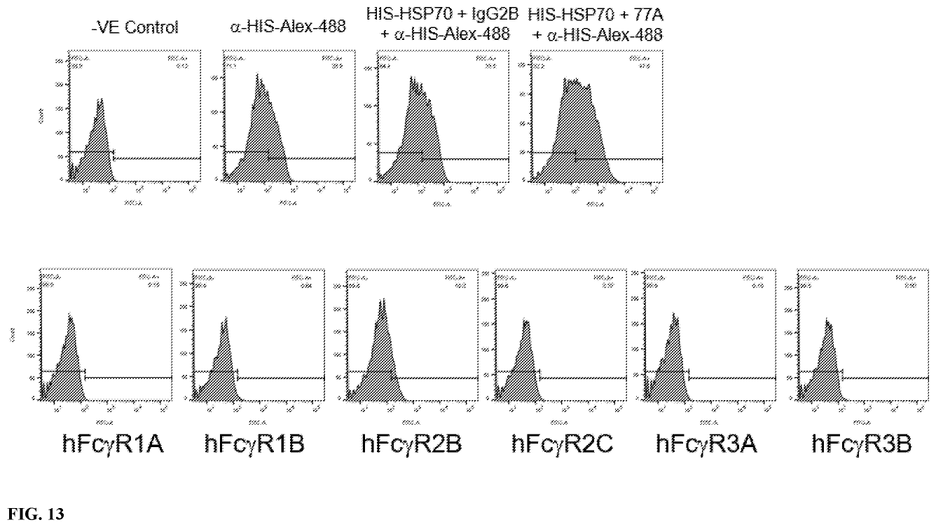

Determining the receptors through which 77A/HSP70 complexes are taken up by DCs could help identify other immune effectors that would be similarly influenced. HSP70 knock-out HEK 239T cells that express murine FcγR2B were found to take up 77A-bound HSP70. Likewise, HSP70 knock-out HEK 239T cells that express human FcγR2A or human FcγR2B were found to take up 77A-bound HSP70, while the expression of other Fc receptors did not produce this effect (FIG. 13). Human FcγR2A and FcγR2B are expressed in monocytes/macrophages and dendritic cells (Bruhns, 2012).

### Example 6. —DC Uptake of HSP70 Enhances Maturation

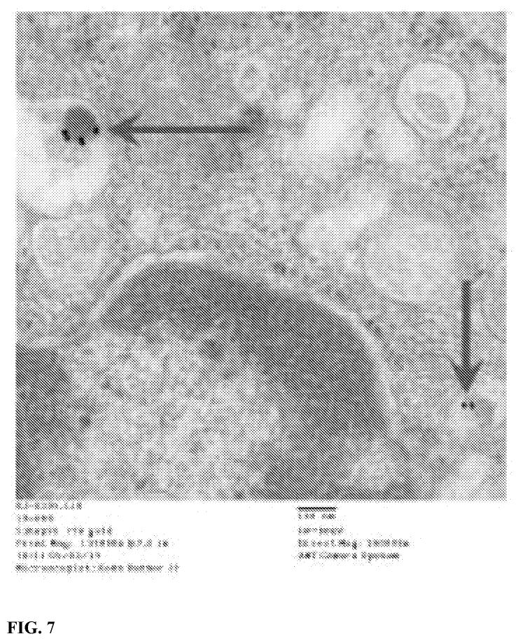

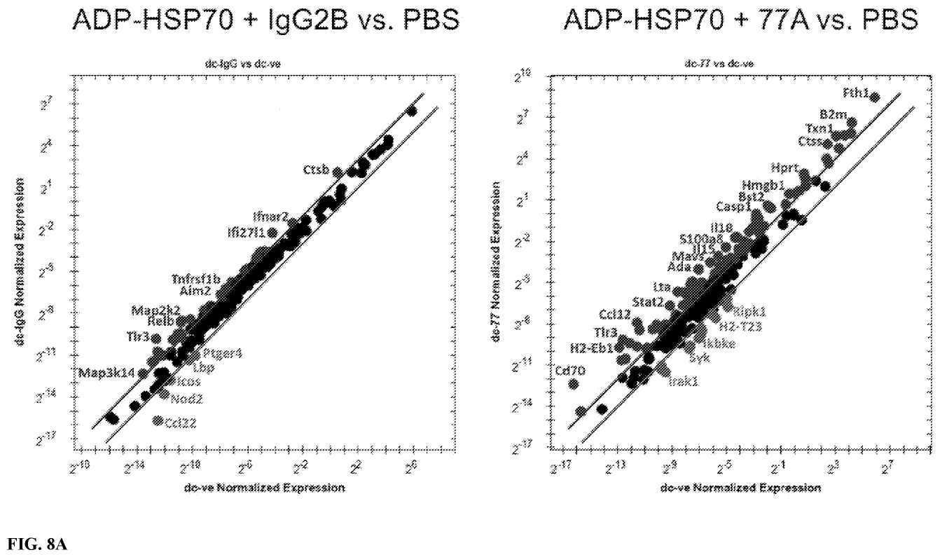

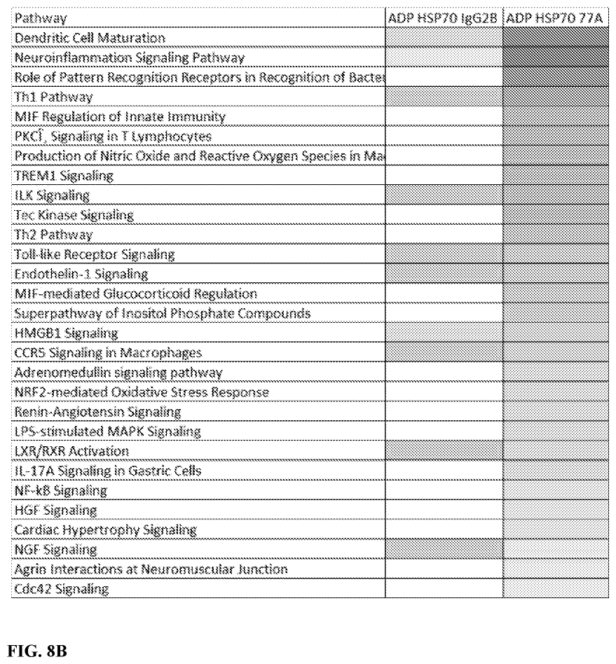

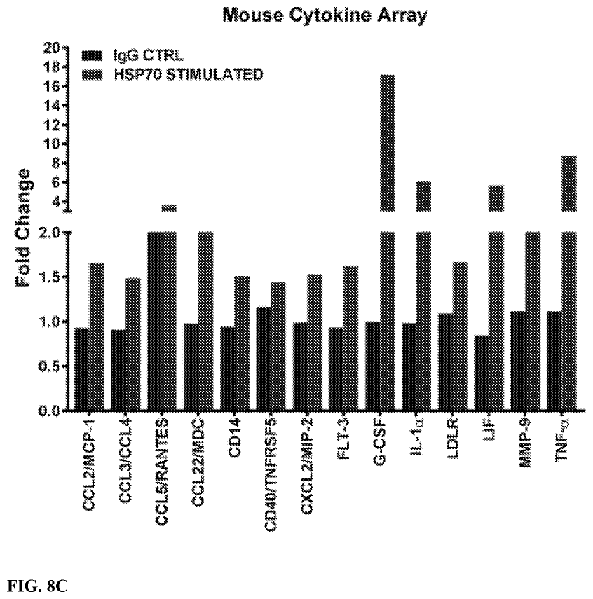

To begin to study the functional consequences of HSP70 uptake by DCs, DC2.4 cells were exposed to HSP70 in the presence of either IgG2B or 77A. RNA was harvested, converted to cDNA, and this was hybridized to the BioRad Immune Response Tier 1-4 qPCR array containing 384 genes. Using a threshold of a 2-fold or more change over controls, IgG2B activated relatively few genes compared to the negative control (FIG. 8A; top panel), while 77A activated transcription of a much larger gene set (FIG. 8A; bottom panel). Ingenuity Pathway Analysis noted that the top activated pathway was one representing dendritic cell maturation (FIG. 8B), reflected in part by the finding that some of the top genes play key roles in DC function, including CD70 (Bourque & Hawiger, 2018), Adenosine deaminase (Casanova et al., 2012), Cathepsin S (Kim et al., 2017), and CC chemokine ligand 5 (Wang et al., 2002). Other activated pathways included a Neuroinflammation signaling pathway, both Th1 and Th2 signaling, and Toll-like receptor (TLR) signaling. In addition, cell culture supernatants from these experiments were collected and analyzed using a cytokine array. Notable changes induced by HSP70 in the presence of 77A versus IgG2B as a control included increases in cytokines that play key roles in DC biology (Turner et al., 2014), including Tumor-necrosis factor (TNF)-α, Granulocyte colony stimulating factor (G-CSF), and Interleukin (IL)-1 (FIG. 8C), among others.

### Example 7. —Activity of 77A in Tumor Models

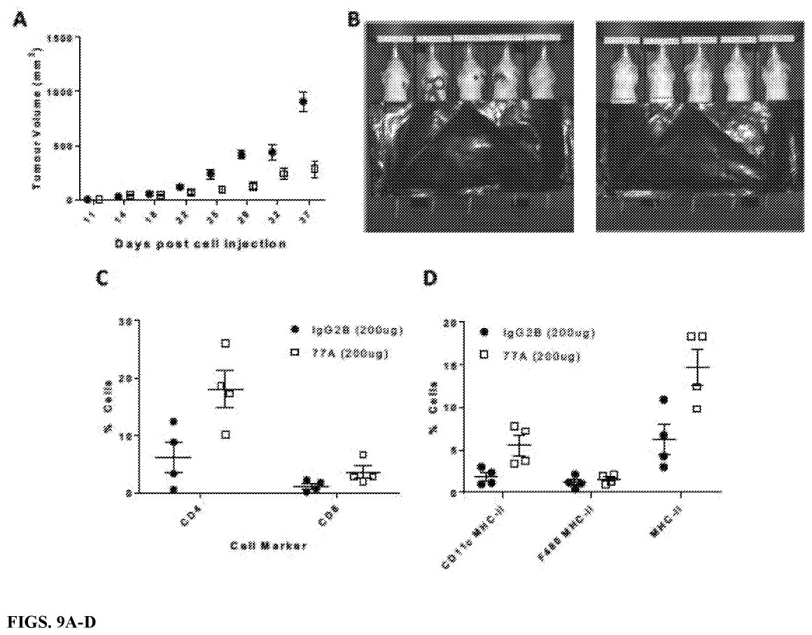

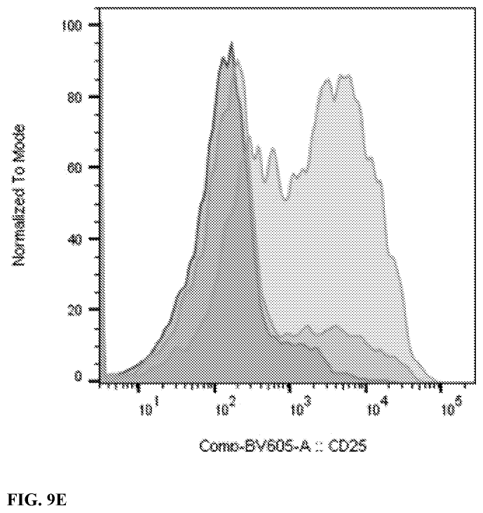

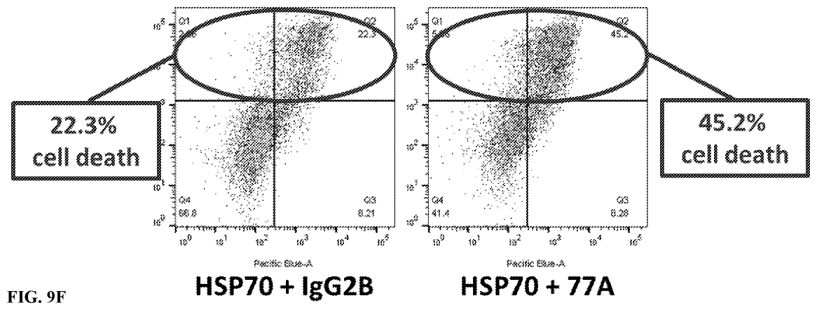

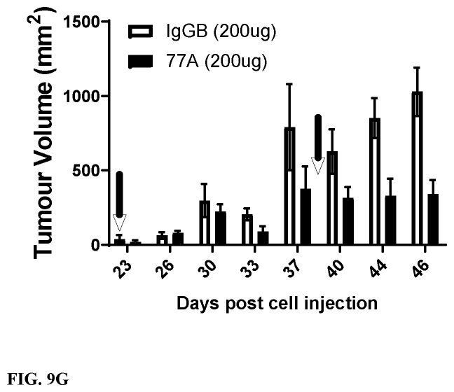

The potential that 77A influenced a general immunity mechanism relevant to many tumors prompted the examination of its activity in a melanoma model and in 4T1 cells, a murine triple-negative breast carcinoma (TNBC) (Chen et al., 2019) model that has been well characterized, is considered immunologically cold (Kim et al., 2014), and does not express surface HSP70. Immune-competent BALB/c mice injected with luc-labeled 4T1 cells into the mammary fat pad and treated with 77A showed slower primary tumor growth versus IgG2B (FIG. 9A). Notably, 77A strongly inhibited the development of metastatic disease to the lung (FIG. 9B) and also to the spleen and liver. Peripheral blood was collected on day 32 from surviving mice and analyzed by multi-parameter flow for T-cell subsets, which showed a significant increase in CD4+ T-cells and a more modest increase in CD8+ T-cells (FIG. 9C). Also, there was an increase noted for MHC class II+ cells in the peripheral blood (FIG. 9D), and cells that were both class II+ and CD11c+, consistent with an increase in murine DCs after 77A treatment. 77A was found to (a) induce uptake of HSP70 into human primary CD4+ and CD8+ T cells (FIG. 9E), (b) enhance T-cell proliferation and expression of maturation markers, including CD69 and HLA DR, and (c) stimulate MHC-independent cytolytic CD4 T-cell activity in a number of tumor models, including A549 human lung carcinoma cells (FIG. 9F).

For the melanoma model, the A375 melanoma model in nude mice was used. The first dose of 77A was given on day 23, and the last dose on day 39. Tumor volume was slower in 77A treated mice (FIG. 9G).

### Example 8. —Utility of 77A to Enhance Efficacy of a 4T1-Based Vaccine Strategy

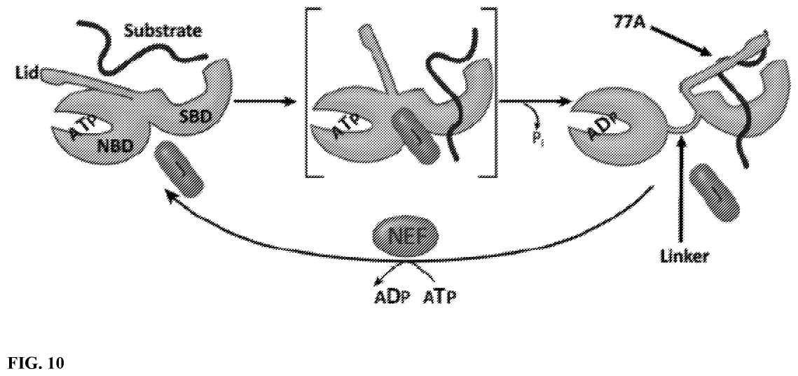

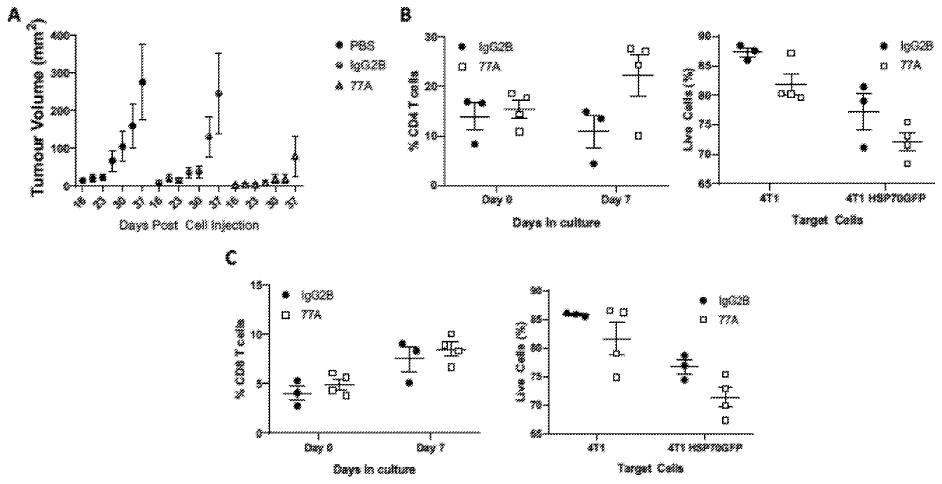

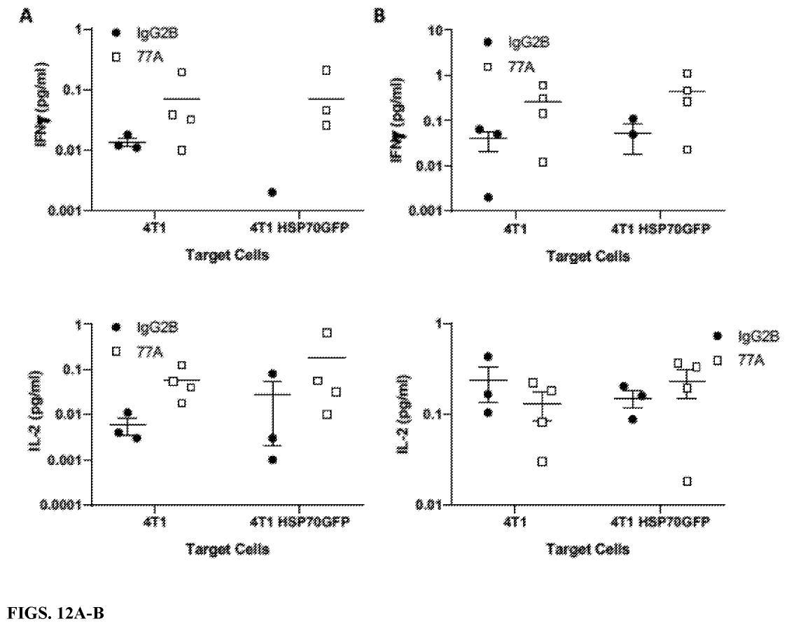

DCs have long been considered attractive targets for the development of vaccine strategies given their roles as professional antigen presenting cells (Gornati et al., 2018). Therefore, the possibility that 77A could increase vaccine efficacy through its ability to enhance HSP70-peptide antigen complex uptake was considered. To test this, 4T1 cells expressing HSP70-GFP were used to purify ADP-HSP70-peptide complexes over an ADP-agarose column, and BALB/c mice were injected with PBS, or with the ADP-HSP70-peptide vaccine with IgG2B or 77A. The focus was on ADP-HSP70 as, compared to ATP-HSP70, ADP-HSP70 is the form that has higher affinity for substrates, including tumor-derived peptides, and is therefore more immunogenic (Greene et al., 1995; Peng et al., 1997; Craig & Marszalek, 2017) (see FIG. 10 for an overview of the fundamentals of the HSP70 machinery). On day 0, mice were orthotopically injected with viable 4T1-luc labeled, HSP70-GFP-expressing cells, and the development of tumors was monitored by whole animal imaging. Vaccination with ADP-HSP-peptide complexes in the presence of IgG2B did not slow tumor growth in mice after challenge with live 4T1 cells compared to the PBS control (FIG. 11A), but vaccination with 77A did slow growth substantially. Splenocytes isolated from these mice at day 37 were then exposed to irradiated 4T1 cells for 7 days, and showed an increase in CD4+ and, to a lesser extent, CD8+ T-cells (FIGS. 11B & FIG. 11C; left panels). Also, these cells showed enhanced cytotoxicity after 7 days against 4T1 cells expressing HSP70-GFP, or 4T1 cells without HSP70-GFP (FIGS. 11B & FIG. 11C; right panels), supporting the possible development of cytotoxic T lymphocytes (CTLs) recognizing both HSP70-GFP-derived antigens and other tumor-derived antigens. This possibility was also tested by evaluating the ability of these T-cells to elaborate interferon (IFN)-γ and IL2. Putative CD4+ CTLs from 77A-vaccinated mice showed increased IFNγ and IL2 secretion when they were exposed to 4T1 or 4T1-HSP70-GFP cells compared with the IgG2B controls (FIG. 12A). In the case of CD8+ CTLs (FIG. 12B), these also showed increased IFNγ secretion when they were exposed to either 4T1 or 4T1-HSP70-GFP cells after vaccination with ADP-HSP70 complexes and 77A, but the results were less striking for IL-2 production. Together, these data suggest that 77A can enhance antigen uptake by DCs and produce more robust downstream CD4+ and CD8+ T-cell responses.

### Example 9. —Identification of Tumor Targets for the Clone 77A HSP70 mAb

Pegylated liposomal doxorubicin (PLD; Doxil®) was chosen as the first agent to combine with 77A since it has regulatory approval for breast cancer therapy (Lao et al., 2013)], is active against other malignancies (Lyseng-Williamson et al., 2013), and causes ICD (Kroemer et al., 2013). PLD with IgG2B or 77A was evaluated in BALB/c mice orthotopically injected with 4T1 cells. Compared with PLD+IgG2B, PLD+77A induced a greater reduction and delay in tumor growth (FIG. 15A), and ⅘ mice were cured with PLD+77A (defined as lack of recurrence at day 100), while this was the case for only ⅕ PLD+IgG2B controls. Also, this combination was evaluated in a CT26-based immune-competent colon carcinoma model, where a greater reduction and delay in tumor growth was again seen. Notably, by day 81, ⅗ PLD+77A mice remained alive and without tumor recurrence, while only ⅕ IgG2B+PLD mice were alive, and that individual had measurable disease (FIG. 15B).

### Example 10. —Anti-HSP70 Antibody Epitope Binning

Epitope binning experiments were conducted to determine the extent to which different, unrelated anti-HSP70 antibodies interfered or blocked the binding of 77A to HSP70.

To conduct binning experiments, antibodies were set up in binning pairs. The Octet platform was used in the sandwich configuration. Analysis was performed using the Data Analysis HT software. The first antibody for each pair was loaded onto the dip-and-read sensor surface, and then the sensor was dipped into the HSP70 solution to load the antigen. Finally, the sensor was dipped into the second antibody for each pair and the response was measured. Results for each pair were tabulated into a pairwise matrix.

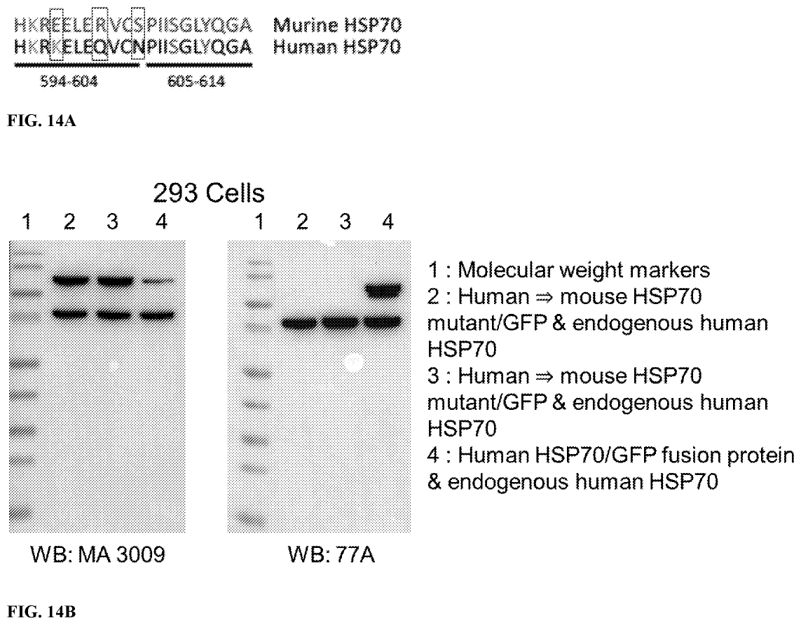

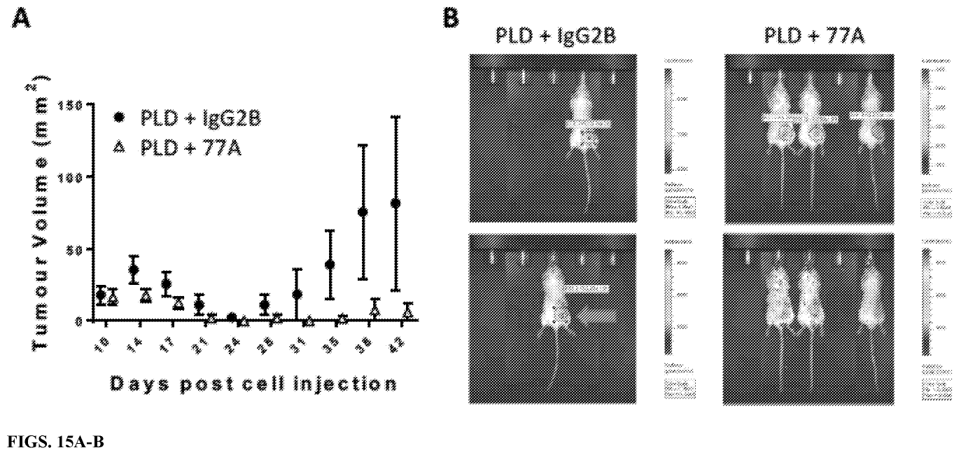

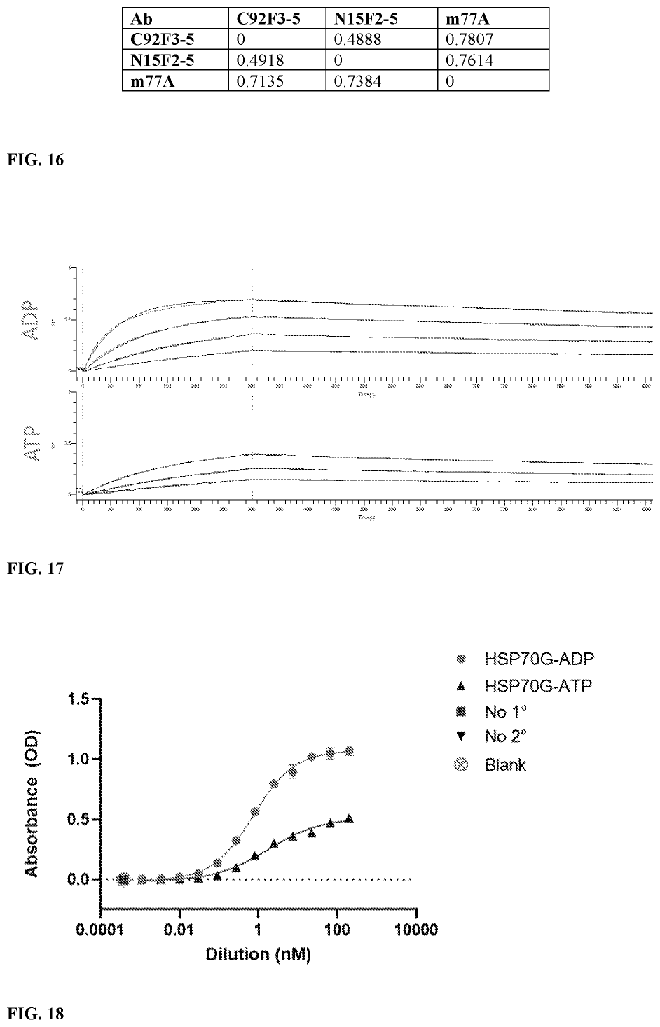

Results are depicted in FIG. 16. The numbers in FIG. 16 reflect the percent of maximal binding in the presence of the potentially competing antibody. As expected, all antibodies competed with themselves. As depicted, 77A does not compete with C92F3-5 (Fisher Scientific) or N15F2-5 (Enzo Life Sciences).

### Example 11. —Binding Kinetics for ADP Vs ATP-Bound HSP70

77A binding to either the ATP or ADP-bound forms of HSP70 was characterized using biolayer interferometry (BLI) instrumentation and ELISA.

HSP70 was expressed in SF9 cells. Affinity chromatography using resins selective for ADP or ATP-binding proteins were used to enrich bulk preparations of the correspondingly bound HSP70 recombinant protein. Protein concentrations, determined after elution, were measured so that equal concentrations could be used in subsequent assays. For BLI-based assays, antibodies and reagents were diluted out onto microwell plates and loaded into the instrument. Antibodies being tested were immobilized onto dip-and-read sensors and then observed for kinetics during binding to ATP or ADP-enriched HSP70. For ELISA, the relevant enriched HSP70 proteins were directly coated onto ELISA plate wells overnight. Test antibodies were then serially diluted across the plate wells, and detected using an appropriate secondary antibody-HRP conjugate. The plates were developed using a TMB substrate, and the O.D. stabilized using an acid stop solution. BLI sensorgram trace data is shown in FIG. 17. As depicted, there was a superior response (nm shift) during the antigen association step for ADP-enriched HSP70 in contrast to ATP-enriched HSP70. Binding kinetics are shown in Table 4. As depicted, 77A bound ADP-enriched HSP70 with a ≥4-fold greater affinity (KD) than ATP-enriched HSP70.

TABLE 4
77A Binding Kinetics for ADP- vs ATP-bound HSP70

| HSP70 | | | | | | | | | | |
|---|---|---|---|---|---|---|---|---|---|---|
| (Conc | | KD | KD | ka | ka | kdis | kdis | | Full | Full |
| μM) | Resp. | (M) | Error | (1/Ms) | Error | (1/s) | Error | Rmax | X\hat{ }2 | R\hat{ }2 |
| ADP | 0.6942 | 5.42E−09 | 3.37E−11 | 5.38E+04 | 1.85E+02 | 2.92E−04 | 1.51E−06 | 0.7056 | 0.114 | 0.9987 |
| (300) | | | | | | | | | | |
| ADP | 0.5313 | 5.42E−09 | 3.37E−11 | 5.38E+04 | 1.85E+02 | 2.92E−04 | 1.51E−06 | 0.5955 | 0.114 | 0.9987 |
| (150) | | | | | | | | | | |
| ADP | 0.3564 | 5.42E−09 | 3.37E−11 | 5.38E+04 | 1.85E+02 | 2.92E−04 | 1.51E−06 | 0.5216 | 0.114 | 0.9987 |
| (75) | | | | | | | | | | |
| ADP | 0.1988 | 5.42E−09 | 3.37E−11 | 5.38E+04 | 1.85E+02 | 2.92E−04 | 1.51E−06 | 0.4538 | 0.114 | 0.9987 |
| (37.5) | | | | | | | | | | |
| ATP | 0.3887 | 2.02E−08 | 1.61E−10 | 1.88E+04 | 1.17E+02 | 3.80E−04 | 1.89E−06 | 0.4937 | 0.0296 | 0.9985 |
| (300) | | | | | | | | | | |
| ATP | 0.2458 | 2.02E−08 | 1.61E−10 | 1.88E+04 | 1.17E+02 | 3.80E−04 | 1.89E−06 | 0.4655 | 0.0296 | 0.9985 |
| (150) | | | | | | | | | | |
| ATP | 0.1406 | 2.02E−08 | 1.61E−10 | 1.88E+04 | 1.17E+02 | 3.80E−04 | 1.89E−06 | 0.448 | 0.0296 | 0.9985 |
| (75) | | | | | | | | | | |

ELISA data, shown in FIG. 18, depicted the same trend, in which 77A had higher maximal absorbance with the ADP-enriched HSP70 than the ATP-enriched HSP70. 77A also had a lower EC50 value with the ADP-enriched HSP70 (0.7372 nM) than the ATP-enriched HSP70 (1.774 nM).

Together, these results show that 77A preferentially binds to the ADP-HSP70 protein relative to the ATP-HSP70 protein.

### Example 12. —77A Activity in a Murine CT-26 Tumor Model

This Example describes the testing of the 77A antibody, alone and in combination with an anti-CTLA4 antibody, in a murine CT-26 colorectal adenocarcinoma cachexia model.

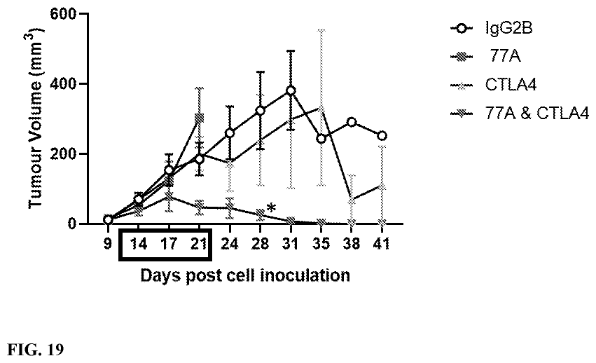

CT26 cells were inoculated subcutaneously in nude BALB/c mice. Treatment was started when the tumors reached ˜80 mm³ average volume. Mice were administered 10 mg/kg of 77A, isotype control (IgG2B), and/or anti-CTLA4 antibody on days 14, 17, and 21. Tumor volume is shown in FIG. 19. As depicted, the combination of 77A and anti-CTLA-4 antibody had the greatest effect on tumor volume, and the combination significantly reduced tumor volume relative to isotype control. The combination therapy completely eradicated the tumors in all 5 treated animals.

### Example 13. —Humanization

This Example describes the generation of humanized variants of the 77A antibody.

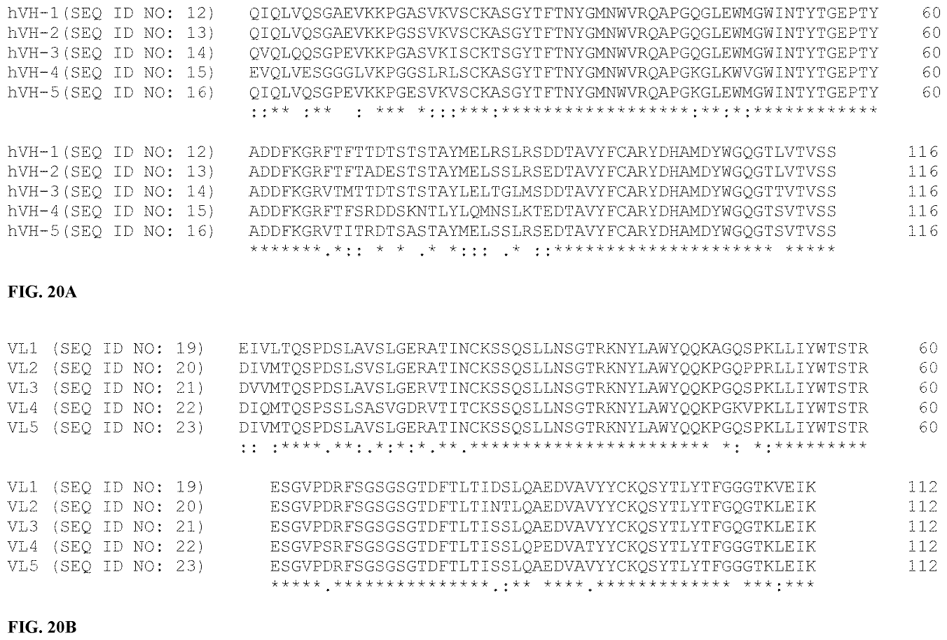

The murine immunoglobulin family subgroup for the 77A antibody was determined, and the antigen binding sequences were then modeled and grafted into compatible potential frameworks of relevant human immunoglobulin families for both the heavy and light chains. Backmutations were employed where necessary. Five humanized heavy chain variants (hVH-1 through hVH-5, as shown in Table 5), and five humanized light chain variants (hVL-1 through hVL-5, as shown in Table 5) were generated. A sequence alignment of hVH-1 through hVH-5 is depicted in FIG. 20A, and a sequence alignment of hVL-1 through hVL-5 is depicted in FIG. 20B.

TABLE 5
Humanized variable region sequences

| VH/VL | SEQ | |
|---|---|---|
| Name | ID NO | Amino Acid Sequence |
| hVH-1 | 12 | QIQLVQSGAEVKKPGASVKVSCKASGYTFTNYGMNWVRQAPGQGLE |
| | | WMGWINTYTGEPTYADDFKGRFTFTTDTSTSTAYMELRSLRSDDTA |
| | | VYFCARYDHAMDYWGQGTLVTVSS |
| hVH-2 | 13 | QIQLVQSGAEVKKPGSSVKVSCKASGYTFTNYGMNWVRQAPGQGLE |
| | | WMGWINTYTGEPTYADDFKGRFTFTADESTSTAYMELSSLRSEDTA |
| | | VYFCARYDHAMDYWGQGTLVTVSS |
| hVH-3 | 14 | QVQLQQSGPEVKKPGASVKISCKTSGYTFTNYGMNWVRQAPGQGLE |
| | | WMGWINTYTGEPTYADDFKGRVTMTTDTSTSTAYLELTGLMSDDTA |
| | | VYFCARYDHAMDYWGQGTTVTVSS |
| hVH-4 | 15 | EVQLVESGGGLVKPGGSLRLSCKASGYTFTNYGMNWVRQAPGKGLK |
| | | WVGWINTYTGEPTYADDFKGRFTFSRDDSKNTLYLQMNSLKTEDTA |
| | | VYFCARYDHAMDYWGQGTSVTVSS |
| hVH-5 | 16 | QIQLVQSGPEVKKPGESVKVSCKASGYTFTNYGMNWVRQAPGKGLE |
| | | WMGWINTYTGEPTYADDFKGRVTITRDTSASTAYMELSSLRSEDTA |
| | | VYFCARYDHAMDYWGQGTSVTVSS |
| hVL-1 | 19 | EIVLTQSPDSLAVSLGERATINCKSSQSLLNSGTRKNYLAWYQQKA |
| | | GQSPKLLIYWTSTRESGVPDRFSGSGSGTDFTLTIDSLQAEDVAVY |
| | | YCKQSYTLYTFGGGTKVEIK |
| hVL-2 | 20 | DIVMTQSPDSLSVSLGERATINCKSSQSLLNSGTRKNYLAWYQQKP |
| | | GQPPRLLIYWTSTRESGVPDRFSGSGSGTDFTLTINTLQAEDVAVY |
| | | YCKQSYTLYTFGQGTKLEIK |
| hVL-3 | 21 | DVVMTQSPDSLAVSLGERVTINCKSSQSLLNSGTRKNYLAWYQQKP |
| | | GQSPKLLIYWTSTRESGVPDRFSGSGSGTDFTLTISSLQAEDVAVY |
| | | YCKQSYTLYTFGQGTKLEIK |
| hVL-4 | 22 | DIQMTQSPSSLSASVGDRVTITCKSSQSLLNSGTRKNYLAWYQQKP |
| | | GKVPKLLIYWTSTRESGVPSRFSGSGSGTDFTLTISSLQPEDVATY |
| | | YCKQSYTLYTFGGGTKLEIK |
| hVL-5 | 23 | DIVMTQSPDSLAVSLGERATINCKSSQSLLNSGTRKNYLAWYQQKP |
| | | GQSPKLLIYWTSTRESGVPDRFSGSGSGTDFTLTISSLQAEDVAVY |
| | | YCKQSYTLYTFGGGTKLEIK |

Antibodies including combinations of each humanized heavy and light chain were made and characterized. These antibodies were referred to as h77A-1 through h77A-25, and corresponding amino acid sequences are depicted in Table 6.

TABLE 6
Humanized 77A variants

| | | | SEQ ID NO | | | | | | | |
|---|---|---|---|---|---|---|---|---|---|---|
| | | | | VH | VH | VH | | VL | VL | VL |
| Name | | | | CDR | CDR | CDR | | CDR | CDR | CDR |
| Antibody | VH | VL | VH | 1 | 2 | 3 | VL | 1 | 2 | 3 |
| 77A | VH | VL | 7 | 1 | 2 | 3 | 8 | 4 | 5 | 6 |
| h77A-1 | hVH-1 | hVL-1 | 12 | 1 | 2 | 3 | 19 | 4 | 5 | 6 |
| h77A-2 | hVH-1 | hVL-2 | 12 | 1 | 2 | 3 | 20 | 4 | 5 | 6 |
| h77A-3 | hVH-1 | hVL-3 | 12 | 1 | 2 | 3 | 21 | 4 | 5 | 6 |
| h77A-4 | hVH-1 | hVL-4 | 12 | 1 | 2 | 3 | 22 | 4 | 5 | 6 |
| h77A-5 | hVH-1 | hVL-5 | 12 | 1 | 2 | 3 | 23 | 4 | 5 | 6 |
| h77A-6 | hVH-2 | hVL-1 | 13 | 1 | 2 | 3 | 19 | 4 | 5 | 6 |
| h77A-7 | hVH-2 | hVL-2 | 13 | 1 | 2 | 3 | 20 | 4 | 5 | 6 |
| h77A-8 | hVH-2 | hVL-3 | 13 | 1 | 2 | 3 | 21 | 4 | 5 | 6 |
| h77A-9 | hVH-2 | hVL-4 | 13 | 1 | 2 | 3 | 22 | 4 | 5 | 6 |
| h77A-10 | hVH-2 | hVL-5 | 13 | 1 | 2 | 3 | 23 | 4 | 5 | 6 |
| h77A-11 | hVH-3 | hVL-1 | 14 | 1 | 2 | 3 | 19 | 4 | 5 | 6 |
| h77A-12 | hVH-3 | hVL-2 | 14 | 1 | 2 | 3 | 20 | 4 | 5 | 6 |
| h77A-13 | hVH-3 | hVL-3 | 14 | 1 | 2 | 3 | 21 | 4 | 5 | 6 |
| h77A-14 | hVH-3 | hVL-4 | 14 | 1 | 2 | 3 | 22 | 4 | 5 | 6 |
| h77A-15 | hVH-3 | hVL-5 | 14 | 1 | 2 | 3 | 23 | 4 | 5 | 6 |
| h77A-16 | hVH-4 | hVL-1 | 15 | 1 | 2 | 3 | 19 | 4 | 5 | 6 |
| h77A-17 | hVH-4 | hVL-2 | 15 | 1 | 2 | 3 | 20 | 4 | 5 | 6 |
| h77A-18 | hVH-4 | hVL-3 | 15 | 1 | 2 | 3 | 21 | 4 | 5 | 6 |
| h77A-19 | hVH-4 | hVL-4 | 15 | 1 | 2 | 3 | 22 | 4 | 5 | 6 |
| h77A-20 | hVH-4 | hVL-5 | 15 | 1 | 2 | 3 | 23 | 4 | 5 | 6 |
| h77A-21 | hVH-5 | hVL-1 | 16 | 1 | 2 | 3 | 19 | 4 | 5 | 6 |
| h77A-22 | hVH-5 | hVL-2 | 16 | 1 | 2 | 3 | 20 | 4 | 5 | 6 |
| h77A-23 | hVH-5 | hVL-3 | 16 | 1 | 2 | 3 | 21 | 4 | 5 | 6 |
| h77A-24 | hVH-5 | hVL-4 | 16 | 1 | 2 | 3 | 22 | 4 | 5 | 6 |
| h77A-25 | hVH-5 | hVL-5 | 16 | 1 | 2 | 3 | 23 | 4 | 5 | 6 |

Humanized variants were characterized for binding using biolayer interferometry (BLI). Chimeric antibodies including the murine 77A antigen binding region and human Fe regions were also tested. Antibodies and reagents were diluted out onto microwell plates and loaded into the instrument. Diluted antibodies were first immobilized onto dip-and-read sensors, and then measurements for baselines were acquired. Subsequently, the sensors were dipped into wells containing the antigen in solution, or buffer alone. The change in number of molecules bound to the sensor was quantified by measurement of the shift in the interference pattern of light during each step of the protocol. Mathematical modeling was performed based on those values and the concentrations of reagents used in the assay and used to calculate kinetics values.

Results are shown in Table 7. As depicted, all of the tested humanized variants had affinity measurements within 2 to 5-fold of the parental murine antibody.

TABLE 7
Humanized 77A antibodies binding kinetics

| | Capture | | | | | | | Rₘₐₓ |
|---|---|---|---|---|---|---|---|---|
| | level | kₐ | k_{d} | K_{D} | K_{D} | | | at |
| Antibody | (nm) | (m⁻¹s⁻¹) | (s⁻¹) | (M) | (nM) | R² | X² | 33.3 nM |
| 77A | 0.834 | 6.74E+04 | 8.08E−05 | 1.20E−09 | 1.2 | 0.9967 | 0.1175 | 0.146 |
| Chimera | 0.836 | 7.16E+04 | 9.94E−05 | 1.39E−09 | 1.39 | 0.9979 | 0.1813 | 0.2441 |
| IgG1 | | | | | | | | |
| Chimera | 0.786 | 7.13E+04 | 1.29E−04 | 1.81E−09 | 1.81 | 0.9973 | 0.2251 | 0.2397 |
| IgG4 | | | | | | | | |
| Chimera | 0.824 | 7.37E+04 | 1.12E−04 | 1.52E−09 | 1.52 | 0.9985 | 0.1527 | 0.2662 |
| IgG2 | | | | | | | | |
| h77A-1 | 0.819 | 6.62E+04 | 1.73E−04 | 2.61E−09 | 2.61 | 0.9972 | 0.2055 | 0.2409 |
| h77A-2 | 0.863 | 5.99E+04 | 3.08E−04 | 5.13E−09 | 5.13 | 0.9967 | 0.2669 | 0.2574 |
| h77A-3 | 0.806 | 6.23E+04 | 3.05E−04 | 4.90E−09 | 4.9 | 0.9978 | 0.1294 | 0.2158 |
| h77A-4 | 0.777 | 3.74E+04 | 2.87E−04 | 7.66E−09 | 7.66 | 0.9976 | 0.133 | 0.2462 |
| h77A-5 | 0.812 | 5.14E+04 | 3.53E−04 | 6.87E−09 | 6.87 | 0.9983 | 0.0856 | 0.239 |
| h77A-6 | 0.76 | 5.31E+04 | 1.40E−04 | 2.64E−09 | 2.64 | 0.9976 | 0.2202 | 0.2779 |
| h77A-7 | 0.789 | 4.87E+04 | 4.88E−04 | 1.00E−08 | 10 | 0.9956 | 0.1637 | 0.2465 |
| h77A-9 | 0.83 | 3.92E+04 | 3.48E−04 | 8.89E−09 | 8.89 | 0.9952 | 0.2576 | 0.2308 |
| h77A-10 | 0.854 | 5.77E+04 | 4.09E−04 | 7.10E−09 | 7.1 | 0.9943 | 0.3562 | 0.2578 |
| h77A-11 | 0.781 | 4.46E+04 | 1.89E−04 | 4.23E−09 | 4.23 | 0.9966 | 0.1939 | 0.2206 |
| h77A-12 | 0.78 | 4.45E+04 | 4.46E−04 | 1.00E−08 | 10 | 0.9966 | 0.1605 | 0.2252 |
| h77A-13 | 0.775 | 4.47E+04 | 3.96E−04 | 8.85E−09 | 8.85 | 0.9953 | 0.1987 | 0.1937 |
| h77A-14 | 0.81 | 4.11E+04 | 6.16E−04 | 1.50E−08 | 15 | 0.9933 | 0.1716 | 0.193 |
| h77A-15 | 0.777 | 4.76E+04 | 5.13E−04 | 1.08E−08 | 10.8 | 0.9977 | 0.1126 | 0.2158 |
| h77A-16 | 0.742 | 4.40E+04 | 4.60E−04 | 1.05E−08 | 10.5 | 0.9832 | 0.1968 | 0.0937 |
| h77A-21 | 0.77 | 4.17E+04 | 5.45E−04 | 1.31E−08 | 13.1 | 0.9926 | 0.1633 | 0.1492 |
| h77A-22 | 0.798 | 3.64E+04 | 3.02E−04 | 8.31E−09 | 8.31 | 0.9816 | 0.2804 | 0.1043 |
| h77A-23 | 0.799 | 4.99E+04 | 4.47E−04 | 8.96E−09 | 8.96 | 0.9759 | 0.2499 | 0.078 |
| h77A-24 | 0.815 | 3.18E+04 | 2.94E−04 | 9.26E−09 | 9.26 | 0.9801 | 0.9801 | 0.1012 |
| h77A-25 | 0.81 | 4.73E+04 | 5.03E−04 | 1.06E−08 | 10.6 | 0.9723 | 0.2785 | 0.0976 |

### Example 14. —HSP70 Uptake Mediated by Humanized 77A Variants

This Example describes the evaluation of Fc receptor involvement in uptake of HSP70 mediated by humanized 77A variants.

293HSP70KO cells were transfected with vectors encoding a panel of mouse Fc receptors or human Fc Receptors for 24 hours. Transfections were performed using JetPrime in 10 cm dishes with 2.5 μg (for mouse Fc receptors) or 1.42 μg (for human Fc receptors) of each vector.

Next, HSP70GFP (BME Free; 5 μg/ml) and GFP-Nanobody Alexa-488 (1:1000) were added alone or in combination with antibody (1 pg/ml) for 1 hour at 37° C. Cells were then analyzed by FACs.

Antibodies tested were: IgG2 isotype control, 77A (murine), a chimeric 77A with a human IgG1 Fc domain, a chimeric 77A with a human IgG2 Fc domain, a chimeric 77a with a human IgG4 domain, and humanized variants h77A-1 (including hVH-1 and hVL-1), h77A-6 (including hVH-2 and hVL-1), and h77A-11 (including hVH-3 and hVL-1), each as described in Example 12. The humanized antibodies h77A-1, h77A-6, and h77A-11 were each tested with a human IgG2 Fc domain.

Mouse Fc receptors tested were: FcγR1 (Origene Catalog No. MR225268), FcγR2B (Origene Catalog No. MR204036), FcγR3 (Origene Catalog No. MR203404), and FcγR4 (Origene Catalog No. MR203178).

Human FC receptors tested were: FcγR1A (Origene Catalog No. RC207487), FcγR1B (Origene Catalog No. RC219204), FcγR2A (Origene Catalog No. RC205786), FcγR2B (Origene Catalog No. RC211982), FcγR2C (Origene Catalog No. RC213460), FcγR3A (Origene Catalog No. SC124061), and FcγR3B (Origene Catalog No. RC204749).

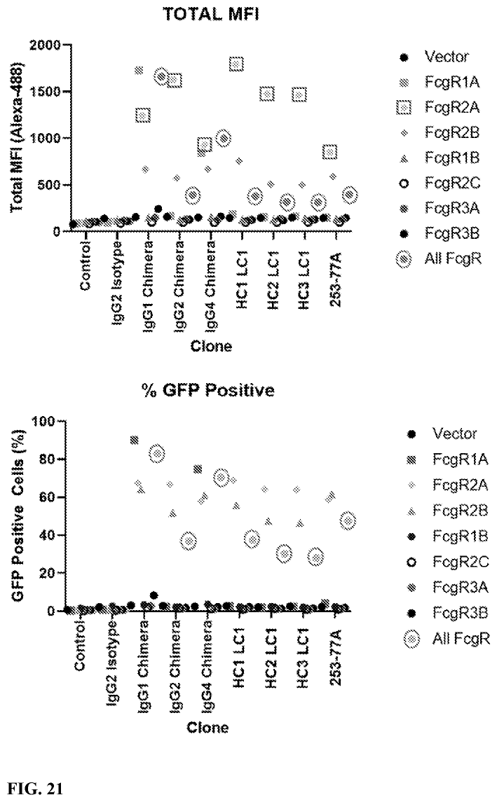

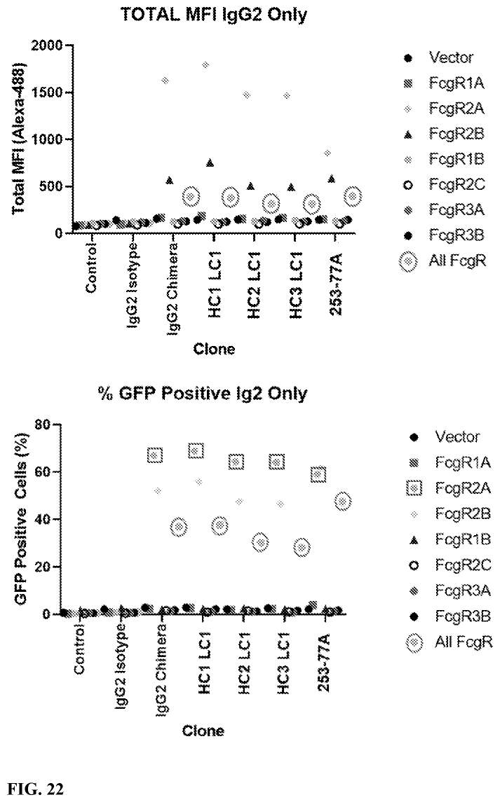

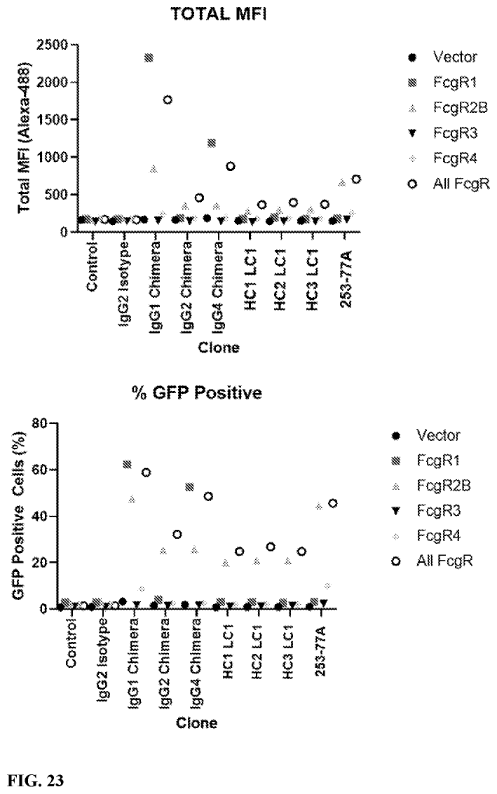

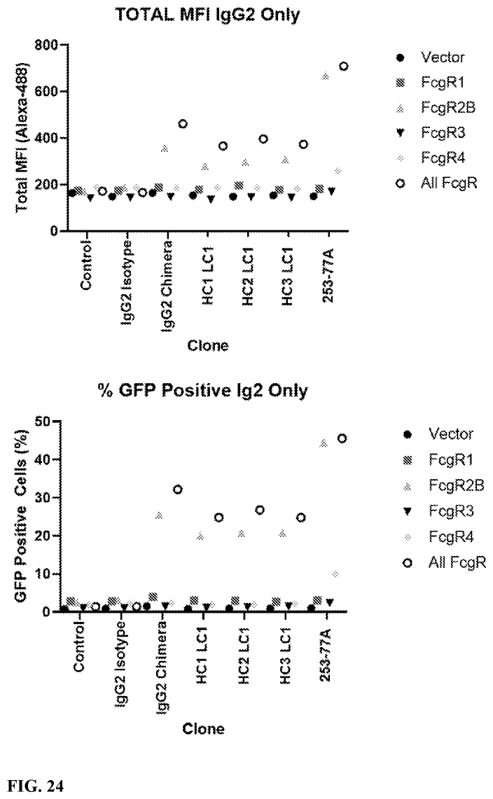

Results are depicted in FIGS. 21-24. A summary of the results with human FcγR is depicted in Table 8. The results demonstrated that all three humanized antibodies as well as the chimeric and parental murine 77A antibodies were able to mediate the uptake of HSP70 through the FcγR2A and FcγR2B receptors. The chimeric IgG1 and IgG4 antibodies also mediated HSP70 uptake through the FcγR1A receptor. A summary of the results with murine FcγR is depicted in Table 9. The experiments demonstrated that all three humanized antibodies were able to mediate the uptake of HSP70 through the murine FcγR2B receptor. The chimeric IgG1 antibody facilitated uptake through the FcγR1, FcγR2B, and FcγR4 receptors while the IgG4 chimera utilized the FcγR1 and FcγR2B receptors for HSP70 uptake. The parental murine antibody promoted the uptake of through receptors FcγR2B and FcγR4.

TABLE 8
Summary of Uptake Experiments with Human FcγR

| Antibody | FcγR1A | FcγR1B | FcγR2A | FcγR2B | FcγR2C | FcγR3A | FcγR3B |
|---|---|---|---|---|---|---|---|
| IgG1 | + | − | + | + | − | − | − |
| Chimera | | | | | | | |
| IgG2 | − | − | + | + | − | − | − |
| Chimera | | | | | | | |
| IgG4 | + | − | + | + | − | − | − |
| Chimera | | | | | | | |
| h77A-1 | − | − | + | + | − | − | − |
| h77A-6 | − | − | + | + | − | − | − |
| h77A-11 | − | − | + | + | − | − | − |
| 77A | − | − | + | + | − | − | − |

TABLE 9
Summary of Uptake Experiments with Mouse FcγR

| Antibody | FcγR1 | FcγR2B | FcγR3 | FcγR4 |
|---|---|---|---|---|
| IgG1 | + | + | − | + |
| Chimera | | | | |
| IgG2 | − | + | − | − |
| Chimera | | | | |
| IgG4 | + | + | − | − |
| Chimera | | | | |
| h77A-1 | − | + | − | − |
| h77A-6 | − | + | − | − |
| h77A-11 | − | + | − | − |
| 77A | − | + | − | + |

### Example 15. —HSP70 Dendritic Cell Uptake Mediated by Humanized 77A Variants

This Example describes the uptake of HSP70 by dendritic cells (DCs) mediated by humanized 77A variants.

The Blood Dendritic Cell Isolation Kit II (Miltenyi Biotec) was used to isolate dendritic cells from human buffy coat. Briefly, B cells and monocytes were magnetically labeled and depleted using a cocktail of CD19 and CD14 MicroBeads. Subsequently, pre-enriched dendritic cells in the non-magnetic flow-through fraction were magnetically labeled and enriched using a cocktail of antibodies against the dendritic cell markers CD304 (BDCA-4/Neuropilin-1), CD141 (BDCA-3), and CD1c (BDCA-1). Fractions collected included plasmacytoid dendritic cells, type-1 myeloid dendritic cells (MDC1s), type-2 myeloid dendritic cells (MDC2s), and the unlabeled flow through fraction representing the non-DC fraction. The highly pure enriched cell fraction includes: plasmacytoid dendritic cells, type-1 myeloid dendritic cells (MDC1s), and type-2 myeloid dendritic cells (MDC2s).

Cells were incubated with HSP70GFP (BME Free; 5 μg/ml) and GFP-Nanobody Alexa-488 (1:1000) were added alone or in combination with antibody (1 pg/ml) for 1 hour at 4° C. Cells were then stained with or for Ghost Violet 450, CD11C, CD19, CD14, CD80, CD86, CD141, CD1C, and CD303.

Antibodies tested were: IgG2 isotype control, 77A (murine), a chimeric 77A with a human IgG1 Fc domain, a chimeric 77A with a human IgG2 Fc domain, a chimeric 77a with a human IgG4 domain, and humanized variants h77A-1 (including hVH-1 and hVL-1), h77A-6 (including hVH-2 and hVL-1), and h77A-11 (including hVH-3 and hVL-1), each as described in Example 12. The humanized antibodies h77A-1, h77A-6, and h77A-11 were each tested with a human IgG2 Fc domain.

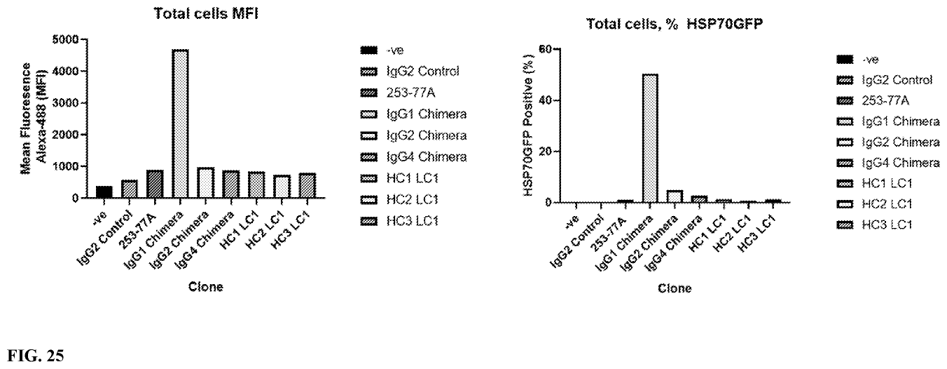

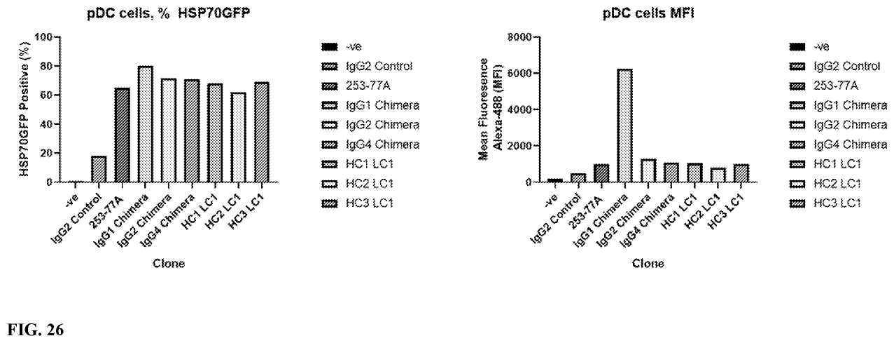

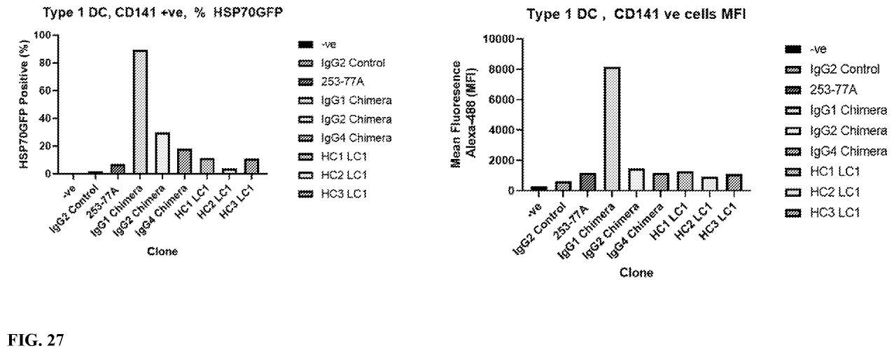

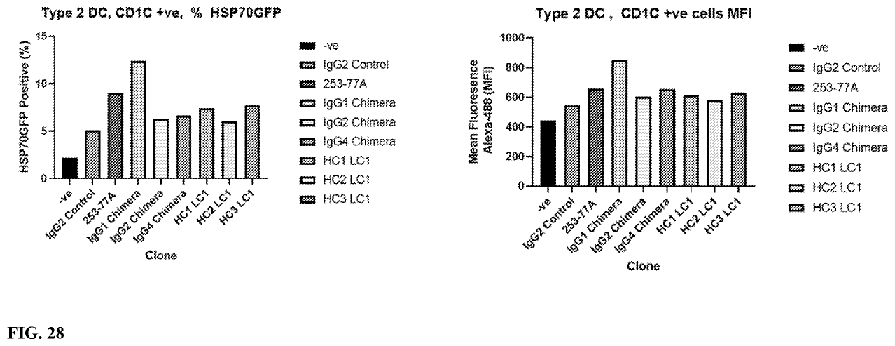

Results are depicted in FIGS. 25-28. Together, the results showed that human IgG2 isoforms of 77A mediated the uptake of HSP70 into primary human DCs, preferentially targeting plasmacytoid and type 2 peripheral blood DCs. Lesser uptake was observed for type 1 peripheral blood DCs. In addition, the IgG1 chimera only induced uptake into the unlabeled flow through fraction (FIG. 25).

### Example 16. —Further 77A Variants

h77A-1, including the combination of hVH-1 and hVL-1 (as described in Example 12) was selected for further optimization.

To optimize these sequences, a large library including variants of hVH-1 and hVL-1 was generated. Subsequently, an in silico model of antibody-antigen binding was generated and antibody-antigen binding for the full library was computationally simulated. The top 95 antibodies are referred to as h77A-1.1 through h77A-1.95, and are described in Table 10 (Kabat CDR Sequences) and Table 11 (IMGT CDR sequences). Amino acid sequences of CDR variants included in h77A-1.1 through h77A-1.95 are provided in Table 12, and amino acid sequences of variable region variants included in h77A-1.1 through h77A-1.95 are provided in Table 13. Additional antibody variants h77A-1.96, h77A-1.97, h77A-1.98 (also described in Tables 10 and 11) were also made and tested.

TABLE 10
h77A-1 Variants (Kabat CDR Sequences)

| | | | SEQ ID NO | | | | | | | |
|---|---|---|---|---|---|---|---|---|---|---|
| | | | | VH | VH | VH | | VL | VL | VL |
| Name | | | | CDR | CDR | CDR | | CDR | CDR | CDR |
| Antibody | VH | VL | VH | 1 | 2 | 3 | VL | 1 | 2 | 3 |
| h77A-1.1 | hVH-1.1 | hVL-1.1 | 26 | 164 | 2 | 3 | 105 | 4 | 5 | 6 |
| h77A-1.2 | hVH-1.2 | hVL-1.1 | 27 | 1 | 2 | 3 | 105 | 4 | 5 | 6 |
| h77A-1.3 | hVH-1.3 | hVL-1.1 | 28 | 1 | 2 | 170 | 105 | 4 | 5 | 6 |
| h77A-1.4 | hVH-1.4 | hVL-1.1 | 29 | 1 | 2 | 3 | 105 | 4 | 5 | 6 |
| h77A-1.5 | hVH-1.5 | hVL-1.2 | 30 | 1 | 2 | 3 | 106 | 4 | 5 | 162 |
| h77A-1.6 | hVH-1.6 | hVL-1.1 | 31 | 1 | 2 | 3 | 105 | 4 | 5 | 6 |
| h77A-1.7 | hVH-1.7 | hVL-1.1 | 32 | 1 | 2 | 171 | 105 | 4 | 5 | 6 |
| h77A-1.8 | hVH-1.5 | hVL-1.3 | 30 | 1 | 2 | 3 | 107 | 4 | 5 | 6 |
| h77A-1.9 | hVH-1.8 | hVL-1.1 | 33 | 1 | 2 | 172 | 105 | 4 | 5 | 6 |
| h77A-1.10 | hVH-1.9 | hVL-1.1 | 34 | 1 | 2 | 3 | 105 | 4 | 5 | 6 |
| h77A-1.11 | hVH-1.5 | hVL-1.4 | 30 | 1 | 2 | 3 | 108 | 4 | 5 | 6 |
| h77A-1.12 | hVH-1.5 | hVL-1.5 | 30 | 1 | 2 | 3 | 109 | 159 | 5 | 6 |
| h77A-1.13 | hVH-1.10 | hVL-1.1 | 35 | 1 | 2 | 173 | 105 | 4 | 5 | 6 |
| h77A-1.14 | hVH-1.11 | hVL-1.1 | 36 | 1 | 2 | 3 | 105 | 4 | 5 | 6 |
| h77A-1.15 | hVH-1.12 | hVL-1.1 | 37 | 1 | 2 | 174 | 105 | 4 | 5 | 6 |
| h77A-1.16 | hVH-1.1 | hVL-1.3 | 26 | 164 | 2 | 3 | 107 | 4 | 5 | 6 |
| h77A-1.17 | hVH-1.13 | hVL-1.1 | 38 | 164 | 2 | 175 | 105 | 4 | 5 | 6 |
| h77A-1.18 | hVH-1.6 | hVL-1.6 | 31 | 1 | 2 | 3 | 110 | 4 | 5 | 6 |
| h77A-1.19 | hVH-1.14 | hVL-1.1 | 39 | 1 | 2 | 3 | 105 | 4 | 5 | 6 |
| h77A-1.20 | hVH-1.15 | hVL-1.1 | 40 | 1 | 2 | 3 | 105 | 4 | 5 | 6 |
| h77A-1.21 | hVH-1.9 | hVL-1.7 | 34 | 1 | 2 | 3 | 111 | 4 | 5 | 6 |
| h77A-1.22 | hVH-1.16 | hVL-1.5 | 41 | 1 | 2 | 3 | 109 | 159 | 5 | 6 |
| h77A-1.23 | hVH-1.5 | hVL-1.8 | 30 | 1 | 2 | 3 | 112 | 4 | 5 | 163 |
| h77A-1.24 | hVH-1.3 | hVL-1.9 | 28 | 1 | 2 | 170 | 113 | 4 | 5 | 6 |
| h77A-1.25 | hVH-1.7 | hVL-1.10 | 32 | 1 | 2 | 171 | 114 | 4 | 5 | 6 |
| h77A-1.26 | hVH-1.17 | hVL-1.1 | 42 | 1 | 2 | 176 | 105 | 4 | 5 | 6 |
| h77A-1.27 | hVH-1.11 | hVL-1.11 | 36 | 1 | 2 | 3 | 115 | 4 | 5 | 6 |
| h77A-1.28 | hVH-1.18 | hVL-1.1 | 43 | 1 | 2 | 172 | 105 | 4 | 5 | 6 |
| h77A-1.29 | hVH-1.7 | hVL-1.5 | 32 | 1 | 2 | 171 | 109 | 159 | 5 | 6 |
| h77A-1.30 | hVH-1.19 | hVL-1.12 | 44 | 1 | 2 | 3 | 116 | 4 | 5 | 6 |
| h77A-1.31 | hVH-1.10 | hVL-1.13 | 35 | 1 | 2 | 173 | 117 | 4 | 5 | 6 |
| h77A-1.32 | hVH-1.20 | hVL-1.1 | 45 | 1 | 2 | 177 | 105 | 4 | 5 | 6 |
| h77A-1.33 | hVH-1.21 | hVL-1.1 | 46 | 164 | 2 | 178 | 105 | 4 | 5 | 6 |
| h77A-1.34 | hVH-1.11 | hVL-1.14 | 36 | 1 | 2 | 3 | 118 | 4 | 5 | 6 |
| h77A-1.35 | hVH-1.22 | hVL-1.11 | 47 | 1 | 2 | 179 | 115 | 4 | 5 | 6 |
| h77A-1.36 | hVH-1.23 | hVL-1.5 | 48 | 1 | 2 | 179 | 109 | 159 | 5 | 6 |
| h77A-1.37 | hVH-1.24 | hVL-1.1 | 49 | 164 | 167 | 174 | 105 | 4 | 5 | 6 |
| h77A-1.38 | hVH-1.25 | hVL-1.1 | 50 | 1 | 2 | 180 | 105 | 4 | 5 | 6 |
| h77A-1.39 | hVH-1.26 | hVL-1.2 | 51 | 1 | 2 | 3 | 106 | 4 | 5 | 162 |
| h77A-1.40 | hVH-1.27 | hVL-1.15 | 52 | 1 | 2 | 181 | 119 | 4 | 5 | 162 |
| h77A-1.41 | hVH-1.28 | hVL-1.4 | 53 | 1 | 2 | 174 | 108 | 4 | 5 | 6 |
| h77A-1.42 | hVH-1.29 | hVL-1.1 | 54 | 1 | 2 | 182 | 105 | 4 | 5 | 6 |
| h77A-1.43 | hVH-1.30 | hVL-1.12 | 55 | 1 | 2 | 3 | 116 | 4 | 5 | 6 |
| h77A-1.44 | hVH-1.31 | hVL-1.12 | 56 | 1 | 2 | 183 | 116 | 4 | 5 | 6 |
| h77A-1.45 | hVH-1.32 | hVL-1.16 | 57 | 1 | 2 | 3 | 120 | 4 | 5 | 163 |
| h77A-1.46 | hVH-1.33 | hVL-1.17 | 58 | 1 | 2 | 3 | 121 | 4 | 5 | 6 |
| h77A-1.47 | hVH-1.34 | hVL-1.18 | 59 | 1 | 2 | 180 | 122 | 4 | 5 | 6 |
| h77A-1.48 | hVH-1.35 | hVL-1.4 | 60 | 1 | 2 | 181 | 108 | 4 | 5 | 6 |
| h77A-1.49 | hVH-1.36 | hVL-1.19 | 61 | 1 | 168 | 184 | 123 | 4 | 5 | 6 |
| h77A-1.50 | hVH-1.37 | hVL-1.10 | 62 | 1 | 2 | 3 | 114 | 4 | 5 | 6 |
| h77A-1.51 | hVH-1.38 | hVL-1.20 | 63 | 165 | 2 | 185 | 124 | 4 | 5 | 6 |
| h77A-1.52 | hVH-1.39 | hVL-1.1 | 64 | 166 | 2 | 170 | 105 | 4 | 5 | 6 |
| h77A-1.53 | hVH-1.40 | hVL-1.21 | 65 | 1 | 2 | 3 | 125 | 4 | 5 | 6 |
| h77A-1.54 | hVH-1.41 | hVL-1.1 | 66 | 164 | 2 | 170 | 105 | 4 | 5 | 6 |
| h77A-1.55 | hVH-1.42 | hVL-1.21 | 67 | 1 | 2 | 171 | 125 | 4 | 5 | 6 |
| h77A-1.56 | hVH-1.43 | hVL-1.22 | 68 | 165 | 2 | 3 | 126 | 4 | 5 | 6 |
| h77A-1.57 | hVH-1.44 | hVL-1.23 | 69 | 1 | 169 | 174 | 127 | 160 | 5 | 6 |
| h77A-1.58 | hVH-1.45 | hVL-1.24 | 70 | 1 | 2 | 184 | 128 | 4 | 5 | 6 |
| h77A-1.59 | hVH-1.46 | hVL-1.13 | 71 | 1 | 2 | 184 | 117 | 4 | 5 | 6 |
| h77A-1.60 | h VH-1.47 | hVL-1.25 | 72 | 1 | 2 | 175 | 129 | 4 | 5 | 6 |
| h77A-1.61 | hVH-1.48 | hVL-1.26 | 73 | 1 | 2 | 3 | 130 | 4 | 5 | 6 |
| h77A-1.62 | h VH-1.49 | hVL-1.27 | 74 | 1 | 2 | 3 | 131 | 4 | 5 | 6 |
| h77A-1.63 | hVH-1.48 | hVL-1.28 | 73 | 1 | 2 | 3 | 132 | 4 | 5 | 6 |
| h77A-1.64 | hVH-1.50 | hVL-1.29 | 75 | 1 | 167 | 3 | 133 | 4 | 5 | 6 |
| h77A-1.65 | hVH-1.51 | hVL-1.30 | 76 | 1 | 169 | 3 | 134 | 4 | 5 | 6 |
| h77A-1.66 | hVH-1.52 | hVL-1.3 | 77 | 166 | 167 | 3 | 107 | 4 | 5 | 6 |
| h77A-1.67 | hVH-1.53 | hVL-1.31 | 78 | 1 | 2 | 3 | 135 | 4 | 5 | 6 |
| h77A-1.68 | hVH-1.54 | hVL-1.32 | 79 | 1 | 2 | 3 | 136 | 4 | 5 | 6 |
| h77A-1.69 | hVH-1.55 | hVL-1.33 | 80 | 1 | 168 | 3 | 137 | 4 | 5 | 6 |
| h77A-1.70 | hVH-1.16 | hVL-1.34 | 41 | 1 | 2 | 3 | 138 | 4 | 5 | 6 |
| h77A-1.71 | hVH-1.56 | hVL-1.35 | 81 | 1 | 2 | 3 | 139 | 4 | 5 | 6 |
| h77A-1.72 | hVH-1.57 | hVL-1.1 | 82 | 166 | 168 | 3 | 105 | 4 | 5 | 6 |
| h77A-1.73 | hVH-1.58 | hVL-1.22 | 83 | 1 | 2 | 3 | 126 | 4 | 5 | 6 |
| h77A-1.74 | hVH-1.59 | hVL-1.36 | 84 | 1 | 2 | 3 | 140 | 4 | 5 | 6 |
| h77A-1.75 | hVH-1.60 | hVL-1.37 | 85 | 1 | 2 | 3 | 141 | 161 | 5 | 6 |
| h77A-1.76 | hVH-1.61 | hVL-1.37 | 86 | 1 | 168 | 3 | 141 | 161 | 5 | 6 |
| h77A-1.77 | hVH-1.62 | hVL-1.13 | 87 | 166 | 2 | 184 | 117 | 4 | 5 | 6 |
| h77A-1.78 | hVH-1.63 | hVL-1.38 | 88 | 1 | 2 | 178 | 142 | 4 | 5 | 163 |
| h77A-1.79 | hVH-1.64 | hVL-1.39 | 89 | 164 | 2 | 170 | 143 | 4 | 5 | 162 |
| h77A-1.80 | hVH-1.65 | hVL-1.40 | 90 | 1 | 2 | 174 | 144 | 4 | 5 | 6 |
| h77A-1.81 | hVH-1.66 | hVL-1.5 | 91 | 166 | 2 | 183 | 109 | 161 | 5 | 6 |
| h77A-1.82 | hVH-1.67 | hVL-1.41 | 92 | 1 | 168 | 3 | 145 | 4 | 5 | 6 |
| h77A-1.83 | hVH-1.68 | hVL-1.42 | 93 | 1 | 2 | 184 | 146 | 4 | 5 | 6 |
| h77A-1.84 | hVH-1.69 | hVL-1.43 | 94 | 165 | 2 | 183 | 147 | 4 | 5 | 6 |
| h77A-1.85 | hVH-1.70 | hVL-1.44 | 95 | 166 | 2 | 183 | 148 | 4 | 5 | 6 |
| h77A-1.86 | hVH-1.71 | hVL-1.45 | 96 | 1 | 168 | 3 | 149 | 161 | 5 | 6 |
| h77A-1.87 | hVH-1.72 | hVL-1.46 | 97 | 164 | 2 | 170 | 150 | 4 | 5 | 6 |
| h77A-1.88 | hVH-1.73 | hVL-1.47 | 98 | 1 | 2 | 172 | 151 | 4 | 5 | 6 |
| h77A-1.89 | h VH-1.74 | hVL-1.48 | 99 | 1 | 167 | 3 | 152 | 4 | 5 | 6 |
| h77A-1.90 | hVH-1.75 | hVL-1.32 | 100 | 166 | 167 | 3 | 136 | 4 | 5 | 6 |
| h77A-1.91 | hVH-1.66 | hVL-1.49 | 91 | 166 | 2 | 183 | 153 | 161 | 5 | 6 |
| h77A-1.92 | hVH-1.76 | hVL-1.50 | 101 | 165 | 2 | 3 | 154 | 161 | 5 | 162 |
| h77A-1.93 | hVH-1.77 | hVL-1.51 | 102 | 1 | 168 | 183 | 155 | 4 | 5 | 6 |
| h77A-1.94 | hVH-1.11 | hVL-1.52 | 36 | 1 | 2 | 3 | 156 | 161 | 5 | 6 |
| h77A-1.95 | hVH-1.78 | hVL-1.53 | 103 | 1 | 2 | 184 | 157 | 4 | 5 | 6 |
| h77A-1.96 | hVH-1 | hVL-1.54 | 12 | 1 | 2 | 3 | 24 | 4 | 5 | 6 |
| h77A-1.97 | hVH-1.79 | hVL-1.54 | 17 | 1 | 2 | 3 | 24 | 4 | 5 | 6 |
| h77A-1.98 | hVH-1.79 | hVL-1 | 17 | 1 | 2 | 3 | 19 | 4 | 5 | 6 |

TABLE 11
h77A-1 Variants (IMGT CDR Sequences)

| | | | SEQ ID NO | | | | | | | |
|---|---|---|---|---|---|---|---|---|---|---|
| | | | | VH | VH | VH | | VL | VL | VL |
| Name | | | | CDR | CDR | CDR | | CDR | CDR | CDR |
| Antibody | VH | VL | VH | 1 | 2 | 3 | VL | 1 | 2 | 3 |
| h77A-1.1 | hVH-1.1 | hVL-1.1 | 26 | 192 | 196 | 3 | 105 | 186 | 191 | 6 |
| h77A-1.2 | hVH-1.2 | hVL-1.1 | 27 | 193 | 197 | 3 | 105 | 186 | 191 | 6 |
| h77A-1.3 | hVH-1.3 | hVL-1.1 | 28 | 193 | 196 | 170 | 105 | 186 | 191 | 6 |
| h77A-1.4 | hVH-1.4 | hVL-1.1 | 29 | 193 | 198 | 3 | 105 | 186 | 191 | 6 |
| h77A-1.5 | hVH-1.5 | hVL-1.2 | 30 | 193 | 196 | 3 | 106 | 186 | 191 | 162 |
| h77A-1.6 | hVH-1.6 | hVL-1.1 | 31 | 193 | 196 | 3 | 105 | 186 | 191 | 6 |
| h77A-1.7 | hVH-1.7 | hVL-1.1 | 32 | 193 | 196 | 171 | 105 | 186 | 191 | 6 |
| h77A-1.8 | hVH-1.5 | hVL-1.3 | 30 | 193 | 196 | 3 | 107 | 186 | 191 | 6 |
| h77A-1.9 | hVH-1.8 | hVL-1.1 | 33 | 193 | 196 | 172 | 105 | 186 | 191 | 6 |
| h77A-1.10 | hVH-1.9 | hVL-1.1 | 34 | 193 | 196 | 3 | 105 | 186 | 191 | 6 |
| h77A-1.11 | hVH-1.5 | hVL-1.4 | 30 | 193 | 196 | 3 | 108 | 186 | 191 | 6 |
| h77A-1.12 | hVH-1.5 | hVL-1.5 | 30 | 193 | 196 | 3 | 109 | 187 | 191 | 6 |
| h77A-1.13 | hVH-1.10 | hVL-1.1 | 35 | 193 | 196 | 173 | 105 | 186 | 191 | 6 |
| h77A-1.14 | hVH-1.11 | hVL-1.1 | 36 | 193 | 199 | 3 | 105 | 186 | 191 | 6 |
| h77A-1.15 | hVH-1.12 | hVL-1.1 | 37 | 193 | 196 | 174 | 105 | 186 | 191 | 6 |
| h77A-1.16 | hVH-1.1 | hVL-1.3 | 26 | 192 | 196 | 3 | 107 | 186 | 191 | 6 |
| h77A-1.17 | hVH-1.13 | hVL-1.1 | 38 | 192 | 196 | 175 | 105 | 186 | 191 | 6 |
| h77A-1.18 | hVH-1.6 | hVL-1.6 | 31 | 193 | 196 | 3 | 110 | 186 | 191 | 6 |
| h77A-1.19 | hVH-1.14 | hVL-1.1 | 39 | 193 | 200 | 3 | 105 | 186 | 191 | 6 |
| h77A-1.20 | hVH-1.15 | hVL-1.1 | 40 | 193 | 201 | 3 | 105 | 186 | 191 | 6 |
| h77A-1.21 | hVH-1.9 | hVL-1.7 | 34 | 193 | 196 | 3 | 111 | 186 | 191 | 6 |
| h77A-1.22 | hVH-1.16 | hVL-1.5 | 41 | 193 | 201 | 3 | 109 | 187 | 191 | 6 |
| h77A-1.23 | hVH-1.5 | hVL-1.8 | 30 | 193 | 196 | 3 | 112 | 188 | 191 | 163 |
| h77A-1.24 | hVH-1.3 | hVL-1.9 | 28 | 193 | 196 | 170 | 113 | 189 | 191 | 6 |
| h77A-1.25 | hVH-1.7 | hVL-1.10 | 32 | 193 | 196 | 171 | 114 | 186 | 191 | 6 |
| h77A-1.26 | hVH-1.17 | hVL-1.1 | 42 | 193 | 196 | 176 | 105 | 186 | 191 | 6 |
| h77A-1.27 | hVH-1.11 | hVL-1.11 | 36 | 193 | 199 | 3 | 115 | 186 | 191 | 6 |
| h77A-1.28 | hVH-1.18 | hVL-1.1 | 43 | 193 | 196 | 172 | 105 | 186 | 191 | 6 |
| h77A-1.29 | hVH-1.7 | hVL-1.5 | 32 | 193 | 196 | 171 | 109 | 187 | 191 | 6 |
| h77A-1.30 | hVH-1.19 | hVL-1.12 | 44 | 193 | 196 | 3 | 116 | 186 | 191 | 6 |
| h77A-1.31 | hVH-1.10 | hVL-1.13 | 35 | 193 | 196 | 173 | 117 | 186 | 191 | 6 |
| h77A-1.32 | hVH-1.20 | hVL-1.1 | 45 | 193 | 197 | 177 | 105 | 186 | 191 | 6 |
| h77A-1.33 | hVH-1.21 | hVL-1.1 | 46 | 192 | 196 | 178 | 105 | 186 | 191 | 6 |
| h77A-1.34 | hVH-1.11 | hVL-1.14 | 36 | 193 | 199 | 3 | 118 | 186 | 191 | 6 |
| h77A-1.35 | hVH-1.22 | hVL-1.11 | 47 | 193 | 196 | 179 | 115 | 186 | 191 | 6 |
| h77A-1.36 | hVH-1.23 | hVL-1.5 | 48 | 193 | 196 | 179 | 109 | 187 | 191 | 6 |
| h77A-1.37 | hVH-1.24 | hVL-1.1 | 49 | 192 | 202 | 174 | 105 | 186 | 191 | 6 |
| h77A-1.38 | hVH-1.25 | hVL-1.1 | 50 | 193 | 196 | 180 | 105 | 186 | 191 | 6 |
| h77A-1.39 | hVH-1.26 | hVL-1.2 | 51 | 193 | 201 | 3 | 106 | 186 | 191 | 162 |
| h77A-1.40 | hVH-1.27 | hVL-1.15 | 52 | 193 | 196 | 181 | 119 | 186 | 191 | 162 |
| h77A-1.41 | hVH-1.28 | hVL-1.4 | 53 | 193 | 196 | 174 | 108 | 186 | 191 | 6 |
| h77A-1.42 | hVH-1.29 | hVL-1.1 | 54 | 193 | 196 | 182 | 105 | 186 | 191 | 6 |
| h77A-1.43 | hVH-1.30 | hVL-1.12 | 55 | 193 | 196 | 3 | 116 | 186 | 191 | 6 |
| h77A-1.44 | hVH-1.31 | hVL-1.12 | 56 | 193 | 196 | 183 | 116 | 186 | 191 | 6 |
| h77A-1.45 | hVH-1.32 | hVL-1.16 | 57 | 193 | 196 | 3 | 120 | 186 | 191 | 163 |
| h77A-1.46 | hVH-1.33 | hVL-1.17 | 58 | 193 | 196 | 3 | 121 | 186 | 191 | 6 |
| h77A-1.47 | hVH-1.34 | hVL-1.18 | 59 | 193 | 196 | 180 | 122 | 186 | 191 | 6 |
| h77A-1.48 | hVH-1.35 | hVL-1.4 | 60 | 193 | 196 | 181 | 108 | 186 | 191 | 6 |
| h77A-1.49 | hVH-1.36 | hVL-1.19 | 61 | 193 | 203 | 184 | 123 | 186 | 191 | 6 |
| h77A-1.50 | hVH-1.37 | hVL-1.10 | 62 | 193 | 199 | 3 | 114 | 186 | 191 | 6 |
| h77A-1.51 | hVH-1.38 | hVL-1.20 | 63 | 194 | 196 | 185 | 124 | 189 | 191 | 6 |
| h77A-1.52 | hVH-1.39 | hVL-1.1 | 64 | 195 | 197 | 170 | 105 | 186 | 191 | 6 |
| h77A-1.53 | hVH-1.40 | hVL-1.21 | 65 | 193 | 204 | 3 | 125 | 186 | 191 | 6 |
| h77A-1.54 | hVH-1.41 | hVL-1.1 | 66 | 192 | 205 | 170 | 105 | 186 | 191 | 6 |
| h77A-1.55 | hVH-1.42 | hVL-1.21 | 67 | 193 | 206 | 171 | 125 | 186 | 191 | 6 |
| h77A-1.56 | hVH-1.43 | hVL-1.22 | 68 | 194 | 196 | 3 | 126 | 186 | 191 | 6 |
| h77A-1.57 | hVH-1.44 | hVL-1.23 | 69 | 193 | 207 | 174 | 127 | 190 | 191 | 6 |
| h77A-1.58 | hVH-1.45 | hVL-1.24 | 70 | 193 | 196 | 184 | 128 | 186 | 191 | 6 |
| h77A-1.59 | hVH-1.46 | hVL-1.13 | 71 | 193 | 206 | 184 | 117 | 186 | 191 | 6 |
| h77A-1.60 | hVH-1.47 | hVL-1.25 | 72 | 193 | 196 | 175 | 129 | 186 | 191 | 6 |
| h77A-1.61 | hVH-1.48 | hVL-1.26 | 73 | 193 | 196 | 3 | 130 | 186 | 191 | 6 |
| h77A-1.62 | hVH-1.49 | hVL-1.27 | 74 | 193 | 196 | 3 | 131 | 186 | 191 | 6 |
| h77A-1.63 | hVH-1.48 | hVL-1.28 | 73 | 193 | 196 | 3 | 132 | 189 | 191 | 6 |
| h77A-1.64 | hVH-1.50 | hVL-1.29 | 75 | 193 | 202 | 3 | 133 | 186 | 191 | 6 |
| h77A-1.65 | hVH-1.51 | hVL-1.30 | 76 | 193 | 207 | 3 | 134 | 186 | 191 | 6 |
| h77A-1.66 | hVH-1.52 | hVL-1.3 | 77 | 195 | 202 | 3 | 107 | 186 | 191 | 6 |
| h77A-1.67 | hVH-1.53 | hVL-1.31 | 78 | 193 | 196 | 3 | 135 | 186 | 191 | 6 |
| h77A-1.68 | hVH-1.54 | hVL-1.32 | 79 | 193 | 199 | 3 | 136 | 186 | 191 | 6 |
| h77A-1.69 | hVH-1.55 | hVL-1.33 | 80 | 193 | 208 | 3 | 137 | 186 | 191 | 6 |
| h77A-1.70 | hVH-1.16 | hVL-1.34 | 41 | 193 | 201 | 3 | 138 | 186 | 191 | 6 |
| h77A-1.71 | hVH-1.56 | hVL-1.35 | 81 | 193 | 196 | 3 | 139 | 186 | 191 | 6 |
| h77A-1.72 | hVH-1.57 | hVL-1.1 | 82 | 195 | 209 | 3 | 105 | 186 | 191 | 6 |
| h77A-1.73 | hVH-1.58 | hVL-1.22 | 83 | 193 | 196 | 3 | 126 | 186 | 191 | 6 |
| h77A-1.74 | hVH-1.59 | hVL-1.36 | 84 | 193 | 196 | 3 | 140 | 186 | 191 | 6 |
| h77A-1.75 | hVH-1.60 | hVL-1.37 | 85 | 193 | 201 | 3 | 141 | 187 | 191 | 6 |
| h77A-1.76 | hVH-1.61 | hVL-1.37 | 86 | 193 | 209 | 3 | 141 | 187 | 191 | 6 |
| h77A-1.77 | hVH-1.62 | hVL-1.13 | 87 | 195 | 206 | 184 | 117 | 186 | 191 | 6 |
| h77A-1.78 | hVH-1.63 | hVL-1.38 | 88 | 193 | 210 | 178 | 142 | 186 | 191 | 163 |
| h77A-1.79 | hVH-1.64 | hVL-1.39 | 89 | 192 | 197 | 170 | 143 | 186 | 191 | 162 |
| h77A-1.80 | hVH-1.65 | hVL-1.40 | 90 | 193 | 210 | 174 | 144 | 188 | 191 | 6 |
| h77A-1.81 | hVH-1.66 | hVL-1.5 | 91 | 195 | 196 | 183 | 109 | 187 | 191 | 6 |
| h77A-1.82 | hVH-1.67 | hVL-1.41 | 92 | 193 | 209 | 3 | 145 | 186 | 191 | 6 |
| h77A-1.83 | hVH-1.68 | hVL-1.42 | 93 | 193 | 196 | 184 | 146 | 188 | 191 | 6 |
| h77A-1.84 | hVH-1.69 | hVL-1.43 | 94 | 194 | 196 | 183 | 147 | 186 | 191 | 6 |
| h77A-1.85 | hVH-1.70 | hVL-1.44 | 95 | 195 | 196 | 183 | 148 | 188 | 191 | 6 |
| h77A-1.86 | hVH-1.71 | hVL-1.45 | 96 | 193 | 209 | 3 | 149 | 187 | 191 | 6 |
| h77A-1.87 | hVH-1.72 | hVL-1.46 | 97 | 192 | 211 | 170 | 150 | 186 | 191 | 6 |
| h77A-1.88 | hVH-1.73 | hVL-1.47 | 98 | 193 | 210 | 172 | 151 | 189 | 191 | 6 |
| h77A-1.89 | hVH-1.74 | hVL-1.48 | 99 | 193 | 202 | 3 | 152 | 186 | 191 | 6 |
| h77A-1.90 | hVH-1.75 | hVL-1.32 | 100 | 195 | 202 | 3 | 136 | 186 | 191 | 6 |
| h77A-1.91 | hVH-1.66 | hVL-1.49 | 91 | 195 | 196 | 183 | 153 | 187 | 191 | 6 |
| h77A-1.92 | hVH-1.76 | hVL-1.50 | 10 | 194 | 206 | 3 | 154 | 187 | 191 | 162 |
| h77A-1.93 | hVH-1.77 | hVL-1.51 | 102 | 193 | 208 | 183 | 155 | 186 | 191 | 6 |
| h77A-1.94 | hVH-1.11 | hVL-1.52 | 36 | 193 | 199 | 3 | 156 | 187 | 191 | 6 |
| h77A-1.95 | hVH-1.78 | hVL-1.53 | 103 | 193 | 199 | 184 | 157 | 186 | 191 | 6 |
| h77A-1.96 | hVH-1 | hVL-1.54 | 193 | 196 | 3 | 193 | 24 | 186 | 191 | 6 |
| h77A-1.97 | hVH-1.79 | hVL-1.54 | 193 | 196 | 3 | 193 | 24 | 186 | 191 | 6 |
| h77A-1.98 | hVH-1.79 | hVL-1 | 193 | 196 | 3 | 193 | 19 | 186 | 191 | 6 |

TABLE 12
CDR Amino Acid Sequences

| CDR | SEQ ID NO | Amino Acid Sequence |
|---|---|---|
| VLCDR1 | 159 | QSLFNSGTRKNY |
| VLCDR1 | 160 | QSLVNSGTRKNY |
| VLCDR1 | 161 | QSLFNSGTRKNY |
| VLCDR3 | 162 | KQSYNLYT |
| VLCDR3 | 163 | KQSYSLYT |
| VHCDR1 | 164 | GYTFTKYG |
| VHCDR1 | 165 | GYSFTNYG |
| VHCDR1 | 166 | GYIFTNYG |
| VHCDR2 | 167 | INTYTGES |
| VHCDR2 | 168 | INTYTGET |
| VHCDR2 | 169 | INTYTGEA |
| VHCDR3 | 170 | ARYDHRMDY |
| VHCDR3 | 171 | ARYDHEMDY |
| VHCDR3 | 172 | ARYDHTMDY |
| VHCDR3 | 173 | ARYDHPMDY |
| VHCDR3 | 174 | ARYDHVMDY |
| VHCDR3 | 175 | ARYDHSMDY |
| VHCDR3 | 176 | TRYDHAMDY |
| VHCDR3 | 177 | ARYDHDMDY |
| VHCDR3 | 178 | ARYDHNMDY |
| VHCDR3 | 179 | ARYDHHMDY |
| VHCDR3 | 180 | ARYDHLMDY |
| VHCDR3 | 181 | ARYDHYMDY |
| VHCDR3 | 182 | TRYDHRMDY |
| VHCDR3 | 183 | VRYDHAMDY |
| VHCDR3 | 184 | GRYDHAMDY |
| VHCDR3 | 185 | ARYDHGMDY |
| VLCDR1 | 186 | KSSQSLLNSGTRKNYLA |
| VLCDR1 | 187 | KSSQSLFNSGTRKNYLA |
| VLCDR1 | 188 | KSSQSLLNSGTRKNYLS |
| VLCDR1 | 189 | KSSQSLLNSGTRKNYLT |
| VLCDR1 | 190 | KSSQSLVNSGTRKNYLA |
| VLCDR2 | 191 | WTSTRES |
| VHCDR1 | 192 | GYTFTKYGMN |
| VHCDR1 | 193 | GYTFTNYGMN |
| VHCDR1 | 194 | GYSFTNYGMN |
| VHCDR1 | 195 | GYIFTNYGMN |
| VHCDR2 | 196 | WINTYTGEPTYADDFKG |
| VHCDR2 | 197 | WINTYTGEPRYADDFKG |
| VHCDR2 | 198 | WINTYTGEPIYADDFKG |
| VHCDR2 | 199 | WINTYTGEPTYTDDFKG |
| VHCDR2 | 200 | WINTYTGEPTYVDDFKG |
| VHCDR2 | 201 | WINTYTGEPTYSDDFKG |
| VHCDR2 | 202 | WINTYTGESTYADDFKG |
| VHCDR2 | 203 | WINTYTGETTYGDDFKG |
| VHCDR2 | 204 | WINTYTGEPKYTDDFKG |
| VHCDR2 | 205 | WINTYTGEPKYGDDFKG |
| VHCDR2 | 206 | WINTYTGEPTYGDDFKG |
| VHCDR2 | 207 | WINTYTGEATYADDFKG |
| VHCDR2 | 208 | WINTYTGETTYTDDFKG |
| VHCDR2 | 209 | WINTYTGETTYADDFKG |
| VHCDR2 | 210 | WINTYTGEPKYADDFKG |
| VHCDR2 | 211 | WINTYTGEPRYVDDFKG |

TABLE 13
Variable Region Amino Acid Sequences

| VH/VL | SEQ | |
|---|---|---|
| Name | ID NO | Amino Acid Sequence |
| hVH-1.1 | 26 | QIQLVQSGAEVKKPGASVKVSCKASGYTFTKYGMNWVRQAPGQGLEWMGW |
| | | INTYTGEPTYADDFKGRFTFTTDTSTSTAYMELRSLRSDDTAVYFCARYD |
| | | HAMDYWGQGTLVTVSS |
| hVH-1.2 | 27 | QIQLVQSGAEVKKPGASVKVSCKASGYTFTNYGMNWVRQAPGQGLEWMGW |
| | | INTYTGEPRYADDFKGRFTFTTDTSTSTAYMELRSLRSDDTAVYFCARYD |
| | | HAMDYWGQGTLVTVSS |
| hVH-1.3 | 28 | QIQLVQSGAEVKKPGASVKVSCKASGYTFTNYGMNWVRQAPGQGLEWMGW |
| | | INTYTGEPTYADDFKGRFTFTTDTSTSTAYMELRSLRSDDTAVYFCARYD |
| | | HRMDYWGQGTLVTVSS |
| hVH-1.4 | 29 | QIQLVQSGAEVKKPGASVKVSCKASGYTFTNYGMNWVRQAPGQGLEWMGW |
| | | INTYTGEPIYADDFKGRFTFTTDTSTSTAYMELRSLRSDDTAVYFCARYD |
| | | HAMDYWGQGTLVTVSS |
| hVH-1.5 | 30 | QIQLVQSGAEVKKPGASVKVSCKASGYTFTNYGMNWVRQAPGQGLEWMGW |
| | | INTYTGEPTYADDFKGRFTFTTDTSTSTAYMELRSLRSDDTAVYFCARYD |
| | | HAMDYWGQGTLVTVSS |
| hVH-1.6 | 31 | QIQLVQSGAEVKKPGASVKVSCKASGYTFTNYGMNWVRQAPGQGLEWMGW |
| | | INTYTGEPTYADDFKGRFTFTTDTSTRTAYMELRSLRSDDTAVYFCARYD |
| | | HAMDYWGQGTLVTVSS |
| hVH-1.7 | 32 | QIQLVQSGAEVKKPGASVKVSCKASGYTFTNYGMNWVRQAPGQGLEWMGW |
| | | INTYTGEPTYADDFKGRFTFTTDTSTSTAYMELRSLRSDDTAVYFCARYD |
| | | HEMDYWGQGTLVTVSS |
| hVH-1.8 | 33 | QIQLVQSGAEVKKPGASVKVSCKASGYTFTNYGMNWVRQAPGQGLEWMGW |
| | | INTYTGEPTYADDFKGRFTFTTDTSTSTAYMELRSLRSDDTAVYFCARYD |
| | | HTMDYWGQGTLVTVSS |
| hVH-1.9 | 34 | QIQLVQSGDEVKKPGASVKVSCKASGYTFTNYGMNWVRQAPGQGLEWMGW |
| | | INTYTGEPTYADDFKGRFTFTTDTSTSTAYMELRSLRSDDTAVYFCARYD |
| | | HAMDYWGQGTLVTVSS |
| hVH-1.10 | 35 | QIQLVQSGAEVKKPGASVKVSCKASGYTFTNYGMNWVRQAPGQGLEWMGW |
| | | INTYTGEPTYADDFKGRFTFTTDTSTSTAYMELRSLRSDDTAVYFCARYD |
| | | HPMDYWGQGTLVTVSS |
| hVH-1.11 | 36 | QIQLVQSGAEVKKPGASVKVSCKASGYTFTNYGMNWVRQAPGQGLEWMGW |
| | | INTYTGEPTYTDDEKGRFTFTTDTSTSTAYMELRSLRSDDTAVYFCARYD |
| | | HAMDYWGQGTLVTVSS |
| hVH-1.12 | 37 | QIQLVQSGAEVKKPGASVKVSCKASGYTFTNYGMNWVRQAPGQGLEWMGW |
| | | INTYTGEPTYADDFKGRFTFTTDTSTSTAYMELRSLRSDDTAVYFCARYD |
| | | HVMDYWGQGTLVTVSS |
| hVH-1.13 | 38 | QIQLVQSGAEVKKPGASVKVSCKASGYTFTKYGMNWVRQAPGQGLEWMGW |
| | | INTYTGEPTYADDEKGRFTFTTDTSTSTAYMELRSLRSDDTAVYFCARYD |
| | | HSMDYWGQGTLVTVSS |
| hVH-1.14 | 39 | QIQLVQSGAEVKKPGASVKVSCKASGYTFTNYGMNWVRQAPGQGLEWMGW |
| | | INTYTGEPTYVDDFKGRFTFTTDTSTSTAYMDLRSLRSDDTAVYFCARYD |
| | | HAMDYWGQGTLVTVSS |
| hVH-1.15 | 40 | QIQLVQSGDEVKKPGASVKVSCKASGYTFTNYGMNWVRQAPGQGLEWMGW |
| | | INTYTGEPTYSDDFKGRFTFTTDTSTSTAYMELRSLRSDDTAVYFCARYD |
| | | HAMDYWGQGTLVTVSS |
| hVH-1.16 | 41 | QIQLVQSGAEVKKPGASVKVSCKASGYTFTNYGMNWVRQAPGQGLEWMGW |
| | | INTYTGEPTYSDDFKGRFTFTTDTSTSTAYMELRSLRSDDTAVYFCARYD |
| | | HAMDYWGQGTLVTVSS |
| hVH-1.17 | 42 | QIQLVQSGAEVKKPGASVKVSCKASGYTFTNYGMNWVRQAPGQGLEWMGW |
| | | INTYTGEPTYADDFKGRFTFTTDTSTRTAYMELRSLRSDDTAVYFCTRYD |
| | | HAMDYWGQGTLVTVSS |
| hVH-1.18 | 43 | QIQLVQSGTEVKKPGASVKVSCKASGYTFTNYGMNWVRQAPGQGLEWMGW |
| | | INTYTGEPTYADDFKGRFTFTTDTSTSTAYMELRSLRSDDTAVYFCARYD |
| | | HTMDYWGQGTLVTVSS |
| hVH-1.19 | 44 | QIQLVQSGAEVKKPGASVKVSCKASGYTFTNYGMNWVRQAPGQGLEWMGW |
| | | INTYTGEPTYADDFKGRFTFTTDTSTSTAYMEVRSLRSDDTAVYFCARYD |
| | | HAMDYWGQGTLVTVSS |
| hVH-1.20 | 45 | QIQLVQSGAEVKKPGASVKVSCKASGYTFTNYGMNWVRQAPGQGLEWMGW |
| | | INTYTGEPRYADDFKGRFTFTTDTSTSTAYMELRSLRSDDTAVYFCARYD |
| | | HDMDYWGQGTLVTVSS |
| hVH-1.21 | 46 | QIQLVQSGAEVKKPGASVKVSCKASGYTFTKYGMNWVRQAPGQGLEWMGW |
| | | INTYTGEPTYADDFKGRFTFTTDTSTSTAYMELRSLRSDDTAVYFCARYD |
| | | HNMDYWGQGTLVTVSS |
| hVH-1.22 | 47 | QIQLVQSGTEVKKPGASVKVSCKASGYTFTNYGMNWVRQAPGQGLEWMGW |
| | | INTYTGEPTYADDFKGRFTFTTDTSTSTAYMELRSLRSDDTAVYFCARYD |
| | | HHMDYWGQGTLVTVSS |
| hVH-1.23 | 48 | QIQLVQSGAEVKKPGASVKVSCKASGYTFTNYGMNWVRQAPGQGLEWMGW |
| | | INTYTGEPTYADDFKGRFTFTTDTSTTTAYMELRSLRSDDTAVYFCARYD |
| | | HHMDYWGQGTLVTVSS |
| hVH-1.24 | 49 | QIQLVQSGAEVKKPGASVKVSCKASGYTFTKYGMNWVRQAPGQGLEWMGW |
| | | INTYTGESTYADDEKGRFTFTTDTSTSTAYMELRSLRSDDTAVYFCARYD |
| | | HVMDYWGQGTLVTVSS |
| hVH-1.25 | 50 | QIQLVQSGTEVKKPGASVKVSCKASGYTFTNYGMNWVRQAPGQGLEWMGW |
| | | INTYTGEPTYADDFKGRFTFTTDTSTRTAYMELRSLRSDDTAVYFCARYD |
| | | HLMDYWGQGTLVTVSS |
| hVH-1.26 | 51 | QIQLVQSGAEVKKPGASVKVSCKASGYTFTNYGMNWVRQAPGQGLEWMGW |
| | | INTYTGEPTYSDDFKGRFTFTTDTSTRTAYMELRSLRSDDTAVYFCARYD |
| | | HAMDYWGQGTLVTVSS |
| hVH-1.27 | 52 | QIQLVQSGAEVKKPGASVKVSCKASGYTFTNYGMNWVRQAPGQGLEWMGW |
| | | INTYTGEPTYADDFKGRFTFTTDTSTSTAYMELRSLRSDDTAVYFCARYD |
| | | HYMDYWGQGTLVTVSS |
| hVH-1.28 | 53 | QIQLVQSGAEVKKPGASVKVSCKASGYTFTNYGMNWVRQAPGQGLEWMGW |
| | | INTYTGEPTYADDFKGRFTFTTDTSTTTAYMELRSLRSDDTAVYFCARYD |
| | | HVMDYWGQGTLVTVSS |
| hVH-1.29 | 54 | QIHLVQSGAEVKKPGASVKVSCKASGYTFTNYGMNWVRQAPGQGLEWMGW |
| | | INTYTGEPTYADDFKGRFTFTTDTSTSTAYMELRSLRSDDTAVYFCTRYD |
| | | HRMDYWGQGTLVTVSS |
| hVH-1.30 | 55 | QIHLVQSGAEVKKPGASVKVSCKASGYTFTNYGMNWVRQAPGQGLEWMGW |
| | | INTYTGEPTYADDFKGRFTFTTDTSTSTVYMELRSLRSDDTAVYFCARYD |
| | | HAMDYWGQGTLVTVSS |
| hVH-1.31 | 56 | QIQLVQSGAEVKKPGASVKVSCKASGYTFTNYGMNWVRQAPGQGLEWMGW |
| | | INTYTGEPTYADDEKGRFTFTTDTSTSTAYMELRSLRSDDTAVYFCVRYD |
| | | HAMDYWGQGSLVTVSS |
| hVH-1.32 | 57 | QIQLVQSGVEVKKPGASVKVSCKASGYTFTNYGMNWVRQAPGQGLEWMGW |
| | | INTYTGEPTYADDFKGRFTFTTDTSTSTVYMELRSLRSDDTAVYFCARYD |
| | | HAMDYWGQGTLVTVSS |
| hVH-1.33 | 58 | QIQLVQSGVEVKKPGASVKVSCKASGYTFTNYGMNWVRQAPGQGLEWMGW |
| | | INTYTGEPTYADDFKGRFTFTTDTSTSTAYMELRSLRSDDTAVYFCARYD |
| | | HAMDYWGQGTLVTVSS |
| hVH-1.34 | 59 | QIQLVQSGTEVKKPGASVKVSCKASGYTFTNYGMNWVRQAPGQGLEWMGW |
| | | INTYTGEPTYADDFKGRFTFTTDTSTSTAYMELRSLRSDDTAVYFCARYD |
| | | HLMDYWGQGTLVTVSS |
| hVH-1.35 | 60 | QIQLVQSGAEVKKPGASVKVSCKASGYTFTNYGMNWVRQAPGQGLEWMGW |
| | | INTYTGEPTYADDFKGRFTFTTDTSTSTAYMDLRSLRSDDTAVYFCARYD |
| | | HYMDYWGQGTLVTVSS |
| hVH-1.36 | 61 | QIQLVQSGAEVKKPGASVKVSCKASGYTFTNYGMNWVRQAPGQGLEWMGW |
| | | INTYTGETTYGDDFKGRFTFTTDTSTSTAYMELRSLRSDDTAVYFCGRYD |
| | | HAMDYWGQGTLVTVSS |
| hVH-1.37 | 62 | QIHLVQSGVEVKKPGASVKVSCKASGYTFTNYGMNWVRQAPGQGLEWMGW |
| | | INTYTGEPTYTDDFKGRFTFTTDTSTSTAYMELRSLRSDDTAVYFCARYD |
| | | HAMDYWGQGTLVTVSS |
| hVH-1.38 | 63 | QIQLVQSGAEVKKPGASVKVSCKASGYSFTNYGMNWVRQAPGQGLEWMGW |
| | | INTYTGEPTYADDFKGRFTFTTDTSTSTAYMELRSLRSDDTAVYFCARYD |
| | | HGMDYWGQGTLVTVSS |
| hVH-1.39 | 64 | QIQLVQSGVEVKKPGASVKVSCKASGYIFTNYGMNWVRQAPGQGLEWMGW |
| | | INTYTGEPRYADDFKGRFTFTTDTSTSTAYMELRSLRSDDTAVYFCARYD |
| | | HRMDYWGQGTLVTVSS |
| hVH-1.40 | 65 | QIQLVQSGAEVKKPGASVKVSCKASGYTFTNYGMNWVRQAPGQGLEWMGW |
| | | INTYTGEPKYTDDFKGRFTFTTDTSTRTAYMELRSLRSDDTAVYFCARYD |
| | | HAMDYWGQGTLVTVSS |
| hVH-1.41 | 66 | QIQLVQSGAEVKKPGASVKVSCKASGYTFTKYGMNWVRQAPGQGLEWMGW |
| | | INTYTGEPKYGDDEKGRFTFTTDTSTSTAYMELRSLRSDDTAVYFCARYD |
| | | HRMDYWGQGTLVTVSS |
| hVH-1.42 | 67 | QIQLVQSGSEVKKPGASVKVSCKASGYTFTNYGMNWVRQAPGQGLEWMGW |
| | | INTYTGEPTYGDDFKGRFTFTTDTSTSTAYMELRSLRSDDTAVYFCARYD |
| | | HFMDYWGQGTLVTVSS |
| hVH-1.43 | 68 | QIQLVQSGTEVKKPGASVKVSCKASGYSFTNYGMNWVRQAPGQGLEWMGW |
| | | INTYTGEPTYADDFKGRFTFTTDTSTTTAYMELRSLRSDDTAVYFCARYD |
| | | HAMDYWGQGTLVTVSS |
| hVH-1.44 | 69 | QIQLVQSGAEVKKPGASVKVSCKASGYTFTNYGMNWVRQAPGQGLEWMGW |
| | | INTYTGEATYADDFKGRFTFTTDTSTSTAYMELRSLRSDDTAVYFCARYD |
| | | HVMDYWGQGTLVTVSS |
| hVH-1.45 | 70 | QIQLVQSGSEVKKPGASVKVSCKASGYTFTNYGMNWVRQAPGQGLEWMGW |
| | | INTYTGEPTYADDFKGRFTFTTDTSTSTAYMELRSLRSDDTAVYFCGRYD |
| | | HAMDYWGQGTLVTVSS |
| hVH-1.46 | 71 | QIQLVQSGPEVKKPGASVKVSCKASGYTFTNYGMNWVRQAPGQGLEWMGW |
| | | INTYTGEPTYGDDFKGRFTFTTDTSTSTAYMELRSLRSDDTAVYFCGRYD |
| | | HAMDYWGQGTLVTVSS |
| hVH-1.47 | 72 | QIQLVQSGSEVKKPGASVKVSCKASGYTFTNYGMNWVRQAPGQGLEWMGW |
| | | INTYTGEPTYADDEKGRFTFTTDTSTSTAYMELRSLRSDDTAVYFCARYD |
| | | HSMDYWGQGTLVTVSS |
| hVH-1.48 | 73 | QIQLVQSGAEVKKPGASVKVSCKASGYTFTNYGMNWVRQAPGQGLEWMGW |
| | | INTYTGEPTYADDFKGRFTFTTDTSTSTAYMELRSLRSDDTAVYFCARYD |
| | | HAMDYWGQGSLVTVSS |
| hVH-1.49 | 74 | QIQLVQSGAEVKKPGASVKVSCKASGYTFTNYGMNWVRQAPGQGLEWMGW |
| | | INTYTGEPTYADDFKGRFTFTTDTSTTTGYMELRSLRSDDTAVYFCARYD |
| | | HAMDYWGQGTLVTVSS |
| hVH-1.50 | 75 | QIQLVQSGAEVKKPGASVKVSCKASGYTFTNYGMNWVRQAPGQGLEWMGW |
| | | INTYTGESTYADDFKGRFTFTTDTSTSTAYMELRSLRSDDTAVYFCARYD |
| | | HAMDYWGQGTLVTVSS |
| hVH-1.51 | 76 | QIQLVQSGAEVKKPGASVKVSCKASGYTFTNYGMNWVRQAPGQGLEWMGW |
| | | INTYTGEATYADDFKGRFTFTTDTSTSTAYMELRSLRSDDTAVYFCARYD |
| | | HAMDYWGQGSLVTVSS |
| hVH-1.52 | 77 | QIQLVQSGAEVKKPGASVKVSCKASGYIFTNYGMNWVRQAPGQGLEWMGW |
| | | INTYTGESTYADDFKGRFTFTTDTSTSTVYMELRSLRSDDTAVYFCARYD |
| | | HAMDYWGQGTLVTVSS |
| hVH-1.53 | 78 | QIQLVQSGAEVKKPGASVKVSCKASGYTFTNYGMNWVRQAPGQGLEWMGW |
| | | INTYTGEPTYADDFKGRFTFTTDTSTSTAYMDLRSLRSDDTAVYFCARYD |
| | | HAMDYWGQGTLVTVSS |
| hVH-1.54 | 79 | QIHLVQSGDEVKKPGASVKVSCKASGYTFTNYGMNWVRQAPGQGLEWMGW |
| | | INTYTGEPTYTDDFKGRFTFTTDTSTSTAYMELRSLRSDDTAVYFCARYD |
| | | HAMDYWGQGTLVTVSS |
| hVH-1.55 | 80 | QIQLVQSGAEVKKPGASVKVSCKASGYTFTNYGMNWVRQAPGQGLEWMGW |
| | | INTYTGETTYTDDEKGRFTFTTDTSTSTAYMELRSLRSDDTAVYFCARYD |
| | | HAMDYWGQGTLVTVSS |
| hVH-1.56 | 81 | QIQLVQSGSEVKKPGASVKVSCKASGYTFTNYGMNWVRQAPGQGLEWMGW |
| | | INTYTGEPTYADDFKGRFTFTTDTSTSTAYMDLRSLRSDDTAVYFCARYD |
| | | HAMDYWGQGTLVTVSS |
| hVH-1.57 | 82 | QIQLVQSGPEVKKPGASVKVSCKASGYIFTNYGMNWVRQAPGQGLEWMGW |
| | | INTYTGETTYADDFKGRFTFTTDTSTTTVYMELRSLRSDDTAVYFCARYD |
| | | HAMDYWGQGTLVTVSS |
| hVH-1.58 | 83 | QIHLVQSGDEVKKPGASVKVSCKASGYTFTNYGMNWVRQAPGQGLEWMGW |
| | | INTYTGEPTYADDFKGRFTFTTDTSTSTGYMELRSLRSDDTAVYFCARYD |
| | | HAMDYWGQGSLVTVSS |
| hVH-1.59 | 84 | QIQLVQSGPEVKKPGASVKVSCKASGYTFTNYGMNWVRQAPGQGLEWMGW |
| | | INTYTGEPTYADDFKGRFTFTTDTSTTTGYMELRSLRSDDTAVYFCARYD |
| | | HAMDYWGQGTLVTVSS |
| hVH-1.60 | 85 | QIQLVQSGVEVKKPGASVKVSCKASGYTFTNYGMNWVRQAPGQGLEWMGW |
| | | INTYTGEPTYSDDFKGRFTFTTDTSTSTAYMEVRSLRSDDTAVYFCARYD |
| | | HAMDYWGQGTLVTVSS |
| hVH-1.61 | 86 | QIQLVQSGVEVKKPGASVKVSCKASGYTFTNYGMNWVRQAPGQGLEWMGW |
| | | INTYTGETTYADDFKGRFTFTTDTSTSTAYMEVRSLRSDDTAVYFCARYD |
| | | HAMDYWGQGTLVTVSS |
| hVH-1.62 | 87 | QIQLVQSGPEVKKPGASVKVSCKASGYIFTNYGMNWVRQAPGQGLEWMGW |
| | | INTYTGEPTYGDDFKGRFTFTTDTSTSTAYMELRSLRSDDTAVYFCGRYD |
| | | HAMDYWGQGTLVTVSS |
| hVH-1.63 | 88 | QIHLVQSGAEVKKPGASVKVSCKASGYTFTNYGMNWVRQAPGQGLEWMGW |
| | | INTYTGEPKYADDFKGRFTFTTDTSTSTAYMELRSLRSDDTAVYFCARYD |
| | | HNMDYWGQGTLVTVSS |
| hVH-1.64 | 89 | QIQLVQSGAEVKKPGASVKVSCKASGYTFTKYGMNWVRQAPGQGLEWMGW |
| | | INTYTGEPRYADDEKGRFTFTTDTSTSTAYMELRSLRSDDTAVYFCARYD |
| | | HRMDYWGQGTLVTVSS |
| hVH-1.65 | 90 | QIQLVQSGAEVKKPGASVKVSCKASGYTFTNYGMNWVRQAPGQGLEWMGW |
| | | INTYTGEPKYADDFKGRFTFTTDTSTSTAYMELRSLRSDDTAVYFCARYD |
| | | HVMDYWGQGTLVTVSS |
| hVH-1.66 | 91 | QIHLVQSGAEVKKPGASVKVSCKASGYIFTNYGMNWVRQAPGQGLEWMGW |
| | | INTYTGEPTYADDFKGRFTFTTDTSTSTVYMELRSLRSDDTAVYFCVRYD |
| | | HAMDYWGQGTLVTVSS |
| hVH-1.67 | 92 | QIQLVQSGTEVKKPGASVKVSCKASGYTFTNYGMNWVRQAPGQGLEWMGW |
| | | INTYTGETTYADDFKGRFTFTTDTSTSTAYMDLRSLRSDDTAVYFCARYD |
| | | HAMDYWGQGTLVTVSS |
| hVH-1.68 | 93 | QIHLVQSGPEVKKPGASVKVSCKASGYTFTNYGMNWVRQAPGQGLEWMGW |
| | | INTYTGEPTYADDFKGRFTFTTDTSTSTAYMELRSLRSDDTAVYFCGRYD |
| | | HAMDYWGQGTLVTVSS |
| hVH-1.69 | 94 | QIHLVQSGAEVKKPGASVKVSCKASGYSFTNYGMNWVRQAPGQGLEWMGW |
| | | INTYTGEPTYADDFKGRFTFTTDTSTSTAYMELRSLRSDDTAVYFCVRYD |
| | | HAMDYWGQGTLVTVSS |
| hVH-1.70 | 95 | QIHLVQSGAEVKKPGASVKVSCKASGYIFTNYGMNWVRQAPGQGLEWMGW |
| | | INTYTGEPTYADDFKGRFTFTTDTSTSTAYMELRSLRSDDTAVYFCVRYD |
| | | HAMDYWGQGTLVTVSS |
| hVH-1.71 | 96 | QIQLVQSGVEVKKPGASVKVSCKASGYTFTNYGMNWVRQAPGQGLEWMGW |
| | | INTYTGETTYADDFKGRFTFTTDTSTSTAYMELRSLRSDDTAVYFCARYD |
| | | HAMDYWGQGTLVTVSS |
| hVH-1.72 | 97 | QIQLVQSGAEVKKPGASVKVSCKASGYTFTKYGMNWVRQAPGQGLEWMGW |
| | | INTYTGEPRYVDDFKGRFTFTTDTSTSTAYMELRSLRSDDTAVYFCARYD |
| | | HRMDYWGQGTLVTVSS |
| hVH-1.73 | 98 | QIQLVQSGAEVKKPGASVKVSCKASGYTFTNYGMNWVRQAPGQGLEWMGW |
| | | INTYTGEPKYADDEKGRFTFTTDTSTRTAYMELRSLRSDDTAVYFCARYD |
| | | HTMDYWGQGTLVTVSS |
| hVH-1.74 | 99 | QIQLVQSGAEVKKPGASVKVSCKASGYTFTNYGMNWVRQAPGQGLEWMGW |
| | | INTYTGESTYADDFKGRFTFTTDTSTTTAYMELRSLRSDDTAVYFCARYD |
| | | HAMDYWGQGTLVTVSS |
| hVH-1.75 | 100 | QIQLVQSGAEVKKPGASVKVSCKASGYIFTNYGMNWVRQAPGQGLEWMGW |
| | | INTYTGESTYADDFKGRFTFTTDTSTTTGYMELRSLRSDDTAVYFCARYD |
| | | HAMDYWGQGTLVTVSS |
| hVH-1.76 | 101 | QIQLVQSGAEVKKPGASVKVSCKASGYSFTNYGMNWVRQAPGQGLEWMGW |
| | | INTYTGEPTYGDDFKGRFTFTTDTSTSTAYMELRSLRSDDTAVYFCARYD |
| | | HAMDYWGQGTLVTVSS |
| hVH-1.77 | 102 | QIQLVQSGAEVKKPGASVKVSCKASGYTFTNYGMNWVRQAPGQGLEWMGW |
| | | INTYTGETTYTDDFKGRFTFTTDTSTSTAYMELRSLRSDDTAVYFCVRYD |
| | | HAMDYWGQGTLVTVSS |
| hVH-1.78 | 103 | QIQLVQSGAEVKKPGASVKVSCKASGYTFTNYGMNWVRQAPGQGLEWMGW |
| | | INTYTGEPTYTDDFKGRFTFTTDTSTSTAYMELRSLRSDDTAVYFCGRYD |
| | | HAMDYWGQGTLVTVSS |
| hVH-1.79 | 17 | QIQLVQSGAEVKKPGASVKVSCKASGYTFTNYGMNWVRQAPGQGLE |
| | | WMGWINTYTGEPTYADDFKGRFTMTTDTSTSTAYMELRSLRSDDTA |
| | | VYFCARYDHAMDYWGQGTLVTVSS |
| hVL-1.1 | 105 | EIVLTQSPDSLAVSLGERATINCKSSQSLLNSGTRKNYLAWYQQKAGQSP |
| | | KLLIYWTSTRESGVPDRFSGSGSGTDFTLTIDSLQAEDVAVYYCKQSYTL |
| | | YTFGGGTKVEIK |
| hVL-1.2 | 106 | EIVLTQSPDSLAVSLGERATINCKSSQSLLNSGTRKNYLAWYQQKAGQSP |
| | | KLLIYWTSTRESGVPDRFSGSGSGTDFTLTIDSLQAEDVAVYYCKQSYNL |
| | | YTFGGGTKVEIK |
| hVL-1.3 | 107 | EIVLTQSPDSLAVSLGERATIKCKSSQSLLNSGTRKNYLAWYQQKAGQSP |
| | | KLLIYWTSTRESGVPDRFSGSGSGTDFTLTIDSLQAEDVAVYYCKQSYTL |
| | | YTFGGGTKVEIK |
| hVL-1.4 | 108 | EIVLTQSPDSLAVSLGERATINCKSSQSLLNSGTRKNYLAWYQQKPGQSP |
| | | KLLIYWTSTRESGVPDRFSGSGSGTDFTLTIDSLQAEDVAVYYCKQSYTL |
| | | YTFGGGTKVEIK |
| hVL-1.5 | 109 | EIVLTQSPDSLAVSLGERATINCKSSQSLFNSGTRKNYLAWYQQKAGQSP |
| | | KLLIYWTSTRESGVPDRFSGSGSGTDFTLTIDSLQAEDVAVYYCKQSYTL |
| | | YTFGGGTKVEIK |
| hVL-1.6 | 110 | EIVLTQSPDSLAVSLGERATINCKSSQSLLNSGTRKNYLAWYQQKAGQSP |
| | | KLVIYWTSTRESGVPDRFSGSGSGTDFTLTIDSLQAEDVAVYYCKQSYTL |
| | | YTFGGGTKVEIK |
| hVL-1.7 | 111 | EIVLTQSPDSLAVSLGERATINCKSSQSLLNSGTRKNYLAWYQQKAGQSP |
| | | KLLIYWTSTRESGVPDRFSGSGSGTDFTLSIDSLQAEDVAVYYCKQSYTL |
| | | YTFGGGTKVEIK |
| hVL-1.8 | 112 | EIVLTQSPDSLAVSLGERATINCKSSQSLLNSGTRKNYLSWYQQKAGQSP |
| | | KLLIYWTSTRESGVPDRFSGSGSGTDFTLTIDSLQAEDVAVYYCKQSYSL |
| | | YTFGGGTKVEIK |
| hVL-1.9 | 113 | EIVLTQSPDSLAVSLGERATINCKSSQSLLNSGTRKNYLTWYQQKAGQSP |
| | | KLLIYWTSTRESGVPDRFSGSGSGTDFTLTIDSLQAEDVAVYYCKQSYTL |
| | | YTFGGGTKVEIK |
| hVL-1.10 | 114 | EIVLTQSPDSLAVSLGERATINCKSSQSLLNSGTRKNYLAWYQQKAGQSP |
| | | KLLIYWTSTRESGVPDRFSGSGSGTDFTLTIDSLQAEDVAIYYCKQSYTL |
| | | YTFGGGTKVEIK |
| hVL-1.11 | 115 | EIVLTQSPDSLTVSLGERATINCKSSQSLLNSGTRKNYLAWYQQKAGQSP |
| | | KLLIYWTSTRESGVPDRFSGSGSGTDFTLTIDSLQAEDVAVYYCKQSYTL |
| | | YTFGGGTKVEIK |
| hVL-1.12 | 116 | EIVLTQSPDSLAVSLGERATINCKSSQSLLNSGTRKNYLAWYQQKAGQSP |
| | | KLIIYWTSTRESGVPDRFSGSGSGTDFTLTIDSLQAEDVAVYYCKQSYTL |
| | | YTFGGGTKVEIK |
| hVL-1.13 | 117 | EIVLTQSPDSLAVSLGERATINCKSSQSLLNSGTRKNYLAWYQQKAGQSP |
| | | KLLIYWTSTRESGVPDRFSGSGSGTDFTLTIDRLQAEDVAVYYCKQSYTL |
| | | YTFGGGTKVEIK |
| hVL-1.14 | 118 | EIVLTQSPDSLTVSLGERATINCKSSQSLLNSGTRKNYLAWYQQKAGQSP |
| | | NLLIYWTSTRESGVPDRFSGSGSGTDFTLTIDSLQAEDVAVYYCKQSYTL |
| | | YTFGGGTKVEIK |
| hVL-1.15 | 119 | EIVLTQSPDSLTVSLGERATINCKSSQSLLNSGTRKNYLAWYQQKAGQSP |
| | | KLLIYWTSTRESGVPDRFSGSGSGTDFTLTIDSLQAEDVAVYYCKQSYNL |
| | | YTFGGGTKVEIK |
| hVL-1.16 | 120 | EIVLTQSPDSLAVSLGERATINCKSSQSLLNSGTRKNYLAWYQQKAGQSP |
| | | KLLIYWTSTRESGVPDRFSGSGSGTDFTLTIDSLQAEDVAVYYCKQSYSL |
| | | YTFGGGTKVEIK |
| hVL-1.17 | 121 | EIVLTQSPDSLAVSLGERATIKCKSSQSLLNSGTRKNYLAWYQKKAGQSP |
| | | KLLIYWTSTRESGVPDRFSGSGSGTDFTLTIDSLQAEDVAVYYCKQSYTL |
| | | YTFGGGTKVEIK |
| hVL-1.18 | 122 | EIVLTQSPDSLAVSLGERATINCKSSQSLLNSGTRKNYLAWYQQKAGQSP |
| | | KLLIYWTSTRESGVPDRFSGSGSGTDFTLTIDSLQTEDVAVYYCKQSYTL |
| | | YTFGGGTKVEIK |
| hVL-1.19 | 123 | EIVLTQSPDSLAVSLGERATINCKSSQSLLNSGTRKNYLAWYQQKAGQSP |
| | | KLLIYWTSTRESGVPDRFSASGSGTDFTLTIDSLQAEDVAVYYCKQSYTL |
| | | YTFGGGTKVEIK |
| hVL-1.20 | 124 | EIVLTQSPDSLAVSLGERATINCKSSQSLLNSGTRKNYLTWYQQKAGQSP |
| | | KLLIYWTSTRESGVPDRFSGSGSGTDFTLTIDSLQAEDVALYYCKQSYTL |
| | | YTFGGGTKVEIK |
| hVL-1.21 | 125 | EIVLTQSPDSLSVSLGERATINCKSSQSLLNSGTRKNYLAWYQQKAGQSP |
| | | KLLIYWTSTRESGVPDRFSGSGSGTDFTLTIDSLQAEDVAVYYCKQSYTL |
| | | YTFGGGTKVEIK |
| hVL-1.22 | 126 | EIVLTQSPDSLAVSLGERATINCKSSQSLLNSGTRKNYLAWYQQKSGQSP |
| | | KLLIYWTSTRESGVPDRFSGSGSGTDFTLTIDSLQAEDVAVYYCKQSYTL |
| | | YTFGGGTKVEIK |
| hVL-1.23 | 127 | EIVLTQSPDSLAVSLGERATINCKSSQSLVNSGTRKNYLAWYQQKAGQSP |
| | | KLLIYWTSTRESGVPDRFSGSGSGTDFTLTIDSLQTEDVAVYYCKQSYTL |
| | | YTFGGGTKVEIK |
| hVL-1.24 | 128 | EIVLTQSPDSLAVSLGERATIKCKSSQSLLNSGTRKNYLAWYQQKAGQSP |
| | | KLLIYWTSTRESGVPDRFSGSGSGTDFTLTIDSLQAEDVALYYCKQSYTL |
| | | YTFGGGTKVEIK |
| hVL-1.25 | 129 | EIVLTQSPDSLAVSLGERATINCKSSQSLLNSGTRKNYLAWYQQKAGQSP |
| | | KLIIYWTSTRESGVPDRFSGSGSGTDFTLTIDSLQAEDVALYYCKQSYTL |
| | | YTFGGGTKVEIK |
| hVL-1.26 | 130 | EIVLTQSPDSLAVSLGERATINCKSSQSLLNSGTRKNYLAWYQKKSGQSP |
| | | KLLIYWTSTRESGVPDRFSGSGSGTDFTLSIDSLQAEDVAVYYCKQSYTL |
| | | YTFGGGTKVEIK |
| hVL-1.27 | 131 | EIVLTQSPDSLAVSLGERATIKCKSSQSLLNSGTRKNYLAWYQQKAGQSP |
| | | KLLIYWTSTRESGVPDRFSASGSGTDFTLTIDSLQAEDVAVYYCKQSYTL |
| | | YTFGGGTKVEIK |
| hVL-1.28 | 132 | EIVLTQSPDSLAVSLGERATINCKSSQSLLNSGTRKNYLTWYQQKSGQSP |
| | | KLLIYWTSTRESGVPDRFSGSGSGTDFTLTIDSLQAEDVAIYYCKQSYTL |
| | | YTFGGGTKVEIK |
| hVL-1.29 | 133 | EIVLTQSPDSLAVSLGERATINCKSSQSLLNSGTRKNYLAWYQKKAGQSP |
| | | KLLIYWTSTRESGVPDRFSGSGSGTDFTLSIDSLQAEDVAIYYCKQSYTL |
| | | YTFGGGTKVEIK |
| hVL-1.30 | 134 | EIVLTQSPDSLAVSLGERATINCKSSQSLLNSGTRKNYLAWYQQKSGQSP |
| | | KLLIYWTSTRESGVPDRFSGSGSGTDFTLTIDSLQAEDVAIYYCKQSYTL |
| | | YTFGGGTKVEIK |
| hVL-1.31 | 135 | EIVLTQSPDSLSVSLGERATINCKSSQSLLNSGTRKNYLAWYQQKAGQSP |
| | | NLLIYWTSTRESGVPDRFSGSGSGTDFTLSIDSLQAEDVAVYYCKQSYTL |
| | | YTFGGGTKVEIK |
| hVL-1.32 | 136 | EIVLTQSPDSLSVSLGERATINCKSSQSLLNSGTRKNYLAWYQQKAGQSP |
| | | NLLIYWTSTRESGVPDRFSGSGSGTDFTLTIDSLQAEDVAVYYCKQSYTL |
| | | YTFGGGTKVEIK |
| hVL-1.33 | 137 | EIVLTQSPDSLTVSLGERATINCKSSQSLLNSGTRKNYLAWYQQKAGQSP |
| | | NLLIYWTSTRESGVPDRFSGSGSGTDFTLTIDSLQAEDVALYYCKQSYTL |
| | | YTFGGGTKVEIK |
| hVL-1.34 | 138 | EIVLTQSPDSLSVSLGERATINCKSSQSLLNSGTRKNYLAWYQKKAGQSP |
| | | KLLIYWTSTRESGVPDRFSGSGSGTDFTLTIDRLQAEDVALYYCKQSYTL |
| | | YTFGGGTKVEIK |
| hVL-1.35 | 139 | EIVLTQSPDSLSVSLGERATIKCKSSQSLLNSGTRKNYLAWYQQKAGQSP |
| | | KLLIYWTSTRESGVPDRFSGSGSGTDFTLTIDSLQTEDVAVYYCKQSYTL |
| | | YTFGGGTKVEIK |
| hVL-1.36 | 140 | EIVLTQSPDSLAVSLGERATIKCKSSQSLLNSGTRKNYLAWYQQKAGQSP |
| | | KLLIYWTSTRESGVPDRFSGSGSGTDFTLTIDSLQAEDVAIYYCKQSYTL |
| | | YTFGGGTKVEIK |
| hVL-1.37 | 141 | EIVLTQSPDSLAVSLGERATINCKSSQSLFNSGTRKNYLAWYQQKAGQSP |
| | | KLLIYWTSTRESGVPDRFSGSGSGTDFTLSIDSLQAEDVAVYYCKQSYTL |
| | | YTFGGGTKVEIK |
| hVL-1.38 | 142 | EIVLTQSPDSLAVSLGERATINCKSSQSLLNSGTRKNYLAWYQQKAGQSP |
| | | NLLIYWTSTRESGVPDRFSGSGSGTDFTLTIDSLQAEDVAVYYCKQSYSL |
| | | YTFGGGTKVEIK |
| hVL-1.39 | 143 | EIVLTQSPDSLAVSLGERATINCKSSQSLLNSGTRKNYLAWYQQKPGQSP |
| | | KLLIYWTSTRESGVPDRFSGSGSGTDFTLTIDSLQAEDVAVYYCKQSYNL |
| | | YTFGGGTKVEIK |
| hVL-1.40 | 144 | EIVLTQSPDSLAVSLGERATINCKSSQSLLNSGTRKNYLSWYQQKPGQSP |
| | | KLIIYWTSTRESGVPDRFSGSGSGTDFTLTIDSLQAEDVAVYYCKQSYTL |
| | | YTFGGGTKVEIK |
| hVL-1.41 | 145 | EIVLTQSPDSLAVSLGERATINCKSSQSLLNSGTRKNYLAWYQQKSGQSP |
| | | KLLIYWTSTRESGVPDRFSGSGSGTDFTLTIDSLQTEDVAIYYCKQSYTL |
| | | YTFGGGTKVEIK |
| hVL-1.42 | 146 | EIVLTQSPDSLAVSLGERATINCKSSQSLLNSGTRKNYLSWYQQKAGQSP |
| | | NLVIYWTSTRESGVPDRFSGSGSGTDFTLTIDSLQAEDVAVYYCKQSYTL |
| | | YTFGGGTKVEIK |
| hVL-1.43 | 147 | EIVLTQSPDSLAVSLGERATINCKSSQSLLNSGTRKNYLAWYQKKSGQSP |
| | | NLLIYWTSTRESGVPDRFSGSGSGTDFTLTIDSLQAEDVAVYYCKQSYTL |
| | | YTFGGGTKVEIK |
| hVL-1.44 | 148 | EIVLTQSPDSLAVSLGERATINCKSSQSLLNSGTRKNYLSWYQKKSGQSP |
| | | KLLIYWTSTRESGVPDRFSGSGSGTDFTLTIDSLQAEDVAVYYCKQSYTL |
| | | YTFGGGTKVEIK |
| hVL-1.45 | 149 | EIVLTQSPDSLAVSLGERATINCKSSQSLFNSGTRKNYLAWYQKKAGQSP |
| | | KLLIYWTSTRESGVPDRFSGSGSGTDFTLSIDRLQAEDVAVYYCKQSYTL |
| | | YTFGGGTKVEIK |
| hVL-1.46 | 150 | EIVLTQSPDSLTVSLGERATINCKSSQSLLNSGTRKNYLAWYQQKPGQSP |
| | | KLLIYWTSTRESGVPDRFSGSGSGTDFTLTIDSLQAEDVAVYYCKQSYTL |
| | | YTFGGGTKVEIK |
| hVL-1.47 | 151 | EIVLTQSPDSLAVSLGERATIKCKSSQSLLNSGTRKNYLTWYQQKAGQSP |
| | | NLLIYWTSTRESGVPDRFSGSGSGTDFTLTIDSLQAEDVAVYYCKQSYTL |
| | | YTFGGGTKVEIK |
| hVL-1.48 | 152 | EIVLTQSPDSLSVSLGERATINCKSSQSLLNSGTRKNYLAWYQQKSGQSP |
| | | NLVIYWTSTRESGVPDRFSGSGSGTDFTLTIDSLQAEDVAVYYCKQSYTL |
| | | YTFGGGTKVEIK |
| hVL-1.49 | 153 | EIVLTQSPDSLSVSLGERATINCKSSQSLFNSGTRKNYLAWYQQKAGQSP |
| | | KLLIYWTSTRESGVPDRFSGSGSGTDFTLTIDSLQAEDVAVYYCKQSYTL |
| | | YTFGGGTKVEIK |
| hVL-1.50 | 154 | EIVLTQSPDSLTVSLGERATINCKSSQSLFNSGTRKNYLAWYQKKAGQSP |
| | | KLLIYWTSTRESGVPDRFSGSGSGTDFTLTIDSLQAEDVAVYYCKQSYNL |
| | | YTFGGGTKVEIK |
| hVL-1.51 | 155 | EIVLTQSPDSLAVSLGERATINCKSSQSLLNSGTRKNYLAWYQQKPGQSP |
| | | KLLIYWTSTRESGVPDRFSASGSGTDFTLTIDSLQAEDVAIYYCKQSYTL |
| | | YTFGGGTKVEIK |
| hVL-1.52 | 156 | EIVLTQSPDSLAVSLGERATINCKSSQSLFNSGTRKNYLAWYQQKAGQSP |
| | | KLIIYWTSTRESGVPDRFSASGSGTDFTLSIDRLQAEDVAVYYCKQSYTL |
| | | YTFGGGTKVEIK |
| hVL-1.53 | 157 | EIVLTQSPDSLSVSLGERATINCKSSQSLLNSGTRKNYLAWYQQKAGQSP |
| | | KLLIYWTSTRESGVPDRFSASGSGTDFTLTIDRLQAEDVALYYCKQSYTL |
| | | YTFGGGTKVEIK |
| hVL-1.54 | 24 | DIVMTQSPDSLAVSLGERATINCKSSQSLLNSGTRKNYLAWYQQKP |
| | | GQSPKLLIYWTSTRESGVPDRFSGSGSGTDFTLTISSLQAEDVAVY |
| | | YCKQSYTLYTFGQGTKLEIK |

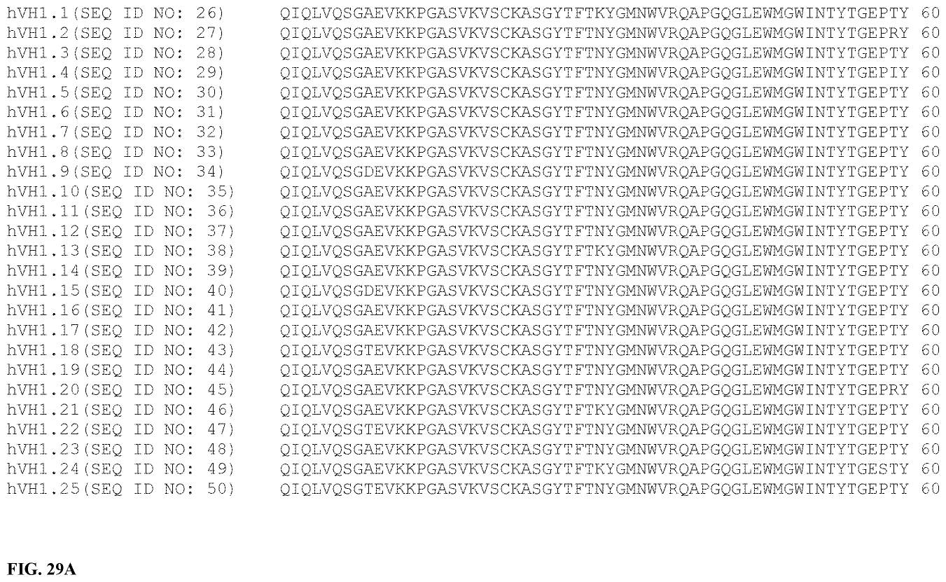

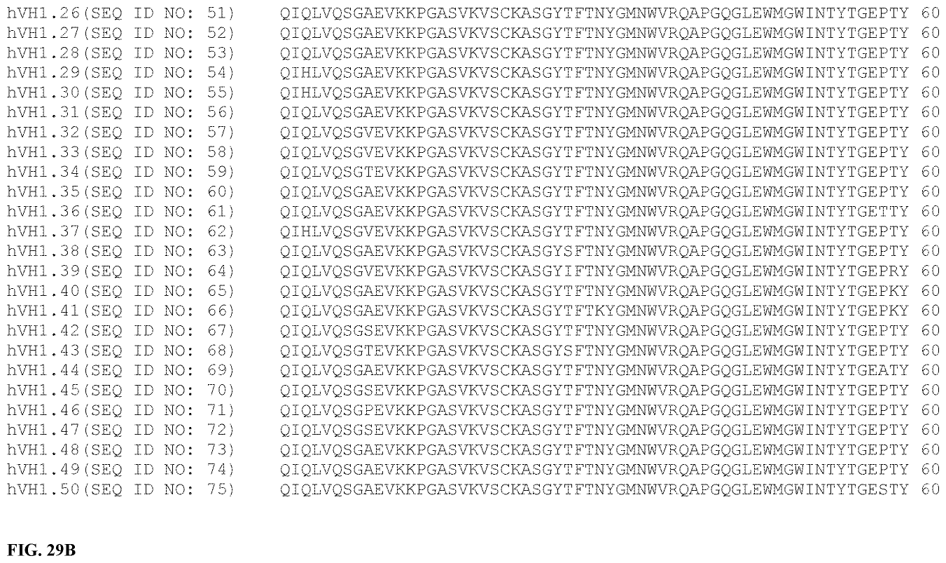

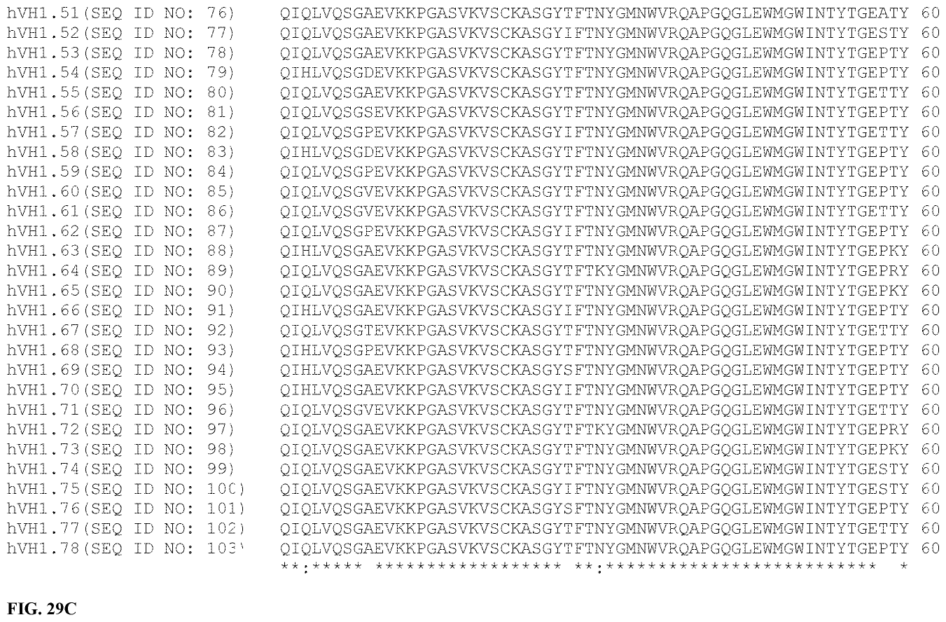

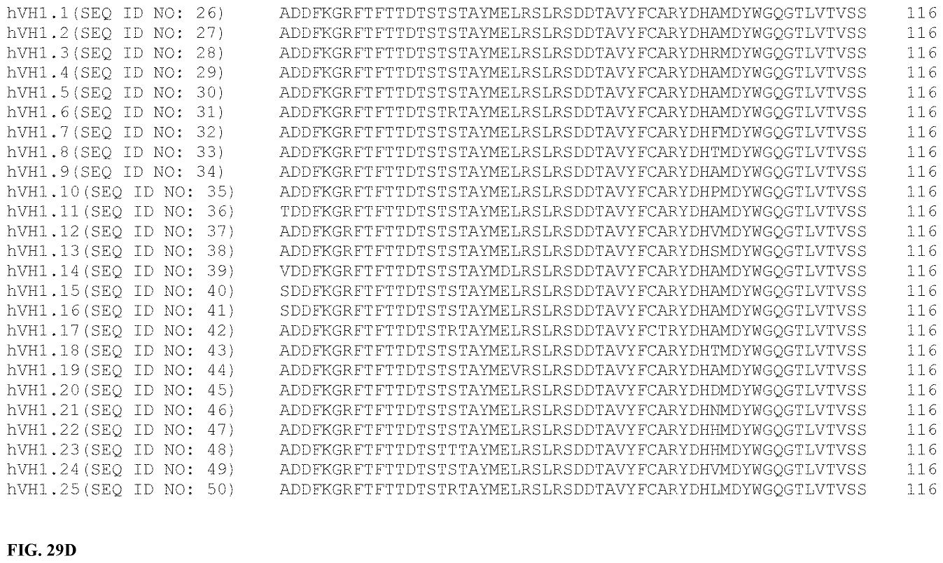

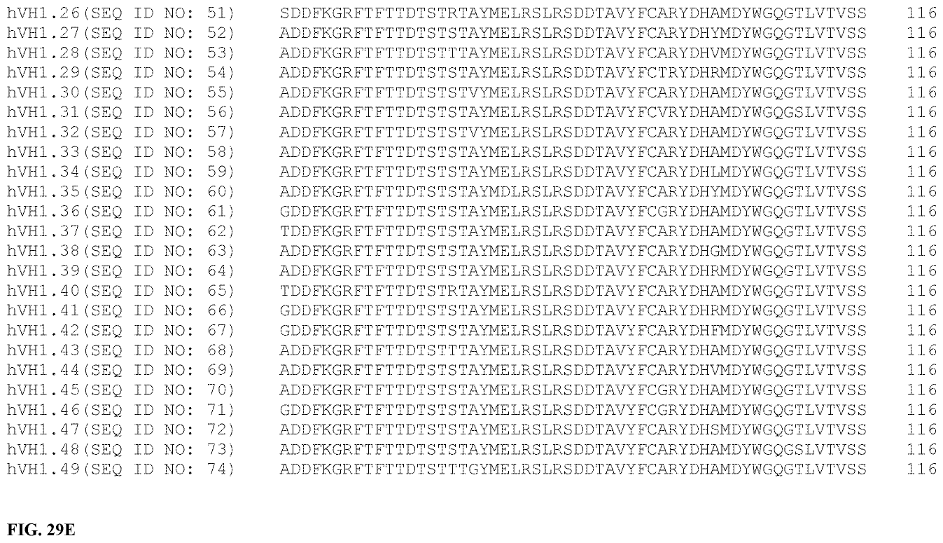

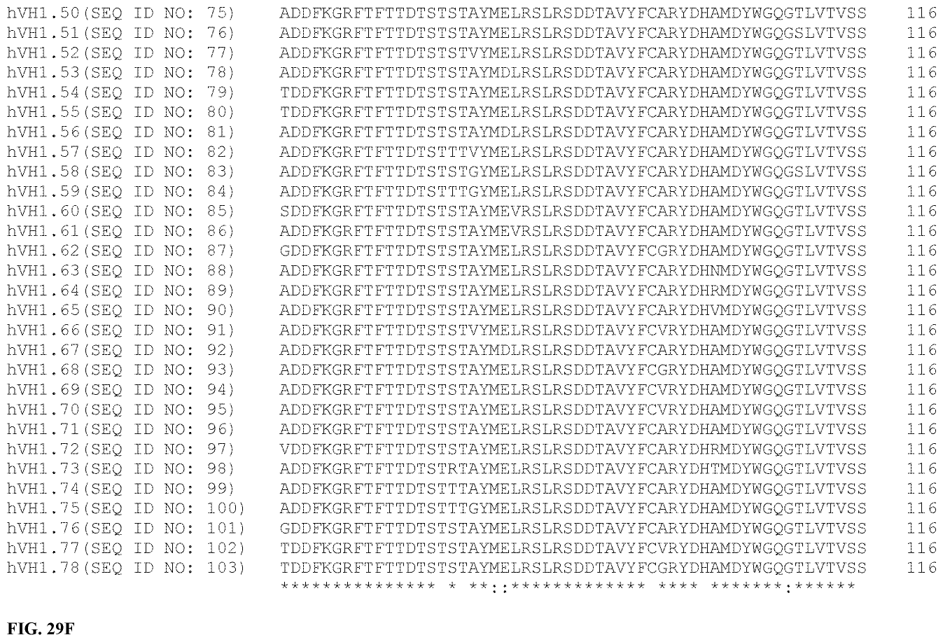

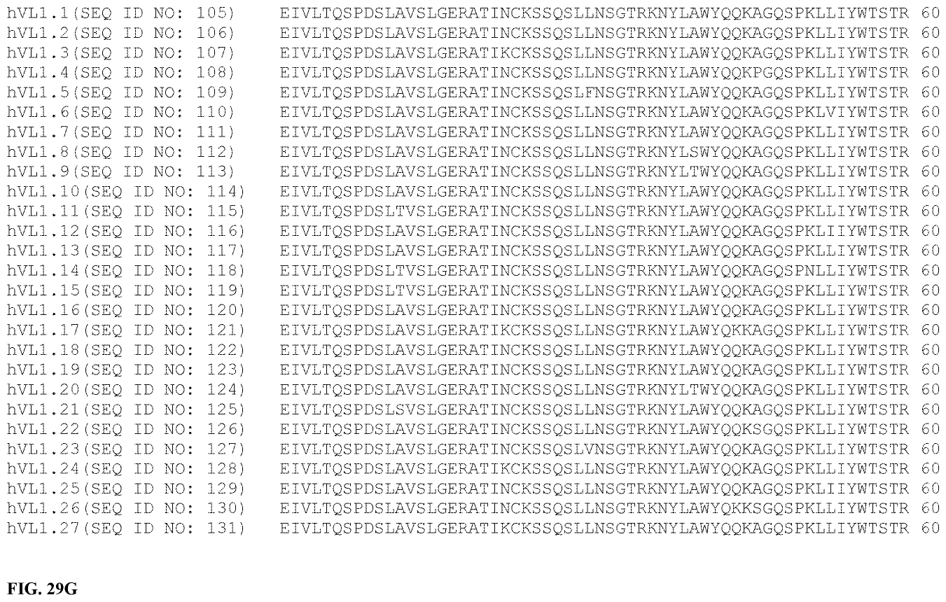

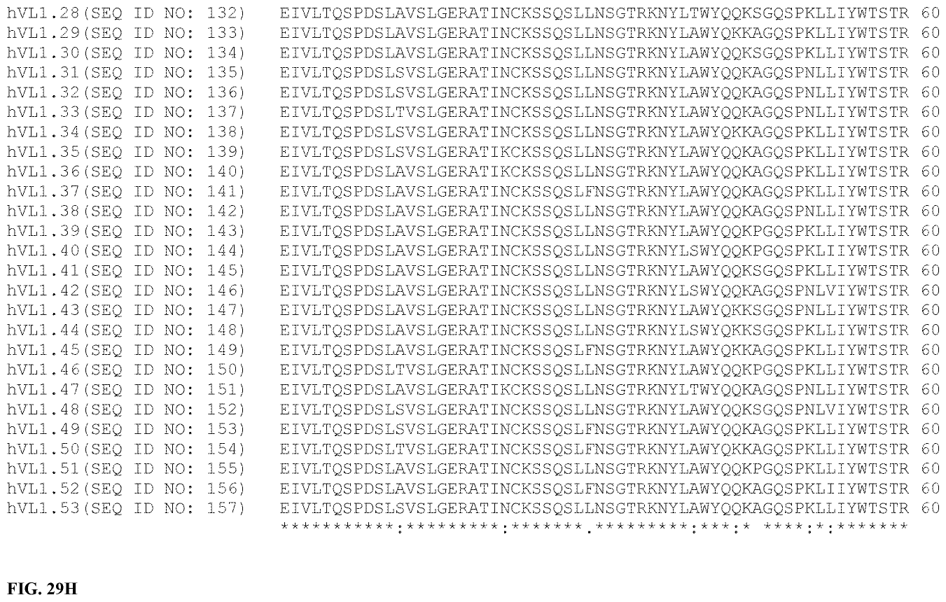

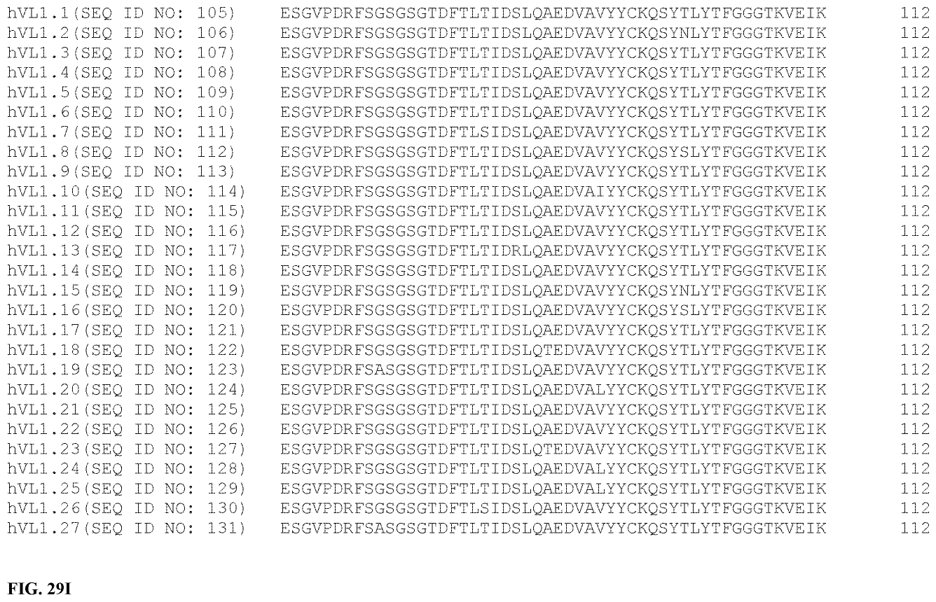

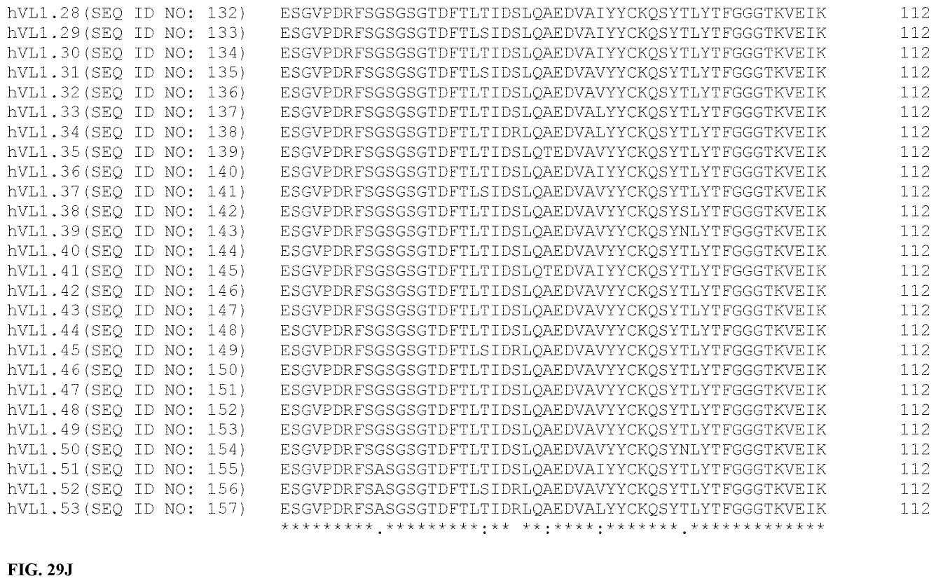

A sequence alignment of humanized variants hVH-1.1 through hVH-1.78 is depicted in FIGS. 29A-F, and hVL-1.1 through hVL-1.53 in FIGS. 29G-J.

DNA coding for the amino acid sequence of the variants were synthesized and cloned into a mammalian transient expression plasmid. Variants were expressed using a CHO based transient expression system and the resulting antibody containing cell culture supernatants were clarified by centrifugation and filtration. Variants were purified from cell culture supernatants via affinity chromatography. Purified antibodies were buffer exchanged into phosphate buffered saline solution. The purity of the resulting antibodies was determined to be >95%, as judged by reducing and denaturing SDS-PAGE gels. Antibody concentration was determined by measuring absorbance at 280 nm.

Binding assays were performed as follows. Antibody variants were immobilized using anti-human Fc onto the surface of a series of biosensors at 0.15 g/ml. Antigen, at 100 nM, was passed over the surface to generate a binding response. Binding data for the antibody:antigen interactions were collected at 25° C. on the biosensors. Results for select antibody variants are shown in Table 14. In Table 14, X=610 represents captured antigen value (nM) at the beginning of the dissociation phase (measured at 610 seconds after the biosensor was initially contacted with antigen) and X=1495 represents captured antigen value (nM) at the end of the dissociation phase (measured at 1495 seconds after the biosensor was initially contacted with antigen). In Table 14, antibodies with an X=610 value greater than 0.2 and a % dissociation less than 10% are shown in bold.

TABLE 14
Binding Assays

| | Capture | X = | X = | % |
|---|---|---|---|---|
| Antibody | Level | 610 | 1495 | Diss |
| h77A-1 | 0.656 | 0.2740 | 0.2531 | 7.63 |
| h77A-1.1 | 0.645 | 0.2729 | 0.2623 | 3.88 |
| h77A-1.2 | 0.700 | 0.0496 | 0.0505 | −1.81 |
| h77A-1.3 | 0.735 | 0.2546 | 0.1816 | 28.67 |
| h77A-1.4 | 0.705 | 0.2583 | 0.1763 | 31.75 |
| h77A-1.5 | 0.706 | 0.2569 | 0.1919 | 25.30 |
| h77A-1.6 | 0.684 | 0.2901 | 0.2710 | 6.58 |
| h77A-1.7 | 0.710 | 0.2182 | 0.1524 | 30.16 |
| h77A-1.8 | 0.742 | 0.3331 | 0.3044 | 8.62 |
| h77A-1.9 | 0.692 | 0.2778 | 0.2269 | 18.32 |
| h77A-1.10 | 0.723 | 0.3162 | 0.2885 | 8.76 |
| h77A-1.11 | 0.713 | 0.3006 | 0.2684 | 10.71 |
| h77A-1.12 | 0.701 | 0.2997 | 0.2855 | 4.74 |
| h77A-1.13 | 0.721 | 0.1133 | 0.0981 | 13.42 |
| h77A-1.14 | 0.701 | 0.3123 | 0.2877 | 7.88 |
| h77A-1.15 | 0.721 | 0.2921 | 0.1956 | 33.04 |
| h77A-1.16 | 0.713 | 0.3048 | 0.2949 | 3.25 |
| h77A-1.17 | 0.741 | 0.3267 | 0.3037 | 7.04 |
| h77A-1.18 | 0.688 | 0.2841 | 0.2638 | 7.15 |
| h77A-1.19 | 0.705 | 0.3138 | 0.2864 | 8.73 |
| h77A-1.20 | 0.712 | 0.3111 | 0.2736 | 12.05 |
| h77A-1.21 | 0.691 | 0.2977 | 0.2679 | 10.01 |
| h77A-1.22 | 0.747 | 0.3309 | 0.3094 | 6.50 |
| h77A-1.23 | 0.758 | 0.3217 | 0.2444 | 24.03 |
| h77A-1.24 | 0.604 | 0.1601 | 0.1063 | 33.60 |
| h77A-1.25 | 0.726 | 0.2198 | 0.1687 | 23.25 |
| h77A-1.26 | 0.705 | 0.2961 | 0.261 | 11.85 |
| h77A-1.27 | 0.698 | 0.2922 | 0.2725 | 6.74 |
| h77A-1.28 | 0.696 | 0.273 | 0.2217 | 18.79 |
| h77A-1.29 | 1.032 | 0.3705 | 0.2656 | 28.31 |
| h77A-1.30 | 0.692 | 0.2843 | 0.2698 | 5.10 |
| h77A-1.31 | 0.688 | 0.1108 | 0.1147 | −3.52 |
| h77A-1.32 | 0.651 | 0.0256 | 0.0462 | −80.47 |
| h77A-1.33 | 0.700 | 0.2359 | 0.1873 | 20.60 |
| h77A-1.35 | 0.707 | 0.1665 | 0.1592 | 4.38 |
| h77A-1.36 | 0.702 | 0.1868 | 0.1738 | 6.96 |
| h77A-1.37 | 0.659 | 0.2385 | 0.1842 | 22.77 |
| h77A-1.38 | 0.546 | 0.0543 | 0.0486 | 10.50 |
| h77A-1.39 | 0.701 | 0.2269 | 0.1793 | 20.98 |
| h77A-1.40 | 0.679 | 0.0918 | 0.0809 | 11.87 |
| h77A-1.41 | 0.713 | 0.2754 | 0.1811 | 34.24 |
| h77A-1.42 | 0.721 | 0.2155 | 0.1433 | 33.50 |
| h77A-1.43 | 0.678 | 0.2466 | 0.1946 | 21.09 |
| h77A-1.45 | 0.698 | 0.2643 | 0.1748 | 33.86 |
| h77A-1.46 | 0.689 | 0.275 | 0.2579 | 6.22 |
| h77A-1.47 | 0.740 | 0.0917 | 0.0792 | 13.63 |
| h77A-1.48 | 0.666 | 0.1875 | 0.1229 | 34.45 |
| h77A-1.50 | 0.644 | 0.2639 | 0.2342 | 11.25 |
| h77A-1.52 | 0.681 | 0.0062 | 0.0133 | −114.52 |
| h77A-1.53 | 0.643 | 0.1222 | 0.1086 | 11.13 |
| h77A-1.54 | 0.665 | 0.0068 | 0.0096 | −41.18 |
| h77A-1.55 | 0.619 | 0.1545 | 0.1133 | 26.67 |
| h77A-1.57 | 0.646 | 0.1704 | 0.1262 | 25.94 |
| h77A-1.59 | 0.652 | 0.2509 | 0.2263 | 9.80 |
| h77A-1.61 | 0.639 | 0.2539 | 0.237 | 6.66 |
| h77A-1.62 | 0.579 | 0.2199 | 0.1928 | 12.32 |
| h77A-1.63 | 0.563 | 0.2257 | 0.1937 | 14.18 |
| h77A-1.64 | 0.572 | 0.2086 | 0.1648 | 21.00 |
| h77A-1.65 | 0.612 | 0.2513 | 0.1997 | 20.53 |
| h77A-1.66 | 0.665 | 0.23 | 0.1561 | 32.13 |
| h77A-1.70 | 0.674 | 0.2761 | 0.2441 | 11.59 |
| h77A-1.71 | 0.676 | 0.2899 | 0.2669 | 7.93 |
| h77A-1.72 | 0.636 | 0.1701 | 0.1165 | 31.51 |
| h77A-1.74 | 0.665 | 0.2934 | 0.2668 | 9.07 |
| h77A-1.75 | 0.682 | 0.3053 | 0.289 | 5.34 |
| h77A-1.76 | 0.670 | 0.2767 | 0.2431 | 12.14 |
| h77A-1.77 | 0.689 | 0.2285 | 0.1542 | 32.52 |
| h77A-1.79 | 0.683 | 0.0131 | 0.018 | −37.40 |
| h77A-1.80 | 0.630 | 0.0181 | 0.0197 | −8.84 |
| h77A-1.81 | 0.653 | 0.2644 | 0.1847 | 30.14 |
| h77A-1.85 | 0.616 | 0.2523 | 0.2117 | 16.09 |
| h77A-1.86 | 0.522 | 0.1908 | 0.1639 | 14.10 |
| h77A-1.91 | 0.637 | 0.2541 | 0.1762 | 30.66 |
| h77A-1.92 | 0.620 | 0.2346 | 0.1586 | 32.40 |
| h77A-1.94 | 0.660 | 0.2978 | 0.2822 | 5.24 |
| h77A-1.96 | 0.749 | 0.3288 | 0.2669 | 18.83 |
| h77A-1.97 | 0.645 | 0.1793 | 0.1305 | 27.22 |
| h77A-1.98 | 0.675 | 0.2325 | 0.1522 | 34.54 |

Further kinetics assays were conducted for select antibody variants. Assays were performed as follows. Antibody variants were immobilized using anti-human Fc onto the surface of a series of biosensors. Antigen was passed over the surface to generate a binding response. Binding data for the antibody: antigen interactions were collected at 25° C. on the biosensors. A dilution series of the antigen was used in the association step, in order to fit results globally and get the best values for ka, kd, and KD. The response data for the binding of antigen to the surface immobilized antibody were fitted to a 1:1 binding model. Kinetic parameters are summarized in Table 1S.

TABLE 15
Binding Kinetics

| | Capture | | | | K_{D} | | | Rₘₐₓ |
|---|---|---|---|---|---|---|---|---|
| | level | kₐ | k_{d} | K_{D} | Steady | | | at 33.3 |
| Antibody | (nm) | (m⁻¹s⁻¹) | (s⁻¹) | (nM) | State | R² | X² | nM |
| h77A-1 | 0.726 | 5.47E+04 | 1.31E−04 | 2.39 | 1.20E−09 | 0.9981 | 0.2056 | 0.2942 |
| h77A-1.1 | 0.68 | 8.85E+04 | 6.11E−05 | 0.69 | 3.20E−11 | 0.9969 | 0.2685 | 0.2271 |
| h77A-1.6 | 0.678 | 5.42E+04 | 1.14E−04 | 2.11 | 1.80E−09 | 0.9983 | 0.1792 | 0.2735 |
| h77A-1.8 | 0.7 | 5.48E+04 | 7.42E−05 | 1.35 | 3.10E−10 | 0.9984 | 0.188 | 0.2984 |
| h77A-1.10 | 0.677 | 5.84E+04 | 1.45E−04 | 2.48 | 2.60E−09 | 0.9981 | 0.1872 | 0.2679 |
| h77A-1.12 | 0.784 | 5.17E+04 | 7.37E−05 | 1.43 | 1.10E−09 | 0.9984 | 0.2097 | 0.3106 |
| h77A-1.14 | 0.704 | 6.31E+04 | 1.24E−04 | 1.97 | 7.70E−10 | 0.9978 | 0.1844 | 0.2516 |
| h77A-1.16 | 0.75 | 5.40E+04 | 6.84E−05 | 1.27 | 1.30E−09 | 0.9986 | 0.1422 | 0.2774 |
| h77A-1.17 | 0.686 | 6.14E+04 | 9.24E−05 | 1.51 | 1.30E−09 | 0.9972 | 0.3139 | 0.2752 |
| h77A-1.18 | 0.639 | 6.32E+04 | 9.10E−05 | 1.44 | 8.10E−10 | 0.9974 | 0.2789 | 0.2549 |
| h77A-1.19 | 0.71 | 8.85E+04 | 1.11E−04 | 1.26 | 1.20E−09 | 0.9983 | 0.2233 | 0.2722 |
| h77A-1.22 | 0.779 | 6.69E+04 | 7.64E−05 | 1.14 | 8.10E−10 | 0.9985 | 0.2631 | 0.3227 |
| h77A-1.27 | 0.746 | 7.64E+04 | 1.08E−04 | 1.41 | 8.90E−10 | 0.9986 | 0.2141 | 0.295 |
| h77A-1.30 | 0.744 | 5.67E+04 | 1.09E−04 | 1.93 | 8.60E−10 | 0.9984 | 0.1831 | 0.2812 |
| h77A-1.46 | 0.754 | 5.48E+04 | 1.23E−04 | 2.24 | 1.20E−09 | 0.9987 | 0.1571 | 0.2816 |
| h77A-1.59 | 0.708 | 4.08E+04 | 1.20E−04 | 2.93 | 7.40E−10 | 0.9987 | 0.0944 | 0.2583 |
| h77A-1.61 | 0.65 | 4.21E+04 | 1.23E−04 | 2.91 | 2.10E−09 | 0.9984 | 0.1083 | 0.2361 |
| h77A-1.71 | 0.747 | 4.90E+04 | 1.23E−04 | 2.51 | 1.30E−09 | 0.9988 | 0.1362 | 0.2911 |
| h77A-1.74 | 0.699 | 7.78E+04 | 1.25E−04 | 1.61 | 1.70E−09 | 0.9979 | 0.2514 | 0.2562 |
| h77A-1.75 | 0.748 | 8.81E+04 | 2.43E−05 | 0.28 | 2.30E−11 | 0.9972 | 0.4938 | 0.2906 |
| h77A-1.94 | 0.654 | 5.99E+04 | 1.11E−04 | 1.85 | 1.30E−09 | 0.9976 | 0.1953 | 0.2325 |

### Example 17. —77A IgG1 Antibodies

This Example describes the generation of full-length antibodies including h77A-1, an IgG1 heavy chain constant region (or variant thereof), and a kappa light chain constant region.

h77A-1, including the combination of hVH-1 and hVL-1 (as described in Example 12) was fused to the constant region of the IgG1 heavy chain G1m(3) and light chain Km(3) allotypes (G1m3 variant). In addition to the wild type G1m(3)-Km(3) constant regions, Fc variants were created that included the IgG1 G236A mutation (GA variant) to increase binding to FcγR2a, and the IgG1 G236A/A330 μL/I332E mutations (GAALIE variant) to increase binding to FcγR3a. Antibodies were expressed in 293 or CHO cells.

Antibodies are described in Table 16, and amino acid sequences of the antibodies are depicted in Table 17.

TABLE 16
h77A-1 Antibodies

| | | HC SEQ | | LC SEQ |
|---|---|---|---|---|
| Antibody Name | HC Name | ID NO | LC Name | ID NO |
| h77A-1-G1m3 | hVH-1- | 242 | hVL-1-Km3 | 249 |
| (“G1m3”) | G1m3 | | | |
| h77A-1-G1m3-GA | hVH-1- | 243 | hVL-1-Km3 | 249 |
| (“G1m3-GA”) | G1m3-GA | | | |
| h77A-1-G1m3- | hVH-1- | 244 | hVL-1-Km3 | 249 |
| GAALIE | G1m3- | | | |
| (“G1m3-GAALIE”) | GAALIE | | | |

TABLE 17
Amino Acid Sequences

| HC/LC | SEQ | |
|---|---|---|
| Name | ID NO | Amino Acid Sequence |
| hVH-1- | 242 | QIQLVQSGAEVKKPGASVKVSCKASGYTFTNYGMNWVRQAPGQGLEWMG |
| G1m3 | | WINTYTGEPTYADDFKGRFTFTTDTSTSTAYMELRSLRSDDTAVYFCAR |
| | | YDHAMDYWGQGTLVTVSSASTKGPSVFPLAPSSKSTSGGTAALGCLVKD |
| | | YFPEPVTVSWNSGALTSGVHTFPAVLQSSGLYSLSSVVTVPSSSLGTQT |
| | | YICNVNHKPSNTKVDKRVEPKSCDKTHTCPPCPAPELLGGPSVFLFPPK |
| | | PKDTLMISRTPEVTCVVVDVSHEDPEVKFNWYVDGVEVHNAKTKPREEQ |
| | | YNSTYRVVSVLTVLHQDWLNGKEYKCKVSNKALPAPIEKTISKAKGQPR |
| | | EPQVYTLPPSREEMTKNQVSLTCLVKGFYPSDIAVEWESNGQPENNYKT |
| | | TPPVLDSDGSFFLYSKLTVDKSRWQQGNVESCSVMHEALHNHYTQKSLS |
| | | LSPGK |
| hVH-1- | 243 | QIQLVQSGAEVKKPGASVKVSCKASGYTFTNYGMNWVRQAPGQGLEWMG |
| G1m3- | | WINTYTGEPTYADDFKGRFTFTTDTSTSTAYMELRSLRSDDTAVYFCAR |
| GA | | YDHAMDYWGQGTLVTVSSASTKGPSVFPLAPSSKSTSGGTAALGCLVKD |
| | | YFPEPVTVSWNSGALTSGVHTFPAVLQSSGLYSLSSVVTVPSSSLGTQT |
| | | YICNVNHKPSNTKVDKRVEPKSCDKTHTCPPCPAPELLAGPSVFLFPPK |
| | | PKDTLMISRTPEVTCVVVDVSHEDPEVKFNWYVDGVEVHNAKTKPREEQ |
| | | YNSTYRVVSVLTVLHQDWLNGKEYKCKVSNKALPAPIEKTISKAKGQPR |
| | | EPQVYTLPPSREEMTKNQVSLTCLVKGFYPSDIAVEWESNGQPENNYKT |
| | | TPPVLDSDGSFFLYSKLTVDKSRWQQGNVFSCSVMHEALHNHYTQKSLS |
| | | LSPGK |
| hVH-1- | 244 | QIQLVQSGAEVKKPGASVKVSCKASGYTFTNYGMNWVRQAPGQGLEWMG |
| G1m3- | | WINTYTGEPTYADDFKGRFTFTTDTSTSTAYMELRSLRSDDTAVYFCAR |
| GAALIE | | YDHAMDYWGQGTLVTVSSASTKGPSVFPLAPSSKSTSGGTAALGCLVKD |
| | | YFPEPVTVSWNSGALTSGVHTFPAVLQSSGLYSLSSVVTVPSSSLGTQT |
| | | YICNVNHKPSNTKVDKRVEPKSCDKTHTCPPCPAPELLAGPSVFLFPPK |
| | | PKDTLMISRTPEVTCVVVDVSHEDPEVKFNWYVDGVEVHNAKTKPREEQ |
| | | YNSTYRVVSVLTVLHQDWLNGKEYKCKVSNKALPLPEEKTISKAKGQPR |
| | | EPQVYTLPPSREEMTKNQVSLTCLVKGFYPSDIAVEWESNGQPENNYKT |
| | | TPPVLDSDGSFFLYSKLTVDKSRWQQGNVFSCSVMHEALHNHYTQKSLS |
| | | LSPGK |
| hVL-1- | 249 | EIVLTQSPDSLAVSLGERATINCKSSQSLLNSGTRKNYLAWYQQKAGQS |
| Km3 | | PKLLIYWTSTRESGVPDRFSGSGSGTDFTLTIDSLQAEDVAVYYCKQSY |
| | | TLYTFGGGTKVEIKRTVAAPSVFIFPPSDEQLKSGTASVVCLLNNFYPR |
| | | EAKVQWKVDNALQSGNSQESVTEQDSKDSTYSLSSTLTLSKADYEKHKV |
| | | YACEVTHQGLSSPVTKSFNRGEC |

### Example 18. —77A High Molecular Weight Immune Complex Formation

This Example describes the formation of high molecular weight immune complexes upon binding of 77A variants to HSP70.

Analytical size exclusion chromatography (SEC) was used to examine the co-localization of h77A-1-G1m3 (“G1m3”; as described in Example 17), and HSP70. To enable detection of each protein, h77A-1-G1m3 was labeled with CF647 dye (“G1m3-CF647”) and HSP70 was fused to GFP (“HSP70-GFP”).

Unless indicated otherwise, a Superose 6 Increase 10/300 GL column was used for all experiments. The retention time of the column was calibrated using a custom protein standard to determine void volume, milestone molecular weights, and evaluate the linearity of elution times. The mobile phase was PBS, pH 7.4 and run at a flow rate of 0.75 ml/min. The pressure maximum was set to 650 psi, with a run time of 35 min. Detection of complexes was performed with the onboard PDA detector set to detect absorbance in the range of 180-800 nm. Fluorescence for GFP was detected using an excitation of 488 nm and emission at 510 nm. Fluorescence for CF647 was detected using excitation at 650 nm and emission at 665 nm. Analysis was performed using the LabSolutions analysis software to obtain peak area, peak height, retention time, and all detected wavelengths. Retention times were used to predicted approximate molecular weights. Differences in kinetics, complex formation, and antibody binding interactions were determined to be negligible between the recombinant wild-type and GFP-fusion form of HSP70.

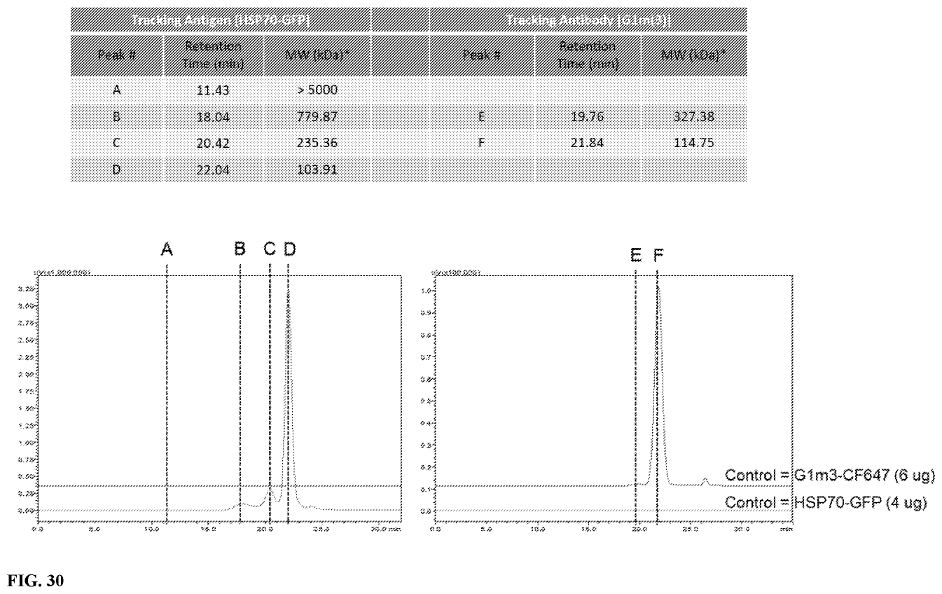

To set a baseline retention time, and demonstrate monomeric conformations of individual proteins, individual samples of G1m3-CF647 and HSP70-GFP were run through the SEC column. Detection of peaks was measured using the fluorescence from GFP for HSP70, or from CF647 for the antibody. Results are depicted in FIG. 30.

G1m3-CF647 was then incubated with or without HSP70-GFP and run through the SEC column. Each sample was run using an injection volume of 50 microliters containing a mass of 3-30 micrograms of protein. To determine if both antibody and HSP70 were co-localizing, initially a single complexing reaction was performed using G1m3-CF647 and HSP70-GFP at a molar ratio of 1:1. The same sample was run twice, first detecting fluorescence from GFP, and then again from CF647.

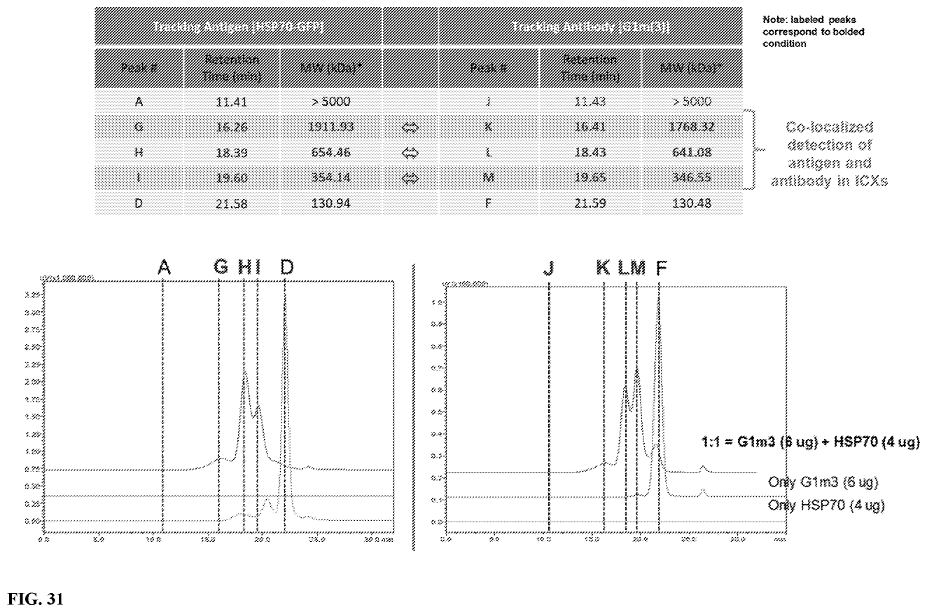

The results of the SEC analysis show that both G1m3-CF647 and HSP70-GFP co-localized to high molecular weight complexes (FIG. 31). The resulting pattern of peaks indicated that rather than a broad range of sizes, discreet and ordered peaks reproducibly formed. Observed retention times were reproducible, suggesting that the higher molecular weight complexes are stable.

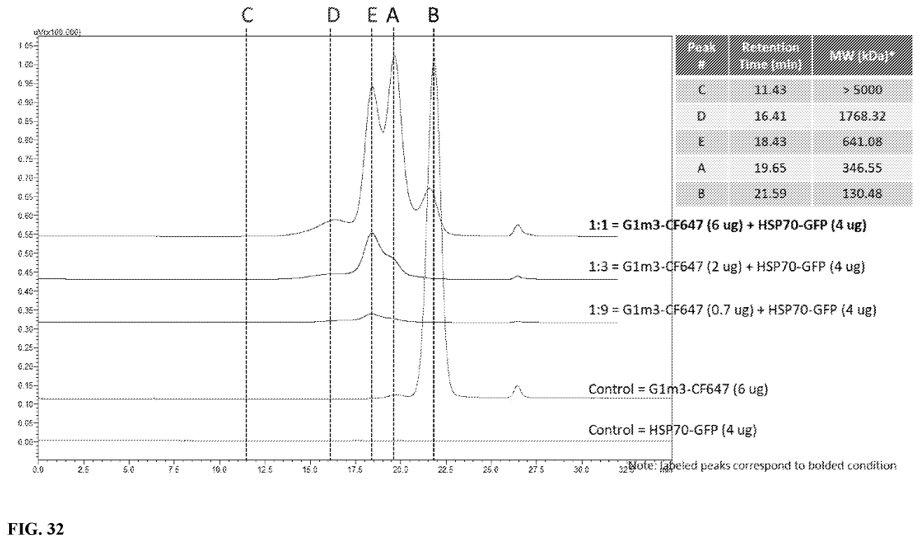

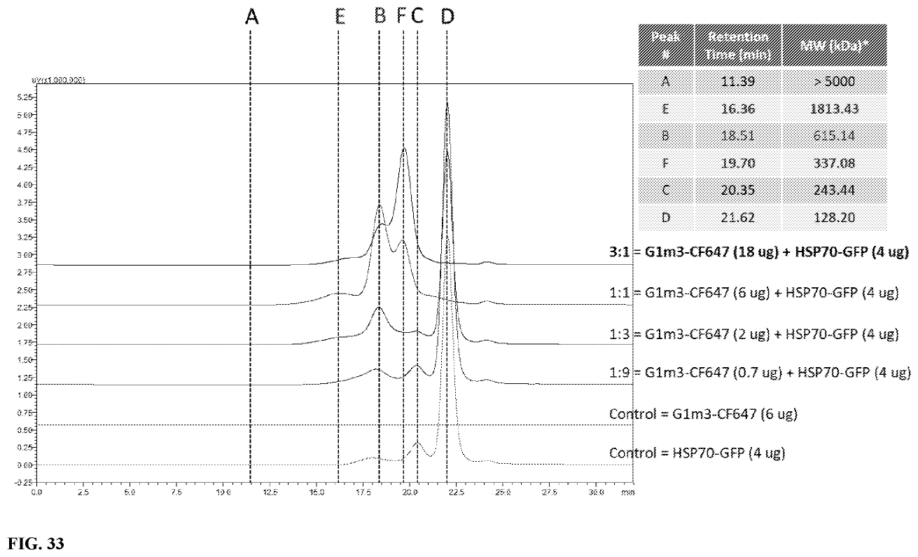

Additionally, complexing reactions were performed at several different molar ratios of G1m3-CF647 to HSP70-GFP (1:9, 1:3, 1:1, and 3:1). These samples were then run individually first to detect fluorescence from CF647 (FIG. 32), and then again to detect fluorescence from GFP (FIG. 33). The formation of peaks was dose-responsive when tracking either HSP70-GFP (FIG. 32) or G1m3-CF647 (FIG. 33).

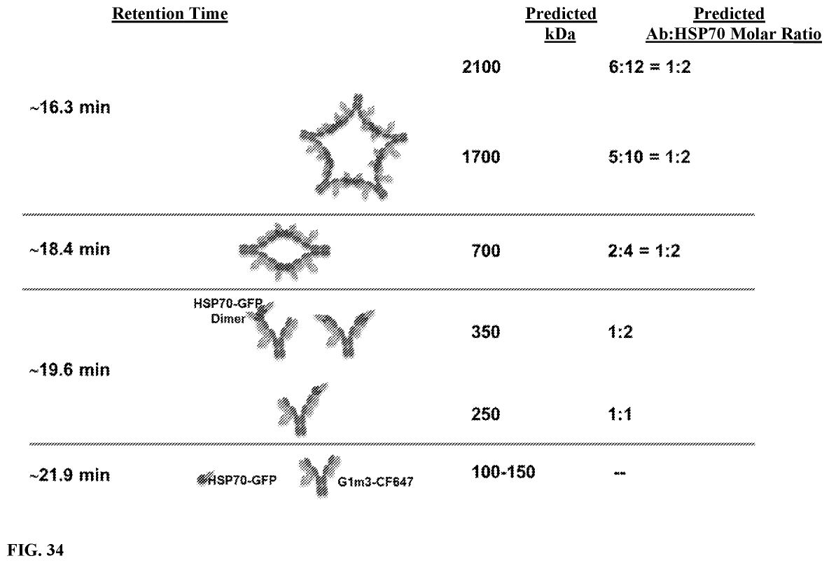

The composition of the high molecular weight immune complexes was predicted based on the estimated molecular weights of each peak and the molecular weights of G1m3-CF647 and HSP70-GFP. Results are shown in FIG. 34.

In summary, these results demonstrate that 77A variants, e.g., h77A-1-G1m3, are able to robustly and reproducibly form defined high molecular weight immune complexes upon binding to HSP70.

### Example 19. —77A:HSP70 Complex Fc Receptor Binding

This Example describes Fc receptor binding by 77 antibody and HSP70 complexes.

As described in Example 18 above, 77A antibody variants are able to form high molecular weight immune complexes upon binding to HSP70. To examine engagement of Fc receptors by these complexes, as compared to the antibody alone, 77A antibody variants were evaluated in ELISA FcγR binding assays.

Briefly, ELISA plate wells were coated overnight using Fc receptor at 0.3 ug/mL in 50 mM carbonate-bicarbonate buffer. The following day the plate was washed three times using 1× PBS-T buffer and blocked for 1 hour at room temperature using SuperBlock. Dilutions of antibody were mixed with a constant amount of HSP70 and allowed to complex for 1 hour at room temperature. The plate was washed again as before, then the dilution series of either pre-complexed antibody/HSP70 or antibody alone were added to the wells. The plate was incubated for 1 hour at room temperature. The plate was again washed as before, and goat Fab fragment anti-human conjugated to HRP was diluted 1:3000 and added to the wells for 15 minutes at room temperature. The plate was again washed, and TMB added to each well. The substrate was then stopped using Stop Solution of sulfuric acid. The absorbances were then read at 450 nm, 540 nm, 900 nm, and 999 nm.

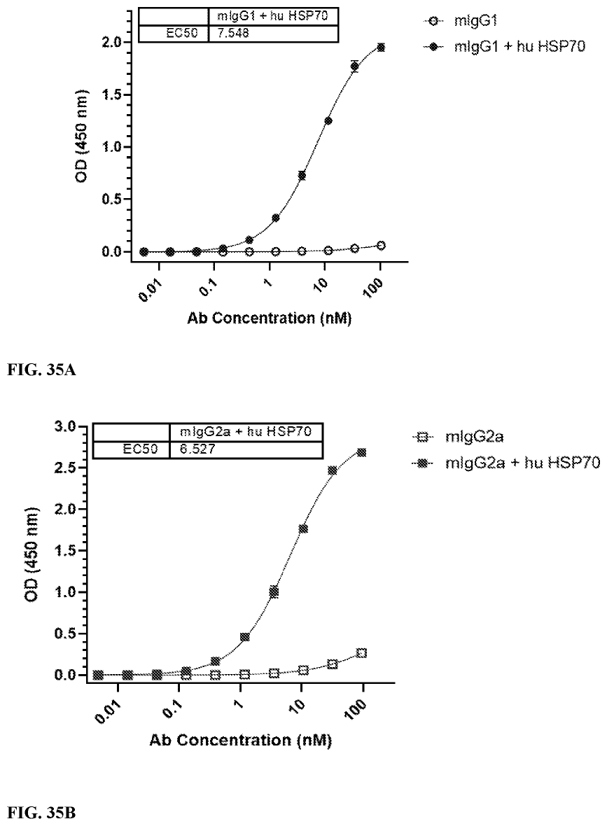

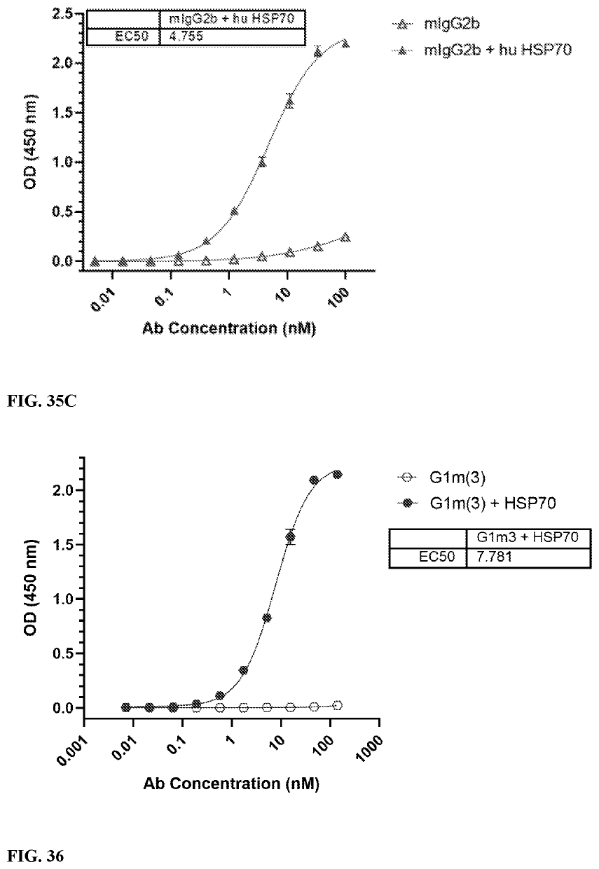

Results are shown in FIGS. 35 and 36. Antibodies tested included murine 77A in murine IgG1 (FIG. 35A), IgG2a (FIG. 35B), and IgG2b (FIG. 35C) formats, as well as humanized h77A-1-G1m3 (as described in Example 17; FIG. 36). In contrast to antibody alone, antibody:HSP70 complexes bound more strongly to Fc receptors. It is contemplated that the presence of multiple antibodies in the high molecular weight immune complexes results in increased avidity. Binding to FcγR was not dependent on the Fc isotype. Binding activity was observed for both the murine and humanized antibodies.

Fc receptor binding results were confirmed in BLI assays using antibody alone or in complex with HSP70. For BLI-based assays, antibodies and reagents were diluted out onto microwell plates. For the formation of complex, antibody and HSP70 were mixed together at a molar ratio of 1:1 and allowed to complex for 60 minutes at 30° C. with single-orbital shaking at 1000 rpm. For antibody alone, the same concentration of antibody was used without HSP70. After incubation, BLI dip-and-read sensors were loaded with 10 μg/mL of relevant human or mouse Fc receptor. The newly loaded sensors were then dipped into the microwells containing either the antibody alone or the microwells containing the newly formed immune complexes for 300 seconds. The sensors were then dipped into buffer alone to measure dissociation from the Fc receptor for 720 seconds.

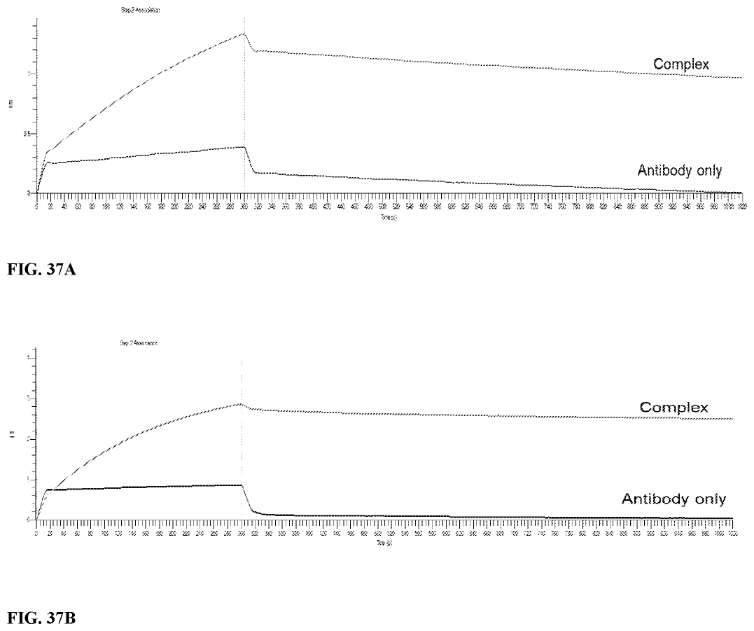

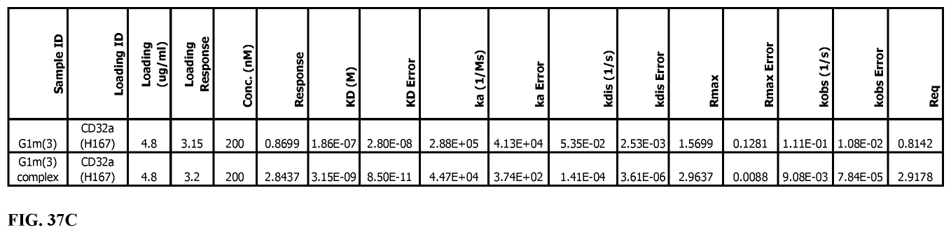

Results for murine 77A and humanized h77A-1-G1m3 are shown in FIG. 37. Again, for murine (FIG. 37A) and humanized (FIG. 37B) 77A, stronger binding was observed for the complex. Analysis was performed using the Octet Analysis Studio software to determine summary kinetics for h77A-1-G1m3. Results are depicted in FIG. 37C, and show enhanced magnitude of binding response, and an improved off-rate, for the antibody:HSP70 complex relative to antibody alone.

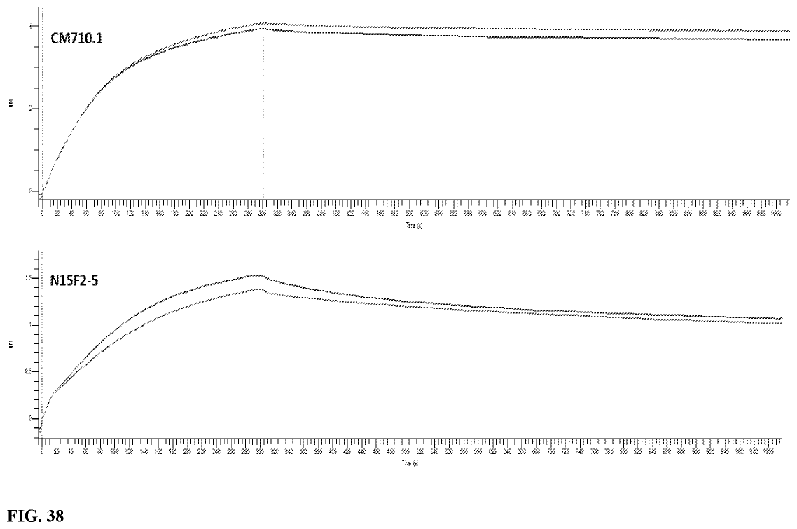

Results for commercially available anti-HSP70 antibodies CM710.1 and N15F2-5 are shown in FIG. 38. For CM710.1 and N15F2-5, incubation with HSP70 did not result in a substantial difference in Fc receptor binding, suggesting these antibodies do not form high molecular weight immune complexes upon binding to HSP70.

Analytical size exclusion chromatography was used to further explore the capability of commercially available anti-HSP70 antibodies CM710.1 and N15F2-5 to form high molecular weight immune complexes.

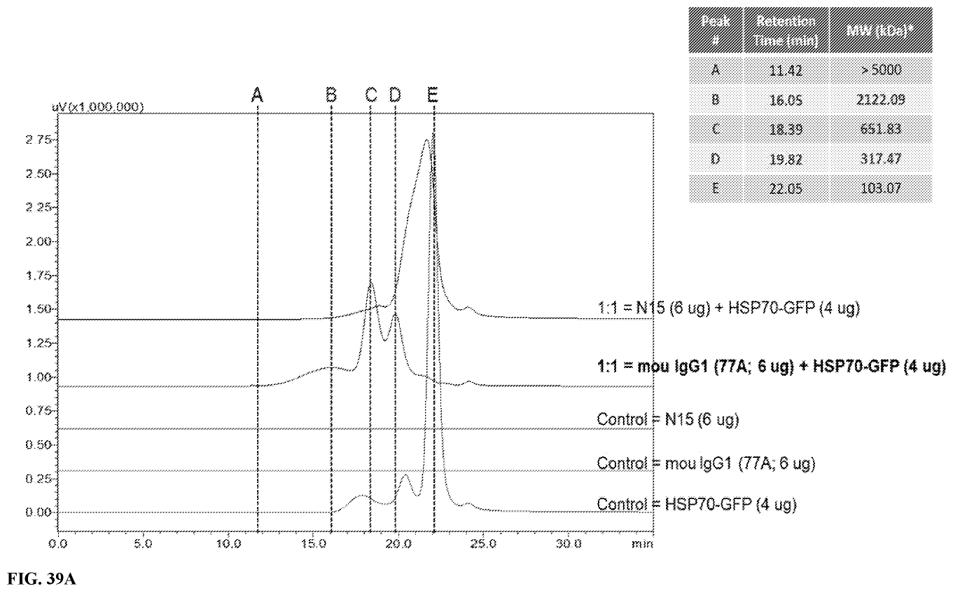

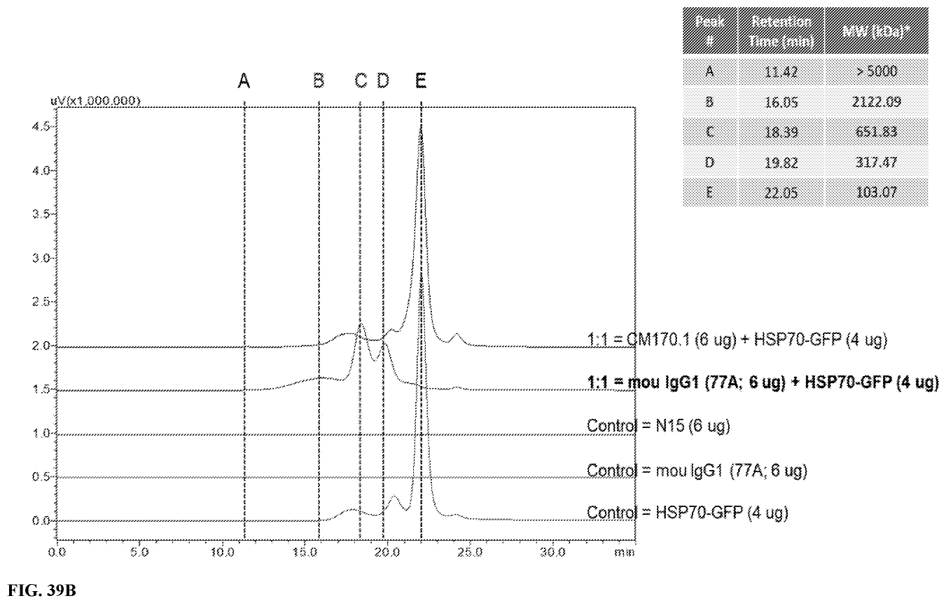

Experiments were conducted substantially as described in Example 18. Briefly, antibodies (either murine 77A in a murine IgG1 format, CM710.1, or N15F2-5) were labeled with CF647 dye and HSP70 was fused to GFP (“HSP70-GFP”). Detection of peaks was measured using the fluorescence from GFP for HSP70, or from CF647 for the antibody. A complexing reaction was performed using antibody and HSP70-GFP at a molar ratio of 1:1. The same sample was then run on the SEC column twice, first detecting fluorescence from GFP, and then again from CF647. Results for murine 77A and N15F2-5 are shown in FIG. 39A and results for murine 77A and CM710.1 are shown in FIG. 39B. As depicted, peaks corresponding to high molecular weight immune complexes were observed for murine 77A but not CM710.1 or N15F2-5.

Together, these results show that 77A antibody variants form high molecular weight immune complexes upon binding to HSP70, and these high molecular weight immune complexes bind more strongly to Fc receptors than antibody alone. The ability to form high molecular weight immune complexes is not simply a function of HSP70 binding, as commercially available anti-HSP70 antibodies CM710.1 or N15F2-5 do not appear to form high molecular weight immune complexes. Moreover, complex formation occurs independently of antibody isotype, and for both murine and humanized 77A antibodies.

### Example 20. —77A:HSP70 Complex Cellular Uptake

This Example describes cellular uptake of 77 antibody and HSP70 complexes.

As described in Example 18 above, 77A antibody variants are able to form high molecular weight immune complexes upon binding to HSP70. A FACS-based assay was developed to determine the extent that cells are able to internalize these complexes.

In brief, complexes were first formed by incubating a fixed amount of HSP70-GFP with varying amounts of antibody in PBS+1% BSA buffer in the dark at room temperature for 30 minutes. Where indicated, to enhance the GFP signal in proteolytically active cells, an anti-GFP nanobody labeled with Alexa 488 dye (GFP-booster) was added at 1:1000 ratio to HSP70-GFP to the complexing reaction. Anti-GFP nanobody did not interfere with complex formation.

In parallel, for proteolytically active cells such as APC (antigen presenting cells), cells were pre-treated with 10 μM of commercially available proteasome inhibitor compound MG-132 for three hours at 37° C. When adherent cells were assayed, they were gently lifted using 0.05% Trypsin, and quickly quenched in complete media. Adherent cells were pelleted to remove remaining trypsin, and resuspended at the concentration of 1×10⁶ cells/mL in fresh complete media (containing 10 μM MG-132 if needed). For suspension cells, they were first gently pelleted at 300× g for 5 minutes and resuspended in fresh complete media (containing 10 μM MG-132 if needed) at 1×10⁶ cells/mL. Cells were subsequently aliquoted at 1×10⁵ cells into each well of a 96-well plate, and maintained at 4° C. going forward. To facilitate uptake by 293 cells, which do not endogenously express Fc receptors, cells were transfected in a 10 cm dish using Fugene HD at 3:1 ratio of 8 μg of plasmid DNA encoding the desired Fc receptor. At 48 hours post-transfection, cells were lifted from the plate as described and used in the uptake assay.

Once antibody:HSP70 complexes were formed, cells in plates were pelleted as before, supernatant was removed, and 200 μL of PBS+1% BSA buffer containing the dilutions of complex (and MG-132 if needed) were added to the wells. Cells were gently mixed with the complex, then placed back to 4° C., in the dark, to allow for cells to internalize the complexes for 1 hour. The plate was then spun to pellet cells to remove uninternalized complexes. The supernatants were removed, then cells were resuspended in remaining volume and 1 μL of Zombie Violet viability stain was added to each well. After 15 minutes of staining at 4° C., the wells were washed using PBS, and resuspended in a final volume of 150 μL of PBS and run via FACS. After acquisition was complete, Flowjo software was used for analysis and compensation. After applying compensation and gating out for doublets, cells were gated on live events. The live cell populations were then gated for % GFP-positive events.

In a first assay, antibody:HSP70-GFP complex uptake was assayed in 293 cells transfected with human FcγR2A. Antibodies tested were humanized h77A-1 in a human IgG2 format, and murine 77A in a murine IgG2b^{LALAPG} (“LALAPG”; a reduced Fc receptor binding mutant) format. Results, depicted in FIG. 40, show that complexes formed using the humanized h77A-1 antibody were taken up by 293 cells.

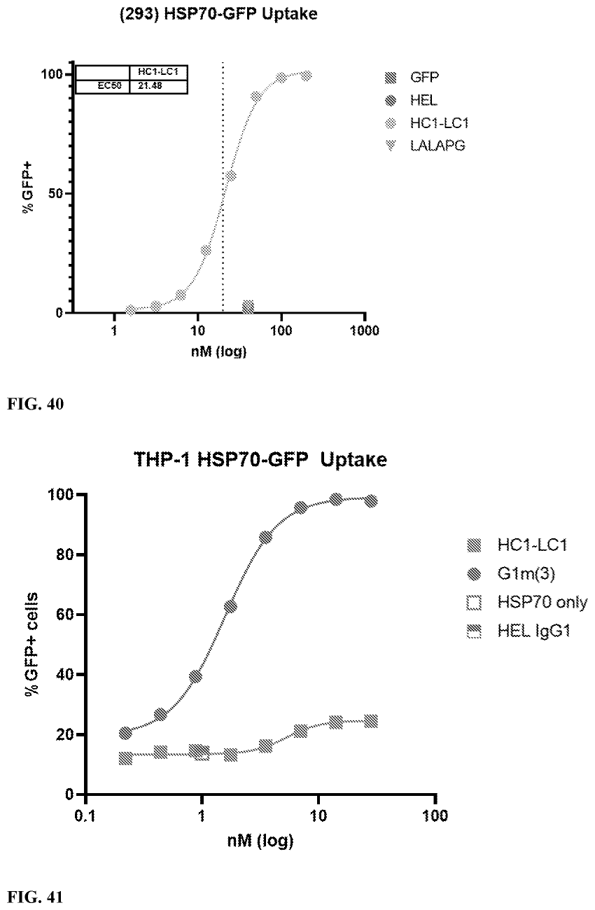

In another assay, antibody:HSP70-GFP complex uptake was assayed in monocytic THP-1 cells. Antibodies tested were humanized h77A-1 in a human IgG2 format, and humanized h77A-1-G1m3. Antibodies were complexed with HSP70-GFP and GFP-booster. Results, depicted in FIG. 41, show a similar trend as seen for 293 cells, with a dose-response uptake, as compared to an unrelated isotype control antibody raised against Hen Egg Lysozyme (anti-HEL-IgG) or as compared to HSP70 alone.

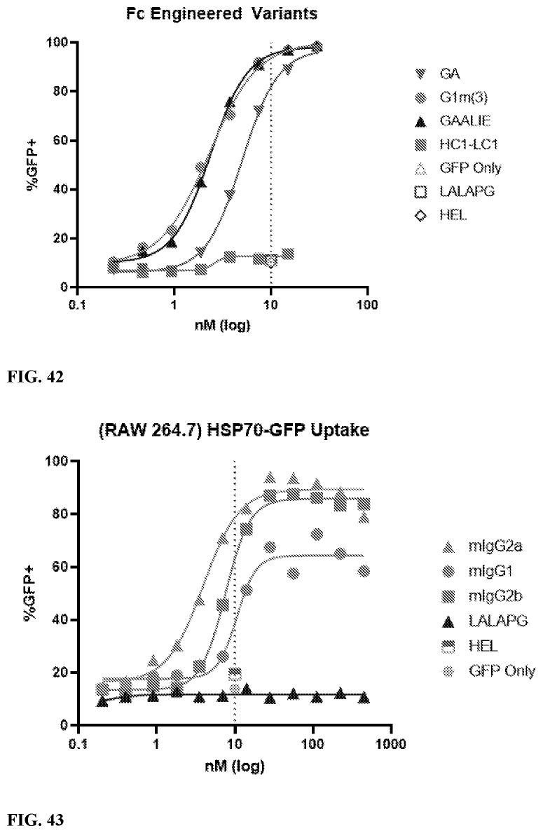

THP-1 cell uptake assays were also performed with h77A-1-G1m3-GA and h77A-1-G1m3-GAALIE (as described in Example 17) in addition to h77A-1 in a human IgG2 format, and h77A-1-G1m3. Results are shown in FIG. 42.

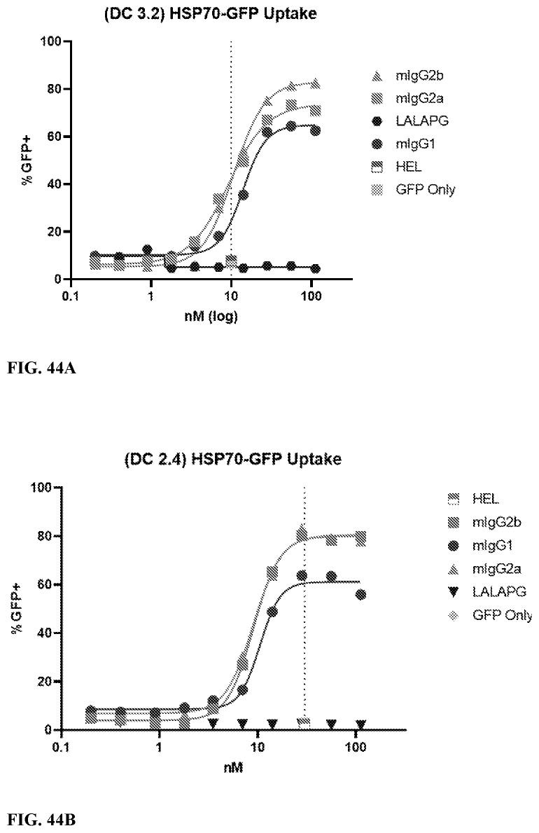

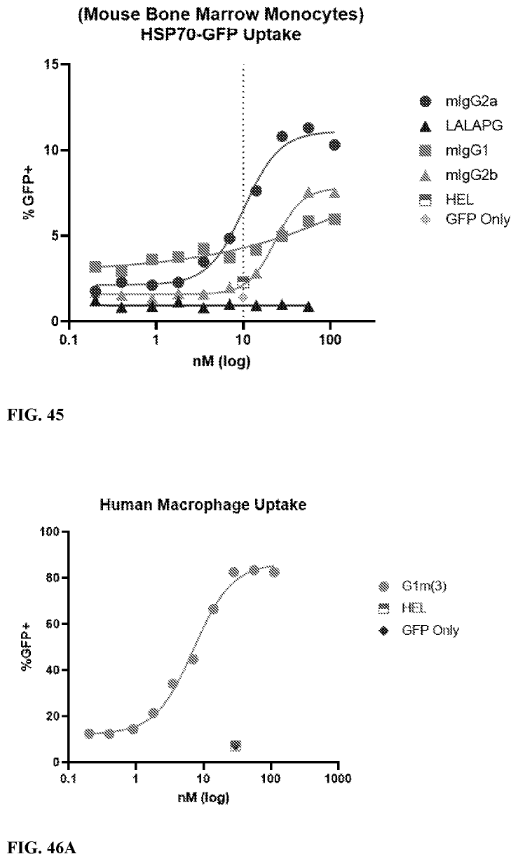

Murine APC-like cell lines were also examined for uptake using murine 77A in murine IgG1, IgG2a, IgG2b, and IgG2b^{LALAPG} formats. Both RAW264.7 macrophage cells (FIG. 43) and DC3.2 and DC2.4 dendritic cells (FIG. 44) showed significant uptake of antibody:HSP70-GFP complexes. Murine bone marrow-derived primary monocytes also showed uptake of complexes above background levels (FIG. 45).

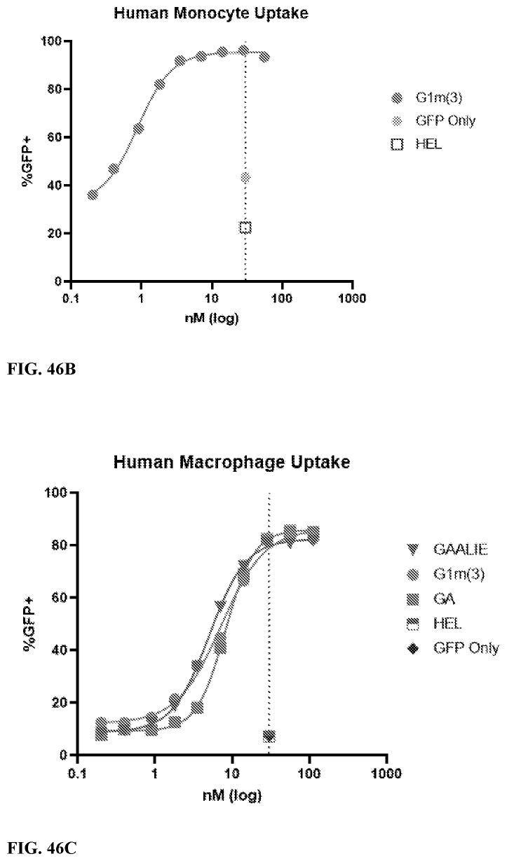

Human primary cells were also examined for uptake using humanized h77A-1-G1m3. Both human macrophage cells (FIG. 46A) and human monocyte cells (FIG. 46B) showed significant uptake of antibody:HSP70-GFP complexes. Human macrophage uptakes assays were also performed with h77A-1-G1m3, h77A-1-G1m3-GA and h77A-1-G1m3-GAALIE. Results are shown in FIG. 46C. In each instance, complexes formed using the humanized h77A-1 antibodies were taken up by cells.

Together, these results show that 77A:HSP70 complexes show a marked enhancement of HSP70 uptake. Cellular uptake was not limited by species. Human and mouse cells showed significant uptake of the complex as compared to HSP70-GFP alone, or with a control antibody. Only the reduced Fc receptor binding LALAPG mutant antibody reproducibly resulted in a background level of cellular uptake.

Consistently, human IgG1 (G1m(3)) isotype appeared to mediate more robust uptake of HSP70-GFP relative to IgG2 isotype. In agreement with the data from human cells, mouse IgG2a, which is functionally similar to human IgG1, had better uptake than the other murine isotypes (IgG1 and IgG2B). In addition, while the Fc variants GA/GAALIE can alter binding engagement of the Fc receptors in monomeric form, the increased avidity of the high molecular weight immune complexes for Fc receptors appears to override any difference in binding observed for the monomeric Fc variants, as comparable cellular uptake was observed for these variants relative to wildtype G1m(3).

Overall, these experiments show that immune complexes can be taken up by APCs and APC-like cells, and that the human IgG1 isotype is superior to IgG2 for mediating this effect.

### Example 21. —77A:HSP70 Complex Cellular Uptake

This Example describes cellular uptake of 77 antibody and HSP70 complexes.

As described in Example 18 above, 77A antibody variants are able to form high molecular weight immune complexes upon binding to HSP70. The commercially available anti-HSP70 antibodies N15F2-5 (Enzo Scientific) and CM170.1 (Klinikum rechts der Isar, Technical University of Munich) bind to HSP70, but are unable to stimulate the formation of high order complexes. The ability of N15F2-5, and CM170.1 to mediate the uptake of HSP70-GFP into murine dendritic and macrophage cells was tested, and compared to murine 77A in murine IgG1, IgG2a, and IgG2b formats. Antibodies were tested in a FACS-based uptake assay designed to detect and quantitate GFP in live cells.

Briefly, immune complexes were generated using recombinant HSP70-GFP (1 mg/ml) and anti-HSP70 antibodies (5 mg/ml). An anti-GFP Nanobody-Alexa-488 (gb2AF488-50, Chromotek) was also added to enhance detection (1:1000 ratio to HSP70-GFP). Anti-Hen Egg Lysozyme (anti-HEL) antibodies were used as matched negative controls. Immune complexes were generated by incubation of proteins for 1 hour at room temperature with agitation prior to addition to murine dendritic cells (DC3.2) or murine macrophages (MH-S) at room temperature for 1 hour. The cells were then washed three times in PBS prior to analysis by flow cytometry. After data acquisition was complete, Flowjo software was used for analysis and compensation. After applying compensation and gating out for doublets, cells were gated on live events. The live cells populations were then gated for % GFP-positive events.

As shown in FIGS. 47 and 48, there was no HSP70-GFP uptake mediated by the negative control anti-HEL isotypes (IgG1-HEL, IgG2a-HEL, IgG2b-HEL). Of the antibodies tested, 77A-IgG2a isoform was the most effective in stimulating HSP-GFP uptake into DC3.2 and MH-S cells (78.3% and 66% live cells were GFP positive). 77A-IgG2b isotype mediated HSP70-GFP uptake to a lesser extent (4.85% and 16% GFP positivity, respectively). The 77A-IgG1 isotype and the 77A-IgG2b^{LALAPG} mutant, with severely diminished FcγR binding ability, were weakly active with MH-S cells only. The commercially available anti-HSP70 N15-2 and CM170.1 antibodies were inactive in these assays, consistent with their inability to simulate immune complex formation upon binding to HSP70.

### Example 22. —Primary Cell Uptake of Engineered Fc Variants of 77A

This Example describes uptake of engineered Fc variants of 77A antibody and HSP70 complexes by human primary immune cells.

As described above, the humanized antibody 77A targets soluble HSP70 and was specifically altered in the portion of the sequence in the IgG1 Fc region. These engineered mutations to the encoded amino acid sequence resulted in two new Fc domain variants (G236A) and (G236A/A330 μL/I332E) (under the EU Numbering system), described as “77A^{GA}” and “77A^{GAALIE},” respectively. In addition to these variants, the original antibody sequence with wild-type human IgG1 Fc domain was also retained, herein referred to as “WT”. The experiments described below were conducted to characterize binding to human FcγR2A, and determine the functional impact on the uptake of antibody:HSP70 complexes by human primary antigen presenting cells for each of the antibody variants.

ELISA assays were performed to assess binding to coated FcγR2A by either the monomeric antibody or the antibody in complex with HSP70. Briefly, the assay was performed by coating plates with recombinant human FcγR2A overnight at 0.3 μg/mL. Plates were then washed three times with 300 μL of 1×PBST, and then blocked using 150 L of SuperBlock per well for 1 hr. Washing was repeated as before, then the dilution series of any of the antibody variants (referred to as “monomeric antibody”) or any of the or antibody immuno-complexes (hereafter referred to as “77A ICX”) were added. To make each dilution series, the following was performed: HSP70 was held constant, and first prepared at a 2× concentration of 200 nM. Antibody was first prepared at 600 nM (2× starting concentration) for the first well, and then serially diluted in three-fold increments using 1× PBST. For use in the assay as monomeric antibody, 80 μL of each 2× antibody dilution was added to 80 μL of 1×PBST and mixed well by pipette to make a 1× dilution series, with the highest concentration being 300 nM. To form the 77A ICX, 80 μL of each 2× antibody dilution was added to 80 μL of 2× HSP70 and mixed well by pipette. Complexes were allowed to form for 1 hour at room temperature, resulting in a ratio series starting at 3:1 of antibody:HSP70 and decreasing with each step of antibody dilution. After complex formation, 50 μL of each sample in the dilution series of monomeric antibody, or 77A ICX, was added to the plate along with the appropriate controls. The plate was incubated for 1 hour before washing as described above. Next, 50 μL of secondary antibody against human Fab, and conjugated to HRP, used at a dilution of 1:5000 in 1× PBST was added to the wells. The plate was then incubated for 15 minutes at room temperature. The plate was again washed as before and then 50 μL of TMB substrate solution was added. To stop the reactions, ELISA stop solution containing sulfuric acid was added to the wells. Plates were then read on a plate reader at 450 and 540 nm wavelengths.

Uptake of HSP70-GFP by antigen presenting cells was evaluated using a FACS-based assay. The various primary cells used were commercially sourced, pre-purified, and acquired frozen in cryovials from Stemcell Technologies, Inc. At time of use, cells were thawed, washed and pelleted to remove cryomedia, and then resuspended at 1×10⁶ cells/mL into the recommended culture media with appropriate supplements as required. At the time of resuspension, cells began treatment with 10 μM of the MG-132 proteosome inhibitor. The addition of MG-132 was required to prevent loss of the GFP signal due to the proteosome degradation of internalized ICX. The cells were treated for 3.5 hours while incubated in 5% CO2 at 37° C. in culture media. Formation of immune complexes (ICX) were performed in PBS+1% BSA with each of the antibodies, 50 nM HSP70-GFP, 10 μM MG-132, and a 1:220 dilution of nanobody against GFP conjugated with Alexa488. For each antibody series, the amount of antibody was titrated down while holding everything else constant. The highest concentration of antibody used was cell type dependent as indicated in the figures. These mixtures were incubated in the dark for 30 minutes at room temperature to allow complexes to form, then pre-chilled on ice. Cell suspensions were plated out at 1.5×10⁵ cells per well in a 96-well round bottom plate and pelleted. The plate was decanted to remove supernatant, and cooled on ice. Cells were gently resuspended in each well with the pre-chilled buffer containing complexed antibody:HSP70-GFP mixtures, and incubated at 4° C. for 1 hour. After the incubation, cells were pelleted, decanted supernatant, and resuspended in FACS staining buffer. The cells were pelleted again, decanted, and 1 μL of viability stain was added to the residual volume of each well in the plate. The cells were stained on ice in the dark for 40 minutes before adding FACS staining buffer to wash cells and pelleting again. The cells were washed an additional time and after decanting, cells were resuspended in 400 μL PBS. The resulting cell suspensions were acquired on cytometer and gated on live events. Compensation was performed using amine reactive compensation (ArC) beads for the viability stain, and UltraComp beads for the GFP channel.

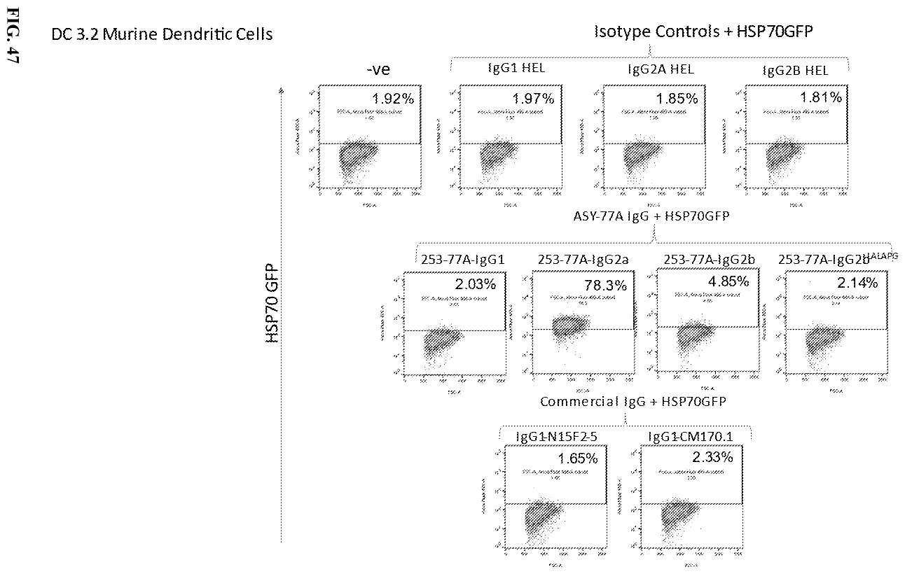

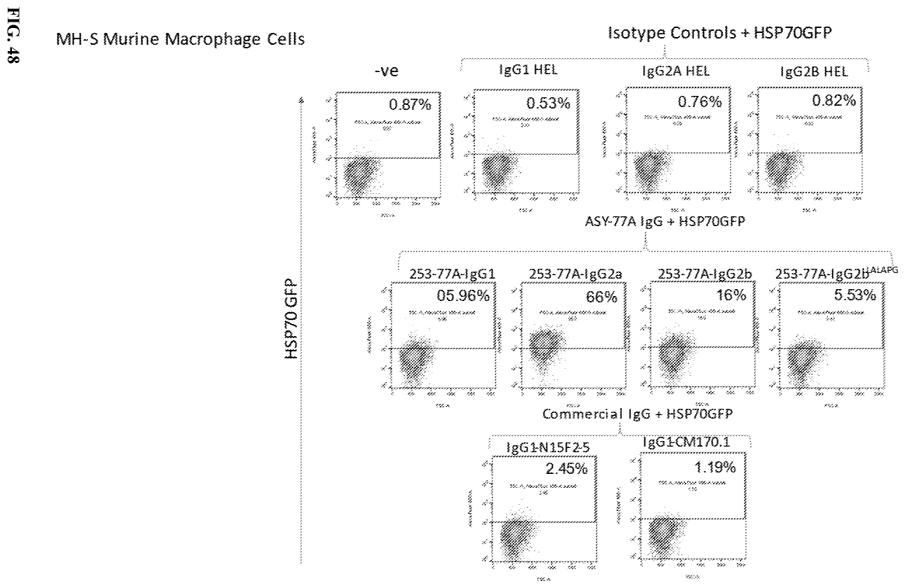

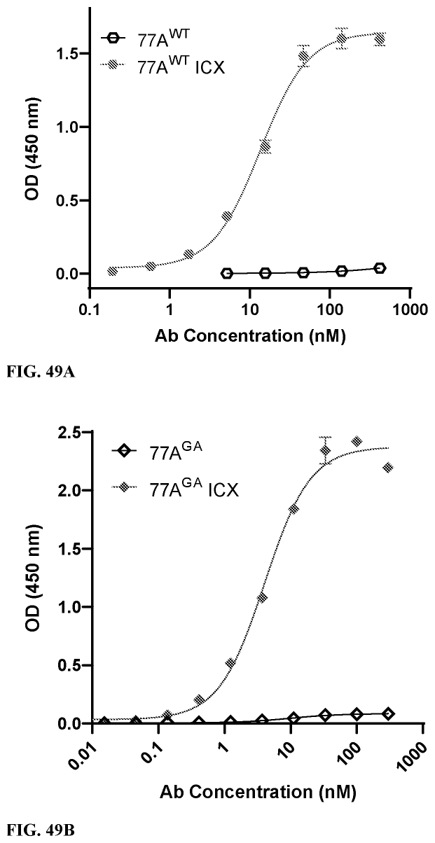

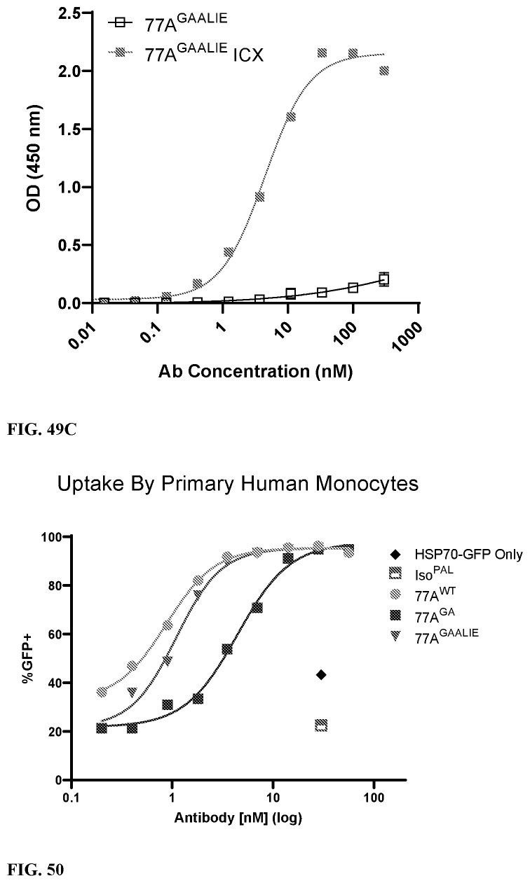

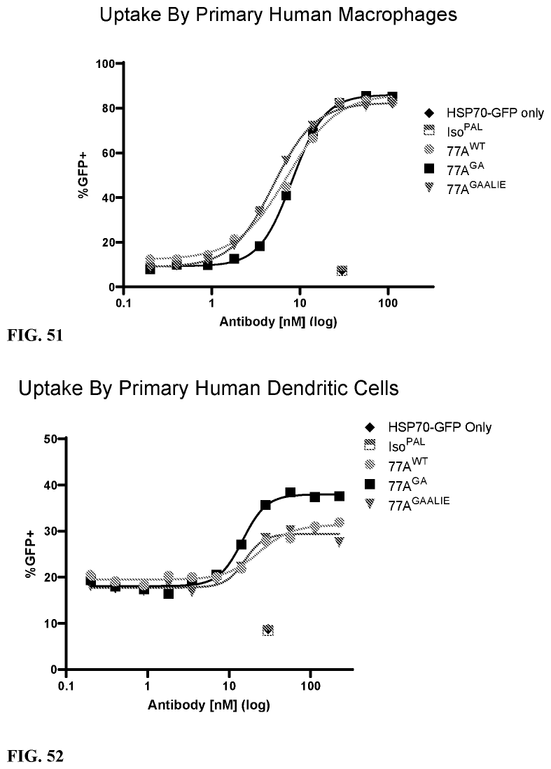

The interactions with recombinant FcγR2A were weak for the antibody in monomeric form; however, in complex with HSP70 the avidity for the receptor was greatly increased (FIG. 49A). Similar patterns of binding were observed for 77A^{WT}, 77A^{GA}, and 77A^{GAALIE} variants on recombinant FcγR2A (FIG. 49B). Internalization was observed in the FACS-based assays, with uptake greatest at the higher ratios (FIGS. 50, 51, and 52). Monocytes had comparable uptake between the 77A^{WT} and 77A^{GAALIE} variants, but was reduced with the 77A^{GA} variant (FIG. 50). Macrophages had largely overlapping uptake curves for all variants (FIG. 51). Lastly, immature dendritic cells demonstrated both an improved EC50 uptake value, and increased amount of uptake from the 77A^{GA} variant as compared to 77A^{WT} and 77A^{GAALIE} (FIG. 52).

The results of the in vitro ELISA experiments showed that all three variants of the antibody (77A^{WT}, 77A^{GA}, and 77A^{GAALIE}) were capable of interacting with FcγR2A to comparable extents in vitro. In the cell-based uptake assays, the increased binding of the antibody:HSP70 complex to the lower affinity FcγR allowed for increased amounts of internalization to occur. The amount of internalization was potentiated by the presence of the 77A^{GA} and/or 77A^{GAALIE} mutations, relative to 77A^{WT}, depending on the antigen-presenting cell type. Both of the Fc engineered variants were able to demonstrate a comparable or enhanced ability for uptake, relative to 77A^{WT}, on macrophage and dendritic cells. Only on monocytes did 77A^{WT} show a higher level of uptake than the 77A^{GA} or 77A^{GAALIE} variants.

### Example 23. —77A Wildtype and Engineered Fc Antibody Activation of Primary Human Immune Cells

This Example describes activation of primary human cells by engineered Fc variants of 77A antibody in complex with HSP70.

The 77A antibody in complex with HSP70 (subsequently referred to as the ICX), was tested for the eliciting of activating responses from primary immune cell subsets. These studies compared the capability of ICX formed using the wild-type (77A^{WT}), the 77A^{GA}, or the 77A^{GAALIE} IgG1 Fc engineered humanized antibody variants to elicit responses during in vitro assays. The experiments were performed using various lymphoid and myeloid lineages of primary human cells enriched from donor PBMC.

Human peripheral blood mononuclear cells (PBMC) from adult healthy donors, age 18-65 years old, were obtained for each experiment. PBMC were first isolated using centrifugation separation on a density-gradient. The buffy coat layer was then pelleted and washed three times in PBS by centrifugation and resuspension. The resulting PBMC were used as input for follow-up assays, while unused PBMC were cryopreserved in cryomedia, then stored in the vapor phase of liquid nitrogen. When needed, frozen PBMC were thawed quickly at 37° C., resuspended in culture media and pelleted to remove cryomedia. For culturing conditions, PBMC were resuspended in complete culture media (RPMI 1640 media supplemented with 10% FBS, 55 μM 2-mercaptoethanol, 10 mM HEPES) and incubated at 37° C. with 5% CO2. For each PBMC subset utilized, Miltenyi isolation kits were used to selectively enrich for the desired target population of CD4+ T cells, CD8+ T cells, NK cells, or monocytic populations from bulk PBMC. For isolation of immature dendritic cells (iDC), monocytes were first isolated using the enrichment kits described, and then plated at 1×10⁶ cells/mL into the appropriate vessel size needed in splenocyte media (RPMI 1640 supplemented with 10% FBS, 10 mM HEPES, 55 M 2-mercaptoethanol, 1× Pen/Strep). In order to generate immature dendritic cells, monocytes were first cultured with rhIL-4 (250 U/mL) and rhGM-CSF (1,000 U/mL) to stimulate differentiation. The media was replenished after 48 hours, or if acidification was observed. After five days of stimulation, monocyte-derived immature dendritic cells were counted. ICX was prepared fresh for each experiment in culture media, and performed by incubation of a 1:1 mixture of HSP70 at 40 μg/mL and antibody at 80 μg/mL to get a 2× concentration of complex. Complexes were allowed to form for 1 hour at room temperature before use in an assay. After complexing, 0.75 mL of this newly formed ICX was added to 0.75 mL of cells in culture for a final concentration in the wells of 10 μg/mL of HSP70 and 20 μg/mL of antibody. Conditions lacking a portion of the ICX were prepared as described above without the indicated component. When added, rhIL-2 was used in culture conditions at 10 ng/mL. For all experiments performed, assays with dendritic cells or NK cells were cultured in splenocyte media while T-cells (CD4⁺ or CD8⁺) were in culture media. For proliferation assays, T-cells were labeled for 30 minutes in the presence of 4 μM CFSE before quenching excess dye and washing with culture media. For assay conditions that were performed with rhIL-2 in culture conditions, rhIL-2 was used at 10 ng/mL. As a positive control, Staphylococcal Enterotoxin B was added to culture conditions at 2 μg/mL. As an isotype control, a non-binding irrelevant human IgG1 antibody (Iso^{PAL}) was used. For both cytokine and CFSE proliferation assays, cells were first resuspended in media and then various treatment conditions subsequently were added to wells. Cells were then cultured for 5 days (CFSE) or 6 days (cytokine array) in the conditions indicated. At assay endpoint for CFSE proliferation assays, cells were pelleted and resuspended in PBS, then stained for viability before acquiring on a BD LSRFortessa cytometer. Live events were gated for analysis using FlowJo analysis software. For phenotypic characterization of cells via FACS, T-cells were stained for viability, CD45, CD8a, and CD4 expression accordingly. The activation markers were stained for by using α-human CD25 and α-human HLA-DP/DR/DQ FACS reagents before acquiring on the cytometer. For endpoint analysis of cytokine array assays, media was subsequently collected and profiled using the Bio-RAD Bio-Plex Multiplex System with the Bio-Plex Pro Human Cytokine Screening Panel, 48-plex kit. Data was acquired and analyzed using the Bio-Plex Data Pro software. Observed intensities values were normalized to the media only control, and heatmaps were generated.

The CD8 T-cell FACS assay for CFSE dilution demonstrated that proliferation was occurring in several experimental conditions, along with the positive control (FIG. 53). No proliferation was observed in the negative control condition. Upon exposure to the formed ICX, the wildtype, GA, and GAALIE variants all elicited comparable levels of proliferation as compared to the media alone condition, from a subset of T-cells in culture. These effects of the ICXs were observed directly on CD8 T-cells and did not require sequential exposure of the ICX first to APC before eliciting the observed CD8+ T-cell proliferative response. Addition of IL-2 alone, or IL-2 with uncomplexed HSP70 (and a non-binding isotype), was not sufficient to drive significant levels of proliferation. In the phenotypic FACS analysis that measured the activation of CD8+ T cells in response, co-incubation of cells with ICX resulted in more than three times the level of double-positive HLA-DR and CD25 cells achieved with HSP70 alone (FIG. 54). Cytokine array results were consistent with the functional data and demonstrated that ICX with any of the Fc variants resulted in enhanced, but comparable, levels of activating Th-1 biased cytokines. This was further potentiated by the addition of rhIL-2 in culture (Table 18). Cytokine arrays were quantified by measuring the fluorescence intensity specific for each cytokine and normalizing the resultant fluorescent intensity value to the intensity of media only control. The incubation of 77A-HSP70 ICXs also stimulated cytokine synthesis and release from primary human CD4+ and NK cells in the presence and absence of additional IL-2 stimulation (Tables 19 and 20, respectively). No significant differences were detected between the different 77A Fc domain variants.

TABLE 18
Fluorescence intensity values of CD8+ T-cell Cytokine Array

| | | | | | | | CD8 | CD8 | CD8 | CD8 | | |
|---|---|---|---|---|---|---|---|---|---|---|---|---|
| | | | | CD8 | CD8 | | HSP70 | HSP70 | HSP70 | HSP70 | | |
| | | | CD8 | HSP70 | HSP70 | CD8 | & | & IgG1 | & IgG1 | & IgG1 | | |
| | | | HSP70 | & | & | HSP70 | IgG1 | 77A | 77A | 77A | | |
| | | | & | IgG1 | IgG1 | & | Isotype | WT | GA | GAALIE | | |
| | CD8 | CD8 | IgG1 | 77A | 77A | IgG1 | & | & | & | & | CD8 | CD8 |
| | Media | HSP70 | Isotype | WT | GA | GAALIE | IL-2 | IL-2 | IL-2 | IL-2 | SEB | IL-2 |
| Hu IL-10 | 29 | 18 | 31 | 29 | 25 | 26 | 119 | 116 | 57 | 77 | 25 | 46 |
| Hu IL-12 | 46 | 46 | 46 | 51 | 51 | 51 | 155 | 167 | 167 | 200 | 46 | 143 |
| (p 40) | | | | | | | | | | | | |
| Hu IL-12 | 17 | 14 | 22 | 24 | 23 | 20 | 21 | 22 | 00 | 25 | 19 | 20 |
| (p 70) | | | | | | | | | | | | |
| Hu IL-13 | 25 | 25 | 25 | 25 | 25 | 25 | 28 | 52 | 81 | 148 | 25 | 35 |
| Hu IL-15 | 1158 | 1121 | 1218 | 1223 | 1223 | 1260 | 1192 | 1246 | 1274 | 1300 | 1249 | 1324 |
| Hu IL-16 | 197 | 332 | 208 | 198 | 195 | 225 | 299 | 309 | 232 | 479 | 409 | 217 |
| Hu IL- | 24 | 22 | 24 | 45 | 43 | 42 | 55 | 90 | 67 | 103 | 19 | 42 |
| 17A | | | | | | | | | | | | |
| Hu IL-18 | 39 | 27 | 43 | 45 | 44 | 47 | 42 | 47 | 45 | 45 | 49 | 52 |
| Hu IL-1a | 52 | 34 | 72 | 106 | 74 | 72 | 89 | 120 | 88 | 120 | 38 | 139 |
| Hu IL-1b | 4 | 4 | 8 | 40 | 22 | 22 | 11 | 43 | 22 | 34 | 4 | 4 |
| Hu IL-1ra | 584 | 251 | 1417 | 584 | 612 | 553 | 691 | 939 | 738 | 864 | 226 | 1072 |
| Hu IL-2 | 18 | 19 | 16 | 26 | 24 | 24 | 29 | 29 | 29 | 29 | 16 | 29 |
| Hu IL-2Ra | 29 | 35 | 39 | 48 | 49 | 49 | 104 | 120 | 98 | 208 | 36 | 81 |
| Hu IL-3 | 2 | 2 | 3 | 2 | 3 | 2 | 3 | 4 | 3 | 4 | 1 | 3 |
| Hu IL-4 | 6 | 7 | 9 | 20 | 18 | 17 | 24 | 26 | 24 | 30 | 7 | 24 |
| Hu IL-5 | 1095 | 1006 | 1301 | 1150 | 1108 | 1283 | 1138 | 1154 | 1175 | 1192 | 1289 | 1371 |
| Hu IL-6 | 26 | 33 | 92 | 412 | 292 | 280 | 108 | 322 | 205 | 351 | 21 | 34 |
| Hu IL-7 | 42 | 14 | 35 | 13 | 14 | 13 | 58 | 35 | 35 | 96 | 14 | 35 |
| Hu IL-8 | 175 | 181 | 337 | 2288 | 2022 | 2059 | 974 | 1854 | 1262 | 2163 | 45 | 387 |
| Hu IL-9 | 84 | 75 | 86 | 97 | 95 | 107 | 118 | 131 | 112 | 211 | 84 | 116 |
| Hu IP-10 | 145 | 107 | 151 | 107 | 115 | 111 | 158 | 135 | 125 | 161 | 109 | 170 |
| Hu b-NGF | 76 | 77 | 60 | 65 | 73 | 66 | 65 | 71 | 63 | 74 | 57 | 66 |
| Hu | 15 | 19 | 23 | 40 | 35 | 33 | 35 | 47 | 37 | 52 | 19 | 29 |
| CTACK | | | | | | | | | | | | |
| Hu | 9 | 8 | 10 | 11 | 10 | 10 | 10 | 10 | 10 | 11 | 9 | 12 |
| Eotaxin | | | | | | | | | | | | |
| Hu FGF | 88 | 102 | 123 | 187 | 158 | 169 | 210 | 224 | 199 | 255 | 88 | 212 |
| basic | | | | | | | | | | | | |
| Hu G-CSF | 556 | 576 | 538 | 2458 | 1973 | 1954 | 2709 | 3082 | 2853 | 4632 | 364 | 2622 |
| Hu GM- | 8 | 5 | 10 | 20 | 15 | 17 | 54 | 65 | 56 | 100 | 7 | 45 |
| CSF | | | | | | | | | | | | |
| Hu GRO-a | 2099 | 2075 | 2068 | 2220 | 2295 | 2292 | 2164 | 2323 | 2307 | 2604 | 1988 | 2325 |
| Hu HGF | 97 | 110 | 110 | 186 | 135 | 164 | 231 | 264 | 231 | 312 | 30 | 231 |
| Hu IFN-a2 | 14 | 14 | 15 | 34 | 29 | 25 | 32 | 41 | 35 | 49 | 15 | 35 |
| Hu IFN-g | 20 | 17 | 23 | 25 | 25 | 27 | 100 | 116 | 57 | 197 | 23 | 69 |
| Hu LIF | 73 | 78 | 78 | 173 | 144 | 167 | 175 | 214 | 167 | 272 | 62 | 198 |
| Hu MCP- | 252 | 313 | 388 | 204 | 347 | 484 | 62 | 73 | 72 | 93 | 28 | 72 |
| 1 (MCAF) | | | | | | | | | | | | |
| Hu MCP- | 9 | 8 | 9 | 14 | 14 | 13 | 13 | 11 | 13 | 16 | 7 | 13 |
| 3 | | | | | | | | | | | | |
| Hu M- | 24 | 20 | 25 | 25 | 28 | 26 | 49 | 54 | 41 | 67 | 26 | 38 |
| CSF | | | | | | | | | | | | |
| Hu MIF | 844 | 1689 | 844 | 844 | 827 | 856 | 1205 | 1237 | 952 | 2269 | 1971 | 935 |
| Hu MIG | 83 | 40 | 106 | 73 | 66 | 52 | 192 | 108 | 106 | 136 | 23 | 200 |
| Hu MIP- | 18 | 21 | 16 | 114 | 98 | 87 | 135 | 159 | 156 | 577 | 9 | 137 |
| 1a | | | | | | | | | | | | |
| Hu MIP- | 103 | 107 | 101 | 260 | 231 | 292 | 463 | 531 | 549 | 1094 | 96 | 463 |
| 1b | | | | | | | | | | | | |
| Hu PDGF- | 368 | 343 | 376 | 376 | 372 | 400 | 408 | 415 | 392 | 501 | 388 | 452 |
| bb | | | | | | | | | | | | |
| Hu | 190 | 227 | 141 | 149 | 179 | 176 | 294 | 286 | 253 | 644 | 212 | 284 |
| RANTES | | | | | | | | | | | | |
| Hu SCF | 15 | 9 | 13 | 33 | 25 | 24 | 36 | 40 | 34 | 53 | 8 | 34 |
| Hu SCGF- | 71908 | 80925 | 70528 | 69660 | 75709 | 76214 | 53035 | 53424 | 66736 | 71047 | 67853 | 67383 |
| b | | | | | | | | | | | | |
| Hu SDF- | 187 | 163 | 198 | 203 | 198 | 187 | 223 | 258 | 218 | 267 | 187 | 223 |
| 1a | | | | | | | | | | | | |
| Hu TNF-a | 130 | 140 | 156 | 542 | 428 | 489 | 761 | 962 | 768 | 1337 | 195 | 615 |
| Hu TNF-b | 55 | 65 | 75 | 85 | 81 | 81 | 206 | 209 | 211 | 437 | 77 | 211 |
| Hu | 32 | 39 | 33 | 55 | 47 | 30 | 67 | 83 | 60 | 103 | 26 | 56 |
| TRAIL | | | | | | | | | | | | |
| Hu VEGF | 1089 | 1030 | 1181 | 1198 | 1176 | 1212 | 995 | 1051 | 1198 | 1165 | 1146 | 1294 |

TABLE 19
Fluorescence intensity values of NK cell Cytokine Array

| | | | | | | | | NK | NK | | | |
|---|---|---|---|---|---|---|---|---|---|---|---|---|
| | | | | NK | NK | | NK | HSP70 | HSP70 | | | |
| | | | NK | HSP70 | HSP70 | NK | HSP70 | & | & | NK | | |
| | | | HSP70 | & | & | HSP70 | & | IgG1 | IgG1 | HSP70 | | |
| | | | & | IgG1 | IgG1 | & | IgG1 | 77A | 77A | & | | |
| | NK | NK | IgG1 | 77A | 77A | IgG1 | Isotype | WT | GA | IgG1 | NK | NK |
| | Media | HSP70 | Isotype | WT | GA | [symbol] | [symbol] | [symbol] | [symbol] | 77A | SEB | IL-2 |
| Hu IL-10 | 9 | 11 | 9 | 11 | 9 | 9 | 79 | 9 | 9 | 11 | 79 | 182 |
| Hu IL-12 | 393 | 871 | 436 | 436 | 998 | 737 | 737 | 737 | 737 | 998 | 436 | 393 |
| (p 40) | | | | | | | | | | | | |
| Hu IL-12 | 74 | 101 | 101 | 101 | 88 | 74 | 122 | 122 | 88 | 54 | 74 | 74 |
| (p 70) | | | | | | | | | | | | |
| Hu IL-13 | 10 | 10 | 34 | 11 | 10 | 10 | 10 | 11 | 11 | 10 | 11 | 10 |
| Hu IL-15 | 664 | 1334 | 737 | 664 | 1687 | 1334 | 1967 | 1967 | 1524 | 664 | 664 | 737 |
| Hu IL-16 | 89 | 99 | 99 | 89 | 89 | 89 | 333 | 284 | 284 | 89 | 89 | 89 |
| Hu IL-17A | 330 | 236 | 188 | 236 | 330 | 188 | 259 | 283 | 330 | 283 | 188 | 236 |
| Hu IL-18 | 40 | 44 | 40 | 40 | 64 | 83 | 44 | 44 | 102 | 44 | 40 | 44 |
| Hu IL-1a | 77 | 220 | 77 | 77 | 356 | 220 | 423 | 220 | 289 | 220 | 220 | 220 |
| Hu IL-1b | 30 | 3 | 30 | 23 | 23 | 30 | 14 | 37 | 30 | 30 | 14 | 30 |
| Hu IL-1ra | 741 | 1958 | 1596 | 1199 | 741 | 1596 | 1596 | 1596 | 741 | 1596 | 667 | 667 |
| Hu IL-2 | 55 | 33 | 37 | 33 | 33 | 33 | 7650 | 7342 | 8939 | 33 | 37 | 33 |
| Hu IL-2Ra | 26 | 69 | 208 | 114 | 114 | 26 | 305 | 208 | 114 | 114 | 208 | 26 |
| Hu IL-3 | 11 | 9 | 9 | 11 | 11 | 14 | 15 | 11 | 11 | 13 | 13 | 9 |
| Hu IL-4 | 15 | 17 | 15 | 15 | 28 | 28 | 15 | 28 | 28 | 15 | 15 | 15 |
| Hu IL-5 | 226 | 1442 | 251 | 226 | 1880 | 1442 | 989 | 989 | 1442 | 226 | 754 | 226 |
| Hu IL-6 | 6 | 23 | 50 | 37 | 63 | 50 | 37 | 63 | 6 | 23 | 6 | 6 |
| Hu IL-7 | 118 | 118 | 118 | 118 | 118 | 106 | 846 | 106 | 118 | 106 | 118 | 633 |
| Hu IL-8 | 42 | 87 | 51 | 51 | 190 | 190 | 106 | 209 | 134 | 60 | 47 | 51 |
| Hu IL-9 | 36 | 251 | 168 | 36 | 333 | 251 | 251 | 415 | 415 | 36 | 83 | 40 |
| Hu IP-10 | 213 | 26 | 213 | 26 | 29 | 26 | 309 | 29 | 213 | 29 | 213 | 29 |
| Hu b-NGF | 94 | 94 | 94 | 94 | 94 | 104 | 104 | 94 | 94 | 94 | 94 | 94 |
| Hu CTACK | 344 | 344 | 344 | 344 | 344 | 344 | 344 | 344 | 344 | 344 | 344 | 382 |
| Hu Eotaxin | 14 | 7 | 6 | 7 | 47 | 71 | 34 | 28 | 34 | 6 | 7 | 14 |
| Hu FGF | 306 | 306 | 306 | 339 | 339 | 512 | 306 | 512 | 339 | 339 | 306 | 339 |
| basic | | | | | | | | | | | | |
| Hu G-CSF | 121 | 321 | 121 | 321 | 676 | 1162 | 4632 | 4293 | 4157 | 224 | 109 | 503 |
| Hu GM- | 13 | 13 | 13 | 34 | 13 | 29 | 175 | 154 | 113 | 13 | 15 | 13 |
| CSF | | | | | | | | | | | | |
| Hu GRO-a | 1422 | 3581 | 1279 | 1279 | 3581 | 3581 | 3131 | 3581 | 3378 | 2513 | 1422 | 1279 |
| Hu HGF | 828 | 1126 | 828 | 658 | 1126 | 461 | 415 | 658 | 828 | 828 | 461 | 461 |
| Hu IFN-a2 | 127 | 50 | 56 | 50 | 56 | 56 | 56 | 50 | 50 | 50 | 127 | 56 |
| Hu IFN-g | 127 | 31 | 28 | 28 | 127 | 31 | 501 | 351 | 229 | 55 | 28 | 28 |
| Hu LIF | 569 | 399 | 569 | 310 | 399 | 569 | 216 | 650 | 569 | 216 | 216 | 569 |
| Hu MCP-1 | 36 | 75 | 121 | 36 | 59 | 36 | 40 | 75 | 36 | 59 | 40 | 40 |
| (MCAF) | | | | | | | | | | | | |
| Hu MCP-3 | 45 | 10 | 10 | 45 | 11 | 45 | 10 | 11 | 11 | 60 | 11 | 11 |
| Hu M-CSF | 10 | 69 | 34 | 10 | 10 | 69 | 83 | 69 | 11 | 34 | 69 | 10 |
| Hu MIF | 727 | 1011 | 727 | 727 | 1011 | 808 | 870 | 1011 | 1178 | 727 | 727 | 727 |
| Hu MIP-1a | 0 | 11 | 0 | 7 | 41 | 101 | 112 | 112 | 112 | 0 | 0 | 6 |
| Hu MIP-1b | 28 | 176 | 53 | 47 | 356 | 1961 | 179400 | 107000 | 7668 | 28 | 13 | 13 |
| Hu PDGF- | 367 | 367 | 367 | 367 | 367 | 45 | 880 | 40 | 634 | 45 | 45 | 45 |
| bb | | | | | | | | | | | | |
| Hu | 669 | 743 | 669 | 669 | 1024 | 1024 | 2840 | 2512 | 2925 | 669 | 669 | 669 |
| RANTES | | | | | | | | | | | | |
| Hu SCF | 155 | 155 | 155 | 155 | 172 | 155 | 155 | 172 | 172 | 155 | 172 | 172 |
| Hu SCGF-b | 10180 | 111541 | 10180 | 5814 | 108253 | 925300 | 51431 | 56471 | 56471 | 1270 | 5814 | 14534 |
| Hu SDF-1a | 129 | 300 | 129 | 69 | 41] | 356 | 328 | 411 | 411 | 244 | 244 | 187 |
| Hu TNF-a | 180 | 162 | 180 | 162 | 162 | 442 | 1755 | 1244 | 869 | 180 | 162 | 442 |
| Hu TNF-b | 44 | 189 | 44 | a | 116 | 10 | 651 | 573 | 651 | 9 | 9 | 44 |
| Hu TRAIL | 119 | 119 | 58 | 119 | 119 | 119 | 168 | 119 | 119 | 58 | 7 | 58 |
| Hu VEGF | 299 | 1953 | 216 | 299 | 2147 | 1721 | 1253 | 1253 | 1487 | 299 | 380 | 216 |

[symbol]

indicates data missing or illegible when filed

TABLE 20
Fluorescence intensity values of CD4+ T cell Cytokine Array

| | | | | CD4 | CD4 | | | CD4 | CD4 | CD4 | | |
|---|---|---|---|---|---|---|---|---|---|---|---|---|
| | | | | HSP70 | HSP70 | CD4 | | HSP70 | HSP70 | HSP70 | | |
| | | | CD4 | & | & | HSP70 | CD4 | & | & | & | | |
| | | | HSP70 | IgG1 | IgG1 | & | HSP70 | IgG1 | IgG1 | IgG1 | | |
| | CD4 | CD4 | & | 77A | 77A | IgG1 | & | 77A | 77A | 77A | CD4 | CD4 |
| | Media | HSP70 | IgG1 | WT | [symbol] | [symbol] | IgG1 | [symbol] | [symbol] | [symbol] | SEB | IL-2 |
| Hu IL-10 | 51 | 67 | 53 | 1037 | 539 | 428 | 4004 | 1654 | 1180 | 1355 | 1000 | 3535 |
| Hu IL-12 | 285 | 302 | 249 | 822 | 712 | 916 | 1404 | 1282 | 1725 | 1349 | 403 | 1314 |
| (p 40) | | | | | | | | | | | | |
| Hu IL-12 | 23 | 26 | 22 | 115 | 107 | 64 | 110 | 43 | 43 | 55 | 33 | 285 |
| (p 70) | | | | | | | | | | | | |
| Hu IL-13 | 28 | 25 | 25 | 20 | 19 | 16 | 3338 | 140 | 535 | 596 | 574 | 5765 |
| Hu IL-15 | 1300 | 1479 | 1279 | 1620 | 1488 | 1409 | 1300 | 1664 | 1702 | 1704 | 1636 | 1494 |
| Hu IL-16 | 354 | 313 | 427 | 481 | 381 | 389 | 386 | 407 | 463 | 429 | 1246 | 372 |
| Hu IL-17A | 184 | 135 | 122 | 983 | 1165 | 791 | 1072 | 18188 | 31961 | 30273 | 2660 | 1182 |
| Hu IL-18 | 74 | 72 | 58 | 140 | 99 | 100 | 85 | 127 | 123 | 131 | 62 | 102 |
| Hu IL-1a | 141 | 148 | 123 | 1996 | 883 | 664 | 22 | 1343 | 921 | 1167 | 232 | 241 |
| Hu IL-1b | 14 | 12 | 13 | 2887 | 1058 | 814 | 31 | 2084 | 1349 | 1626 | 29 | 29 |
| Hu IL-1ra | 28034 | 28431 | 25085 | 15617 | 21936 | 23271 | 23417 | 12764 | 24215 | 24114 | 1463 | 33107 |
| Hu IL-2 | 86 | 97 | 76 | 159 | 147 | 133 | 7768 | 5633 | 3434 | 9072 | 112 | 9839 |
| Hu IL-2Ra | 120 | 119 | 99 | 419 | 396 | 391 | 1739 | 1143 | 2537 | 1989 | 2638 | 2223 |
| Hu IL-3 | 6 | 7 | 6 | 10 | 8 | 7 | 13 | 25 | 30 | 40 | 56 | 14 |
| Hu IL-4 | 35 | 35 | 35 | 69 | 58 | 54 | 156 | 73 | 77 | 75 | 45 | 263 |
| Hu IL-5 | 1119 | 1341 | 1080 | 1575 | 1448 | 1369 | 1770 | 1646 | 1656 | 1669 | 1502 | 2851 |
| Hu IL-6 | 107 | 97 | 105 | 8954 | 8310 | 8158 | 509 | 8362 | 9148 | 9051 | 145 | 413 |
| Hu IL-7 | 48 | 48 | 75 | 93 | 96 | 90 | 133 | 177 | 177 | 210 | 113 | 162 |
| Hu IL-8 | 40895 | 69667 | 22306 | 742545 | 210524 | 816800 | 96155 | 816800 | 816800 | 816800 | 4959 | 334903 |
| Hu IL-9 | 174 | 211 | 155 | 347 | 351 | 336 | 1220 | 591 | 1214 | 1128 | 543 | 1355 |
| Hu IP-10 | 4401 | 9016 | 4248 | 5053 | 5690 | 5726 | 8023 | 3389 | 11459 | 9641 | 3951 | 9967 |
| Hu b-NGF | 84 | 102 | 60 | 101 | 88 | 77 | 73 | 98 | 113 | 117 | 95 | 108 |
| Hu | 43 | 45 | 46 | 572 | 494 | 444 | 83 | 548 | 524 | 546 | 66 | 83 |
| CTACK | | | | | | | | | | | | |
| Hu | 12 | 14 | 11 | 17 | 15 | 15 | 19 | 20 | 20 | 21 | 17 | 21 |
| Eotaxin | | | | | | | | | | | | |
| Hu FGF | 276 | 291 | 279 | 419 | 364 | 352 | 334 | 435 | 440 | 436 | 348 | 350 |
| basic | | | | | | | | | | | | |
| Hu G-CSF | 4696 | 4602 | 4716 | 13844 | 8754 | 7098 | 5222 | 14353 | 11719 | 12708 | 5326 | 5091 |
| Hu GM- | 50 | 41 | 33 | 487 | 435 | 322 | 1753 | 1231 | 2235 | 2690 | 6832 | 3156 |
| CSF | | | | | | | | | | | | |
| Hu GRO-a | 2626 | 2778 | 2362 | 150379 | 150379 | 150379 | 3078 | 136708 | 150379 | 150379 | 3093 | 3394 |
| Hu HGF | 569 | 804 | 652 | 713 | 602 | 582 | 652 | 790 | 810 | 807 | 515 | 689 |
| Hu IFN-a2 | 53 | 48 | 48 | 84 | 72 | 65 | 67 | 96 | 93 | 93 | 65 | 72 |
| Hu IFN-g | 69 | 70 | 59 | 3243 | 3163 | 1957 | 4967 | 8534 | 9131 | 10776 | 10042 | 8458 |
| Hu LIF | 267 | 292 | 253 | 593 | 495 | 473 | 2785 | 956 | 1837 | 1658 | 5127 | 3839 |
| Hu MCP-1 | 2419 | 2901 | 2296 | 2104 | 1958 | 2283 | 1670 | 1853 | 1910 | 1967 | 224 | 1523 |
| (MCAF) | | | | | | | | | | | | |
| Hu MCP-3 | 101 | 100 | 69 | 2336 | 1525 | 914 | 25 | 1827 | 2948 | 2455 | 25 | 34 |
| Hu M-CSF | 321 | 307 | 259 | 236 | 365 | 382 | 526 | 357 | 546 | 515 | 199 | 589 |
| Hu MIF | 1839 | 1633 | 2034 | 2856 | 2480 | 2359 | 3659 | 3053 | 6914 | 4447 | 16777 | 4265 |
| Hu MIG | 1667 | 2859 | 2985 | 2012 | 2021 | 2316 | 5216 | 3529 | 7997 | 8078 | 629 | 4820 |
| Hu MIP- | 1455 | 2198 | 1432 | 1809 | 1561 | 1626 | 1840 | 1614 | 2599 | 2747 | 1725 | 2235 |
| 1a | | | | | | | | | | | | |
| Hu MIP- | 2783 | 3146 | 2225 | 7356 | 6687 | 4705 | 7356 | 7356 | 7356 | 7356 | 7356 | 7356 |
| 1b | | | | | | | | | | | | |
| Hu | 615 | 633 | 585 | 788 | 708 | 708 | 731 | 827 | 899 | 919 | 711 | 801 |
| PDGF-bb | | | | | | | | | | | | |
| Hu | 493 | 600 | 353 | 561 | 682 | 685 | 7101 | 1013 | 3298 | 4791 | 1390 | 24880 |
| RANTES | | | | | | | | | | | | |
| Hu SCF | 64 | 71 | 59 | 171 | 147 | 128 | 137 | 213 | 220 | 214 | 89 | 147 |
| Hu SCGF- | 133926 | 136215 | 93954 | 87362 | 112815 | 101526 | 73392 | 62431 | 70355 | 91336 | 57312 | 99381 |
| b | | | | | | | | | | | | |
| Hu SDF- | 260 | 313 | 282 | 411 | 402 | 379 | 518 | 502 | 577 | 611 | 508 | 547 |
| 1a | | | | | | | | | | | | |
| Hu TNF-a | 12069 | 14696 | 8733 | 51356 | 49059 | 38543 | 48325 | 35157 | 439380 | 483318 | 17908 | 88268 |
| Hu TNF-b | 331 | 349 | 282 | 584 | 626 | 558 | 1539 | 707 | 1375 | 1467 | 3730 | 2360 |
| Hu TRAIL | 87 | 100 | 64 | 656 | 506 | 429 | 218 | 690 | 706 | 699 | 275 | 261 |
| Hu VEGF | 1146 | 1372 | 1079 | 1342 | 1230 | 1193 | 1037 | 1238 | 1332 | 1420 | 1355 | 1273 |

[symbol]

indicates data missing or illegible when filed

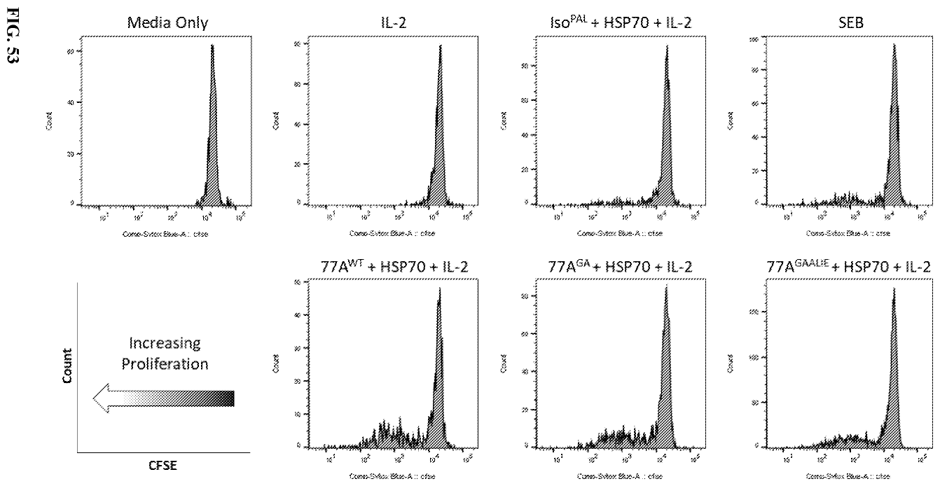

77A ICXs also stimulated the activation of primary immature dendritic cells as measured by the increased expression of CD83 activation marker CD11c dendritic cells (FIG. 55). The magnitude of dendritic cell activation by ICXs was similar to that observed with the positive control TNF-α+ionomycin combination.

The results of these studies demonstrate an objective response to exposure of human primary immune cells to the 77A ICX. While some conditions required supplementation with IL-2 to observe the fully augmented response, each experiment showed a discernable effect of the ICX to elicit activating responses. The wildtype, GA, and GAALIE variants generally showed comparable levels of in vitro proliferation and cytokine activation. Surprisingly, while HSP70 alone was sufficient to partially elicit some of these responses, cytokine array data and phenotypic co-expression of HLA-DR and CD25 indicated that ICX were far more effective in augmenting CD8+ T-cell activation, resulting in a Th-1 type biased response.

These data demonstrate that the formation of HSP70:77A immune complexes elicit responses through objective increased levels of activation and maturation of key immune cells of the innate and adaptive immune system.

### Example 24. —In Vivo Activity in EG7 Model of Engineered Fc Variants of 77A

This Example describes in vivo activity of engineered Fc variants of 77A antibody in complex with HSP70.

To investigate efficacy and impact on survival by the humanized 77A with wild type IgG1 antibody 77A^{WT} and engineered Fc GA and GAALIE variants (77A^{GA} and 77A^{GAALIE}, respectively), an in vivo study was performed using the modified C57BL/6 murine host, and the disseminated EG7-OVA mouse T lymphoma tumor model which was modified to overexpress the firefly luciferase and the chicken ovalbumin genes (OVA). This study focused on evaluating the impact on mice already bearing established and disseminated tumors with HSP70-OVA:77A immune complexes (ICX) that were pre-formed in vitro using HSP70-OVA and each of the tested antibodies.

The C57BL/6 background genetically modified mice, genotype B6.Cg-Del(1Fcgr2b-Fcgr3)1Rav Fcgr1<tm1Hoga>Tg(FCGR1A) #Jgjw Tg(FCGR2A)11Mkz Tg (FCGR2B) #Rav Tg(FCGR3A)1156Rav Tg(FCGR3B)1373Rav, used in this study ranged in age from 12 to 16 weeks, and were housed in an animal barrier facility. Upon experiment initiation, female mice were injected intravenously with 1×10⁶ EG7-OVA cells suspended in 100 μL PBS on day 0. The mice were imaged twice weekly for luciferase activity using the IVIS 200 imager. Randomization was performed on day 7 based on luciferase signal intensity into 5 groups of 15 female mice receiving different treatments. HSP70-OVA:77A immune complexes used for immunization were formed fresh for each injection and performed with each of the engineered Fc variants: GA (77A^{GA}), GAALIE (77A^{GAALIE}), or wildtype (77A^{WT}) antibody as compared to a non-binding irrelevant human IgG1 isotype (Iso^{PAL}) and non-treated PBS controls. To generate the complexes used for immunizations, each antibody was independently used in the complex formation reaction. This was accomplished using 100 μL of antibody at 160 ng/mL and mixing with 100 μL of HSP70-OVA at 200 ng/mL resulting in 200 μL of PBS containing a total of 20 ng of HSP70-OVA and 16 ng of specific antibody. The mixture was incubated for 1 hour at room temperature, to allow complex formation to readily occur, before injecting mice with 200 μL of mixture. On days 7 and 13, mice were injected with the complex of HSP70-OVA:77A subcutaneously on the hind limb flank. On day 20, mice were boosted with the same complex injected via intraperitoneal (IP) administration. No further interventions were performed. Mice continued to be monitored daily for health status, and imaging was performed twice weekly. Mice were marked as a death in survival data if they died during the study or were euthanized after meeting protocol endpoint criteria for displaying an ataxic moribund condition.

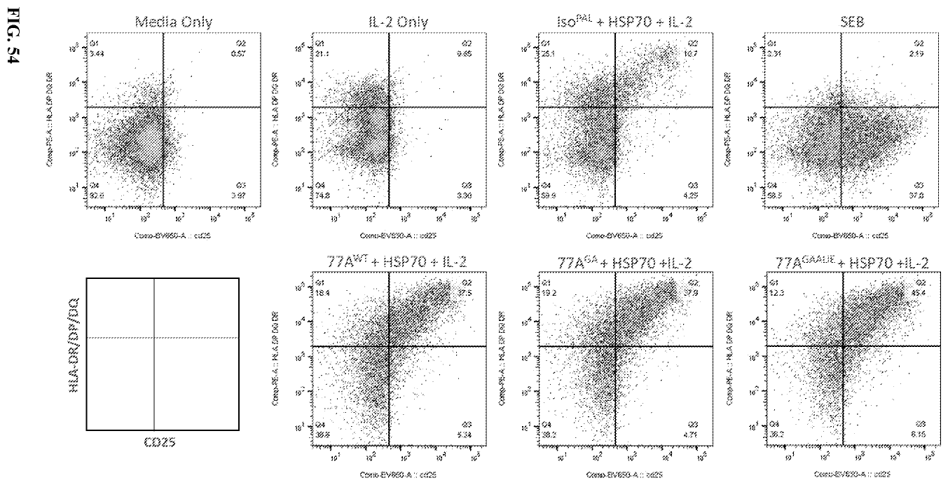

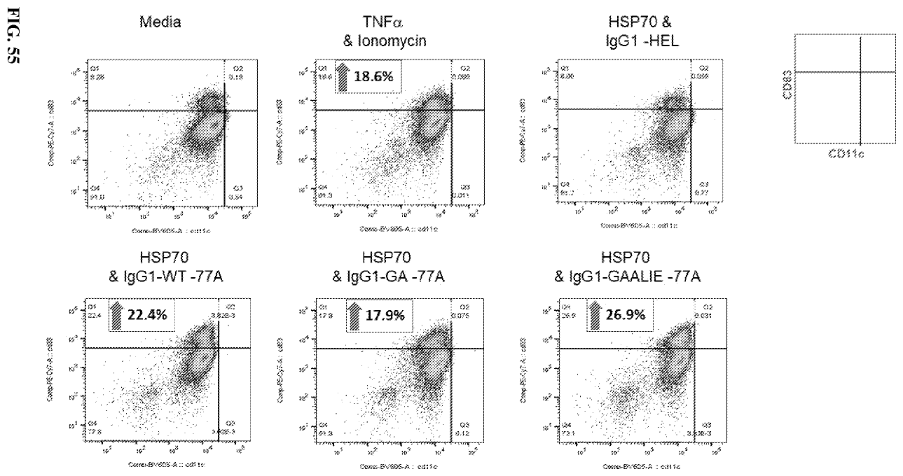

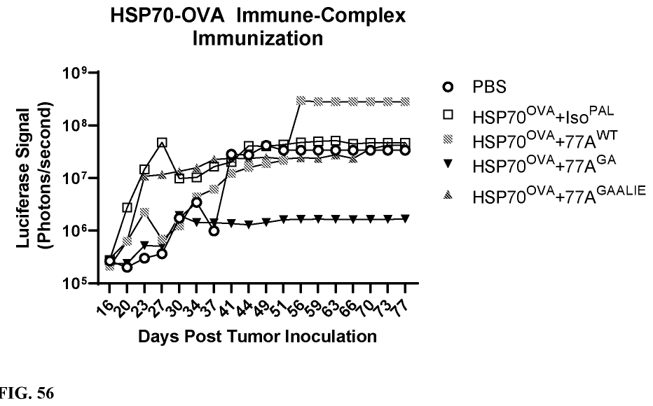

Luciferase activity resulting from tumor burden continued to increase for the first 30 days following inoculation in most groups, at which time signals largely plateaued, suggesting tumor stasis (FIG. 56). To measure accumulated tumor burden in the various groups, luciferase values of dead animals were carried forward. Due to variations in retention of luciferase activity over time, no statistical significance could be attributed to any group.

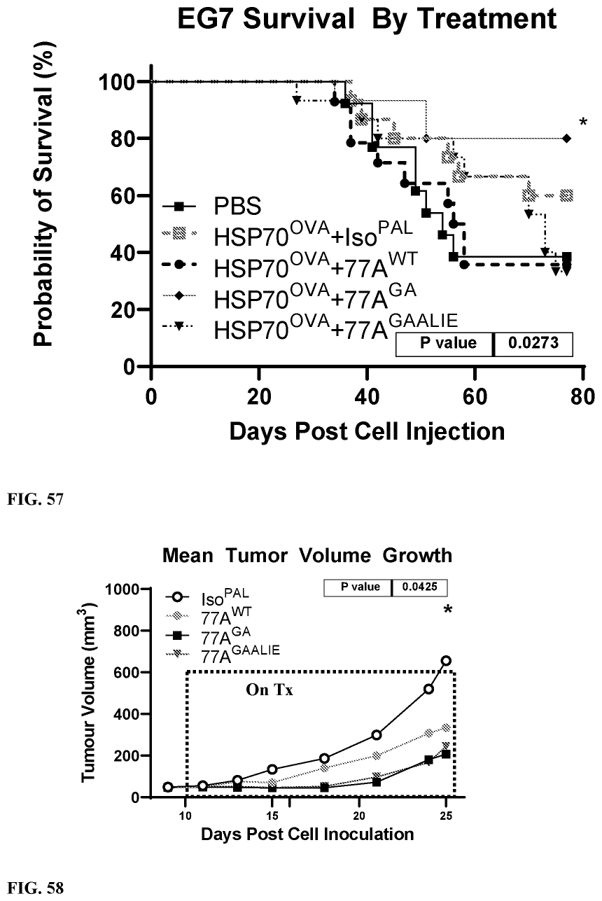

For survival analysis, Kaplan-Meier curves were generated (FIG. 57). As shown, the GA group achieved statistical significance for survival as compared to the isotype control (Iso^{PAL}), with a p-value of 0.0273 in the Logrank test. The humanized 77A antibody with the GA mutations had a superior impact to overall survival as compared to the isotype control antibody. In this study, however, the other groups failed to reach a statistically significant p-value. The data demonstrated the proof of concept that immunizations with 77A:HSP70-OVA immune complexes were successfully able to provide antigenic cross-priming of the complexed proteins in a prime-boost fashion to ovalbumin antigens expressed by the EG7-OVA tumors, resulting in anti-tumor activity. The elicited immune response of 77A^{GA} was able to provide a survival benefit over the other antibodies tested.

### Example 25. —In Vivo Activity in E0771 Murine Model of Engineered Fc Variants of 77A

This Example describes in vivo activity of engineered Fc variants of 77A antibody in an E0771 mouse model.

The anti-tumor activity of 77A with wild-type human IgG1 Fc domain and the engineered Fc variants 77A^{GA} and 77A^{GAALIE} was tested. The experiment was performed in the modified C57BL/6 murine host in which the murine FcγRs were replaced by their human counterparts using the E0771 syngeneic breast tumor model modified to overexpress human recombinant HSP70-GFP, referred to herein as E0771-HSP70GFP.

The C57BL/6 background genetically modified mice, B6.Cg-Del(1Fcgr2b-Fcgr3)1Rav Fcgr1<tm1Hoga>Tg(FCGR1A) #Jgjw Tg(FCGR2A)11Mkz Tg (FCGR2B) #Rav Tg(FCGR3A)1156Rav Tg(FCGR3B)1373Rav, used in this study ranged in age from 12 to 16 weeks of age, and were housed in an animal barrier facility. Upon experiment initiation, female mice were injected with 5×10⁵ cells suspended in PBS into the hind flank subcutaneously on day 0. Mice were then randomized at day 10 after tumor growth was confirmed, into four groups of 10 mice per treatment group. The mice were dosed with either the human isotype control IgG1 antibody (Iso^{PAL}), or each of the humanized antibodies, at 1 mg/kg in 100 μL in PBS via tail vein route injection. Dosing was carried out twice weekly for a total of six doses. Tumor volumes were obtained by caliper measurement and plotted as mean±SEM. The mice were also observed for overall survival and Kaplan-Meier curves were generated.

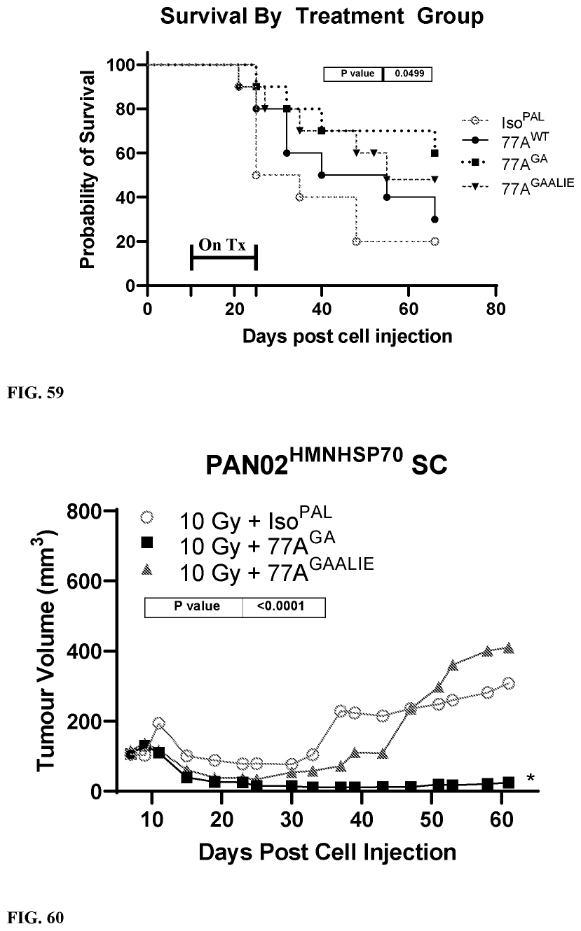

As shown in FIG. 58, the humanized 77A antibody with the GA mutation in its Fc domain (77^{GA}) had superior performance over the wildtype (77A^{WT}), and the 77A^{GAALIE} antibody, which appeared to have similar responses in tumor growth inhibition. Statistical significance for inhibition of tumor growth was observed by day 25 for the GA variant versus isotype control (Iso^{PAL}). Significance was calculated using a one-way ANOVA, with Kruskal-Wallis test for multiple comparisons. The resulting adjusted p-value calculated for multiple comparisons was 0.0244 for GA vs isotype control. While the GAALIE variant antibody also appeared to have improved activity over the wild-type, it failed to reach statistical significance due to group sizes. The GA antibody demonstrated a statistically significant improvement on survival and reached significance with a p-value of 0.0499 in the Logrank test (FIG. 59).

These data demonstrate that the engineered 77A Fc variants both appeared to have improved anti-tumor activity over the wild type. The 77A^{GA} variants provided statistically significant tumor growth inhibition, and survival benefit over the wild-type antibody. These data indicate that despite identical antigen binding regions, the engineered 77A^{GA} and 77A^{GAALIE} Fc domains with different FcγR binding profiles, versus wild-type IgG1, elicited stronger immune responses in humanized FcγR mice, as compared to the wild-type 77A antibody.

### Example 26. —In Vivo Activity in PANO2 Murine Model with Irradiation of Engineered Fc Variants of 77A

This Example describes in vivo activity of engineered Fc variants of 77A antibody in a PANO2 irradiated mouse model.

This study was used to determine the role of increased immunogenic cell death (ICD) in a murine tumor model stimulated with targeted irradiation (inducing HSP70), and resultant activity on the anti-tumor activity of 77A, which binds to and mediates the uptake of tumor-derived extracellular HSP70 into antigen-presenting cells (APCs). Wild-type IgG1 human antibody (77A^{WT}) was engineered in the constant region of the Fc domain to generate the 77A^{GA} or 77A^{GAALIE} variants. This study evaluated the efficacy and impact on survival of wild type, and the engineered 77A^{GA} and 77A^{GAALIE} IgG1 Fc domain variants against the human isotype control (Iso^{PAL}). The experiment was performed in vivo using a modified C57BL/6 murine host, and the PANO2 syngeneic pancreatic ductal adenocarcinoma (PDAC) tumor model after a single dose of radiation directed at the tumor. In the PANO2 cell line, the murine HSP70 gene was replaced by its human counterpart through CRISPR mediated site-specific recombination and also contained a separately integrated luciferase coding sequence. This engineered PANO2 cell line is referred to herein as PANO2-CRE2luc.

The C57BL/6 background genetically modified mice, B6.Cg-Del(1Fcgr2b-Fcgr3)1Rav Fcgr1<tm1Hoga>Tg(FCGR1A) #Jgjw Tg(FCGR2A)11Mkz Tg (FCGR2B) #Rav Tg(FCGR3A)1156Rav Tg(FCGR3B)1373Rav, used in this study ranged in age from 12 to 16 weeks of age, and were housed in an animal barrier facility. Upon experiment initiation, male mice were injected with 1×10⁶ tumor cells suspended in PBS into the hind flank subcutaneously on day 0. On day 8, mice were first imaged by CT scan to confirm tumor boundaries and orientation, then irradiated with 10 Gy of X-ray radiation. On day 9, mice were randomized by tumor volumes into three treatment groups of 10 mice per group. The mice were dosed with either Iso^{PAL}, or each of the 77A^{GA} or 77A^{GAALIE} humanized variant antibodies, at 1 mg/kg dose in 100 μL in PBS via tail vein injection. Dosing was carried out twice weekly for a total of six doses. Tumor volumes were obtained by caliper measurement. Data is represented as mean±SEM. Statistically significant tumor growth inhibition was observed for the GA vs isotype (PAL) groups (FIG. 60). Significance was calculated using a two-way ANOVA with Kruskal-Wallis test for multiple comparisons, and reached significance by day 11 (data not shown). A Welch's t-test was performed on data from day 61 comparing 77A^{GA} vs Iso^{PAL}, and achieved a highly significant p-value of <0.0001, as indicated by the (*). However, in this study, 77A^{GAALIE} treatment group did not reach statistical significance.

Tumor volumes initially decreased, as expected, after treatment with irradiation, but began to rebound approaching day 30 for most of the treatment groups. The group treated with the humanized 77A^{GA} variant, however, retained control of tumor volume, and demonstrated statistically significant tumor growth inhibition that persisted up until the study's end point (FIG. 60).

The experiment demonstrates that the anti-tumor effect of X-ray radiation can be further enhanced by the combination with the 77A^{GA} Fc domain variant. Tumor volumes for all groups experienced some regression after irradiation, but irradiation alone was insufficient to elicit a durable response. This responsiveness may have been the result of increased release of tumor-derived extracellular HSP70 as a result of ICD due to the high-dose irradiation. Since irradiation alone was not sufficient to elicit durable anti-tumor responses, these findings highlight the potential utility of 77A to maximize durable responses and tumor inhibition in combination with ICD inducing agents.

All of the methods disclosed and claimed herein can be made and executed without undue experimentation in light of the present disclosure. While the compositions and methods of this invention have been described in terms of preferred embodiments, it will be apparent to those of skill in the art that variations may be applied to the methods and in the steps or in the sequence of steps of the method described herein without departing from the concept, spirit and scope of the invention. More specifically, it will be apparent that certain agents which are both chemically and physiologically related may be substituted for the agents described herein while the same or similar results would be achieved. All such similar substitutes and modifications apparent to those skilled in the art are deemed to be within the spirit, scope and concept of the invention as defined by the appended claims.

### REFERENCES

The following references, to the extent that they provide exemplary procedural or other details supplementary to those set forth herein, are specifically incorporated herein by reference.
Anderson D A, 3rd, Murphy K M, Briseno C G. Development, Diversity, and Function of Dendritic Cells in Mouse and Human. Cold Spring Harb Perspect Biol. 2018; 10(11).
Aprile et al., PLoS ONE, 8:e67962, 2013.
Bennett C L, van Rijn E, Jung S, et al. Inducible ablation of mouse Langerhans cells diminishes but fails to abrogate contact hypersensitivity. J Cell Biol. 2005; 169(4):569-576.
Berges C, Naujokat C, Tinapp S, et al. A cell line model for the differentiation of human dendritic cells. Biochem Biophys Res Commun. 2005; 333(3):896-907.
Binder R J. Heat-shock protein-based vaccines for cancer and infectious disease. Expert Rev Vaccines. 2008; 7(3):383-393.
Bjorklund C C, Ma W, Wang Z Q, et al. Evidence of a role for activation of Wnt/beta-catenin signaling in the resistance of plasma cells to lenalidomide. J Biol Chem. 2011; 286(13):11009-11020.
Boudesco C, Cause S, Jego G, Garrido C. Hsp70: A Cancer Target Inside and Outside the Cell. Methods Mol Biol. 2018; 1709:371-396.
Bourque J, Hawiger D. Immunomodulatory Bonds of the Partnership between Dendritic Cells and T Cells. Crit Rev Immunol. 2018; 38(5):379-401.
Bruhns, Blood, 119:5640, 2012.
Casanova V, Naval-Macabuhay I, Massanella M, et al. Adenosine deaminase enhances the immunogenicity of human dendritic cells from healthy and HIV-infected individuals. PLoS One. 2012; 7(12):e51287.
Chacon J A, Sarnaik A A, Chen J Q, et al. Manipulating the tumor microenvironment ex vivo for enhanced expansion of tumor-infiltrating lymphocytes for adoptive cell therapy. Clin Cancer Res. 2015; 21(3):611-621.
Chatterjee M, Andrulis M, Stuhmer T, et al. The PI3K/Akt signaling pathway regulates the expression of Hsp70, which critically contributes to Hsp90-chaperone function and tumor cell survival in multiple myeloma. Haematologica. 2013; 98(7):1132-1141.
Chatterjee S, Burns T F. Targeting Heat Shock Proteins in Cancer: A Promising Therapeutic Approach. Int J Mol Sci. 2017; 18(9).
Chen N, Ritsma LMA, Vrisekoop N. In vivo characteristics of human and mouse breast tumor cell lines. Exp Cell Res. 2019.
Chen D S, Mellman I. Oncology meets immunology: the cancer-immunity cycle. Immunity. 2013; 39(1):1-10.
Craig E A, Marszalek J. How Do J-Proteins Get Hsp70 to Do So Many Different Things?Trends Biochem Sci. 2017; 42(5):355-368.
Dabaghian M, Latify A M, Tebianian M, et al. Vaccination with recombinant 4×M2e.HSP70c fusion protein as a universal vaccine candidate enhances both humoral and cell-mediated immune responses and decreases viral shedding against experimental challenge of H9N2 influenza in chickens. Vet Microbiol. 2014; 174(1-2):116-126.
Daugaard M, Rohde M, Jaattela M. The heat shock protein 70 family: Highly homologous proteins with overlapping and distinct functions. FEBS Lett. 2007; 581(19):3702-3710.
Davidson E, Doranz B J. A high-throughput shotgun mutagenesis approach to mapping B-cell antibody epitopes. Immunology. 2014; 143(1):13-20.
Demaria O, De Gassart A, Coso S, et al. STING activation of tumor endothelial cells initiates spontaneous and therapeutic antitumor immunity. Proc Natl Acad Sci USA. 2015; 112(50):15408-15413.
Dodd et al., Oncogene, 34:1312, 2015.
Duffield J S, Forbes S J, Constandinou C M, et al. Selective depletion of macrophages reveals distinct, opposing roles during liver injury and repair. J Clin Invest. 2005; 115(1):56-65.
Fu C, Jiang A. Dendritic Cells and CD8 T Cell Immunity in Tumor Microenvironment. Front Immunol. 2018; 9:3059.
Fukaya T, Murakami R, Takagi H, et al. Conditional ablation of CD205+ conventional dendritic cells impacts the regulation of T-cell immunity and homeostasis in vivo. Proc Natl Acad Sci USA. 2012; 109(28):11288-11293.
Gornati L, Zanoni I, Granucci F. Dendritic Cells in the Cross Hair for the Generation of Tailored Vaccines. Front Immunol. 2018; 9:1484.
Greene L E, Zinner R, Naficy S, Eisenberg E. Effect of nucleotide on the binding of peptides to 70-kDa heat shock protein. J Biol Chem. 1995; 270(7):2967-2973.
Guzhova I V, Margulis B A. HSP70-based anti-cancer immunotherapy. Hum Vaccin Immunother. 2016; 12(10):2529-2535.
Hofgaard P O, Jodal H C, Bommert K, et al. A novel mouse model for multiple myeloma (MOPC315.B M) that allows noninvasive spatiotemporal detection of osteolytic disease. PLoS One. 2012; 7(12):e51892.
Hossain M K, Wall K A. Use of Dendritic Cell Receptors as Targets for Enhancing Anti-Cancer Immune Responses. Cancers (Basel). 2019; 11(3).
Huang A Y, Gulden P H, Woods A S, et al. The immunodominant major histocompatibility complex class I-restricted antigen of a murine colon tumor derives from an endogenous retroviral gene product. Proc Natl Acad Sci USA. 1996; 93(18):9730-9735.
Huarte E, Cubillos-Ruiz J R, Nesbeth Y C, et al. Depletion of dendritic cells delays ovarian cancer progression by boosting antitumor immunity. Cancer Res. 2008; 68(18):7684-7691.
Hunt C, Calderwood S. Characterization and sequence of a mouse hsp70 gene and its expression in mouse cell lines. Gene. 1990; 87(2):199-204.
Johnsen G, Elsayed S. Antigenic and allergenic determinants of ovalbumin—III. MHC Ia-binding peptide (OA 323-339) interacts with human and rabbit specific antibodies. Mol Immunol. 1990; 27(9):821-827.
Joly A L, Wettstein G, Mignot G, Ghiringhelli F, Garrido C. Dual role of heat shock proteins as regulators of apoptosis and innate immunity. J Innate Immun. 2010; 2(3):238-247.
Jung S, Unutmaz D, Wong P, et al. In vivo depletion of CD11c+ dendritic cells abrogates priming of CD8+ T cells by exogenous cell-associated antigens. Immunity. 2002; 17(2):211-220.
Kandasamy G, Andreasson C. Hsp70-Hsp110 chaperones deliver ubiquitin-dependent and -independent substrates to the 26S proteasome for proteolysis in yeast. J Cell Sci. 2018; 131(6).
Karttunen J, Sanderson S, Shastri N. Detection of rare antigen-presenting cells by the lacZ T-cell activation assay suggests an expression cloning strategy for T-cell antigens. Proc Natl Acad Sci USA. 1992; 89(13):6020-6024.
Kim S J, Schatzle S, Ahmed S S, et al. Increased cathepsin S in Prdm1(−/−) dendritic cells alters the TFH cell repertoire and contributes to lupus. Nat Immunol. 2017; 18(9):1016-1024.
Kim K, Skora A D, Li Z, et al. Eradication of metastatic mouse cancers resistant to immune checkpoint blockade by suppression of myeloid-derived cells. Proc Natl Acad Sci USA. 2014; 111(32):11774-11779.
Kissenpfennig A, Henri S, Dubois B, et al. Dynamics and function of Langerhans cells in vivo: dermal dendritic cells colonize lymph node areas distinct from slower migrating Langerhans cells. Immunity. 2005; 22(5):643-654.
Kodama A, Tanaka R, Saito M, Ansari A A, Tanaka Y. A novel and simple method for generation of human dendritic cells from unfractionated peripheral blood mononuclear cells within 2 days: its application for induction of HIV-1-reactive CD4(+) T cells in the hu-PBL SCID mice. Front Microbiol. 2013; 4:292.
Koelle D M, Magaret A, McClurkan C L, et al. Phase I dose-escalation study of a monovalent heat shock protein 70-herpes simplex virus type 2 (HSV-2) peptide-based vaccine designed to prime or boost CD8 T-cell responses in HSV-naive and HSV-2-infected subjects. Clin Vaccine Immunol. 2008; 15(5):773-782.
Kroemer G, Galluzzi L, Kepp O, Zitvogel L. Immunogenic cell death in cancer therapy. Annu Rev Immunol. 2013; 31:51-72.
Kuhn D J, Berkova Z, Jones R J, et al. Targeting the insulin-like growth factor-1 receptor to overcome bortezomib resistance in preclinical models of multiple myeloma. Blood. 2012; 120(16):3260-3270.
Kuhn D J, Chen Q, Voorhees P M, et al. Potent activity of carfilzomib, a novel, irreversible inhibitor of the ubiquitin-proteasome pathway, against preclinical models of multiple myeloma. Blood. 2007; 110(9):3281-3290.
Kuhn D J, Hunsucker S A, Chen Q, Voorhees P M, Orlowski M, Orlowski R Z. Targeted inhibition of the immunoproteasome is a potent strategy against models of multiple myeloma that overcomes resistance to conventional drugs and nonspecific proteasome inhibitors. Blood. 2009; 113(19):4667-4676.
Lao J, Madani J, Puertolas T, et al. Liposomal Doxorubicin in the treatment of breast cancer patients: a review. J Drug Deliv. 2013; 2013:456409.
Lee H C, Wang H, Baladandayuthapani V, et al. RNA polymerase I inhibition with CX-5461 as a novel therapeutic strategy to target MYC in multiple myeloma. Br J Haematol. 2016; In press.
Lee Y H, Martin-Orozco N, Zheng P, et al. Inhibition of the B7-H3 immune checkpoint limits tumor growth by enhancing cytotoxic lymphocyte function. Cell Res. 2017; 27(8):1034-1045.
Lewis D J, Wang Y, Huo Z, et al. Effect of vaginal immunization with HIVgp140 and HSP70 on HIV-1 replication and innate and T cell adaptive immunity in women. J Virol. 2014; 88(20):11648-11657.
Li H S, Liu C, Xiao Y, et al. Bypassing STAT3-mediated inhibition of the transcriptional regulator ID2 improves the antitumor efficacy of dendritic cells. Sci Signal. 2016; 9(447):ra94.
Li B, Fu J, Chen P, et al. The Nuclear Factor (Erythroid-derived 2)-like 2 and Proteasome Maturation Protein Axis Mediate Bortezomib Resistance in Multiple Myeloma. J Biol Chem. 2015; 290(50):29854-29868.
Lou Y, Liu C, Lizee G, et al. Antitumor activity mediated by CpG: the route of administration is critical. J Immunother. 2011; 34(3):279-288.
Lu X, Horner J W, Paul E, et al. Effective combinatorial immunotherapy for castration-resistant prostate cancer. Nature. 2017; 543(7647):728-732.
Lyseng-Williamson K A, Duggan S T, Keating G M. Pegylated liposomal doxorubicin: a guide to its use in various malignancies. BioDrugs. 2013; 27(5):533-540.
Ma W, Wang M, Wang Z Q, et al. Effect of long-term storage in TRIzol on microarray-based gene expression profiling. Cancer Epidemiol Biomarkers Prev. 2010; 19(10):2445-2452.
Malczyk A H, Kupke A, Prufer S, et al. A Highly Immunogenic and Protective Middle East Respiratory Syndrome Coronavirus Vaccine Based on a Recombinant Measles Virus Vaccine Platform. J Virol. 2015; 89(22):11654-11667.
Maleki Vareki S. High and low mutational burden tumors versus immunologically hot and cold tumors and response to immune checkpoint inhibitors. J Immunother Cancer. 2018; 6(1):157.
Malviya G, Anzola K L, Podesta E, et al. (99m)Tc-labeled rituximab for imaging B lymphocyte infiltration in inflammatory autoimmune disease patients. Mol Imaging Biol. 2012; 14(5):637-646.
McNulty S, Colaco C A, Blandford L E, Bailey C R, Baschieri S, Todryk S. Heat-shock proteins as dendritic cell-targeting vaccines—getting warmer. Immunology. 2013; 139(4):407-415.
Mitsiades N, Mitsiades C S, Poulaki V, et al. Molecular sequelae of proteasome inhibition in human multiple myeloma cells. Proc Natl Acad Sci USA. 2002; 99(22):14374-14379.
Moore M W, Carbone F R, Bevan M J. Introduction of soluble protein into the class I pathway of antigen processing and presentation. Cell. 1988; 54(6):777-785.
Murshid A, Gong J, Calderwood S K. The role of heat shock proteins in antigen cross presentation. Front Immunol. 2012; 3:63.
Niknam S, Barsoumian H B, Schoenhals J E, et al. Radiation Followed by OX40 Stimulation Drives Local and Abscopal Antitumor Effects in an Anti-PD1-Resistant Lung Tumor Model. Clin Cancer Res. 2018; 24(22):5735-5743.
Nollen E A, Brunsting J F, Song J, Kampinga H H, Morimoto R I. Bag1 functions in vivo as a negative regulator of Hsp70 chaperone activity. Mol Cell Biol. 2000; 20(3):1083-1088.
Park H J, Qin H, Cha S C, et al. Induction of TLR4-dependent CD8+ T cell immunity by murine betadefensin2 fusion protein vaccines. Vaccine. 2011; 29(18):3476-3482.
Peng P, Menoret A, Srivastava P K. Purification of immunogenic heat shock protein 70-peptide complexes by ADP-affinity chromatography. J Immunol Methods. 1997; 204(1):13-21.
Piva L, Tetlak P, Claser C, Karjalainen K, Renia L, Ruedl C. Cutting edge: Clec9A+ dendritic cells mediate the development of experimental cerebral malaria. J Immunol. 2012; 189(3):1128-1132.
Ralph P, Nakoinz I. Antibody-dependent killing of erythrocyte and tumor targets by macrophage-related cell lines: enhancement by PPD and LPS. J Immunol. 1977; 119(3):950-954.
Rampelt H, Mayer M P, Bukau B. Nucleotide Exchange Factors for Hsp70 Chaperones. Methods Mol Biol. 2018; 1709:179-188.
Raschke W C, Baird S, Ralph P, Nakoinz I. Functional macrophage cell lines transformed by Abelson leukemia virus. Cell. 1978; 15(1):261-267.
Rios-Doria J, Durham N, Wetzel L, et al. Doxil synergizes with cancer immunotherapies to enhance antitumor responses in syngeneic mouse models. Neoplasia. 2015; 17(8):661-670.
Robertson J D, Datta K, Biswal S S, Kehrer J P. Heat-shock protein 70 antisense oligomers enhance proteasome inhibitor-induced apoptosis. Biochem J. 1999; 344 Pt 2(1):477-485.
Rotzschke O, Falk K, Stevanovic S, Jung G, Walden P, Rammensee H G. Exact prediction of a natural T cell epitope. Eur J Immunol. 1991; 21(11):2891-2894.
Santema W, Rutten V, Segers R, et al. Postexposure subunit vaccination against chronic enteric mycobacterial infection in a natural host. Infect Immun. 2013; 81(6):1990-1995.
Sharpless N E, Depinho R A. The mighty mouse: genetically engineered mouse models in cancer drug development. Nat Rev Drug Discov. 2006; 5(9):741-754.
Shen Z, Reznikoff G, Dranoff G, Rock K L. Cloned dendritic cells can present exogenous antigens on both MHC class I and class II molecules. J Immunol. 1997; 158(6):2723-2730.
Shvetsov et al. Philos. Trans. R. Soc. Lond. B. Biol. Sci., 373:1738, 2018.
Shevtsov M, Multhoff G. Heat Shock Protein-Peptide and HSP-Based Immunotherapies for the Treatment of Cancer. Front Immunol. 2016; 7:171.
Shtivelman E, Davies M Q, Hwu P, et al. Pathways and therapeutic targets in melanoma. Oncotarget. 2014; 5(7):1701-1752.
Shulman M, Wilde C D, Kohler G. A better cell line for making hybridomas secreting specific antibodies. Nature. 1978; 276(5685):269-270.
Shultz L D, Schweitzer P A, Christianson S W, et al. Multiple defects in innate and adaptive immunologic function in NOD/LtSz-scid mice. J Immunol. 1995; 154(1):180-191.
Singh M, Khong H, Dai Z, et al. Effective innate and adaptive antimelanoma immunity through localized TLR7/8 activation. J Immunol. 2014; 193(9):4722-4731.
Stangl S, Gehrmann M, Riegger J, et al. Targeting membrane heat-shock protein 70 (Hsp70) on tumors by cmHsp70.1 antibody. Proc Natl Acad Sci USA. 2011; 108(2):733-738.
Swiecki M, Gilfillan S, Vermi W, Wang Y, Colonna M. Plasmacytoid dendritic cell ablation impacts early interferon responses and antiviral N K and CD8(+) T cell accrual. Immunity. 2010; 33(6):955-966.
Takagi H, Fukaya T, Eizumi K, et al. Plasmacytoid dendritic cells are crucial for the initiation of inflammation and T cell immunity in vivo. Immunity. 2011; 35(6):958-971.
Tseng W W, Malu S, Zhang M, et al. Analysis of the intratumoral adaptive immune response in well differentiated and dedifferentiated retroperitoneal liposarcoma. Sarcoma. 2015; 2015:547460.
Turner M D, Nedjai B, Hurst T, Pennington D J. Cytokines and chemokines: At the crossroads of cell signalling and inflammatory disease. Biochim Biophys Acta. 2014; 1843(11):2563-2582.
van de Donk N W, Janmaat M L, Mutis T, et al. Monoclonal antibodies targeting CD38 in hematological malignancies and beyond. Immunol Rev. 2016; 270(1):95-112.
van Rooijen N, Hendrikx E. Liposomes for specific depletion of macrophages from organs and tissues. Methods Mol Biol. 2010; 605:189-203.
Vega V L, Rodriguez-Silva M, Frey T, et al. Hsp70 translocates into the plasma membrane after stress and is released into the extracellular environment in a membrane-associated form that activates macrophages. J Immunol. 2008; 180(6):4299-4307.
Veglia F, Gabrilovich D I. Dendritic cells in cancer: the role revisited. Curr Opin Immunol. 2017; 45:43-51.
Wang R, Town T, Gokarn V, Flavell R A, Chandawarkar R Y. HSP70 enhances macrophage phagocytosis by interaction with lipid raft-associated TLR-7 and upregulating p38 MAPK and PI3K pathways. J Surg Res. 2006; 136(1):58-69.
Wang Y, Kelly C G, Singh M, et al. Stimulation of Th1-polarizing cytokines, C-C chemokines, maturation of dendritic cells, and adjuvant function by the peptide binding fragment of heat shock protein 70. J Immunol. 2002; 169(5):2422-2429.
Wesolowski R, Duggan M C, Stiff A, et al. Circulating myeloid-derived suppressor cells increase in patients undergoing neo-adjuvant chemotherapy for breast cancer. Cancer Immunol Immunother. 2017; 66(11):1437-1447.
Willert K, Brown J D, Danenberg E, et al. Wnt proteins are lipid-modified and can act as stem cell growth factors. Nature. 2003; 423(6938):448-452.
Wu et al., Trends Pharmacol. Sci., 38:226, 2017.
Wu C. Systemic Therapy for Colon Cancer. Surg Oncol Clin N Am. 2018; 27(2):235-242.
Ying H, Kimmelman A C, Lyssiotis C A, et al. Oncogenic Kras maintains pancreatic tumors through regulation of anabolic glucose metabolism. Cell. 2012; 149(3):656-670.
Zaheed M, Wilcken N, Willson M L, O'Connell D L, Goodwin A. Sequencing of anthracyclines and taxanes in neoadjuvant and adjuvant therapy for early breast cancer. Cochrane Database Syst Rev. 2019; 2:CD012873.
Zhang X D, Baladandayuthapani V, Lin H, et al. Tight Junction Protein 1 Modulates Proteasome Capacity and Proteasome Inhibitor Sensitivity in Multiple Myeloma via EGFR/JAK1/STAT3 Signaling. Cancer Cell. 2016; 29(5):639-652.
Zhang X, Lee H C, Shirazi F, et al. Protein targeting chimeric molecules specific for bromodomain and extra-terminal motif family proteins are active against pre-clinical models of multiple myeloma. Leukemia. 2018.
Zhuang J, Shirazi F, Singh R K, et al. Ubiquitin-activating enzyme inhibition induces an unfolded protein response and overcomes drug resistance in myeloma. Blood. 2019; 133(14):1572-1584.
Zininga T, Ramatsui L, Shonhai A. Heat Shock Proteins as Immunomodulants. Molecules. 2018; 23(11): E2846.

1. An antibody that binds human HSP70 comprising:
(i) an immunoglobulin heavy chain comprising the amino acid sequence of SEQ ID NO: 242 (hVH-1-G1m3), and an immunoglobulin light chain comprising the amino acid sequence of SEQ ID NO: 249 (hVL-1-Km3);
(ii) an immunoglobulin heavy chain comprising the amino acid sequence of SEQ ID NO: 243 (hVH-1-G1m3-GA), and an immunoglobulin light chain comprising the amino acid sequence of SEQ ID NO: 249 (hVL-1-Km3);
(iii) an immunoglobulin heavy chain comprising the amino acid sequence of SEQ ID NO: 244 (hVH-1-G1m3-GAALIE), and an immunoglobulin light chain comprising the amino acid sequence of SEQ ID NO: 249 (hVL-1-Km3);
(iv) an immunoglobulin heavy chain comprising the amino acid sequence of SEQ ID NO: 245 (hVH-1-G1m3-YTE), and an immunoglobulin light chain comprising the amino acid sequence of SEQ ID NO: 249 (hVL-1-Km3);
(v) an immunoglobulin heavy chain comprising the amino acid sequence of SEQ ID NO: 246 (hVH-1-G1m3-LS), and an immunoglobulin light chain comprising the amino acid sequence of SEQ ID NO: 249 (hVL-1-Km3);
(vi) an immunoglobulin heavy chain comprising the amino acid sequence of SEQ ID NO: 247 (hVH-1-G1m3-DF215), and an immunoglobulin light chain comprising the amino acid sequence of SEQ ID NO: 249 (hVL-1-Km3); or
(vii) an immunoglobulin heavy chain comprising the amino acid sequence of SEQ ID NO: 248 (hVH-1-G1m3-DF228), and an immunoglobulin light chain comprising the amino acid sequence of SEQ ID NO: 249 (hVL-1-Km3).

2. The antibody of claim 1, wherein the antibody binds to human HSP70 with a K_{D} of 50 nM or lower, 10 nM or lower, 5 nM or lower, 1 nM or lower, 0.75 nM or lower, 0.5 nM or lower, 0.1 nM, 0.075 nM, or 0.05 nM or lower, as measured by surface plasmon resonance or bio-layer interferometry.

3. The antibody of claim 1, wherein the antibody is capable of forming a high-order antibody:HSP70 complex.

4. The antibody of claim 3, wherein:
(i) the antibody:HSP70 complex has a molecular weight of at least about 290 kDa, at least about 300 kDa, at least about 580 kDa, at least about 1,450 kDa, or at least about 1,750 kDa;
(ii) the antibody:HSP70 complex has a molecular weight of at least about 300 kDa;
(iii) the antibody:HSP70 complex has a molecular weight of about 290 kDa, about 580 kDa, about 1,450 kDa, or about 1,750 kDa;
(iv) the ratio of antibody to HSP70 in the antibody:HSP70 complex is about 1:2; and/or
(v) the antibody:HSP70 complex comprises: (i) about one antibody molecule and about two HSP70 molecules; (ii) about two antibody molecules and about four HSP70 molecules; (iii) about five antibody molecules and about ten HSP70 molecules; or (iv) about six antibody molecules and about twelve HSP70 molecules.

5-8. (canceled)

9. The antibody of claim 3, wherein the antibody:HSP70 complex binds to a human FcγR with a K_{D} of 10 nM or lower, 5 nM or lower, 1 nM or lower, 0.75 nM or lower, 0.5 nM or lower, 0.1 nM, 0.075 nM, or 0.05 nM or lower, as measured by surface plasmon resonance or bio-layer interferometry.

10. The antibody of claim 9, wherein the FcγR is FcγR is FcgR1, FcgR2a, FcgR2b, FcgR2c, FcgR3a, and/or FcgR3b.

11. The antibody of claim 3, wherein the antibody enhances the uptake of tumor-derived ADP-HSP70-peptide antigen complexes by immune effector cells.

12. The antibody of claim 11, wherein the uptake is mediated by human FcgR1, FcgR2a, FcgR2b, FcgR2c, FcgR3a, and/or FcgR3b.

13. The antibody of claim 3, wherein the antibody:HSP70 complex activates immune effector cells.

14. The antibody of claim 13, wherein the immune effector cells comprise CD8+ T cells, CD4+ T cells, NK cells, and dendritic cells.

15. An antibody that is capable of forming a high-order antibody:HSP70 complex.

16-22. (canceled)

23. The antibody of claim 1, wherein:
(i) the antibody binds to an epitope of HSP70 corresponding to K573-Q601 of SEQ ID NO: 11;
(ii) the antibody binds to one, two, or three of the following residues: H594, K595, and 0601 of SEQ ID NO: 11;
(iii) the antibody binds to one, two, three, four, or five of the following residues: K573, E576, W580, R596, and E598 of SEQ ID NO: 11; and/or
(iv) the antibody binds to an epitope of HSP70 comprising KEWHKREQ (SEQ ID NO: 250).

24-26. (canceled)

27. The antibody of claim 1, wherein the antibody comprises:
(i) an immunoglobulin heavy chain variable region comprising a VHCDR1 comprising the amino acid sequence of SEQ ID NO: 1, a VHCDR2 comprising the amino acid sequence of SEQ ID NO: 2, and a VHCDR3 comprising the amino acid sequence of SEQ ID NO: 3, and an immunoglobulin light chain variable region comprising a VLCDR1 comprising the amino acid sequence of SEQ ID NO: 4, a VLCDR2 comprising the amino acid sequence of SEQ ID NO: 5, and a VLCDR3 comprising the amino acid sequence of SEQ ID NO: 6; or
ii) an immunoglobulin heavy chain variable region comprising the amino acid sequence of SEQ ID NO: 12 (hVH-1), and an immunoglobulin light chain variable region comprising the amino acid sequence of SEQ ID NO: 19 (hVL-1).

28-29. (canceled)

30. A high-order antibody:HSP70 complex comprising: (i) an antibody; and (ii) HSP70.

31-42. (canceled)

43. An isolated nucleic acid comprising a nucleotide sequence encoding the immunoglobulin heavy chain of the antibody of claim 1.

44. An expression vector comprising a nucleic acid comprising a nucleotide sequence encoding the immunoglobulin heavy chain of the antibody of claim 1.

45. A host cell comprising the expression vector of claim 44.

46. A pharmaceutical composition comprising the antibody of claim 1.

47. A method of treating cancer in a subject in need thereof, the method comprising administering to the subject an effective amount of the antibody of claim 1.

48-52. (canceled)

53. A method of enhancing uptake of tumor-derived ADP-HSP70-peptide antigen complexes by immune effector cells, the method comprising contacting the cells with an effective amount of the antibody of claim 1.


SEQUENCE LISTING (251 sequences)

SEQ ID NO: 1 - protein, 8 aa, synthetic construct
  REGION 1..8: note = Engineered polynucleotide
GYTFTNYG

SEQ ID NO: 2 - protein, 8 aa, synthetic construct
  REGION 1..8: note = Engineered polynucleotide
INTYTGEP

SEQ ID NO: 3 - protein, 9 aa, synthetic construct
  REGION 1..9: note = Engineered polynucleotide
ARYDHAMDY

SEQ ID NO: 4 - protein, 12 aa, synthetic construct
  REGION 1..12: note = Engineered polynucleotide
QSLLNSGTRKNY

SEQ ID NO: 5 - skipped

SEQ ID NO: 6 - protein, 8 aa, synthetic construct
  REGION 1..8: note = Engineered polynucleotide
KQSYTLYT

SEQ ID NO: 7 - protein, 116 aa, synthetic construct
  REGION 1..116: note = Engineered polynucleotide
QIQLVQSGPELKKPGETVKISCKASGYTFTNYGMNWVKQAPGKGLKWMGWINTYTGEPTYADDFKGRFAFSLETSATTAYLQINNLKNEDTATYFCARYDHAMDYWGQGTSVTVSS

SEQ ID NO: 8 - protein, 112 aa, synthetic construct
  REGION 1..112: note = Engineered polynucleotide
DIVMSQSPSSLAVSAGEKVTMSCKSSQSLLNSGTRKNYLAWYQQKPGQSPKLLIYWTSTRESGVPDRFTGSGSGTDFTLTINSVQAEDLAVYYCKQSYTLYTFGGGTKLEIK

SEQ ID NO: 9 - DNA, 349 bp, synthetic construct
  misc_feature 1..349: note = Engineered polynucleotide
  source 1..349: mol_type = other DNA
cagatccagttggtgcagtctggacctgagctgaagaagcctggagagacagtcaagatctcctgcaaggcttctgggtataccttcacaaactatggaatgaactgggtgaagcaggctccaggaaagggtttaaagtggatgggctggataaacacctacactggagagccaacatatgctgatgacttcaagggacggtttgccttctctttggaaacctctgccaccactgcctatttgcagatcaacaacctcaaaaatgaggacacggctacatatttctgtgcaaggtacgaccatgctatggactactggggtcaaggaacctcagtcaccgtctcctcag

SEQ ID NO: 10 - DNA, 337 bp, synthetic construct
  misc_feature 1..337: note = Engineered polynucleotide
  source 1..337: mol_type = other DNA
gacattgtgatgtcacagtctccatcctccctggctgtgtcagctggagagaaggtcactatgagctgcaaatccagtcagagtctgctcaacagtggaacccgaaagaactacttggcttggtaccagcagaaaccagggcagtctcctaaactgctgatctactggacatccactagggaatctggggtccctgatcgcttcacaggcagtggatctgggacagatttcactctcaccatcaacagtgtgcaggctgaagacctggcagtttattactgcaagcaatcttatactctgtacacgttcggaggggggaccaagctggaaataaaac

SEQ ID NO: 11 - protein, 641 aa, Homo sapiens
MAKAAAIGIDLGTTYSCVGVFQHGKVEIIANDQGNRTTPSYVAFTDTERLIGDAAKNQVALNPQNTVFDAKRLIGRKFGDPVVQSDMKHWPFQVINDGDKPKVQVSYKGETKAFYPEEISSMVLTKMKEIAEAYLGYPVTNAVITVPAYFNDSQRQATKDAGVIAGLNVLRIINEPTAAAIAYGLDRTGKGERNVLIFDLGGGTFDVSILTIDDGIFEVKATAGDTHLGGEDFDNRLVNHFVEEFKRKHKKDISQNKRAVRRLRTACERAKRTLSSSTQASLEIDSLFEGIDFYTSITRARFEELCSDLFRSTLEPVEKALRDAKLDKAQIHDLVLVGGSTRIPKVQKLLQDFFNGRDLNKSINPDEAVAYGAAVQAAILMGDKSENVQDLLLLDVAPLSLGLETAGGVMTALIKRNSTIPTKQTQIFTTYSDNQPGVLIQVYEGERAMTKDNNLLGRFELSGIPPAPRGVPQIEVTFDIDANGILNVTATDKSTGKANKITITNDKGRLSKEEIERMVQEAEKYKAEDEVQRERVSAKNALESYAFNMKSAVEDEGLKGKISEADKKKVLDKCQEVISWLDANTLAEKDEFEHKRKELEQVCNPIISGLYQGAGGPGPGGFGAQGPKGGSGSGPTIEEVD

SEQ ID NO: 12 - protein, 116 aa, synthetic construct
  REGION 1..116: note = Engineered polynucleotide
QIQLVQSGAEVKKPGASVKVSCKASGYTFTNYGMNWVRQAPGQGLEWMGWINTYTGEPTYADDFKGRFTFTTDTSTSTAYMELRSLRSDDTAVYFCARYDHAMDYWGQGTLVTVSS

SEQ ID NO: 13 - protein, 116 aa, synthetic construct
  REGION 1..116: note = Engineered polynucleotide
QIQLVQSGAEVKKPGSSVKVSCKASGYTFTNYGMNWVRQAPGQGLEWMGWINTYTGEPTYADDFKGRFTFTADESTSTAYMELSSLRSEDTAVYFCARYDHAMDYWGQGTLVTVSS

SEQ ID NO: 14 - protein, 116 aa, synthetic construct
  REGION 1..116: note = Engineered polynucleotide
QVQLQQSGPEVKKPGASVKISCKTSGYTFTNYGMNWVRQAPGQGLEWMGWINTYTGEPTYADDFKGRVTMTTDTSTSTAYLELTGLMSDDTAVYFCARYDHAMDYWGQGTTVTVSS

SEQ ID NO: 15 - protein, 116 aa, synthetic construct
  REGION 1..116: note = Engineered polynucleotide
EVQLVESGGGLVKPGGSLRLSCKASGYTFTNYGMNWVRQAPGKGLKWVGWINTYTGEPTYADDFKGRFTFSRDDSKNTLYLQMNSLKTEDTAVYFCARYDHAMDYWGQGTSVTVSS

SEQ ID NO: 16 - protein, 116 aa, synthetic construct
  REGION 1..116: note = Engineered polynucleotide
QIQLVQSGPEVKKPGESVKVSCKASGYTFTNYGMNWVRQAPGKGLEWMGWINTYTGEPTYADDFKGRVTITRDTSASTAYMELSSLRSEDTAVYFCARYDHAMDYWGQGTSVTVSS

SEQ ID NO: 17 - protein, 116 aa, synthetic construct
  REGION 1..116: note = Engineered polynucleotide
QIQLVQSGAEVKKPGASVKVSCKASGYTFTNYGMNWVRQAPGQGLEWMGWINTYTGEPTYADDFKGRFTMTTDTSTSTAYMELRSLRSDDTAVYFCARYDHAMDYWGQGTLVTVSS

SEQ ID NO: 18 - protein, 116 aa, synthetic construct
  REGION 1..116: note = Engineered polynucleotide
  SITE 1: note = MISC_FEATURE - Xaa is Q or E
  SITE 2: note = MISC_FEATURE - Xaa is I or V
  SITE 5: note = MISC_FEATURE - Xaa is V or Q
  SITE 6: note = MISC_FEATURE - Xaa is Q or E
  SITE 9: note = MISC_FEATURE - Xaa is A, P, or G
  SITE 10: note = MISC_FEATURE - Xaa is E or G
  SITE 11: note = MISC_FEATURE - Xaa is V or L
  SITE 12: note = MISC_FEATURE - Xaa is V or K
  SITE 16: note = MISC_FEATURE - Xaa is A, E, G, or S
  SITE 18: note = MISC_FEATURE - Xaa is V or L
  SITE 19: note = MISC_FEATURE - Xaa is K or R
  SITE 20: note = MISC_FEATURE - Xaa is V, L, or I
  SITE 24: note = MISC_FEATURE - Xaa is A or T
  SITE 43: note = MISC_FEATURE - Xaa is K or Q
  SITE 46: note = MISC_FEATURE - Xaa is E or K
  SITE 48: note = MISC_FEATURE - Xaa is M or V
  SITE 68: note = MISC_FEATURE - Xaa is F or V
  SITE 70: note = MISC_FEATURE - Xaa is F, M, or I
  SITE 71: note = MISC_FEATURE - Xaa is T or S
  SITE 72: note = MISC_FEATURE - Xaa is T, R, or A
  SITE 74: note = MISC_FEATURE - Xaa is T, D, or E
  SITE 76: note = MISC_FEATURE - Xaa is T, A, or K
  SITE 77: note = MISC_FEATURE - Xaa is S or N
  SITE 79: note = MISC_FEATURE - Xaa is L or A
  SITE 81: note = MISC_FEATURE - Xaa is M or L
  SITE 82: note = MISC_FEATURE - Xaa is E or Q
  SITE 83: note = MISC_FEATURE - Xaa is L or M
  SITE 84: note = MISC_FEATURE - Xaa is R, S, T, or N
  SITE 85: note = MISC_FEATURE - Xaa is S or G
  SITE 87: note = MISC_FEATURE - Xaa is R, K, or M
  SITE 88: note = MISC_FEATURE - Xaa is S or T
  SITE 89: note = MISC_FEATURE - Xaa is D or E
  SITE 111: note = MISC_FEATURE - Xaa is L, S, or T
XXQLXXSGXXXXKPGXSXXXSCKXSGYTFTNYGMNWVRQAPGXGLXWXGWINTYTGEPTYADDFKGRXTXXXDXSXXTXYXXXXXLXXXDTAVYFCARYDHAMDYWGQGTXVTVSS

SEQ ID NO: 19 - protein, 112 aa, synthetic construct
  REGION 1..112: note = Engineered polynucleotide
EIVLTQSPDSLAVSLGERATINCKSSQSLLNSGTRKNYLAWYQQKAGQSPKLLIYWTSTRESGVPDRFSGSGSGTDFTLTIDSLQAEDVAVYYCKQSYTLYTFGGGTKVEIK

SEQ ID NO: 20 - protein, 112 aa, synthetic construct
  REGION 1..112: note = Engineered polynucleotide
DIVMTQSPDSLSVSLGERATINCKSSQSLLNSGTRKNYLAWYQQKPGQPPRLLIYWTSTRESGVPDRFSGSGSGTDFTLTINTLQAEDVAVYYCKQSYTLYTFGQGTKLEIK

SEQ ID NO: 21 - protein, 112 aa, synthetic construct
  REGION 1..112: note = Engineered polynucleotide
DVVMTQSPDSLAVSLGERVTINCKSSQSLLNSGTRKNYLAWYQQKPGQSPKLLIYWTSTRESGVPDRFSGSGSGTDFTLTISSLQAEDVAVYYCKQSYTLYTFGQGTKLEIK

SEQ ID NO: 22 - protein, 112 aa, synthetic construct
  REGION 1..112: note = Engineered polynucleotide
DIQMTQSPSSLSASVGDRVTITCKSSQSLLNSGTRKNYLAWYQQKPGKVPKLLIYWTSTRESGVPSRFSGSGSGTDFTLTISSLQPEDVATYYCKQSYTLYTFGGGTKLEIK

SEQ ID NO: 23 - protein, 112 aa, synthetic construct
  REGION 1..112: note = Engineered polynucleotide
DIVMTQSPDSLAVSLGERATINCKSSQSLLNSGTRKNYLAWYQQKPGQSPKLLIYWTSTRESGVPDRFSGSGSGTDFTLTISSLQAEDVAVYYCKQSYTLYTFGGGTKLEIK

SEQ ID NO: 24 - protein, 112 aa, synthetic construct
  REGION 1..112: note = Engineered polynucleotide
DIVMTQSPDSLAVSLGERATINCKSSQSLLNSGTRKNYLAWYQQKPGQSPKLLIYWTSTRESGVPDRFSGSGSGTDFTLTISSLQAEDVAVYYCKQSYTLYTFGQGTKLEIK

SEQ ID NO: 25 - protein, 112 aa, synthetic construct
  REGION 1..112: note = Engineered polynucleotide
  SITE 1: note = MISC_FEATURE - Xaa is E or D
  SITE 2: note = MISC_FEATURE - Xaa is I or V
  SITE 3: note = MISC_FEATURE - Xaa is V or Q
  SITE 4: note = MISC_FEATURE - Xaa is L or M
  SITE 9: note = MISC_FEATURE - Xaa is D or S
  SITE 12: note = MISC_FEATURE - Xaa is A or S
  SITE 13: note = MISC_FEATURE - Xaa is V or A
  SITE 15: note = MISC_FEATURE - Xaa is L or V
  SITE 17: note = MISC_FEATURE - Xaa is E or D
  SITE 19: note = MISC_FEATURE - Xaa is A or V
  SITE 22: note = MISC_FEATURE - Xaa is N or T
  SITE 46: note = MISC_FEATURE - Xaa is A or P
  SITE 48: note = MISC_FEATURE - Xaa is Q or K
  SITE 49: note = MISC_FEATURE - Xaa is S, V, or P
  SITE 51: note = MISC_FEATURE - Xaa is K or R
  SITE 66: note = MISC_FEATURE - Xaa is D or S
  SITE 82: note = MISC_FEATURE - Xaa is S, D, or N
  SITE 83: note = MISC_FEATURE - Xaa is S or T
  SITE 86: note = MISC_FEATURE - Xaa is A or P
  SITE 91: note = MISC_FEATURE - Xaa is V or T
  SITE 105: note = MISC_FEATURE - Xaa is Q or G
  SITE 109: note = MISC_FEATURE - Xaa is L or V
XXXXTQSPXSLXXSXGXRXTIXCKSSQSLLNSGTRKNYLAWYQQKXGXXPXLLIYWTSTRESGVPXRFSGSGSGTDFTLTIXXLQXEDVAXYYCKQSYTLYTFGXGTKXEIK

SEQ ID NO: 26 - protein, 116 aa, synthetic construct
  REGION 1..116: note = Engineered polynucleotide
QIQLVQSGAEVKKPGASVKVSCKASGYTFTKYGMNWVRQAPGQGLEWMGWINTYTGEPTYADDFKGRFTFTTDTSTSTAYMELRSLRSDDTAVYFCARYDHAMDYWGQGTLVTVSS

SEQ ID NO: 27 - protein, 116 aa, synthetic construct
  REGION 1..116: note = Engineered polynucleotide
QIQLVQSGAEVKKPGASVKVSCKASGYTFTNYGMNWVRQAPGQGLEWMGWINTYTGEPRYADDFKGRFTFTTDTSTSTAYMELRSLRSDDTAVYFCARYDHAMDYWGQGTLVTVSS

SEQ ID NO: 28 - protein, 116 aa, synthetic construct
  REGION 1..116: note = Engineered polynucleotide
QIQLVQSGAEVKKPGASVKVSCKASGYTFTNYGMNWVRQAPGQGLEWMGWINTYTGEPTYADDFKGRFTFTTDTSTSTAYMELRSLRSDDTAVYFCARYDHRMDYWGQGTLVTVSS

SEQ ID NO: 29 - protein, 116 aa, synthetic construct
  REGION 1..116: note = Engineered polynucleotide
QIQLVQSGAEVKKPGASVKVSCKASGYTFTNYGMNWVRQAPGQGLEWMGWINTYTGEPIYADDFKGRFTFTTDTSTSTAYMELRSLRSDDTAVYFCARYDHAMDYWGQGTLVTVSS

SEQ ID NO: 30 - protein, 116 aa, synthetic construct; identical to SEQ ID NO: 12
  REGION 1..116: note = Engineered polynucleotide

SEQ ID NO: 31 - protein, 116 aa, synthetic construct
  REGION 1..116: note = Engineered polynucleotide
QIQLVQSGAEVKKPGASVKVSCKASGYTFTNYGMNWVRQAPGQGLEWMGWINTYTGEPTYADDFKGRFTFTTDTSTRTAYMELRSLRSDDTAVYFCARYDHAMDYWGQGTLVTVSS

SEQ ID NO: 32 - protein, 116 aa, synthetic construct
  REGION 1..116: note = Engineered polynucleotide
QIQLVQSGAEVKKPGASVKVSCKASGYTFTNYGMNWVRQAPGQGLEWMGWINTYTGEPTYADDFKGRFTFTTDTSTSTAYMELRSLRSDDTAVYFCARYDHFMDYWGQGTLVTVSS

SEQ ID NO: 33 - protein, 116 aa, synthetic construct
  REGION 1..116: note = Engineered polynucleotide
QIQLVQSGAEVKKPGASVKVSCKASGYTFTNYGMNWVRQAPGQGLEWMGWINTYTGEPTYADDFKGRFTFTTDTSTSTAYMELRSLRSDDTAVYFCARYDHTMDYWGQGTLVTVSS

SEQ ID NO: 34 - protein, 116 aa, synthetic construct
  REGION 1..116: note = Engineered polynucleotide
QIQLVQSGDEVKKPGASVKVSCKASGYTFTNYGMNWVRQAPGQGLEWMGWINTYTGEPTYADDFKGRFTFTTDTSTSTAYMELRSLRSDDTAVYFCARYDHAMDYWGQGTLVTVSS

SEQ ID NO: 35 - protein, 116 aa, synthetic construct
  REGION 1..116: note = Engineered polynucleotide
QIQLVQSGAEVKKPGASVKVSCKASGYTFTNYGMNWVRQAPGQGLEWMGWINTYTGEPTYADDFKGRFTFTTDTSTSTAYMELRSLRSDDTAVYFCARYDHPMDYWGQGTLVTVSS

SEQ ID NO: 36 - protein, 116 aa, synthetic construct
  REGION 1..116: note = Engineered polynucleotide
QIQLVQSGAEVKKPGASVKVSCKASGYTFTNYGMNWVRQAPGQGLEWMGWINTYTGEPTYTDDFKGRFTFTTDTSTSTAYMELRSLRSDDTAVYFCARYDHAMDYWGQGTLVTVSS

SEQ ID NO: 37 - protein, 116 aa, synthetic construct
  REGION 1..116: note = Engineered polynucleotide
QIQLVQSGAEVKKPGASVKVSCKASGYTFTNYGMNWVRQAPGQGLEWMGWINTYTGEPTYADDFKGRFTFTTDTSTSTAYMELRSLRSDDTAVYFCARYDHVMDYWGQGTLVTVSS

SEQ ID NO: 38 - protein, 116 aa, synthetic construct
  REGION 1..116: note = Engineered polynucleotide
QIQLVQSGAEVKKPGASVKVSCKASGYTFTKYGMNWVRQAPGQGLEWMGWINTYTGEPTYADDFKGRFTFTTDTSTSTAYMELRSLRSDDTAVYFCARYDHSMDYWGQGTLVTVSS

SEQ ID NO: 39 - protein, 116 aa, synthetic construct
  REGION 1..116: note = Engineered polynucleotide
QIQLVQSGAEVKKPGASVKVSCKASGYTFTNYGMNWVRQAPGQGLEWMGWINTYTGEPTYVDDFKGRFTFTTDTSTSTAYMDLRSLRSDDTAVYFCARYDHAMDYWGQGTLVTVSS

SEQ ID NO: 40 - protein, 116 aa, synthetic construct
  REGION 1..116: note = Engineered polynucleotide
QIQLVQSGDEVKKPGASVKVSCKASGYTFTNYGMNWVRQAPGQGLEWMGWINTYTGEPTYSDDFKGRFTFTTDTSTSTAYMELRSLRSDDTAVYFCARYDHAMDYWGQGTLVTVSS

SEQ ID NO: 41 - protein, 116 aa, synthetic construct
  REGION 1..116: note = Engineered polynucleotide
QIQLVQSGAEVKKPGASVKVSCKASGYTFTNYGMNWVRQAPGQGLEWMGWINTYTGEPTYSDDFKGRFTFTTDTSTSTAYMELRSLRSDDTAVYFCARYDHAMDYWGQGTLVTVSS

SEQ ID NO: 42 - protein, 116 aa, synthetic construct
  REGION 1..116: note = Engineered polynucleotide
QIQLVQSGAEVKKPGASVKVSCKASGYTFTNYGMNWVRQAPGQGLEWMGWINTYTGEPTYADDFKGRFTFTTDTSTRTAYMELRSLRSDDTAVYFCTRYDHAMDYWGQGTLVTVSS

SEQ ID NO: 43 - protein, 116 aa, synthetic construct
  REGION 1..116: note = Engineered polynucleotide
QIQLVQSGTEVKKPGASVKVSCKASGYTFTNYGMNWVRQAPGQGLEWMGWINTYTGEPTYADDFKGRFTFTTDTSTSTAYMELRSLRSDDTAVYFCARYDHTMDYWGQGTLVTVSS

SEQ ID NO: 44 - protein, 116 aa, synthetic construct
  REGION 1..116: note = Engineered polynucleotide
QIQLVQSGAEVKKPGASVKVSCKASGYTFTNYGMNWVRQAPGQGLEWMGWINTYTGEPTYADDFKGRFTFTTDTSTSTAYMEVRSLRSDDTAVYFCARYDHAMDYWGQGTLVTVSS

SEQ ID NO: 45 - protein, 116 aa, synthetic construct
  REGION 1..116: note = Engineered polynucleotide
QIQLVQSGAEVKKPGASVKVSCKASGYTFTNYGMNWVRQAPGQGLEWMGWINTYTGEPRYADDFKGRFTFTTDTSTSTAYMELRSLRSDDTAVYFCARYDHDMDYWGQGTLVTVSS

SEQ ID NO: 46 - protein, 116 aa, synthetic construct
  REGION 1..116: note = Engineered polynucleotide
QIQLVQSGAEVKKPGASVKVSCKASGYTFTKYGMNWVRQAPGQGLEWMGWINTYTGEPTYADDFKGRFTFTTDTSTSTAYMELRSLRSDDTAVYFCARYDHNMDYWGQGTLVTVSS

SEQ ID NO: 47 - protein, 116 aa, synthetic construct
  REGION 1..116: note = Engineered polynucleotide
QIQLVQSGTEVKKPGASVKVSCKASGYTFTNYGMNWVRQAPGQGLEWMGWINTYTGEPTYADDFKGRFTFTTDTSTSTAYMELRSLRSDDTAVYFCARYDHHMDYWGQGTLVTVSS

SEQ ID NO: 48 - protein, 116 aa, synthetic construct
  REGION 1..116: note = Engineered polynucleotide
QIQLVQSGAEVKKPGASVKVSCKASGYTFTNYGMNWVRQAPGQGLEWMGWINTYTGEPTYADDFKGRFTFTTDTSTTTAYMELRSLRSDDTAVYFCARYDHHMDYWGQGTLVTVSS

SEQ ID NO: 49 - protein, 116 aa, synthetic construct
  REGION 1..116: note = Engineered polynucleotide
QIQLVQSGAEVKKPGASVKVSCKASGYTFTKYGMNWVRQAPGQGLEWMGWINTYTGESTYADDFKGRFTFTTDTSTSTAYMELRSLRSDDTAVYFCARYDHVMDYWGQGTLVTVSS

SEQ ID NO: 50 - protein, 116 aa, synthetic construct
  REGION 1..116: note = Engineered polynucleotide
QIQLVQSGTEVKKPGASVKVSCKASGYTFTNYGMNWVRQAPGQGLEWMGWINTYTGEPTYADDFKGRFTFTTDTSTRTAYMELRSLRSDDTAVYFCARYDHLMDYWGQGTLVTVSS

SEQ ID NO: 51 - protein, 116 aa, synthetic construct
  REGION 1..116: note = Engineered polynucleotide
QIQLVQSGAEVKKPGASVKVSCKASGYTFTNYGMNWVRQAPGQGLEWMGWINTYTGEPTYSDDFKGRFTFTTDTSTRTAYMELRSLRSDDTAVYFCARYDHAMDYWGQGTLVTVSS

SEQ ID NO: 52 - protein, 116 aa, synthetic construct
  REGION 1..116: note = Engineered polynucleotide
QIQLVQSGAEVKKPGASVKVSCKASGYTFTNYGMNWVRQAPGQGLEWMGWINTYTGEPTYADDFKGRFTFTTDTSTSTAYMELRSLRSDDTAVYFCARYDHYMDYWGQGTLVTVSS

SEQ ID NO: 53 - protein, 116 aa, synthetic construct
  REGION 1..116: note = Engineered polynucleotide
QIQLVQSGAEVKKPGASVKVSCKASGYTFTNYGMNWVRQAPGQGLEWMGWINTYTGEPTYADDFKGRFTFTTDTSTTTAYMELRSLRSDDTAVYFCARYDHVMDYWGQGTLVTVSS

SEQ ID NO: 54 - protein, 116 aa, synthetic construct
  REGION 1..116: note = Engineered polynucleotide
QIHLVQSGAEVKKPGASVKVSCKASGYTFTNYGMNWVRQAPGQGLEWMGWINTYTGEPTYADDFKGRFTFTTDTSTSTAYMELRSLRSDDTAVYFCTRYDHRMDYWGQGTLVTVSS

SEQ ID NO: 55 - protein, 116 aa, synthetic construct
  REGION 1..116: note = Engineered polynucleotide
QIHLVQSGAEVKKPGASVKVSCKASGYTFTNYGMNWVRQAPGQGLEWMGWINTYTGEPTYADDFKGRFTFTTDTSTSTVYMELRSLRSDDTAVYFCARYDHAMDYWGQGTLVTVSS

SEQ ID NO: 56 - protein, 116 aa, synthetic construct
  REGION 1..116: note = Engineered polynucleotide
QIQLVQSGAEVKKPGASVKVSCKASGYTFTNYGMNWVRQAPGQGLEWMGWINTYTGEPTYADDFKGRFTFTTDTSTSTAYMELRSLRSDDTAVYFCVRYDHAMDYWGQGSLVTVSS

SEQ ID NO: 57 - protein, 116 aa, synthetic construct
  REGION 1..116: note = Engineered polynucleotide
QIQLVQSGVEVKKPGASVKVSCKASGYTFTNYGMNWVRQAPGQGLEWMGWINTYTGEPTYADDFKGRFTFTTDTSTSTVYMELRSLRSDDTAVYFCARYDHAMDYWGQGTLVTVSS

SEQ ID NO: 58 - protein, 116 aa, synthetic construct
  REGION 1..116: note = Engineered polynucleotide
QIQLVQSGVEVKKPGASVKVSCKASGYTFTNYGMNWVRQAPGQGLEWMGWINTYTGEPTYADDFKGRFTFTTDTSTSTAYMELRSLRSDDTAVYFCARYDHAMDYWGQGTLVTVSS

SEQ ID NO: 59 - protein, 116 aa, synthetic construct
  REGION 1..116: note = Engineered polynucleotide
QIQLVQSGTEVKKPGASVKVSCKASGYTFTNYGMNWVRQAPGQGLEWMGWINTYTGEPTYADDFKGRFTFTTDTSTSTAYMELRSLRSDDTAVYFCARYDHLMDYWGQGTLVTVSS

SEQ ID NO: 60 - protein, 116 aa, synthetic construct
  REGION 1..116: note = Engineered polynucleotide
QIQLVQSGAEVKKPGASVKVSCKASGYTFTNYGMNWVRQAPGQGLEWMGWINTYTGEPTYADDFKGRFTFTTDTSTSTAYMDLRSLRSDDTAVYFCARYDHYMDYWGQGTLVTVSS

SEQ ID NO: 61 - protein, 116 aa, synthetic construct
  REGION 1..116: note = Engineered polynucleotide
QIQLVQSGAEVKKPGASVKVSCKASGYTFTNYGMNWVRQAPGQGLEWMGWINTYTGETTYGDDFKGRFTFTTDTSTSTAYMELRSLRSDDTAVYFCGRYDHAMDYWGQGTLVTVSS

SEQ ID NO: 62 - protein, 116 aa, synthetic construct
  REGION 1..116: note = Engineered polynucleotide
QIHLVQSGVEVKKPGASVKVSCKASGYTFTNYGMNWVRQAPGQGLEWMGWINTYTGEPTYTDDFKGRFTFTTDTSTSTAYMELRSLRSDDTAVYFCARYDHAMDYWGQGTLVTVSS

SEQ ID NO: 63 - protein, 116 aa, synthetic construct
  REGION 1..116: note = Engineered polynucleotide
QIQLVQSGAEVKKPGASVKVSCKASGYSFTNYGMNWVRQAPGQGLEWMGWINTYTGEPTYADDFKGRFTFTTDTSTSTAYMELRSLRSDDTAVYFCARYDHGMDYWGQGTLVTVSS

SEQ ID NO: 64 - protein, 116 aa, synthetic construct
  REGION 1..116: note = Engineered polynucleotide
QIQLVQSGVEVKKPGASVKVSCKASGYIFTNYGMNWVRQAPGQGLEWMGWINTYTGEPRYADDFKGRFTFTTDTSTSTAYMELRSLRSDDTAVYFCARYDHRMDYWGQGTLVTVSS

SEQ ID NO: 65 - protein, 116 aa, synthetic construct
  REGION 1..116: note = Engineered polynucleotide
QIQLVQSGAEVKKPGASVKVSCKASGYTFTNYGMNWVRQAPGQGLEWMGWINTYTGEPKYTDDFKGRFTFTTDTSTRTAYMELRSLRSDDTAVYFCARYDHAMDYWGQGTLVTVSS

SEQ ID NO: 66 - protein, 116 aa, synthetic construct
  REGION 1..116: note = Engineered polynucleotide
QIQLVQSGAEVKKPGASVKVSCKASGYTFTKYGMNWVRQAPGQGLEWMGWINTYTGEPKYGDDFKGRFTFTTDTSTSTAYMELRSLRSDDTAVYFCARYDHRMDYWGQGTLVTVSS

SEQ ID NO: 67 - protein, 116 aa, synthetic construct
  REGION 1..116: note = Engineered polynucleotide
QIQLVQSGSEVKKPGASVKVSCKASGYTFTNYGMNWVRQAPGQGLEWMGWINTYTGEPTYGDDFKGRFTFTTDTSTSTAYMELRSLRSDDTAVYFCARYDHFMDYWGQGTLVTVSS

SEQ ID NO: 68 - protein, 116 aa, synthetic construct
  REGION 1..116: note = Engineered polynucleotide
QIQLVQSGTEVKKPGASVKVSCKASGYSFTNYGMNWVRQAPGQGLEWMGWINTYTGEPTYADDFKGRFTFTTDTSTTTAYMELRSLRSDDTAVYFCARYDHAMDYWGQGTLVTVSS

SEQ ID NO: 69 - protein, 116 aa, synthetic construct
  REGION 1..116: note = Engineered polynucleotide
QIQLVQSGAEVKKPGASVKVSCKASGYTFTNYGMNWVRQAPGQGLEWMGWINTYTGEATYADDFKGRFTFTTDTSTSTAYMELRSLRSDDTAVYFCARYDHVMDYWGQGTLVTVSS

SEQ ID NO: 70 - protein, 116 aa, synthetic construct
  REGION 1..116: note = Engineered polynucleotide
QIQLVQSGSEVKKPGASVKVSCKASGYTFTNYGMNWVRQAPGQGLEWMGWINTYTGEPTYADDFKGRFTFTTDTSTSTAYMELRSLRSDDTAVYFCGRYDHAMDYWGQGTLVTVSS

SEQ ID NO: 71 - protein, 116 aa, synthetic construct
  REGION 1..116: note = Engineered polynucleotide
QIQLVQSGPEVKKPGASVKVSCKASGYTFTNYGMNWVRQAPGQGLEWMGWINTYTGEPTYGDDFKGRFTFTTDTSTSTAYMELRSLRSDDTAVYFCGRYDHAMDYWGQGTLVTVSS

SEQ ID NO: 72 - protein, 116 aa, synthetic construct
  REGION 1..116: note = Engineered polynucleotide
QIQLVQSGSEVKKPGASVKVSCKASGYTFTNYGMNWVRQAPGQGLEWMGWINTYTGEPTYADDFKGRFTFTTDTSTSTAYMELRSLRSDDTAVYFCARYDHSMDYWGQGTLVTVSS

SEQ ID NO: 73 - protein, 116 aa, synthetic construct
  REGION 1..116: note = Engineered polynucleotide
QIQLVQSGAEVKKPGASVKVSCKASGYTFTNYGMNWVRQAPGQGLEWMGWINTYTGEPTYADDFKGRFTFTTDTSTSTAYMELRSLRSDDTAVYFCARYDHAMDYWGQGSLVTVSS

SEQ ID NO: 74 - protein, 116 aa, synthetic construct
  REGION 1..116: note = Engineered polynucleotide
QIQLVQSGAEVKKPGASVKVSCKASGYTFTNYGMNWVRQAPGQGLEWMGWINTYTGEPTYADDFKGRFTFTTDTSTTTGYMELRSLRSDDTAVYFCARYDHAMDYWGQGTLVTVSS

SEQ ID NO: 75 - protein, 116 aa, synthetic construct
  REGION 1..116: note = Engineered polynucleotide
QIQLVQSGAEVKKPGASVKVSCKASGYTFTNYGMNWVRQAPGQGLEWMGWINTYTGESTYADDFKGRFTFTTDTSTSTAYMELRSLRSDDTAVYFCARYDHAMDYWGQGTLVTVSS

SEQ ID NO: 76 - protein, 116 aa, synthetic construct
  REGION 1..116: note = Engineered polynucleotide
QIQLVQSGAEVKKPGASVKVSCKASGYTFTNYGMNWVRQAPGQGLEWMGWINTYTGEATYADDFKGRFTFTTDTSTSTAYMELRSLRSDDTAVYFCARYDHAMDYWGQGSLVTVSS

SEQ ID NO: 77 - protein, 116 aa, synthetic construct
  REGION 1..116: note = Engineered polynucleotide
QIQLVQSGAEVKKPGASVKVSCKASGYIFTNYGMNWVRQAPGQGLEWMGWINTYTGESTYADDFKGRFTFTTDTSTSTVYMELRSLRSDDTAVYFCARYDHAMDYWGQGTLVTVSS

SEQ ID NO: 78 - protein, 116 aa, synthetic construct
  REGION 1..116: note = Engineered polynucleotide
QIQLVQSGAEVKKPGASVKVSCKASGYTFTNYGMNWVRQAPGQGLEWMGWINTYTGEPTYADDFKGRFTFTTDTSTSTAYMDLRSLRSDDTAVYFCARYDHAMDYWGQGTLVTVSS

SEQ ID NO: 79 - protein, 116 aa, synthetic construct
  REGION 1..116: note = Engineered polynucleotide
QIHLVQSGDEVKKPGASVKVSCKASGYTFTNYGMNWVRQAPGQGLEWMGWINTYTGEPTYTDDFKGRFTFTTDTSTSTAYMELRSLRSDDTAVYFCARYDHAMDYWGQGTLVTVSS

SEQ ID NO: 80 - protein, 116 aa, synthetic construct
  REGION 1..116: note = Engineered polynucleotide
QIQLVQSGAEVKKPGASVKVSCKASGYTFTNYGMNWVRQAPGQGLEWMGWINTYTGETTYTDDFKGRFTFTTDTSTSTAYMELRSLRSDDTAVYFCARYDHAMDYWGQGTLVTVSS

SEQ ID NO: 81 - protein, 116 aa, synthetic construct
  REGION 1..116: note = Engineered polynucleotide
QIQLVQSGSEVKKPGASVKVSCKASGYTFTNYGMNWVRQAPGQGLEWMGWINTYTGEPTYADDFKGRFTFTTDTSTSTAYMDLRSLRSDDTAVYFCARYDHAMDYWGQGTLVTVSS

SEQ ID NO: 82 - protein, 116 aa, synthetic construct
  REGION 1..116: note = Engineered polynucleotide
QIQLVQSGPEVKKPGASVKVSCKASGYIFTNYGMNWVRQAPGQGLEWMGWINTYTGETTYADDFKGRFTFTTDTSTTTVYMELRSLRSDDTAVYFCARYDHAMDYWGQGTLVTVSS

SEQ ID NO: 83 - protein, 116 aa, synthetic construct
  REGION 1..116: note = Engineered polynucleotide
QIHLVQSGDEVKKPGASVKVSCKASGYTFTNYGMNWVRQAPGQGLEWMGWINTYTGEPTYADDFKGRFTFTTDTSTSTGYMELRSLRSDDTAVYFCARYDHAMDYWGQGSLVTVSS

SEQ ID NO: 84 - protein, 116 aa, synthetic construct
  REGION 1..116: note = Engineered polynucleotide
QIQLVQSGPEVKKPGASVKVSCKASGYTFTNYGMNWVRQAPGQGLEWMGWINTYTGEPTYADDFKGRFTFTTDTSTTTGYMELRSLRSDDTAVYFCARYDHAMDYWGQGTLVTVSS

SEQ ID NO: 85 - protein, 116 aa, synthetic construct
  REGION 1..116: note = Engineered polynucleotide
QIQLVQSGVEVKKPGASVKVSCKASGYTFTNYGMNWVRQAPGQGLEWMGWINTYTGEPTYSDDFKGRFTFTTDTSTSTAYMEVRSLRSDDTAVYFCARYDHAMDYWGQGTLVTVSS

SEQ ID NO: 86 - protein, 116 aa, synthetic construct
  REGION 1..116: note = Engineered polynucleotide
QIQLVQSGVEVKKPGASVKVSCKASGYTFTNYGMNWVRQAPGQGLEWMGWINTYTGETTYADDFKGRFTFTTDTSTSTAYMEVRSLRSDDTAVYFCARYDHAMDYWGQGTLVTVSS

SEQ ID NO: 87 - protein, 116 aa, synthetic construct
  REGION 1..116: note = Engineered polynucleotide
QIQLVQSGPEVKKPGASVKVSCKASGYIFTNYGMNWVRQAPGQGLEWMGWINTYTGEPTYGDDFKGRFTFTTDTSTSTAYMELRSLRSDDTAVYFCGRYDHAMDYWGQGTLVTVSS

SEQ ID NO: 88 - protein, 116 aa, synthetic construct
  REGION 1..116: note = Engineered polynucleotide
QIHLVQSGAEVKKPGASVKVSCKASGYTFTNYGMNWVRQAPGQGLEWMGWINTYTGEPKYADDFKGRFTFTTDTSTSTAYMELRSLRSDDTAVYFCARYDHNMDYWGQGTLVTVSS

SEQ ID NO: 89 - protein, 116 aa, synthetic construct
  REGION 1..116: note = Engineered polynucleotide
QIQLVQSGAEVKKPGASVKVSCKASGYTFTKYGMNWVRQAPGQGLEWMGWINTYTGEPRYADDFKGRFTFTTDTSTSTAYMELRSLRSDDTAVYFCARYDHRMDYWGQGTLVTVSS

SEQ ID NO: 90 - protein, 116 aa, synthetic construct
  REGION 1..116: note = Engineered polynucleotide
QIQLVQSGAEVKKPGASVKVSCKASGYTFTNYGMNWVRQAPGQGLEWMGWINTYTGEPKYADDFKGRFTFTTDTSTSTAYMELRSLRSDDTAVYFCARYDHVMDYWGQGTLVTVSS

SEQ ID NO: 91 - protein, 116 aa, synthetic construct
  REGION 1..116: note = Engineered polynucleotide
QIHLVQSGAEVKKPGASVKVSCKASGYIFTNYGMNWVRQAPGQGLEWMGWINTYTGEPTYADDFKGRFTFTTDTSTSTVYMELRSLRSDDTAVYFCVRYDHAMDYWGQGTLVTVSS

SEQ ID NO: 92 - protein, 116 aa, synthetic construct
  REGION 1..116: note = Engineered polynucleotide
QIQLVQSGTEVKKPGASVKVSCKASGYTFTNYGMNWVRQAPGQGLEWMGWINTYTGETTYADDFKGRFTFTTDTSTSTAYMDLRSLRSDDTAVYFCARYDHAMDYWGQGTLVTVSS

SEQ ID NO: 93 - protein, 116 aa, synthetic construct
  REGION 1..116: note = Engineered polynucleotide
QIHLVQSGPEVKKPGASVKVSCKASGYTFTNYGMNWVRQAPGQGLEWMGWINTYTGEPTYADDFKGRFTFTTDTSTSTAYMELRSLRSDDTAVYFCGRYDHAMDYWGQGTLVTVSS

SEQ ID NO: 94 - protein, 116 aa, synthetic construct
  REGION 1..116: note = Engineered polynucleotide
QIHLVQSGAEVKKPGASVKVSCKASGYSFTNYGMNWVRQAPGQGLEWMGWINTYTGEPTYADDFKGRFTFTTDTSTSTAYMELRSLRSDDTAVYFCVRYDHAMDYWGQGTLVTVSS

SEQ ID NO: 95 - protein, 116 aa, synthetic construct
  REGION 1..116: note = Engineered polynucleotide
QIHLVQSGAEVKKPGASVKVSCKASGYIFTNYGMNWVRQAPGQGLEWMGWINTYTGEPTYADDFKGRFTFTTDTSTSTAYMELRSLRSDDTAVYFCVRYDHAMDYWGQGTLVTVSS

SEQ ID NO: 96 - protein, 116 aa, synthetic construct
  REGION 1..116: note = Engineered polynucleotide
QIQLVQSGVEVKKPGASVKVSCKASGYTFTNYGMNWVRQAPGQGLEWMGWINTYTGETTYADDFKGRFTFTTDTSTSTAYMELRSLRSDDTAVYFCARYDHAMDYWGQGTLVTVSS

SEQ ID NO: 97 - protein, 116 aa, synthetic construct
  REGION 1..116: note = Engineered polynucleotide
QIQLVQSGAEVKKPGASVKVSCKASGYTFTKYGMNWVRQAPGQGLEWMGWINTYTGEPRYVDDFKGRFTFTTDTSTSTAYMELRSLRSDDTAVYFCARYDHRMDYWGQGTLVTVSS

SEQ ID NO: 98 - protein, 116 aa, synthetic construct
  REGION 1..116: note = Engineered polynucleotide
QIQLVQSGAEVKKPGASVKVSCKASGYTFTNYGMNWVRQAPGQGLEWMGWINTYTGEPKYADDFKGRFTFTTDTSTRTAYMELRSLRSDDTAVYFCARYDHTMDYWGQGTLVTVSS

SEQ ID NO: 99 - protein, 116 aa, synthetic construct
  REGION 1..116: note = Engineered polynucleotide
QIQLVQSGAEVKKPGASVKVSCKASGYTFTNYGMNWVRQAPGQGLEWMGWINTYTGESTYADDFKGRFTFTTDTSTTTAYMELRSLRSDDTAVYFCARYDHAMDYWGQGTLVTVSS

SEQ ID NO: 100 - protein, 116 aa, synthetic construct
  REGION 1..116: note = Engineered polynucleotide
QIQLVQSGAEVKKPGASVKVSCKASGYIFTNYGMNWVRQAPGQGLEWMGWINTYTGESTYADDFKGRFTFTTDTSTTTGYMELRSLRSDDTAVYFCARYDHAMDYWGQGTLVTVSS

SEQ ID NO: 101 - protein, 116 aa, synthetic construct
  REGION 1..116: note = Engineered polynucleotide
QIQLVQSGAEVKKPGASVKVSCKASGYSFTNYGMNWVRQAPGQGLEWMGWINTYTGEPTYGDDFKGRFTFTTDTSTSTAYMELRSLRSDDTAVYFCARYDHAMDYWGQGTLVTVSS

SEQ ID NO: 102 - protein, 116 aa, synthetic construct
  REGION 1..116: note = Engineered polynucleotide
QIQLVQSGAEVKKPGASVKVSCKASGYTFTNYGMNWVRQAPGQGLEWMGWINTYTGETTYTDDFKGRFTFTTDTSTSTAYMELRSLRSDDTAVYFCVRYDHAMDYWGQGTLVTVSS

SEQ ID NO: 103 - protein, 116 aa, synthetic construct
  REGION 1..116: note = Engineered polynucleotide
QIQLVQSGAEVKKPGASVKVSCKASGYTFTNYGMNWVRQAPGQGLEWMGWINTYTGEPTYTDDFKGRFTFTTDTSTSTAYMELRSLRSDDTAVYFCGRYDHAMDYWGQGTLVTVSS

SEQ ID NO: 104 - protein, 116 aa, synthetic construct
  REGION 1..116: note = Engineered polynucleotide
  SITE 3: note = MISC_FEATURE - Xaa is Q or H
  SITE 9: note = MISC_FEATURE - Xaa is A, D, T, V, S, or P
  SITE 28: note = MISC_FEATURE - Xaa is T, S, or I
  SITE 31: note = MISC_FEATURE - Xaa is N or K
  SITE 58: note = MISC_FEATURE - Xaa is P, S, T, or A
  SITE 59: note = MISC_FEATURE - Xaa is T, R, K, or I
  SITE 61: note = MISC_FEATURE - Xaa is A, T, V, S, or G
  SITE 77: note = MISC_FEATURE - Xaa is S, R, or T
  SITE 79: note = MISC_FEATURE - Xaa is A, V, or G
  SITE 82: note = MISC_FEATURE - Xaa is E or D
  SITE 83: note = MISC_FEATURE - Xaa is L or V
  SITE 97: note = MISC_FEATURE - Xaa is A, T, V, or G
  SITE 102: note = MISC_FEATURE - Xaa is A, R, F, T, P, V, S, D, N, H, L, Y, or G
  SITE 110: note = MISC_FEATURE - Xaa is T or S
QIXLVQSGXEVKKPGASVKVSCKASGYXFTXYGMNWVRQAPGQGLEWMGWINTYTGEXXYXDDFKGRFTFTTDTSTXTXYMXXRSLRSDDTAVYFCXRYDHXMDYWGQGXLVTVSS

SEQ ID NO: 105 - protein, 112 aa, synthetic construct; identical to SEQ ID NO: 19
  REGION 1..112: note = Engineered polynucleotide

SEQ ID NO: 106 - protein, 112 aa, synthetic construct
  REGION 1..112: note = Engineered polynucleotide
EIVLTQSPDSLAVSLGERATINCKSSQSLLNSGTRKNYLAWYQQKAGQSPKLLIYWTSTRESGVPDRFSGSGSGTDFTLTIDSLQAEDVAVYYCKQSYNLYTFGGGTKVEIK

SEQ ID NO: 107 - protein, 112 aa, synthetic construct
  REGION 1..112: note = Engineered polynucleotide
EIVLTQSPDSLAVSLGERATIKCKSSQSLLNSGTRKNYLAWYQQKAGQSPKLLIYWTSTRESGVPDRFSGSGSGTDFTLTIDSLQAEDVAVYYCKQSYTLYTFGGGTKVEIK

SEQ ID NO: 108 - protein, 112 aa, synthetic construct
  REGION 1..112: note = Engineered polynucleotide
EIVLTQSPDSLAVSLGERATINCKSSQSLLNSGTRKNYLAWYQQKPGQSPKLLIYWTSTRESGVPDRFSGSGSGTDFTLTIDSLQAEDVAVYYCKQSYTLYTFGGGTKVEIK

SEQ ID NO: 109 - protein, 112 aa, synthetic construct
  REGION 1..112: note = Engineered polynucleotide
EIVLTQSPDSLAVSLGERATINCKSSQSLFNSGTRKNYLAWYQQKAGQSPKLLIYWTSTRESGVPDRFSGSGSGTDFTLTIDSLQAEDVAVYYCKQSYTLYTFGGGTKVEIK

SEQ ID NO: 110 - protein, 112 aa, synthetic construct
  REGION 1..112: note = Engineered polynucleotide
EIVLTQSPDSLAVSLGERATINCKSSQSLLNSGTRKNYLAWYQQKAGQSPKLVIYWTSTRESGVPDRFSGSGSGTDFTLTIDSLQAEDVAVYYCKQSYTLYTFGGGTKVEIK

SEQ ID NO: 111 - protein, 112 aa, synthetic construct
  REGION 1..112: note = Engineered polynucleotide
EIVLTQSPDSLAVSLGERATINCKSSQSLLNSGTRKNYLAWYQQKAGQSPKLLIYWTSTRESGVPDRFSGSGSGTDFTLSIDSLQAEDVAVYYCKQSYTLYTFGGGTKVEIK

SEQ ID NO: 112 - protein, 112 aa, synthetic construct
  REGION 1..112: note = Engineered polynucleotide
EIVLTQSPDSLAVSLGERATINCKSSQSLLNSGTRKNYLSWYQQKAGQSPKLLIYWTSTRESGVPDRFSGSGSGTDFTLTIDSLQAEDVAVYYCKQSYSLYTFGGGTKVEIK

SEQ ID NO: 113 - protein, 112 aa, synthetic construct
  REGION 1..112: note = Engineered polynucleotide
EIVLTQSPDSLAVSLGERATINCKSSQSLLNSGTRKNYLTWYQQKAGQSPKLLIYWTSTRESGVPDRFSGSGSGTDFTLTIDSLQAEDVAVYYCKQSYTLYTFGGGTKVEIK

SEQ ID NO: 114 - protein, 112 aa, synthetic construct
  REGION 1..112: note = Engineered polynucleotide
EIVLTQSPDSLAVSLGERATINCKSSQSLLNSGTRKNYLAWYQQKAGQSPKLLIYWTSTRESGVPDRFSGSGSGTDFTLTIDSLQAEDVAIYYCKQSYTLYTFGGGTKVEIK

SEQ ID NO: 115 - protein, 112 aa, synthetic construct
  REGION 1..112: note = Engineered polynucleotide
EIVLTQSPDSLTVSLGERATINCKSSQSLLNSGTRKNYLAWYQQKAGQSPKLLIYWTSTRESGVPDRFSGSGSGTDFTLTIDSLQAEDVAVYYCKQSYTLYTFGGGTKVEIK

SEQ ID NO: 116 - protein, 112 aa, synthetic construct
  REGION 1..112: note = Engineered polynucleotide
EIVLTQSPDSLAVSLGERATINCKSSQSLLNSGTRKNYLAWYQQKAGQSPKLIIYWTSTRESGVPDRFSGSGSGTDFTLTIDSLQAEDVAVYYCKQSYTLYTFGGGTKVEIK

SEQ ID NO: 117 - protein, 112 aa, synthetic construct
  REGION 1..112: note = Engineered polynucleotide
EIVLTQSPDSLAVSLGERATINCKSSQSLLNSGTRKNYLAWYQQKAGQSPKLLIYWTSTRESGVPDRFSGSGSGTDFTLTIDRLQAEDVAVYYCKQSYTLYTFGGGTKVEIK

SEQ ID NO: 118 - protein, 112 aa, synthetic construct
  REGION 1..112: note = Engineered polynucleotide
EIVLTQSPDSLTVSLGERATINCKSSQSLLNSGTRKNYLAWYQQKAGQSPNLLIYWTSTRESGVPDRFSGSGSGTDFTLTIDSLQAEDVAVYYCKQSYTLYTFGGGTKVEIK

SEQ ID NO: 119 - protein, 112 aa, synthetic construct
  REGION 1..112: note = Engineered polynucleotide
EIVLTQSPDSLTVSLGERATINCKSSQSLLNSGTRKNYLAWYQQKAGQSPKLLIYWTSTRESGVPDRFSGSGSGTDFTLTIDSLQAEDVAVYYCKQSYNLYTFGGGTKVEIK

SEQ ID NO: 120 - protein, 112 aa, synthetic construct
  REGION 1..112: note = Engineered polynucleotide
EIVLTQSPDSLAVSLGERATINCKSSQSLLNSGTRKNYLAWYQQKAGQSPKLLIYWTSTRESGVPDRFSGSGSGTDFTLTIDSLQAEDVAVYYCKQSYSLYTFGGGTKVEIK

SEQ ID NO: 121 - protein, 112 aa, synthetic construct
  REGION 1..112: note = Engineered polynucleotide
EIVLTQSPDSLAVSLGERATIKCKSSQSLLNSGTRKNYLAWYQKKAGQSPKLLIYWTSTRESGVPDRFSGSGSGTDFTLTIDSLQAEDVAVYYCKQSYTLYTFGGGTKVEIK

SEQ ID NO: 122 - protein, 112 aa, synthetic construct
  REGION 1..112: note = Engineered polynucleotide
EIVLTQSPDSLAVSLGERATINCKSSQSLLNSGTRKNYLAWYQQKAGQSPKLLIYWTSTRESGVPDRFSGSGSGTDFTLTIDSLQTEDVAVYYCKQSYTLYTFGGGTKVEIK

SEQ ID NO: 123 - protein, 112 aa, synthetic construct
  REGION 1..112: note = Engineered polynucleotide
EIVLTQSPDSLAVSLGERATINCKSSQSLLNSGTRKNYLAWYQQKAGQSPKLLIYWTSTRESGVPDRFSASGSGTDFTLTIDSLQAEDVAVYYCKQSYTLYTFGGGTKVEIK

SEQ ID NO: 124 - protein, 112 aa, synthetic construct
  REGION 1..112: note = Engineered polynucleotide
EIVLTQSPDSLAVSLGERATINCKSSQSLLNSGTRKNYLTWYQQKAGQSPKLLIYWTSTRESGVPDRFSGSGSGTDFTLTIDSLQAEDVALYYCKQSYTLYTFGGGTKVEIK

SEQ ID NO: 125 - protein, 112 aa, synthetic construct
  REGION 1..112: note = Engineered polynucleotide
EIVLTQSPDSLSVSLGERATINCKSSQSLLNSGTRKNYLAWYQQKAGQSPKLLIYWTSTRESGVPDRFSGSGSGTDFTLTIDSLQAEDVAVYYCKQSYTLYTFGGGTKVEIK

SEQ ID NO: 126 - protein, 112 aa, synthetic construct
  REGION 1..112: note = Engineered polynucleotide
EIVLTQSPDSLAVSLGERATINCKSSQSLLNSGTRKNYLAWYQQKSGQSPKLLIYWTSTRESGVPDRFSGSGSGTDFTLTIDSLQAEDVAVYYCKQSYTLYTFGGGTKVEIK

SEQ ID NO: 127 - protein, 112 aa, synthetic construct
  REGION 1..112: note = Engineered polynucleotide
EIVLTQSPDSLAVSLGERATINCKSSQSLVNSGTRKNYLAWYQQKAGQSPKLLIYWTSTRESGVPDRFSGSGSGTDFTLTIDSLQTEDVAVYYCKQSYTLYTFGGGTKVEIK

SEQ ID NO: 128 - protein, 112 aa, synthetic construct
  REGION 1..112: note = Engineered polynucleotide
EIVLTQSPDSLAVSLGERATIKCKSSQSLLNSGTRKNYLAWYQQKAGQSPKLLIYWTSTRESGVPDRFSGSGSGTDFTLTIDSLQAEDVALYYCKQSYTLYTFGGGTKVEIK

SEQ ID NO: 129 - protein, 112 aa, synthetic construct
  REGION 1..112: note = Engineered polynucleotide
EIVLTQSPDSLAVSLGERATINCKSSQSLLNSGTRKNYLAWYQQKAGQSPKLIIYWTSTRESGVPDRFSGSGSGTDFTLTIDSLQAEDVALYYCKQSYTLYTFGGGTKVEIK

SEQ ID NO: 130 - protein, 112 aa, synthetic construct
  REGION 1..112: note = Engineered polynucleotide
EIVLTQSPDSLAVSLGERATINCKSSQSLLNSGTRKNYLAWYQKKSGQSPKLLIYWTSTRESGVPDRFSGSGSGTDFTLSIDSLQAEDVAVYYCKQSYTLYTFGGGTKVEIK

SEQ ID NO: 131 - protein, 112 aa, synthetic construct
  REGION 1..112: note = Engineered polynucleotide
EIVLTQSPDSLAVSLGERATIKCKSSQSLLNSGTRKNYLAWYQQKAGQSPKLLIYWTSTRESGVPDRFSASGSGTDFTLTIDSLQAEDVAVYYCKQSYTLYTFGGGTKVEIK

SEQ ID NO: 132 - protein, 112 aa, synthetic construct
  REGION 1..112: note = Engineered polynucleotide
EIVLTQSPDSLAVSLGERATINCKSSQSLLNSGTRKNYLTWYQQKSGQSPKLLIYWTSTRESGVPDRFSGSGSGTDFTLTIDSLQAEDVAIYYCKQSYTLYTFGGGTKVEIK

SEQ ID NO: 133 - protein, 112 aa, synthetic construct
  REGION 1..112: note = Engineered polynucleotide
EIVLTQSPDSLAVSLGERATINCKSSQSLLNSGTRKNYLAWYQKKAGQSPKLLIYWTSTRESGVPDRFSGSGSGTDFTLSIDSLQAEDVAIYYCKQSYTLYTFGGGTKVEIK

SEQ ID NO: 134 - protein, 112 aa, synthetic construct
  REGION 1..112: note = Engineered polynucleotide
EIVLTQSPDSLAVSLGERATINCKSSQSLLNSGTRKNYLAWYQQKSGQSPKLLIYWTSTRESGVPDRFSGSGSGTDFTLTIDSLQAEDVAIYYCKQSYTLYTFGGGTKVEIK

SEQ ID NO: 135 - protein, 112 aa, synthetic construct
  REGION 1..112: note = Engineered polynucleotide
EIVLTQSPDSLSVSLGERATINCKSSQSLLNSGTRKNYLAWYQQKAGQSPNLLIYWTSTRESGVPDRFSGSGSGTDFTLSIDSLQAEDVAVYYCKQSYTLYTFGGGTKVEIK

SEQ ID NO: 136 - protein, 112 aa, synthetic construct
  REGION 1..112: note = Engineered polynucleotide
EIVLTQSPDSLSVSLGERATINCKSSQSLLNSGTRKNYLAWYQQKAGQSPNLLIYWTSTRESGVPDRFSGSGSGTDFTLTIDSLQAEDVAVYYCKQSYTLYTFGGGTKVEIK

SEQ ID NO: 137 - protein, 112 aa, synthetic construct
  REGION 1..112: note = Engineered polynucleotide
EIVLTQSPDSLTVSLGERATINCKSSQSLLNSGTRKNYLAWYQQKAGQSPNLLIYWTSTRESGVPDRFSGSGSGTDFTLTIDSLQAEDVALYYCKQSYTLYTFGGGTKVEIK

SEQ ID NO: 138 - protein, 112 aa, synthetic construct
  REGION 1..112: note = Engineered polynucleotide
EIVLTQSPDSLSVSLGERATINCKSSQSLLNSGTRKNYLAWYQKKAGQSPKLLIYWTSTRESGVPDRFSGSGSGTDFTLTIDRLQAEDVALYYCKQSYTLYTFGGGTKVEIK

SEQ ID NO: 139 - protein, 112 aa, synthetic construct
  REGION 1..112: note = Engineered polynucleotide
EIVLTQSPDSLSVSLGERATIKCKSSQSLLNSGTRKNYLAWYQQKAGQSPKLLIYWTSTRESGVPDRFSGSGSGTDFTLTIDSLQTEDVAVYYCKQSYTLYTFGGGTKVEIK

SEQ ID NO: 140 - protein, 112 aa, synthetic construct
  REGION 1..112: note = Engineered polynucleotide
EIVLTQSPDSLAVSLGERATIKCKSSQSLLNSGTRKNYLAWYQQKAGQSPKLLIYWTSTRESGVPDRFSGSGSGTDFTLTIDSLQAEDVAIYYCKQSYTLYTFGGGTKVEIK

SEQ ID NO: 141 - protein, 112 aa, synthetic construct
  REGION 1..112: note = Engineered polynucleotide
EIVLTQSPDSLAVSLGERATINCKSSQSLFNSGTRKNYLAWYQQKAGQSPKLLIYWTSTRESGVPDRFSGSGSGTDFTLSIDSLQAEDVAVYYCKQSYTLYTFGGGTKVEIK

SEQ ID NO: 142 - protein, 112 aa, synthetic construct
  REGION 1..112: note = Engineered polynucleotide
EIVLTQSPDSLAVSLGERATINCKSSQSLLNSGTRKNYLAWYQQKAGQSPNLLIYWTSTRESGVPDRFSGSGSGTDFTLTIDSLQAEDVAVYYCKQSYSLYTFGGGTKVEIK

SEQ ID NO: 143 - protein, 112 aa, synthetic construct
  REGION 1..112: note = Engineered polynucleotide
EIVLTQSPDSLAVSLGERATINCKSSQSLLNSGTRKNYLAWYQQKPGQSPKLLIYWTSTRESGVPDRFSGSGSGTDFTLTIDSLQAEDVAVYYCKQSYNLYTFGGGTKVEIK

SEQ ID NO: 144 - protein, 112 aa, synthetic construct
  REGION 1..112: note = Engineered polynucleotide
EIVLTQSPDSLAVSLGERATINCKSSQSLLNSGTRKNYLSWYQQKPGQSPKLIIYWTSTRESGVPDRFSGSGSGTDFTLTIDSLQAEDVAVYYCKQSYTLYTFGGGTKVEIK

SEQ ID NO: 145 - protein, 112 aa, synthetic construct
  REGION 1..112: note = Engineered polynucleotide
EIVLTQSPDSLAVSLGERATINCKSSQSLLNSGTRKNYLAWYQQKSGQSPKLLIYWTSTRESGVPDRFSGSGSGTDFTLTIDSLQTEDVAIYYCKQSYTLYTFGGGTKVEIK

SEQ ID NO: 146 - protein, 112 aa, synthetic construct
  REGION 1..112: note = Engineered polynucleotide
EIVLTQSPDSLAVSLGERATINCKSSQSLLNSGTRKNYLSWYQQKAGQSPNLVIYWTSTRESGVPDRFSGSGSGTDFTLTIDSLQAEDVAVYYCKQSYTLYTFGGGTKVEIK

SEQ ID NO: 147 - protein, 112 aa, synthetic construct
  REGION 1..112: note = Engineered polynucleotide
EIVLTQSPDSLAVSLGERATINCKSSQSLLNSGTRKNYLAWYQKKSGQSPNLLIYWTSTRESGVPDRFSGSGSGTDFTLTIDSLQAEDVAVYYCKQSYTLYTFGGGTKVEIK

SEQ ID NO: 148 - protein, 112 aa, synthetic construct
  REGION 1..112: note = Engineered polynucleotide
EIVLTQSPDSLAVSLGERATINCKSSQSLLNSGTRKNYLSWYQKKSGQSPKLLIYWTSTRESGVPDRFSGSGSGTDFTLTIDSLQAEDVAVYYCKQSYTLYTFGGGTKVEIK

SEQ ID NO: 149 - protein, 112 aa, synthetic construct
  REGION 1..112: note = Engineered polynucleotide
EIVLTQSPDSLAVSLGERATINCKSSQSLFNSGTRKNYLAWYQKKAGQSPKLLIYWTSTRESGVPDRFSGSGSGTDFTLSIDRLQAEDVAVYYCKQSYTLYTFGGGTKVEIK

SEQ ID NO: 150 - protein, 112 aa, synthetic construct
  REGION 1..112: note = Engineered polynucleotide
EIVLTQSPDSLTVSLGERATINCKSSQSLLNSGTRKNYLAWYQQKPGQSPKLLIYWTSTRESGVPDRFSGSGSGTDFTLTIDSLQAEDVAVYYCKQSYTLYTFGGGTKVEIK

SEQ ID NO: 151 - protein, 112 aa, synthetic construct
  REGION 1..112: note = Engineered polynucleotide
EIVLTQSPDSLAVSLGERATIKCKSSQSLLNSGTRKNYLTWYQQKAGQSPNLLIYWTSTRESGVPDRFSGSGSGTDFTLTIDSLQAEDVAVYYCKQSYTLYTFGGGTKVEIK

SEQ ID NO: 152 - protein, 112 aa, synthetic construct
  REGION 1..112: note = Engineered polynucleotide
EIVLTQSPDSLSVSLGERATINCKSSQSLLNSGTRKNYLAWYQQKSGQSPNLVIYWTSTRESGVPDRFSGSGSGTDFTLTIDSLQAEDVAVYYCKQSYTLYTFGGGTKVEIK

SEQ ID NO: 153 - protein, 112 aa, synthetic construct
  REGION 1..112: note = Engineered polynucleotide
EIVLTQSPDSLSVSLGERATINCKSSQSLFNSGTRKNYLAWYQQKAGQSPKLLIYWTSTRESGVPDRFSGSGSGTDFTLTIDSLQAEDVAVYYCKQSYTLYTFGGGTKVEIK

SEQ ID NO: 154 - protein, 112 aa, synthetic construct
  REGION 1..112: note = Engineered polynucleotide
EIVLTQSPDSLTVSLGERATINCKSSQSLFNSGTRKNYLAWYQKKAGQSPKLLIYWTSTRESGVPDRFSGSGSGTDFTLTIDSLQAEDVAVYYCKQSYNLYTFGGGTKVEIK

SEQ ID NO: 155 - protein, 112 aa, synthetic construct
  REGION 1..112: note = Engineered polynucleotide
EIVLTQSPDSLAVSLGERATINCKSSQSLLNSGTRKNYLAWYQQKPGQSPKLLIYWTSTRESGVPDRFSASGSGTDFTLTIDSLQAEDVAIYYCKQSYTLYTFGGGTKVEIK

SEQ ID NO: 156 - protein, 112 aa, synthetic construct
  REGION 1..112: note = Engineered polynucleotide
EIVLTQSPDSLAVSLGERATINCKSSQSLFNSGTRKNYLAWYQQKAGQSPKLIIYWTSTRESGVPDRFSASGSGTDFTLSIDRLQAEDVAVYYCKQSYTLYTFGGGTKVEIK

SEQ ID NO: 157 - protein, 112 aa, synthetic construct
  REGION 1..112: note = Engineered polynucleotide
EIVLTQSPDSLSVSLGERATINCKSSQSLLNSGTRKNYLAWYQQKAGQSPKLLIYWTSTRESGVPDRFSASGSGTDFTLTIDRLQAEDVALYYCKQSYTLYTFGGGTKVEIK

SEQ ID NO: 158 - protein, 112 aa, synthetic construct
  REGION 1..112: note = Engineered polynucleotide
  SITE 12: note = MISC_FEATURE - Xaa is A, T, or S
  SITE 22: note = MISC_FEATURE - Xaa is N or K
  SITE 30: note = MISC_FEATURE - Xaa is L, F, or V
  SITE 40: note = MISC_FEATURE - Xaa is A, S, or T
  SITE 44: note = MISC_FEATURE - Xaa is Q or K
  SITE 46: note = MISC_FEATURE - Xaa is A, P, or S
  SITE 51: note = MISC_FEATURE - Xaa is K or N
  SITE 53: note = MISC_FEATURE - Xaa is L, V, or I
  SITE 70: note = MISC_FEATURE - Xaa is G or A
  SITE 80: note = MISC_FEATURE - Xaa is T or S
  SITE 83: note = MISC_FEATURE - Xaa is S or R
  SITE 86: note = MISC_FEATURE - Xaa is A or T
  SITE 91: note = MISC_FEATURE - Xaa is V, I, or L
  SITE 99: note = MISC_FEATURE - Xaa is T, N, or S
EIVLTQSPDSLXVSLGERATIXCKSSQSLXNSGTRKNYLXWYQXKXGQSPXLXIYWTSTRESGVPDRFSXSGSGTDFTLXIDXLQXEDVAXYYCKQSYXLYTFGGGTKVEIK

SEQ ID NO: 159 - protein, 12 aa, synthetic construct
  REGION 1..12: note = Engineered polynucleotide
QSLFNSGTRKNY

SEQ ID NO: 160 - protein, 12 aa, synthetic construct
  REGION 1..12: note = Engineered polynucleotide
QSLVNSGTRKNY

SEQ ID NO: 161 - protein, 12 aa, synthetic construct
  REGION 1..12: note = Engineered polynucleotide
QSLFNSGTRKNY

SEQ ID NO: 162 - protein, 8 aa, synthetic construct
  REGION 1..8: note = Engineered polynucleotide
KQSYNLYT

SEQ ID NO: 163 - protein, 8 aa, synthetic construct
  REGION 1..8: note = Engineered polynucleotide
KQSYSLYT

SEQ ID NO: 164 - protein, 8 aa, synthetic construct
  REGION 1..8: note = Engineered polynucleotide
GYTFTKYG

SEQ ID NO: 165 - protein, 8 aa, synthetic construct
  REGION 1..8: note = Engineered polynucleotide
GYSFTNYG

SEQ ID NO: 166 - protein, 8 aa, synthetic construct
  REGION 1..8: note = Engineered polynucleotide
GYIFTNYG

SEQ ID NO: 167 - protein, 8 aa, synthetic construct
  REGION 1..8: note = Engineered polynucleotide
INTYTGES

SEQ ID NO: 168 - protein, 8 aa, synthetic construct
  REGION 1..8: note = Engineered polynucleotide
INTYTGET

SEQ ID NO: 169 - protein, 8 aa, synthetic construct
  REGION 1..8: note = Engineered polynucleotide
INTYTGEA

SEQ ID NO: 170 - protein, 9 aa, synthetic construct
  REGION 1..9: note = Engineered polynucleotide
ARYDHRMDY

SEQ ID NO: 171 - protein, 9 aa, synthetic construct
  REGION 1..9: note = Engineered polynucleotide
ARYDHFMDY

SEQ ID NO: 172 - protein, 9 aa, synthetic construct
  REGION 1..9: note = Engineered polynucleotide
ARYDHTMDY

SEQ ID NO: 173 - protein, 9 aa, synthetic construct
  REGION 1..9: note = Engineered polynucleotide
ARYDHPMDY

SEQ ID NO: 174 - protein, 9 aa, synthetic construct
  REGION 1..9: note = Engineered polynucleotide
ARYDHVMDY

SEQ ID NO: 175 - protein, 9 aa, synthetic construct
  REGION 1..9: note = Engineered polynucleotide
ARYDHSMDY

SEQ ID NO: 176 - protein, 9 aa, synthetic construct
  REGION 1..9: note = Engineered polynucleotide
TRYDHAMDY

SEQ ID NO: 177 - protein, 9 aa, synthetic construct
  REGION 1..9: note = Engineered polynucleotide
ARYDHDMDY

SEQ ID NO: 178 - protein, 9 aa, synthetic construct
  REGION 1..9: note = Engineered polynucleotide
ARYDHNMDY

SEQ ID NO: 179 - protein, 9 aa, synthetic construct
  REGION 1..9: note = Engineered polynucleotide
ARYDHHMDY

SEQ ID NO: 180 - protein, 9 aa, synthetic construct
  REGION 1..9: note = Engineered polynucleotide
ARYDHLMDY

SEQ ID NO: 181 - protein, 9 aa, synthetic construct
  REGION 1..9: note = Engineered polynucleotide
ARYDHYMDY

SEQ ID NO: 182 - protein, 9 aa, synthetic construct
  REGION 1..9: note = Engineered polynucleotide
TRYDHRMDY

SEQ ID NO: 183 - protein, 9 aa, synthetic construct
  REGION 1..9: note = Engineered polynucleotide
VRYDHAMDY

SEQ ID NO: 184 - protein, 9 aa, synthetic construct
  REGION 1..9: note = Engineered polynucleotide
GRYDHAMDY

SEQ ID NO: 185 - protein, 9 aa, synthetic construct
  REGION 1..9: note = Engineered polynucleotide
ARYDHGMDY

SEQ ID NO: 186 - protein, 17 aa, synthetic construct
  REGION 1..17: note = Engineered polynucleotide
KSSQSLLNSGTRKNYLA

SEQ ID NO: 187 - protein, 17 aa, synthetic construct
  REGION 1..17: note = Engineered polynucleotide
KSSQSLFNSGTRKNYLA

SEQ ID NO: 188 - protein, 17 aa, synthetic construct
  REGION 1..17: note = Engineered polynucleotide
KSSQSLLNSGTRKNYLS

SEQ ID NO: 189 - protein, 17 aa, synthetic construct
  REGION 1..17: note = Engineered polynucleotide
KSSQSLLNSGTRKNYLT

SEQ ID NO: 190 - protein, 17 aa, synthetic construct
  REGION 1..17: note = Engineered polynucleotide
KSSQSLVNSGTRKNYLA

SEQ ID NO: 191 - protein, 7 aa, synthetic construct
  REGION 1..7: note = Engineered polynucleotide
WTSTRES

SEQ ID NO: 192 - protein, 10 aa, synthetic construct
  REGION 1..10: note = Engineered polynucleotide
GYTFTKYGMN

SEQ ID NO: 193 - protein, 10 aa, synthetic construct
  REGION 1..10: note = Engineered polynucleotide
GYTFTNYGMN

SEQ ID NO: 194 - protein, 10 aa, synthetic construct
  REGION 1..10: note = Engineered polynucleotide
GYSFTNYGMN

SEQ ID NO: 195 - protein, 10 aa, synthetic construct
  REGION 1..10: note = Engineered polynucleotide
GYIFTNYGMN

SEQ ID NO: 196 - protein, 17 aa, synthetic construct
  REGION 1..17: note = Engineered polynucleotide
WINTYTGEPTYADDFKG

SEQ ID NO: 197 - protein, 17 aa, synthetic construct
  REGION 1..17: note = Engineered polynucleotide
WINTYTGEPRYADDFKG

SEQ ID NO: 198 - protein, 17 aa, synthetic construct
  REGION 1..17: note = Engineered polynucleotide
WINTYTGEPIYADDFKG

SEQ ID NO: 199 - protein, 17 aa, synthetic construct
  REGION 1..17: note = Engineered polynucleotide
WINTYTGEPTYTDDFKG

SEQ ID NO: 200 - protein, 17 aa, synthetic construct
  REGION 1..17: note = Engineered polynucleotide
WINTYTGEPTYVDDFKG

SEQ ID NO: 201 - protein, 17 aa, synthetic construct
  REGION 1..17: note = Engineered polynucleotide
WINTYTGEPTYSDDFKG

SEQ ID NO: 202 - protein, 17 aa, synthetic construct
  REGION 1..17: note = Engineered polynucleotide
WINTYTGESTYADDFKG

SEQ ID NO: 203 - protein, 17 aa, synthetic construct
  REGION 1..17: note = Engineered polynucleotide
WINTYTGETTYGDDFKG

SEQ ID NO: 204 - protein, 17 aa, synthetic construct
  REGION 1..17: note = Engineered polynucleotide
WINTYTGEPKYTDDFKG

SEQ ID NO: 205 - protein, 17 aa, synthetic construct
  REGION 1..17: note = Engineered polynucleotide
WINTYTGEPKYGDDFKG

SEQ ID NO: 206 - protein, 17 aa, synthetic construct
  REGION 1..17: note = Engineered polynucleotide
WINTYTGEPTYGDDFKG

SEQ ID NO: 207 - protein, 17 aa, synthetic construct
  REGION 1..17: note = Engineered polynucleotide
WINTYTGEATYADDFKG

SEQ ID NO: 208 - protein, 17 aa, synthetic construct
  REGION 1..17: note = Engineered polynucleotide
WINTYTGETTYTDDFKG

SEQ ID NO: 209 - protein, 17 aa, synthetic construct
  REGION 1..17: note = Engineered polynucleotide
WINTYTGETTYADDFKG

SEQ ID NO: 210 - protein, 17 aa, synthetic construct
  REGION 1..17: note = Engineered polynucleotide
WINTYTGEPKYADDFKG

SEQ ID NO: 211 - protein, 17 aa, synthetic construct
  REGION 1..17: note = Engineered polynucleotide
WINTYTGEPRYVDDFKG

SEQ ID NO: 212 - protein, 12 aa, synthetic construct
  REGION 1..12: note = Engineered polynucleotide
  SITE 4: note = MISC_FEATURE - Xaa is L, F, or V
QSLXNSGTRKNY

SEQ ID NO: 213 - protein, 8 aa, synthetic construct
  REGION 1..8: note = Engineered polynucleotide
  SITE 5: note = MISC_FEATURE - Xaa is T, N, S
KQSYXLYT

SEQ ID NO: 214 - protein, 8 aa, synthetic construct
  REGION 1..8: note = Engineered polynucleotide
  SITE 3: note = MISC_FEATURE - Xaa is T, S, or I
  SITE 6: note = MISC_FEATURE - Xaa is N, or K
GYXFTXYG

SEQ ID NO: 215 - protein, 8 aa, synthetic construct
  REGION 1..8: note = Engineered polynucleotide
  SITE 8: note = MISC_FEATURE - Xaa is P, S, T, or A
INTYTGEX

SEQ ID NO: 216 - protein, 9 aa, synthetic construct
  REGION 1..9: note = Engineered polynucleotide
  SITE 1: note = MISC_FEATURE - Xaa is A, T, V, or G
  SITE 6: note = MISC_FEATURE - Xaa is A, R, F, T, P, V, S, D, N, H, L, Y, or G
XRYDHXMDY

SEQ ID NO: 217 - protein, 330 aa, Homo sapiens [sequence omitted]

SEQ ID NO: 218 - protein, 330 aa, Homo sapiens
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 219 - protein, 326 aa, Homo sapiens
ASTKGPSVFPLAPCSRSTSESTAALGCLVKDYFPEPVTVSWNSGALTSGVHTFPAVLQSSGLYSLSSVVTVPSSNFGTQTYTCNVDHKPSNTKVDKTVERKCCVECPPCPAPPVAGPSVFLFPPKPKDTLMISRTPEVTCVVVDVSHEDPEVQFNWYVDGVEVHNAKTKPREEQFNSTFRVVSVLTVVHQDWLNGKEYKCKVSNKGLPAPIEKTISKTKGQPREPQVYTLPPSREEMTKNQVSLTCLVKGFYPSDISVEWESNGQPENNYKTTPPMLDSDGSFFLYSKLTVDKSRWQQGNVFSCSVMHEALHNHYTQKSLSLSPGK

SEQ ID NO: 220 - protein, 377 aa, Homo sapiens
ASTKGPSVFPLAPCSRSTSGGTAALGCLVKDYFPEPVTVSWNSGALTSGVHTFPAVLQSSGLYSLSSVVTVPSSSLGTQTYTCNVNHKPSNTKVDKRVELKTPLGDTTHTCPRCPEPKSCDTPPPCPRCPEPKSCDTPPPCPRCPEPKSCDTPPPCPRCPAPELLGGPSVFLFPPKPKDTLMISRTPEVTCVVVDVSHEDPEVQFKWYVDGVEVHNAKTKPREEQYNSTFRVVSVLTVLHQDWLNGKEYKCKVSNKALPAPIEKTISKTKGQPREPQVYTLPPSREEMTKNQVSLTCLVKGFYPSDIAVEWESSGQPENNYNTTPPMLDSDGSFFLYSKLTVDKSRWQQGNIFSCSVMHEALHNRFTQKSLSLSPGK

SEQ ID NO: 221 - protein, 327 aa, Homo sapiens
ASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPSCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 222 - protein, 106 aa, Homo sapiens
TVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 223 - protein, 107 aa, Homo sapiens [sequence omitted]

SEQ ID NO: 224 - protein, 105 aa, Homo sapiens
GQPKANPTVTLFPPSSEELQANKATLVCLISDFYPGAVTVAWKADGSPVKAGVETTKPSKQSNNKYAASSYLSLTPEQWKSHRSYSCQVTHEGSTVEKTVAPTEC

SEQ ID NO: 225 - protein, 116 aa, synthetic construct
  REGION 1..116: note = Engineered polypeptide
QVQLQQSGSELKKPGASVKVSCKASGYTFTNYGMNWVRQAPGQGLEWMGWINTYTGEPTYADDFKGRFVFSLDTSVSTAYLQISSLKAEDTAVYYCARYDHAMDYWGQGTMVTVSS

SEQ ID NO: 226 - protein, 116 aa, synthetic construct
  REGION 1..116: note = Engineered polypeptide
QVQLVQSGSELKKPGASVKISCKASGYTFTNYGMNWMRRAPGQGLKWMGWINTYTGEPTYADDFKGRFVFSLDTSVNTAFLQISSLQAEDTAVYYCARYDHAMDYWGQGTTVTVSS

SEQ ID NO: 227 - protein, 116 aa, synthetic construct; identical to SEQ ID NO: 14
  REGION 1..116: note = Engineered polypeptide

SEQ ID NO: 228 - protein, 116 aa, synthetic construct
  REGION 1..116: note = Engineered polypeptide
QVHLVQSGSEVKKPGASVKVSCQASGYTFTNYGMNWVRQAPGQGLEWMGWINTYTGEPTYADDFKGRFVFSLDTSVSTAYLQINRLKAEDTAVYFCARYDHAMDYWGQGTLVTVSS

SEQ ID NO: 229 - protein, 116 aa, synthetic construct
  REGION 1..116: note = Engineered polypeptide
QVQLVQSGPELKKPGESVKVSCKASGYTFTNYGMNWVRQAPGRGLKWMGWINTYTGEPTYADDFKGRFVFSLDTSVSTAYLQISSLKAEDTAVYFCARYDHAMDYWGQGTSVTVSS

SEQ ID NO: 230 - protein, 112 aa, synthetic construct; identical to SEQ ID NO: 19
  REGION 1..112: note = Engineered polypeptide

SEQ ID NO: 231 - protein, 112 aa, synthetic construct; identical to SEQ ID NO: 20
  REGION 1..112: note = Engineered polypeptide

SEQ ID NO: 232 - protein, 112 aa, synthetic construct; identical to SEQ ID NO: 21
  REGION 1..112: note = Engineered polypeptide

SEQ ID NO: 233 - protein, 112 aa, synthetic construct
  REGION 1..112: note = Engineered polypeptide
DIVMTQSPVSLAVSLGERATINCKSSQSLLNSGTRKNYLAWYQQKPGQPPKLLIYWTSTRESGVPDRFSGSGSGTDFTLTISSLQAEDVAVYYCKQSYTLYTFGPGTKVDIK

SEQ ID NO: 234 - protein, 112 aa, synthetic construct
  REGION 1..112: note = Engineered polypeptide
DIVMTQSPDSLAVSLGERATINCKSSQSLLNSGTRKNYLAWYQQKPGQPPKLLIYWTSTRESGVPDRFSGSGSGTDFTLTISSLQAEDVAVYYCKQSYTLYTFGGGTKLEIK

SEQ ID NO: 235 - protein, 330 aa, synthetic construct
  REGION 1..330: note = Engineered polypeptide
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 236 - protein, 330 aa, synthetic construct
  REGION 1..330: note = Engineered polypeptide
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLAGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 237 - protein, 330 aa, synthetic construct
  REGION 1..330: note = Engineered polypeptide
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLAGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPLPEEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 238 - protein, 330 aa, synthetic construct
  REGION 1..330: note = Engineered polypeptide
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLYITREPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 239 - protein, 330 aa, synthetic construct
  REGION 1..330: note = Engineered polypeptide
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVLHEALHSHYTQKSLSLSPGK

SEQ ID NO: 240 - protein, 330 aa, synthetic construct
  REGION 1..330: note = Engineered polypeptide
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLQVLHVDWLNGKEYKCKVSNKALPAPIEKTISKAKGQPREPQVYTLPPSREEMTKNQVSLTCLVKGFYPSDIVVEWESNGQPENNYKTTPPVLDSDGSFFLYSKLTVDKSRWQQGNVFSCSVMHEALHNHYTQKSLSLSPGK

SEQ ID NO: 241 - protein, 330 aa, synthetic construct
  REGION 1..330: note = Engineered polypeptide
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRDPEVTCVVVDVSHEDPEVKFNWYVDGVEVHDAKTKPREEQYNSTYRVVSVLRVLHVDWLNGKEYKCKVSNKALPAPIEKTISKAKGQPREPQVYTLPPSREEMTKNQVSLTCLVKGFYPSDIVVEWESNGQPENNYKTTPPVLDSDGSFFLYSKLTVDKSRWQQGNVFSCSVMHEALHNHYTQKSLSLSPGK

SEQ ID NO: 242 - protein, 446 aa, synthetic construct
  REGION 1..446: note = Engineered polypeptide
QIQLVQSGAEVKKPGASVKVSCKASGYTFTNYGMNWVRQAPGQGLEWMGWINTYTGEPTYADDFKGRFTFTTDTSTSTAYMELRSLRSDDTAVYFCARYDHAMDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 243 - protein, 446 aa, synthetic construct
  REGION 1..446: note = Engineered polypeptide
QIQLVQSGAEVKKPGASVKVSCKASGYTFTNYGMNWVRQAPGQGLEWMGWINTYTGEPTYADDFKGRFTFTTDTSTSTAYMELRSLRSDDTAVYFCARYDHAMDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLAGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 244 - protein, 446 aa, synthetic construct
  REGION 1..446: note = Engineered polypeptide
QIQLVQSGAEVKKPGASVKVSCKASGYTFTNYGMNWVRQAPGQGLEWMGWINTYTGEPTYADDFKGRFTFTTDTSTSTAYMELRSLRSDDTAVYFCARYDHAMDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLAGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPLPEEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 245 - protein, 446 aa, synthetic construct
  REGION 1..446: note = Engineered polypeptide
QIQLVQSGAEVKKPGASVKVSCKASGYTFTNYGMNWVRQAPGQGLEWMGWINTYTGEPTYADDFKGRFTFTTDTSTSTAYMELRSLRSDDTAVYFCARYDHAMDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLYITREPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 246 - protein, 446 aa, synthetic construct
  REGION 1..446: note = Engineered polypeptide
QIQLVQSGAEVKKPGASVKVSCKASGYTFTNYGMNWVRQAPGQGLEWMGWINTYTGEPTYADDFKGRFTFTTDTSTSTAYMELRSLRSDDTAVYFCARYDHAMDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVLHEALHSHYTQKSLSLSPGK

SEQ ID NO: 247 - protein, 446 aa, synthetic construct
  REGION 1..446: note = Engineered polypeptide
QIQLVQSGAEVKKPGASVKVSCKASGYTFTNYGMNWVRQAPGQGLEWMGWINTYTGEPTYADDFKGRFTFTTDTSTSTAYMELRSLRSDDTAVYFCARYDHAMDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLQVLHVDWLNGKEYKCKVSNKALPAPIEKTISKAKGQPREPQVYTLPPSREEMTKNQVSLTCLVKGFYPSDIVVEWESNGQPENNYKTTPPVLDSDGSFFLYSKLTVDKSRWQQGNVFSCSVMHEALHNHYTQKSLSLSPGK

SEQ ID NO: 248 - protein, 446 aa, synthetic construct
  REGION 1..446: note = Engineered polypeptide
QIQLVQSGAEVKKPGASVKVSCKASGYTFTNYGMNWVRQAPGQGLEWMGWINTYTGEPTYADDFKGRFTFTTDTSTSTAYMELRSLRSDDTAVYFCARYDHAMDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRDPEVTCVVVDVSHEDPEVKFNWYVDGVEVHDAKTKPREEQYNSTYRVVSVLRVLHVDWLNGKEYKCKVSNKALPAPIEKTISKAKGQPREPQVYTLPPSREEMTKNQVSLTCLVKGFYPSDIVVEWESNGQPENNYKTTPPVLDSDGSFFLYSKLTVDKSRWQQGNVFSCSVMHEALHNHYTQKSLSLSPGK

SEQ ID NO: 249 - protein, 219 aa, synthetic construct
  REGION 1..219: note = Engineered polypeptide
EIVLTQSPDSLAVSLGERATINCKSSQSLLNSGTRKNYLAWYQQKAGQSPKLLIYWTSTRESGVPDRFSGSGSGTDFTLTIDSLQAEDVAVYYCKQSYTLYTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 250 - protein, 8 aa, synthetic construct
  REGION 1..8: note = Engineered polypeptide
KEWHKREQ

SEQ ID NO: 251 - protein, 641 aa, Mus musculus
MAKNTAIGIDLGTTYSCVGVFQHGKVEIIANDQGNRTTPSYVAFTDTERLIGDAAKNQVALNPQNTVFDAKRLIGRKFGDAVVQSDMKHWPFQVVNDGDKPKVQVNYKGESRSFFPEEISSMVLTKMKEIAEAYLGHPVTNAVITVPAYFNDSQRQATKDAGVIAGLNVLRIINEPTAAAIAYGLDRTGKGERNVLIFDLGGGTFDVSILTIDDGIFEVKATAGDTHLGGEDFDNRLVSHFVEEFKRKHKKDISQNKRAVRRLRTACERAKRTLSSSTQASLEIDSLFEGIDFYTSITRARFEELCSDLFRGTLEPVEKALRDAKMDKAQIHDLVLVGGSTRIPKVQKLLQDFFNGRDLNKSINPDEAVAYGAAVQAAILMGDKSENVQDLLLLDVAPLSLGLETAGGVMTALIKRNSTIPTKQTQTFTTYSDNQPGVLIQVYEGERAMTRDNNLLGRFELSGIPPAPRGVPQIEVTFDIDANGILNVTATDKSTGKANKITITNDKGRLSKEEIERMVQEAERYKAEDEVQRDRVAAKNALESYAFNMKSAVEDEGLKGKLSEADKKKVLDKCQEVISWLDSNTLADKEEFVHKREELERVCSPIISGLYQGAGAPGAGGFGAQAPKGASGSGPTIEEVD